(19) United States
(12) Patent Application Publication
(10) Pub. No.: US 2022/0127281 A1
(43) Pub. Date: Apr. 28, 2022

(54) HETEROARYL COMPOUNDS THAT INHIBIT G12C MUTANT RAS PROTEINS

(71) Applicant: AstraZeneca AB, Södertälje (SE)

(72) Inventors: Jason Grant KETTLE, Cambridge (GB); Sharanjeet Kaur BAGAL, Cambridge (GB); Scott BOYD, Cambridge (GB); Andrew John EATHERTON, Cambridge (GB); Shaun Michael FILLERY, Cambridge (GB); Graeme Richard ROBB, Cambridge (GB); Piotr Antoni RAUBO, Cambridge (GB)

(21) Appl. No.: 17/449,974

(22) Filed: Oct. 5, 2021

Related U.S. Application Data
(63) Continuation of application No. 16/611,538, filed on Nov. 7, 2019, now abandoned.
(60) Provisional application No. 62/504,638, filed on May 11, 2017.

(51) Int. Cl. C07D 498/14; C07D 519/00

(52) U.S. Cl. CPC C07D 498/14; C07D 519/00

(57) ABSTRACT

The specification relates to compounds of Formula (I):

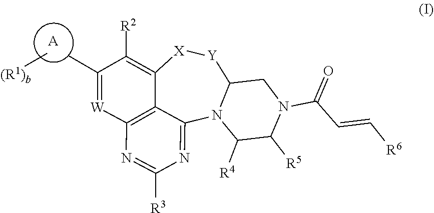

and pharmaceutically acceptable salts thereof. The specification also relates to processes and intermediates used for their preparation, pharmaceutical compositions containing them and their use in the treatment of cell proliferative disorders.

The specification relates to certain heteroaryl compounds and pharmaceutically acceptable salts thereof that inhibit G12C mutant RAS proteins and possess anti-cancer activity. The specification also relates to use of said heteroaryl compounds and pharmaceutically acceptable salts thereof in methods of treatment of the human or animal body, for example in prevention or treatment of cancer. The specification also relates to processes and intermediate compounds involved in the preparation of said heteroaryl compounds and to pharmaceutical compositions containing them.

The KRAS, NRAS and HRAS genes encode a set of closely related small GTPase proteins KRas, NRas and HRas, collectively referred to herein as the Ras proteins or Ras, that share 82-90% overall sequence identity. The Ras proteins are critical components of signalling pathways transmitting signals from cell-surface receptors to regulate cellular proliferation, survival and differentiation. Ras functions as a molecular switch cycling between an inactive GDP-bound state and an active GTP-bound state. The GDP/GTP cycle of Ras is tightly regulated in cells by guanine nucleotide exchange factors (GEFs) such as Sos1 and Sos2, which promote the exchange of GDP for GTP, and GTPase activating proteins (GAPs) such as NF-1 and p120RasGAP which stimulate the intrinsic GTPase activity of Ras hydrolysing GTP to GDP.

The Ras proteins are 188-189 amino acids in length and have a highly conserved N-terminal G-domain containing the p-loop region, which binds nucleotide, and the switch I and switch II regions which are important for regulatory and effector protein interactions. The C-terminal region of the Ras proteins are more divergent and contain elements which regulate the association of Ras with the membrane including the conserved carboxyl terminal CAXX box motif which is necessary for post-translational prenylation modifications. On binding to GTP the switch I and switch 11 regions of Ras undergo a conformational change which enables its interaction and activation of effector proteins to regulate down-stream signalling pathways. The best characterised effector of Ras is the serine/threonine kinase Raf which regulates the activity of the mitogen-activate protein kinase (MAPK) pathway. The PI3K pathway is another important effector pathway down-stream of Ras with the p110 catalytic subunit of the class I phosphoinositide 3-kinases interacting with Ras. Other effectors of Ras including RaIGDS, Tiam1, PLC-ε and Rassf1 have been have also been described (Cox, et al. Nature Reviews Drug Discovery 2014, 13:828-851).

RAS mutations are frequently found in cancer and approximately 30% of all human cancers have a mutation in KRAS, NRAS or HRAS genes. Oncogenic Ras is typically, but not exclusively, associated with mutations at glycine 12, glycine 13 or glutamine 61 of Ras. These residues are located at the active site of Ras and mutations impair intrinsic and/or GAP-catalysed GTPase activity favouring the formation of GTP bound Ras and aberrant activation of down-stream effector pathways. KRAS is the most frequently mutated RAS gene in cancer followed by NRAS and then HRAS. There are several tumour types that exhibit a high frequency of activating mutations in KRAS including pancreatic (˜90% prevalence), colorectal (˜40% prevalence) and non-small cell lung cancer (˜30% prevalence). KRAS mutations are also found in other cancer types including multiple myeloma, uterine cancer, bile duct cancer, stomach cancer, bladder cancer, diffuse large B cell lymphoma, rhabdomyosarcoma, cutaneous squamous cell carcinoma, cervical cancer, testicular germ cell cancer and others.

Glycine to cysteine mutations at residue 12 of Ras (the G12C mutation) is generated from a G. C to T. A base transversion at codon 12, a mutation commonly found in RAS genes that accounts for 14% of all KRAS, 2% of all NRAS and 2% of all HRAS mutations across cancer types. The G12C mutation is particularly enriched in KRAS mutant non-small cell lung cancer with approximately half carrying this mutation, which has been associated with the DNA adducts formed by tobacco smoke. The G12C mutation is not exclusively associated with lung cancer and is found in other RAS mutant cancer types including 8% of all KRAS mutant colorectal cancer.

To date there have been no inhibitors of G12C mutant Ras proteins which have been approved for therapeutic use. Hence there is a need for new inhibitors of G12C mutant Ras proteins that possess the required pharmaceutical properties to be suitable for clinical use. The compounds of the specification have been found to possess anti-tumour activity, being useful in inhibiting the uncontrolled cellular proliferation which arises from malignant disease. The compounds of the specification provide an anti-tumour effect by, as a minimum, acting as inhibitors of G12C mutant Ras proteins.

According to one aspect of the specification there is provided a compound of the Formula (I):

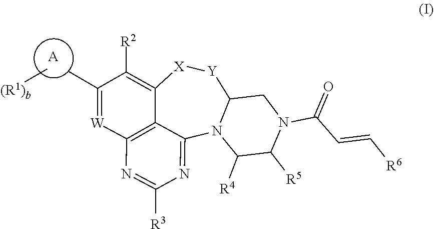

wherein:
Ring A is selected from aryl, monocyclic heteroaryl and bicyclic heteroaryl;
R¹ is independently selected from C₁₋₄alkyl, halo, hydroxy, C₁₋₄alkoxy, C₁₋₃fluoroalkyl, C₁₋₃fluoroalkoxy, cyano, acetylenyl, NR⁷R⁸, C(O)NR⁹R¹⁰, CH₂R¹¹, N═S(O)Me₂, S(O)Me and SO₂R¹²;
b is 0, 1, 2 or 3;

### W is N or CR¹³;

### X is O or NR¹⁴;

Y is CR¹⁵R¹⁶, CR¹⁷R¹⁸CR¹⁹R²⁰, C═O, or C(O)CR²¹R²²;
R² is H, cyano, halo, C₁₋₄alkyl, C₁₋₄alkoxy, C₁₋₃fluoroalkyl, NR²³R²⁴, acetylenyl or CH₂OR²⁵;
R³ is H, C₁₋₃fluoroalkyl, OR²⁶, NR²⁷R²⁸, CH₂R²⁹, SR³⁰ or C(O)R³¹;

### R⁴ is H or Me;

### R⁵ is H or Me;

R⁶ is H or CH₂NMe₂;
R⁷ is H, C₁₋₄alkyl, C(O)C₁₋₃alkyl or CO₂C₁₋₃alkyl;
R¹¹ is hydroxy, cyano, heterocyclyl, NR³²R³³, C(O)NR³⁴R³⁵ or SO₂C₁₋₃alkyl;
R¹² is C₁₋₃alkyl, C₁₋₃fluoroalkyl or NR³⁶R³⁷;
R¹³ is H, C₁₋₄alkyl, halo, C₁₋₃fluoroalkyl or C₁₋₄alkoxy;
R¹⁵, R¹⁶, R¹⁷ and R¹⁸ are independently selected from H and C₁₋₃alkyl;
R¹⁹, R²⁰, R²¹ and R²² are independently selected from H, C₁₋₃alkyl, and fluoro;
R²⁶ is selected from the group consisting of:
H;
C₁₋₄alkyl optionally substituted with 1 or 2 substituents independently selected from hydroxy, C₁₋₃alkoxy, halo, NR³⁸R³⁹, C(O)NR⁴⁰R⁴¹, SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl;
C₃₋₇cycloalkyl optionally substituted with 1 substituent selected from C₁₋₄alkyl, hydroxy and halo;
heterocyclyl optionally substituted with 1 or 2 substituents independently selected from C₁₋₄ alkyl, hydroxy, halo, C(O)Me, C₁₋₃ alkoxy, C₁₋₃fluoroalkyl, C₃₋₇cycloalkyl, heterocyclyl and heteroaryl; and
heteroaryl optionally substituted with 1 substituent selected from C₁₋₄alkyl, hydroxy, halo, cyano and C₁₋₄alkoxy;
   R²⁷ is selected from the group consisting of:
H;
C(O)R⁴²;
C₁₋₄alkyl optionally substituted with 1 or 2 substituents independently selected from hydroxy, C₁₋₃alkoxy, halo, NR⁴³R⁴⁴, C(O)NR⁴⁵R⁴⁸, SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl;
C₃₋₇cycloalkyl optionally substituted with 1 substituent selected from C₁₋₄alkyl, hydroxy and halo;
heterocyclyl optionally substituted with 1 or 2 substituents independently selected from C₁₋₄ alkyl, hydroxy, halo, C(O)Me, C₁₋₃alkoxy, C₁₋₃fluoroalkyl, C₃₋₇cycloalkyl, CH₂cyclopropyl, heterocyclyl and heteroaryl; and
heteroaryl optionally substituted with 1 substituent selected from C₁₋₄alkyl, hydroxy, halo, cyano and C₁₋₄alkoxy;

### R²⁸ is H or Me; or

R²⁷ and R²⁸ taken together with the nitrogen atom to which they are attached form a 4-, 5-, 6- or 7-membered heterocyclic ring, wherein said ring is optionally substituted with 1 or 2 substituents independently selected from C₁₋₄alkyl, hydroxy, halo, C(O)Me, NR⁴⁷R⁴⁸, C₁₋₃ alkoxy, C₁₋₃fluoroalkyl, C₃₋₇cycloalkyl, CH₂cyclopropyl, heterocyclyl and heteroaryl;
R²⁹ is selected from the group consisting of:
H;
NR⁴⁹R⁵⁰;
C₁₋₃alkyl optionally substituted with 1 or 2 substituents independently selected from hydroxy, C₁₋₃alkoxy, halo, NR⁵¹R⁵², C(O)NR⁵³R⁵⁴, SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl;
C₃₋₇cycloalkyl optionally substituted with 1 substituent selected from C₁₋₄alkyl, hydroxy and halo;
heterocyclyl optionally substituted with 1 or 2 substituents independently selected from C₁₋₄ alkyl, hydroxy, halo, C(O)Me, C₁₋₃ alkoxy, C₁₋₃fluoroalkyl, C₃₋₇cycloalkyl, CH₂cyclopropyl, heterocyclyl and heteroaryl; and
heteroaryl optionally substituted with 1 substituent selected from C₁₋₄alkyl, hydroxy, halo, cyano and C₁₋₄alkoxy;
   R³⁰ is selected from the group consisting of:
C₁₋₄alkyl optionally substituted with 1 or 2 substituents independently selected from hydroxy, C₁₋₃alkoxy, halo, NR⁵⁵R⁵⁶, C(O)NR⁵⁷R⁵⁸, SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl;
C₃₋₇cycloalkyl optionally substituted with 1 substituent selected from C₁₋₄alkyl, hydroxy and halo;
heterocyclyl optionally substituted with 1 or 2 substituents independently selected from C₁₋₄ alkyl, hydroxy, halo, C(O)Me, C₁₋₃ alkoxy, C₁₋₃fluoroalkyl, C₃₋₇cycloalkyl, CH₂cyclopropyl, heterocyclyl and heteroaryl; and
heteroaryl optionally substituted with 1 substituent selected from C₁₋₄alkyl, hydroxy, halo, cyano and C₁₋₄alkoxy;
   R³¹ is NR⁵⁹R⁶⁰;
   R⁴² is heteroaryl optionally substituted with 1 or 2 substituents independently selected from C₁₋₄alkyl, hydroxy, halo, cyano and C₁₋₄alkoxy, or is C₁₋₄alkyl optionally substituted with 1 or 2 substituents independently selected from hydroxy, C₁₋₃alkoxy, halo and NR⁶¹R⁶²;
   R⁴⁹ and R⁵¹ are independently selected from H, C₁₋₄alkyl, heterocyclyl and heteroaryl;
   R⁵⁹ and R⁶⁰ are independently selected from H and C₁₋₄alkyl; or
   R⁵⁹ and R⁶⁰ taken together with the nitrogen atom to which they are attached form a 4-, 5- or 6-membered heterocyclic ring, wherein said ring is optionally substituted with 1 substituent selected from C₁₋₄alkyl, hydroxy, halo and C(O)Me;
   R⁸, R⁹, R¹⁰, R¹⁴, R²³, R²⁴, R²⁵, R³², R³³, R³⁴, R³⁵, R³⁶, R³⁷, R³⁸, R³⁹, R⁴⁰, R⁴¹, R⁴³, R⁴⁴, R⁴⁵, R⁴⁶, R⁴⁷, R⁴⁸, R⁵⁰, R⁵², R⁵³, R⁵⁴, R⁵⁵, R⁵⁶, R⁵⁷, R⁵⁸, R⁶¹ and R⁶² are independently selected from H and C₁₋₄alkyl;
   or a pharmaceutically acceptable salt thereof.

In one embodiment there is provided a compound of Formula (I) as defined above.

In one embodiment there is provided a pharmaceutically acceptable salt of a compound of Formula (I).

In one embodiment ring A is aryl.

In one embodiment ring A is phenyl.

In one embodiment ring A is monocyclic heteroaryl.

In one embodiment ring A is monocyclic heteroaryl selected from pyridinyl, pyrimidinyl, pyrazinyl, pyridazinyl, pyrrolyl, pyrazolyl and imidazolyl.

In one embodiment ring A is pyridinyl.

In one embodiment ring A is bicyclic heteroaryl.

In one embodiment ring A is bicyclic heteroaryl selected from the group consisting of:

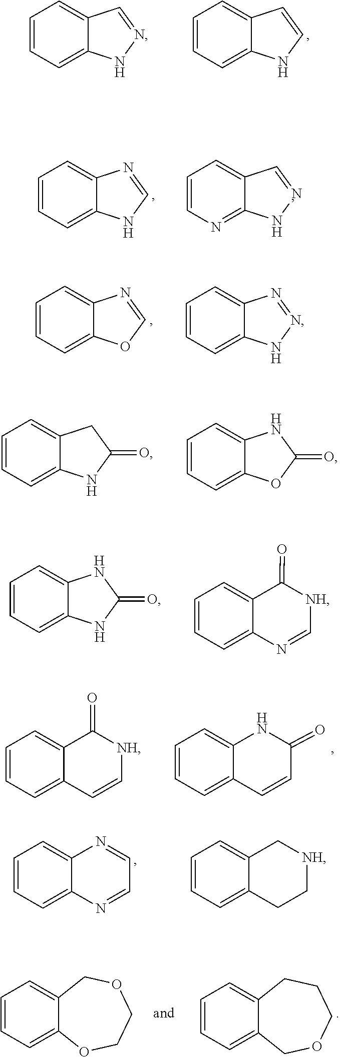

In one embodiment ring A is bicyclic heteroaryl selected from the group consisting of:

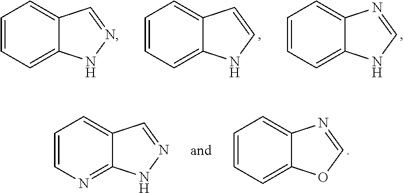

In one embodiment ring A is bicyclic heteroaryl selected from the group consisting of:

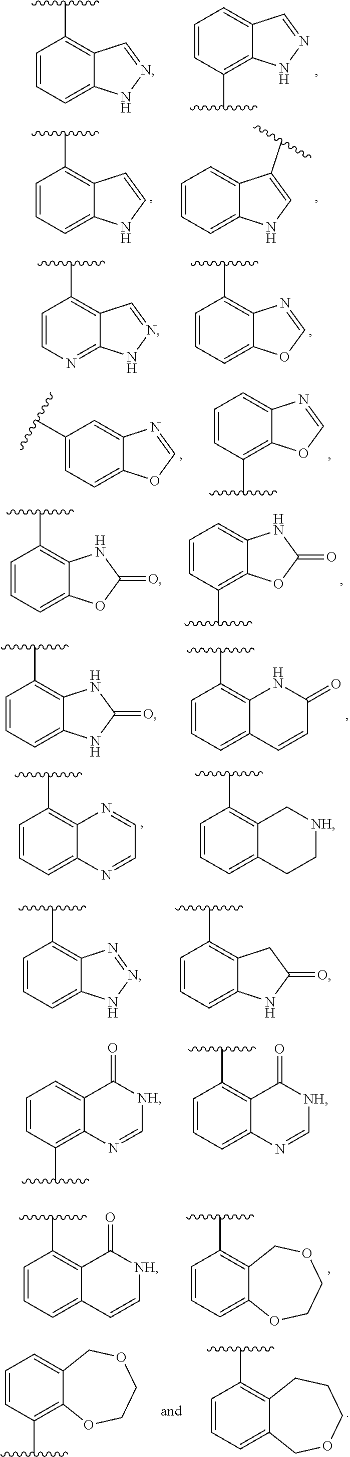

In one embodiment ring A is bicyclic heteroaryl selected from the group consisting of:

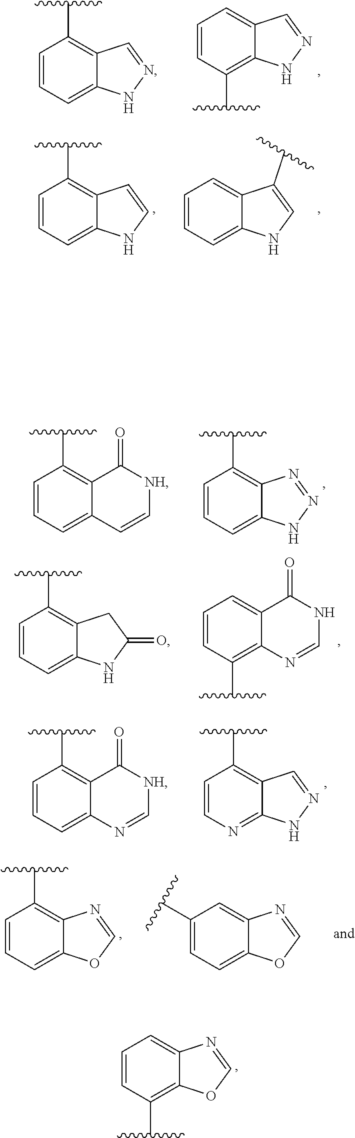

In one embodiment ring A is:

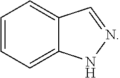

In one embodiment ring A is:

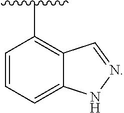 [SMILES: *c1cccc2[nH]ncc12]

In one embodiment R¹ is independently selected from C₁₋₄alkyl, halo, hydroxy, C₁₋₄alkoxy, C₁₋₃ fluoroalkoxy, cyano, NR⁷R⁸, C(O)NR⁹R¹⁰, CH₂R¹¹, N═S(O)Me₂ and SO₂R¹².

In one embodiment R¹ is independently selected from C₁₋₄alkyl, halo, hydroxy, C₁₋₄alkoxy, cyano and NR⁷R⁸.

In one embodiment R¹ is independently selected from methyl, fluoro, chloro, hydroxy, methoxy, OCF₃, cyano, NR⁷R⁸, C(O)NR⁹R¹⁰, CH₂R¹, N═S(O)Me₂ and SO₂R¹².

In one embodiment R¹ is independently selected from methyl, fluoro, chloro, hydroxy, methoxy and cyano.

In one embodiment R¹ is independently selected from methyl, fluoro and hydroxy.

In one embodiment R¹ is methyl.

In one embodiment b is 0, 1 or 2.

In one embodiment b is 1 or 2.

In one embodiment b is 0.

In one embodiment b is 1.

In one embodiment b is 2.

In one embodiment b is 1 and R¹ is methyl.

In one embodiment b is 2 and R¹ is independently selected from methyl, fluoro, chloro, hydroxy, methoxy and cyano.

In one embodiment b is 2 and R¹ is hydroxy and fluoro.

In one embodiment ring A is:

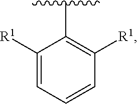

and each R¹ is independently selected from C₁₋₄alkyl, halo, hydroxy, C₁₋₄alkoxy, C₁₋₃fluoroalkoxy, cyano, NR⁷R⁸, C(O)NR⁹R¹⁰ and CH₂R¹¹.

In one embodiment ring A is:

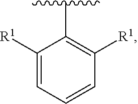

and each R¹ is independently selected from C₁₋₄alkyl, halo, hydroxy, C₁₋₄alkoxy and cyano.

In one embodiment ring A is:

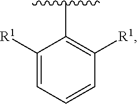

and each R¹ is independently selected from methyl, fluoro chloro, hydroxy, methoxy and cyano.

In one embodiment ring A is:

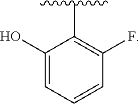 [SMILES: *c1c(O)cccc1F]

In one embodiment ring A is:

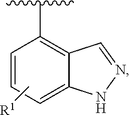

and R¹ is methyl.

In one embodiment ring A is:

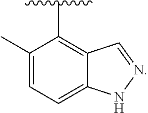 [SMILES: *c1c(C)ccc2[nH]ncc12]

In one embodiment W is N.

In one embodiment W is CR¹³.

In one embodiment W is CR¹³ and R¹³ is H, C₁₋₄alkyl, chloro or fluoro.

In one embodiment W is CR¹³ and R¹³ is H.

In one embodiment W is CR¹³ and R¹³ is chloro.

In one embodiment W is CR¹³ and R¹³ is fluoro.

In one embodiment X is O.

In one embodiment X is NR¹⁴.

In one embodiment Y is CR¹⁵R¹⁶ or CR¹⁷R¹⁸CR¹⁹R²⁰.

In one embodiment Y is CR¹⁵R¹⁶.

In one embodiment Y is CR¹⁷R¹⁸CR¹⁹R²⁰.

In one embodiment Y is CH₂.

In one embodiment Y is CH₂CH₂.

In one embodiment X is O and Y is CH₂.

In one embodiment X is O and Y is CH₂CH₂.

In one embodiment R² is H, cyano, halo or C₁₋₄alkyl.

In one embodiment R² is H or halo.

In one embodiment R² is H or chloro.

In one embodiment R² is H.

In one embodiment R² is chloro.

In one embodiment R³ is H, OR²⁶ or NR²⁷R²⁸.

In one embodiment R³ is H.

In one embodiment R³ is OR²⁶.

In one embodiment R³ is NR²⁷R²⁸.

In one embodiment R⁴ is H.

In one embodiment R⁴ is Me.

In one embodiment R⁵ is H.

In one embodiment R⁵ is Me.

In one embodiment R⁴ is H and R⁵ is H.

In one embodiment R⁶ is H.

In one embodiment R⁷ is H, C(O)Me or CO₂Me.

In one embodiment R⁷ is H.

In one embodiment R⁷ is C(O)Me.

In one embodiment R⁷ is CO₂Me.

In one embodiment R¹¹ is hydroxy, cyano, or C(O)NR³⁴R³⁵.

In one embodiment R¹¹ is hydroxy, cyano, or C(O)NH₂.

In one embodiment R¹¹ is hydroxy.

In one embodiment R¹¹ is cyano.

In one embodiment R¹¹ is C(O)NH₂.

In one embodiment R¹² is C₁₋₃alkyl or NR³⁶R³⁷.

In one embodiment R¹² is C₁₋₃alkyl.

In one embodiment R¹² is Me.

In one embodiment R¹² is NR³⁶R³⁷.

In one embodiment R¹² is NH₂.

In one embodiment R²⁶ is C₁₋₄alkyl optionally substituted with 1 or 2 substituents independently selected from hydroxy, C₁₋₃alkoxy, halo, NR³⁸R³⁹, C(O)NR⁴⁰R⁴¹, SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl.

In one embodiment R²⁶ is methyl optionally substituted with 1 or 2 substituents independently selected from hydroxy, C₁₋₃alkoxy, halo, NR³⁸R³⁹, C(O)NR⁴⁰R⁴¹, SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl.

In one embodiment R²⁶ is ethyl optionally substituted with 1 or 2 substituents independently selected from hydroxy, C₁₋₃alkoxy, halo, NR³⁸R³⁹, C(O)NR⁴⁰R⁴¹, SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄ alkyl, hydroxy, halo, C(O)Me, C₁₋₃alkoxy, C₁₋₃fluoroalkyl, C₃₋₇cycloalkyl, heterocyclyl and heteroaryl.

In one embodiment R²⁶ is C₁₋₄alkyl substituted with heterocyclyl, wherein said heterocyclyl is optionally substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl.

In one embodiment R²⁶ is C₁₋₄alkyl substituted with heterocyclyl, wherein said heterocyclyl is optionally substituted with 1 or 2 substituents independently selected from methyl, hydroxy, fluoro, C(O)Me, methoxy, C₁₋₃fluoroalkyl, cyclopropyl, heterocyclyl and heteroaryl.

In one embodiment R²⁶ is C₁₋₄alkyl substituted with heterocyclyl, wherein said heterocyclyl is optionally substituted with 1 or 2 substituents independently selected from methyl, hydroxy, fluoro, methoxy and cyclopropyl.

In one embodiment R²⁶ is C₁₋₄alkyl substituted with heteroaryl, wherein said heteroaryl is optionally substituted with 1 or 2 substituents independently selected from C₁₋₄alkyl, hydroxy, halo, cyano and C₁₋₄alkoxy.

In one embodiment R²⁶ is C₁₋₄alkyl substituted with heteroaryl, wherein said heteroaryl is optionally substituted with 1 or 2 substituents independently selected from methyl, hydroxy, fluoro, cyano and methoxy.

In one embodiment R²⁶ is C₃₋₇cycloalkyl optionally substituted with 1 substituent selected from C₁₋₄alkyl, hydroxy and halo.

In one embodiment R²⁶ is heterocyclyl optionally substituted with 1 or 2 substituents independently selected from C₁₋₄alkyl, hydroxy, halo, C(O)Me, C₁₋₃ alkoxy, C₁₋₃fluoroalkyl, C₃₋₇cycloalkyl, heterocyclyl and heteroaryl.

In one embodiment R²⁶ is heterocyclyl optionally substituted with 1 or 2 substituents independently selected from methyl, hydroxy, fluoro, C(O)Me, methoxy, C₁₋₃fluoroalkyl and cyclopropyl.

In one embodiment R²⁶ is heteroaryl optionally substituted with 1 substituent independently selected from C₁₋₄alkyl, hydroxy, halo, cyano and C₁₋₄alkoxy.

In one embodiment R²⁶ is heteroaryl optionally substituted with 1 substituent independently selected from methyl, hydroxy, halo, cyano and methoxy.

In one embodiment R²⁷ is H.

In one embodiment R²⁷ is C(O)R⁴².

In one embodiment R²⁷ is C₁₋₄alkyl optionally substituted with 1 or 2 substituents independently selected from hydroxy, C₁₋₃alkoxy, halo, NR⁴³R⁴⁴, C(O)NR⁴⁵R⁴⁶, SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl.

In one embodiment R²⁷ is methyl optionally substituted with 1 or 2 substituents independently selected from hydroxy, C₁₋₃alkoxy, halo, NR⁴³R⁴⁴. C(O)NR⁴⁵R⁴⁶. SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl.

In one embodiment R²⁷ is ethyl optionally substituted with 1 or 2 substituents independently selected from hydroxy, C₁₋₃alkoxy, halo, NR⁴³R⁴⁴, C(O)NR⁴⁵R⁴⁶, SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄ alkyl, hydroxy, halo, C(O)Me, C₁₋₃alkoxy, C₁₋₃fluoroalkyl, C₃₋₇cycloalkyl, heterocyclyl and heteroaryl.

In one embodiment R²⁷ is C₁₋₄alkyl substituted with heterocyclyl, wherein said heterocyclyl is optionally substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl.

In one embodiment R²⁷ is C₁₋₄alkyl substituted with heterocyclyl, wherein said heterocyclyl is optionally substituted with 1 or 2 substituents independently selected from methyl, hydroxy, fluoro, C(O)Me, methoxy, C₁₋₃fluoroalkyl, cyclopropyl, heterocyclyl and heteroaryl.

In one embodiment R²⁷ is C₁₋₄alkyl substituted with heterocyclyl, wherein said heterocyclyl is optionally substituted with 1 or 2 substituents independently selected from methyl, hydroxy, fluoro, methoxy and cyclopropyl.

In one embodiment R²⁷ is C₁₋₄alkyl substituted with heteroaryl, wherein said heteroaryl is optionally substituted with 1 or 2 substituents independently selected from C₁₋₄alkyl, hydroxy, halo, cyano and C₁₋₄alkoxy.

In one embodiment R²⁷ is C₁₋₄alkyl substituted with heteroaryl, wherein said heteroaryl is optionally substituted with 1 or 2 substituents independently selected from methyl, hydroxy, fluoro, cyano and methoxy.

In one embodiment R²⁷ is C₃₋₇cycloalkyl optionally substituted with 1 substituent selected from C₁₋₄alkyl, hydroxy and halo.

In one embodiment R²⁷ is heterocyclyl optionally substituted with 1 or 2 substituents independently selected from C₁₋₄alkyl, hydroxy, halo, C(O)Me, C₁₋₃ alkoxy, C₁₋₃fluoroalkyl, C₃₋₇cycloalkyl, heterocyclyl and heteroaryl.

In one embodiment R²⁷ is heterocyclyl optionally substituted with 1 or 2 substituents independently selected from methyl, hydroxy, fluoro, C(O)Me, methoxy, C₁₋₃fluoroalkyl and cyclopropyl.

In one embodiment R²⁷ is heteroaryl optionally substituted with 1 substituent independently selected from C₁₋₄alkyl, hydroxy, halo, cyano and C₁₋₄alkoxy.

In one embodiment R²⁷ is heteroaryl optionally substituted with 1 substituent independently selected from methyl, hydroxy, halo, cyano and methoxy.

In one embodiment R²⁸ is H.

In one embodiment R²⁸ is Me.

In one embodiment R²⁷ and R²⁸ taken together with the nitrogen atom to which they are attached form a 4-, 5-, 6- or 7-membered heterocyclic ring, wherein said ring is optionally substituted with 1 or 2 substituents independently selected from C₁₋₄alkyl, hydroxy, halo, C(O)Me, NR⁴⁷R⁴⁸, C₁₋₃ alkoxy, C₁₋₃fluoroalkyl, C₃₋₇cycloalkyl, CH₂cyclopropyl, heterocyclyl and heteroaryl.

In one embodiment R²⁷ and R²⁸ taken together with the nitrogen atom to which they are attached form an azetidine ring, wherein said azetidine ring is optionally substituted with 1 or 2 substituents independently selected from C₁₋₄alkyl, hydroxy, halo, C(O)Me, NR⁴⁷R⁴⁸, C₁₋₃ alkoxy, C₁₋₃ fluoroalkyl, C₃₋₇cycloalkyl, CH₂cyclopropyl, heterocyclyl and heteroaryl.

In one embodiment R²⁷ and R²⁸ taken together with the nitrogen atom to which they are attached form an azetidine ring, wherein said azetidine ring is substituted with NR⁴⁷R⁴⁸.

In one embodiment R²⁷ and R²⁸ taken together with the nitrogen atom to which they are attached form a 5-membered heterocyclic ring, wherein said ring is optionally substituted with 1 or 2 substituents independently selected from C₁₋₄alkyl, hydroxy, halo, C(O)Me, NR⁴⁷R⁴⁸, C₁₋₃ alkoxy, C₁₋₃ fluoroalkyl, C₃₋₇cycloalkyl, CH₂cyclopropyl, heterocyclyl and heteroaryl.

In one embodiment R²⁷ and R²⁸ taken together with the nitrogen atom to which they are attached form a 6-membered heterocyclic ring, wherein said ring is optionally substituted with 1 or 2 substituents independently selected from C₁₋₄alkyl, hydroxy, halo, C(O)Me, NR⁴⁷R⁴⁸, C₁₋₃ alkoxy, C₁₋₃ fluoroalkyl, C₃₋₇cycloalkyl, CH₂cyclopropyl, heterocyclyl and heteroaryl.

In one embodiment R²⁷ and R²⁸ taken together with the nitrogen atom to which they are attached form a piperazine ring, wherein said piperazine ring is optionally substituted with 1 or 2 substituents independently selected from C₁₋₄alkyl, hydroxy, halo, C(O)Me, NR⁴⁷R⁴⁸, C₁₋₃ alkoxy, C₁₋₃ fluoroalkyl, C₃₋₇cycloalkyl, CH₂cyclopropyl, heterocyclyl and heteroaryl.

In one embodiment R²⁷ and R²⁸ taken together with the nitrogen atom to which they are attached form a morpholine ring, wherein said morpholine ring is optionally substituted with 1 or 2 substituents independently selected from C₁₋₄alkyl, hydroxy, halo, C(O)Me, NR⁴⁷R⁴⁸, C₁₋₃ alkoxy, C₁₋₃ fluoroalkyl, C₃₋₇cycloalkyl, CH₂cyclopropyl, heterocyclyl and heteroaryl.

In one embodiment R²⁷ and R²⁸ taken together with the nitrogen atom to which they are attached form a 7-membered heterocyclic ring, wherein said ring is optionally substituted with 1 or 2 substituents independently selected from C₁₋₄alkyl, hydroxy, halo, C(O)Me, NR⁴⁷R⁴⁸, C₁₋₃ alkoxy, C₁₋₃ fluoroalkyl, C₃₋₇cycloalkyl, CH₂cyclopropyl, heterocyclyl and heteroaryl.

In one embodiment R²⁹ is NR⁴⁹R⁵⁰.

In one embodiment R²⁹ is C₁₋₃alkyl optionally substituted with 1 or 2 substituents independently selected from hydroxy, C₁₋₃alkoxy, halo, NR⁵¹R⁵², C(O)NR⁵³R⁵⁴, SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl.

In one embodiment R²⁹ is C₃₋₇cycloalkyl optionally substituted with 1 substituent selected from C₁₋₄alkyl, hydroxy and halo.

In one embodiment R²⁹ is heterocyclyl optionally substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, CH₂cyclopropyl, heterocyclyl and heteroaryl.

In one embodiment R²⁹ is heteroaryl optionally substituted with 1 substituent selected from C₁₋₄alkyl, hydroxy, halo, cyano and C₁₋₄alkoxy.

In one embodiment R³⁰ is C₁₋₄alkyl optionally substituted with 1 or 2 substituents independently selected from hydroxy, C₁₋₃alkoxy, halo, NR⁵⁵R⁵⁶, C(O)NR⁵⁷R⁵⁸, SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl.

In one embodiment R³⁰ is C₃₋₇cycloalkyl optionally substituted with 1 substituent selected from C₁₋₄alkyl, hydroxy and halo.

In one embodiment R³⁰ is heterocyclyl optionally substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, CH₂cyclopropyl, heterocyclyl and heteroaryl.

In one embodiment R³⁰ is heteroaryl optionally substituted with 1 substituent selected from C₁₋₄alkyl, hydroxy, halo, cyano and C₁₋₄alkoxy.

In one embodiment R⁴² is heteroaryl optionally substituted with 1 or 2 substituents independently selected from C₁₋₄alkyl, hydroxy, halo, cyano and C₁₋₄alkoxy.

In one embodiment R⁴² is C₁₋₄alkyl optionally substituted with 1 or 2 substituents independently selected from hydroxy, C₁₋₃alkoxy, halo and NR⁶¹R⁶².

In one embodiment there is provided the compound of Formula (Ia):

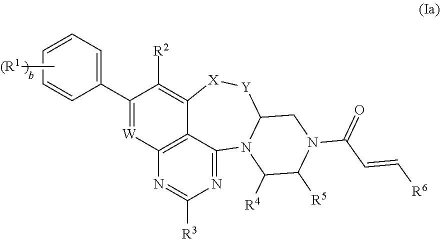

wherein:
R¹ is independently selected from C₁₋₄alkyl, halo, hydroxy, C₁₋₄alkoxy, C₁₋₃fluoroalkyl, C₁₋₃fluoroalkoxy, cyano, acetylenyl, NR⁷R⁸, C(O)NR⁹R¹⁰, CH₂R¹¹, N═S(O)Me₂, S(O)Me and SO₂R¹²;
b is 0, 1, 2 or 3;

### W is N or CR¹³;

### X is O or NR¹⁴;

Y is CR¹⁵R¹⁶, CR¹⁷R¹⁸CR¹⁹R²⁰, C═O, or C(O)CR²¹R²²;
R² is H, cyano, halo, C₁₋₄alkyl, C₁₋₄alkoxy, C₁₋₃fluoroalkyl, NR²³R²⁴, acetylenyl or CH₂OR²⁵;
R³ is H, C₁₋₃fluoroalkyl, OR²⁶, NR²⁷R²⁸, CH₂R²⁹, SR³⁰ or C(O)R³¹;

### R⁴ is H or Me;

### R⁵ is H or Me;

R⁶ is H or CH₂NMe₂;
R⁷ is H, C₁₋₄alkyl, C(O)C₁₋₃alkyl or CO₂C₁₋₃alkyl;
R¹¹ is hydroxy, cyano, heterocyclyl, NR³²R³³, C(O)NR³⁴R³⁵ or SO₂C₁₋₃alkyl;
R¹² is C₁₋₃alkyl, C₁₋₃fluoroalkyl or NR³⁶R³⁷;
R¹³ is H, C₁₋₄alkyl, halo, C₁₋₃fluoroalkyl or C₁₋₄alkoxy;
R¹⁵, R¹⁶, R¹⁷ and R¹⁸ are independently selected from H and C₁₋₃alkyl;
R¹⁹, R²⁰, R²¹ and R²² are independently selected from H, C₁₋₃alkyl, and fluoro;
R²⁶ is selected from the group consisting of:
H;
C₁₋₄alkyl optionally substituted with 1 or 2 substituents independently selected from hydroxy, C₁₋₃alkoxy, halo, NR³⁸R³⁹, C(O)NR⁴⁰R⁴¹, SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl;
C₃₋₇cycloalkyl optionally substituted with 1 substituent selected from C₁₋₄alkyl, hydroxy and halo;
heterocyclyl optionally substituted with 1 or 2 substituents independently selected from C₁₋₄ alkyl, hydroxy, halo, C(O)Me, C₁₋₃ alkoxy, C₁₋₃fluoroalkyl, C₃₋₇cycloalkyl, heterocyclyl and heteroaryl; and
heteroaryl optionally substituted with 1 substituent selected from C₁₋₄alkyl, hydroxy, halo, cyano and C₁₋₄alkoxy;
   R²⁷ is selected from the group consisting of:
H;
C(O)R⁴²;
C₁₋₄alkyl optionally substituted with 1 or 2 substituents independently selected from hydroxy, C₁₋₃alkoxy, halo, NR⁴³R⁴⁴, C(O)NR⁴⁵R⁴⁶, SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl;
C₃₋₇cycloalkyl optionally substituted with 1 substituent selected from C₁₋₄alkyl, hydroxy and halo;
heterocyclyl optionally substituted with 1 or 2 substituents independently selected from C₁₋₄ alkyl, hydroxy, halo, C(O)Me, C₁₋₃ alkoxy, C₁₋₃fluoroalkyl, C₃₋₇cycloalkyl, CH₂cyclopropyl, heterocyclyl and heteroaryl; and
heteroaryl optionally substituted with 1 substituent selected from C₁₋₄alkyl, hydroxy, halo, cyano and C₁₋₄alkoxy;

### R²⁸ is H or Me; or

R²⁷ and R²⁸ taken together with the nitrogen atom to which they are attached form a 4-, 5-, 6- or 7-membered heterocyclic ring, wherein said ring is optionally substituted with 1 or 2 substituents independently selected from C₁₋₄alkyl, hydroxy, halo, C(O)Me, NR⁴⁷R⁴⁸, C₁₋₃alkoxy, C₁₋₃fluoroalkyl, C₃₋₇cycloalkyl, CH₂cyclopropyl, heterocyclyl and heteroaryl;
R²⁹ is selected from the group consisting of:
H;
NR⁴⁹R⁵⁰;
C₁₋₃alkyl optionally substituted with 1 or 2 substituents independently selected from hydroxy, C₁₋₃alkoxy, halo, NR⁵¹R⁵², C(O)NR⁵³R⁵⁴, SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl;
C₃₋₇cycloalkyl optionally substituted with 1 substituent selected from C₁₋₄alkyl, hydroxy and halo;
heterocyclyl optionally substituted with 1 or 2 substituents independently selected from C₁₋₄ alkyl, hydroxy, halo, C(O)Me, C₁₋₃ alkoxy, C₁₋₃fluoroalkyl, C₃₋₇cycloalkyl, CH₂cyclopropyl, heterocyclyl and heteroaryl; and
heteroaryl optionally substituted with 1 substituent selected from C₁₋₄alkyl, hydroxy, halo, cyano and C₁₋₄alkoxy;
   R³⁰ is selected from the group consisting of:
C₁₋₄alkyl optionally substituted with 1 or 2 substituents independently selected from hydroxy, C₁₋₃alkoxy, halo, NR⁵⁵R⁵⁶, C(O)NR⁵⁷R⁵⁸, SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl;
C₃₋₇cycloalkyl optionally substituted with 1 substituent selected from C₁₋₄alkyl, hydroxy and halo;
heterocyclyl optionally substituted with 1 or 2 substituents independently selected from C₁₋₄ alkyl, hydroxy, halo, C(O)Me, C₁₋₃ alkoxy, C₁₋₃fluoroalkyl, C₃₋₇cycloalkyl, CH₂cyclopropyl, heterocyclyl and heteroaryl; and
heteroaryl optionally substituted with 1 substituent selected from C₁₋₄alkyl, hydroxy, halo, cyano and C₁₋₄alkoxy;
   R³¹ is NR⁵⁹R⁶⁰;
   R⁴² is heteroaryl optionally substituted with 1 or 2 substituents independently selected from C₁₋₄alkyl, hydroxy, halo, cyano and C₁₋₄alkoxy, or is C₁₋₄alkyl optionally substituted with 1 or 2 substituents independently selected from hydroxy, C₁₋₃alkoxy, halo and NR⁶¹R⁶²;
   R⁴⁹ and R⁵¹ are independently selected from H, C₁₋₄alkyl, heterocyclyl and heteroaryl;
   R⁵⁹ and R⁶⁰ are independently selected from H and C₁₋₄alkyl; or
   R⁵⁹ and R⁶⁰ taken together with the nitrogen atom to which they are attached form a 4-, 5- or 6-membered heterocyclic ring, wherein said ring is optionally substituted with 1 substituent selected from C₁₋₄alkyl, hydroxy, halo and C(O)Me;
   R⁸, R⁹, R¹⁰, R¹⁴, R²³, R²⁴, R²⁵, R³², R³³, R³⁴, R³⁵, R³⁶, R³⁷, R³⁸, R³⁹, R⁴⁰, R⁴¹, R⁴³, R⁴⁴, R⁴⁵, R⁴⁶, R⁴⁷, R⁴⁸, R⁵⁰, R⁵², R⁵³, R⁵⁴, R⁵⁵, R⁵⁶, R⁵⁷, R⁵⁸, R⁶¹ and R⁶² are independently selected from H and C₁₋₄alkyl;
   or a pharmaceutically acceptable salt thereof.

In one embodiment there is provided a compound of Formula (Ia), or a pharmaceutically acceptable salt thereof, wherein R¹ is independently selected from C₁₋₄alkyl, halo, hydroxy, C₁₋₄alkoxy, C₁₋₃fluoroalkoxy, cyano, NR⁷R⁸, C(O)NR⁹R¹⁰, CH₂R¹¹, N═S(O)Me₂ and SO₂R¹².

In one embodiment there is provided a compound of Formula (Ia), or a pharmaceutically acceptable salt thereof, wherein R¹ is independently selected from C₁₋₄alkyl, halo, hydroxy, C₁₋₄alkoxy, cyano and NR⁷R⁸.

In one embodiment there is provided a compound of Formula (Ia), or a pharmaceutically acceptable salt thereof, wherein R¹ is independently selected from methyl, fluoro, chloro, hydroxy, methoxy, OCF₃, cyano, NR⁷R⁸, C(O)NR⁹R¹⁰, CH₂R¹¹, N═S(O)Me₂ and SO₂R¹².

In one embodiment there is provided a compound of Formula (Ia), or a pharmaceutically acceptable salt thereof, wherein R¹ is independently selected from methyl, fluoro, chloro, hydroxy, methoxy and cyano.

In one embodiment there is provided a compound of Formula (Ia), or a pharmaceutically acceptable salt thereof, wherein R⁶ is H.

In one embodiment there is provided the compound of Formula (Ib):

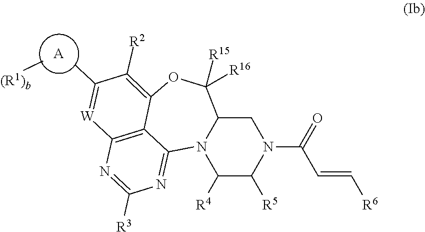

wherein:
Ring A is selected from aryl, monocyclic heteroaryl and bicyclic heteroaryl;
R¹ is independently selected from C₁₋₄alkyl, halo, hydroxy, C₁₋₄alkoxy, C₁₋₃fluoroalkyl, C₁₋₃fluoroalkoxy, cyano, acetylenyl, NR⁷R⁸, C(O)NR⁹R¹⁰, CH₂R¹¹, N═S(O)Me₂, S(O)Me and SO₂R¹²;
b is 0, 1, 2 or 3;

### W is N or CR¹³;

R² is H, cyano, halo, C₁₋₄alkyl, C₁₋₄alkoxy, C₁₋₃fluoroalkyl, NR²³R²⁴, acetylenyl or CH₂OR²⁵;
R³ is H, C₁₋₃fluoroalkyl, OR²⁶, NR²⁷R²⁸, CH₂R²⁹, SR³⁰ or C(O)R³¹;

### R⁴ is H or Me;

### R⁵ is H or Me;

R⁶ is H or CH₂NMe₂;
R⁷ is H, C₁₋₄alkyl, C(O)C₁₋₃alkyl or CO₂C₁₋₃alkyl;
R¹¹ is hydroxy, cyano, heterocyclyl, NR³²R³³, C(O)NR³⁴R³⁵ or SO₂C₁₋₃alkyl;
R¹² is C₁₋₃alkyl, C₁₋₃fluoroalkyl or NR³⁶R³⁷;
R¹³ is H, C₁₋₄alkyl, halo, C₁₋₃fluoroalkyl or C₁₋₄alkoxy;
R¹⁵ and R¹⁶ are independently selected from H and C₁₋₃alkyl;
R²⁶ is selected from the group consisting of:
H;
C₁₋₄alkyl optionally substituted with 1 or 2 substituents independently selected from hydroxy, C₁₋₃alkoxy, halo, NR³⁸R³⁹, C(O)NR⁴⁰R⁴¹, SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl;
C₃₋₇cycloalkyl optionally substituted with 1 substituent selected from C₁₋₄alkyl, hydroxy and halo;
heterocyclyl optionally substituted with 1 or 2 substituents independently selected from C₁₋₄ alkyl, hydroxy, halo, C(O)Me, C₁₋₃ alkoxy, C₁₋₃fluoroalkyl, C₃₋₇cycloalkyl, heterocyclyl and heteroaryl; and
heteroaryl optionally substituted with 1 substituent selected from C₁₋₄alkyl, hydroxy, halo, cyano and C₁₋₄alkoxy;
   R²⁷ is selected from the group consisting of:
H;
C(O)R⁴²;
C₁₋₄alkyl optionally substituted with 1 or 2 substituents independently selected from hydroxy, C₁₋₃alkoxy, halo, NR⁴³R⁴⁴, C(O)NR⁴⁵R⁴⁶, SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl;
C₃₋₇cycloalkyl optionally substituted with 1 substituent selected from C₁₋₄alkyl, hydroxy and halo;
heterocyclyl optionally substituted with 1 or 2 substituents independently selected from C₁₋₄ alkyl, hydroxy, halo, C(O)Me, C₁₋₃ alkoxy, C₁₋₃fluoroalkyl, C₃₋₇cycloalkyl, CH₂cyclopropyl, heterocyclyl and heteroaryl; and
heteroaryl optionally substituted with 1 substituent selected from C₁₋₄alkyl, hydroxy, halo, cyano and C₁₋₄alkoxy;

### R²⁸ is H or Me; or

R²⁷ and R²⁸ taken together with the nitrogen atom to which they are attached form a 4-, 5-, 6- or 7-membered heterocyclic ring, wherein said ring is optionally substituted with 1 or 2 substituents independently selected from C₁₋₄alkyl, hydroxy, halo, C(O)Me, NR⁴⁷R⁴⁸, C₁₋₃alkoxy, C₁₋₃fluoroalkyl, C₃₋₇cycloalkyl, CH₂cyclopropyl, heterocyclyl and heteroaryl;
R²⁹ is selected from the group consisting of:
H;
NR⁴⁹R⁵⁰;
C₁₋₃alkyl optionally substituted with 1 or 2 substituents independently selected from hydroxy, C₁₋₃alkoxy, halo, NR⁵¹R⁵², C(O)NR⁵³R⁵⁴, SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl;
C₃₋₇cycloalkyl optionally substituted with 1 substituent selected from C₁₋₄alkyl, hydroxy and halo;
heterocyclyl optionally substituted with 1 or 2 substituents independently selected from C₁₋₄ alkyl, hydroxy, halo, C(O)Me, C₁₋₃ alkoxy, C₁₋₃fluoroalkyl, C₃₋₇cycloalkyl, CH₂cyclopropyl, heterocyclyl and heteroaryl; and
heteroaryl optionally substituted with 1 substituent selected from C₁₋₄alkyl, hydroxy, halo, cyano and C₁₋₄alkoxy;
   R³⁰ is selected from the group consisting of:
C₁₋₄alkyl optionally substituted with 1 or 2 substituents independently selected from hydroxy, C₁₋₃alkoxy, halo, NR⁵⁵R⁵⁶, C(O)NR⁵⁷R⁵⁸, SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl;
C₃₋₇cycloalkyl optionally substituted with 1 substituent selected from C₁₋₄alkyl, hydroxy and halo;
heterocyclyl optionally substituted with 1 or 2 substituents independently selected from C₁₋₄ alkyl, hydroxy, halo, C(O)Me, C₁₋₃ alkoxy, C₁₋₃fluoroalkyl, C₃₋₇cycloalkyl, CH₂cyclopropyl, heterocyclyl and heteroaryl; and
heteroaryl optionally substituted with 1 substituent selected from C₁₋₄alkyl, hydroxy, halo, cyano and C₁₋₄alkoxy;
   R³¹ is NR⁵⁹R⁶⁰;
   R⁴² is heteroaryl optionally substituted with 1 or 2 substituents independently selected from C₁₋₄alkyl, hydroxy, halo, cyano and C₁₋₄alkoxy, or is C₁₋₄alkyl optionally substituted with 1 or 2 substituents independently selected from hydroxy, C₁₋₃alkoxy, halo and NR⁶¹R⁶²;
   R⁴⁹ and R⁵¹ are independently selected from H, C₁₋₄alkyl, heterocyclyl and heteroaryl;
   R⁵⁹ and R⁶⁰ are independently selected from H and C₁₋₄alkyl; or
   R⁵⁹ and R⁶⁰ taken together with the nitrogen atom to which they are attached form a 4-, 5- or 6-membered heterocyclic ring, wherein said ring is optionally substituted with 1 substituent selected from C₁₋₄alkyl, hydroxy, halo and C(O)Me;
   R⁸, R⁹, R¹⁰, R¹⁴, R²³, R²⁴, R²⁵, R³², R³³, R³⁴, R³⁵, R³⁶, R³⁷, R³⁸, R³⁹, R⁴⁰, R⁴¹, R⁴³, R⁴⁴, R⁴⁵, R⁴⁶, R⁴⁷, R⁴⁸, R⁵⁰, R⁵², R⁵³, R⁵⁴, R⁵⁵, R⁵⁶, R⁵⁷, R⁵⁸, R⁶¹ and R⁶² are independently selected from H and C₁₋₄alkyl; or a pharmaceutically acceptable salt thereof.

In one embodiment there is provided a compound of Formula (Ib), or a pharmaceutically acceptable salt thereof, wherein Ring A is phenyl.

In one embodiment there is provided a compound of Formula (Ib), or a pharmaceutically acceptable salt thereof, wherein Ring A is bicyclic heteroaryl selected from the group consisting of:

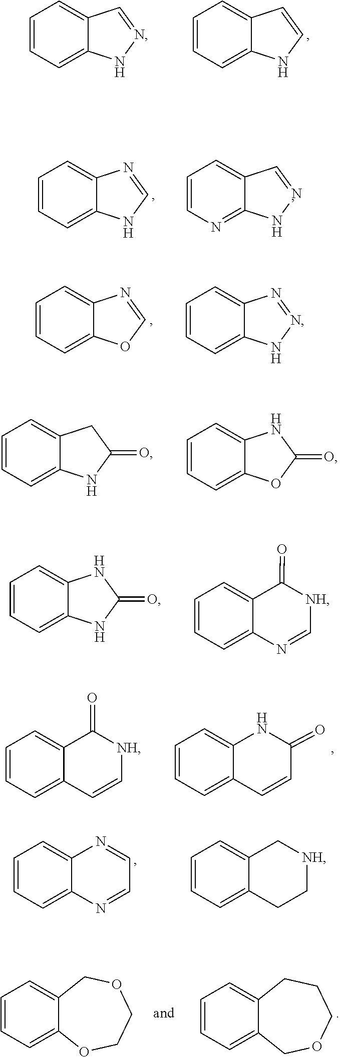

In one embodiment there is provided a compound of Formula (Ib), or a pharmaceutically acceptable salt thereof, wherein Ring A is bicyclic heteroaryl selected from the group consisting of:

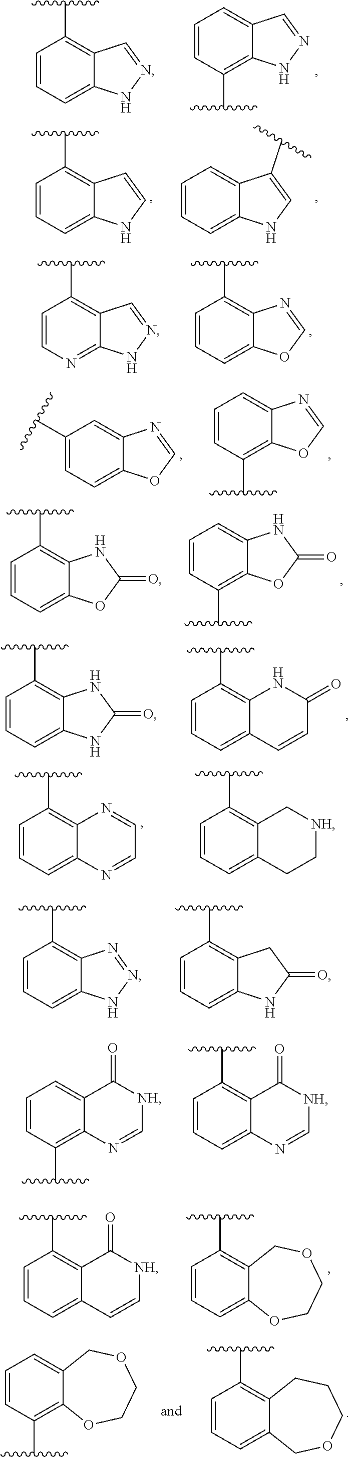

In one embodiment there is provided a compound of Formula (Ib), or a pharmaceutically acceptable salt thereof, wherein R¹⁵ is H and R¹⁶ is H.

In one embodiment there is provided a compound of Formula (Ib), or a pharmaceutically acceptable salt thereof, wherein R⁶ is H.

In one embodiment there is provided the compound of Formula (Ic):

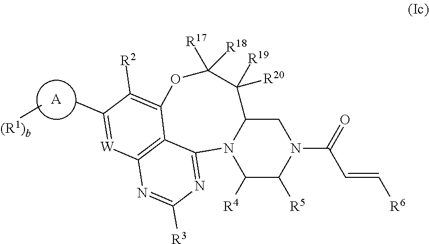

wherein:
Ring A is selected from aryl, monocyclic heteroaryl and bicyclic heteroaryl;
R¹ is independently selected from C₁₋₄alkyl, halo, hydroxy, C₁₋₄alkoxy, C₁₋₃fluoroalkyl, C₁₋₃fluoroalkoxy, cyano, acetylenyl, NR⁷R⁸, C(O)NR⁹R¹⁰, CH₂R¹¹, N═S(O)Me₂, S(O)Me and SO₂R¹²;
b is 0, 1, 2 or 3;

### W is N or CR¹³;

R² is H, cyano, halo, C₁₋₄alkyl, C₁₋₄alkoxy, C₁₋₃fluoroalkyl, NR²³R²⁴, acetylenyl or CH₂OR²⁵;
R³ is H, C₁₋₃fluoroalkyl, OR²⁶, NR²⁷R²⁸, CH₂R²⁹, SR³⁰ or C(O)R³¹;

### R⁴ is H or Me;

### R⁶ is H or Me;

R⁶ is H or CH₂NMe₂;
R⁷ is H, C₁₋₄alkyl, C(O)C₁₋₃alkyl or CO₂C₁₋₃alkyl;
R¹¹ is hydroxy, cyano, heterocyclyl, NR³²R³³, C(O)NR³⁴R³⁵ or SO₂C₁₋₃alkyl;
R¹² is C₁₋₃alkyl, C₁₋₃fluoroalkyl or NR³⁶R³⁷;
R¹³ is H, C₁₋₄alkyl, halo, C₁₋₃fluoroalkyl or C₁₋₄alkoxy;
R¹⁷ and R¹⁸ are independently selected from H and C₁₋₃alkyl;
R¹⁹ and R²⁰ are independently selected from H, C₁₋₃alkyl, and fluoro;
R²⁶ is selected from the group consisting of:
H;
C₁₋₄alkyl optionally substituted with 1 or 2 substituents independently selected from hydroxy, C₁₋₃alkoxy, halo, NR³⁸R³⁹, C(O)NR⁴⁰R⁴¹, SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl;
C₃₋₇cycloalkyl optionally substituted with 1 substituent selected from C₁₋₄alkyl, hydroxy and halo;
heterocyclyl optionally substituted with 1 or 2 substituents independently selected from C₁₋₄ alkyl, hydroxy, halo, C(O)Me, C₁₋₃alkoxy, C₁₋₃fluoroalkyl, C₃₋₇cycloalkyl, heterocyclyl and heteroaryl; and
heteroaryl optionally substituted with 1 substituent selected from C₁₋₄alkyl, hydroxy, halo, cyano and C₁₋₄alkoxy;
   R²⁷ is selected from the group consisting of:
H;
C(O)R⁴²;
C₁₋₄alkyl optionally substituted with 1 or 2 substituents independently selected from hydroxy, C₁₋₃alkoxy, halo, NR⁴³R⁴⁴, C(O)NR⁴⁵R⁴⁶, SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl;
C₃₋₇cycloalkyl optionally substituted with 1 substituent selected from C₁₋₄alkyl, hydroxy and halo;
heterocyclyl optionally substituted with 1 or 2 substituents independently selected from C₁₋₄ alkyl, hydroxy, halo, C(O)Me, C₁₋₃ alkoxy, C₁₋₃fluoroalkyl, C₃₋₇cycloalkyl, CH₂cyclopropyl, heterocyclyl and heteroaryl; and
heteroaryl optionally substituted with 1 substituent selected from C₁₋₄alkyl, hydroxy, halo, cyano and C₁₋₄alkoxy;

### R²⁸ is H or Me; or

R²⁷ and R²⁸ taken together with the nitrogen atom to which they are attached form a 4-, 5-, 6- or 7-membered heterocyclic ring, wherein said ring is optionally substituted with 1 or 2 substituents independently selected from C₁₋₄alkyl, hydroxy, halo, C(O)Me, NR⁴⁷R⁴⁸, C₁₋₃alkoxy, C₁₋₃fluoroalkyl, C₃₋₇cycloalkyl, CH₂cyclopropyl, heterocyclyl and heteroaryl;
R²⁹ is selected from the group consisting of:
H;
NR⁴⁹R⁵⁰;
C₁₋₃alkyl optionally substituted with 1 or 2 substituents independently selected from hydroxy, C₁₋₃alkoxy, halo, NR⁵¹R⁵², C(O)NR⁵³R⁵⁴, SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl;
C₃₋₇cycloalkyl optionally substituted with 1 substituent selected from C₁₋₄alkyl, hydroxy and halo;
heterocyclyl optionally substituted with 1 or 2 substituents independently selected from C₁₋₄ alkyl, hydroxy, halo, C(O)Me, C₁₋₃ alkoxy, C₁₋₃fluoroalkyl, C₃₋₇cycloalkyl, CH₂cyclopropyl, heterocyclyl and heteroaryl; and
heteroaryl optionally substituted with 1 substituent selected from C₁₋₄alkyl, hydroxy, halo, cyano and C₁₋₄alkoxy;
   R³⁰ is selected from the group consisting of:
C₁₋₄alkyl optionally substituted with 1 or 2 substituents independently selected from hydroxy, C₁₋₃alkoxy, halo, NR⁵⁵R⁵⁶, C(O)NR⁵⁷R⁵⁸, SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl;
C₃₋₇cycloalkyl optionally substituted with 1 substituent selected from C₁₋₄alkyl, hydroxy and halo;
heterocyclyl optionally substituted with 1 or 2 substituents independently selected from C₁₋₄ alkyl, hydroxy, halo, C(O)Me, C₁₋₃ alkoxy, C₁₋₃fluoroalkyl, C₃₋₇cycloalkyl, CH₂cyclopropyl, heterocyclyl and heteroaryl; and
heteroaryl optionally substituted with 1 substituent selected from C₁₋₄alkyl, hydroxy, halo, cyano and C₁₋₄alkoxy;
   R³¹ is NR⁵⁹R⁶⁰;
   R⁴² is heteroaryl optionally substituted with 1 or 2 substituents independently selected from C₁₋₄alkyl, hydroxy, halo, cyano and C₁₋₄alkoxy, or is C₁₋₄alkyl optionally substituted with 1 or 2 substituents independently selected from hydroxy, C₁₋₃alkoxy, halo and NR⁶¹R⁶²;
   R⁴⁹ and R⁵¹ are independently selected from H, C₁₋₄alkyl, heterocyclyl and heteroaryl;
   R⁵⁹ and R⁶⁰ are independently selected from H and C₁₋₄alkyl; or
   R⁵⁹ and R⁶⁰ taken together with the nitrogen atom to which they are attached form a 4-, 5- or 6-membered heterocyclic ring, wherein said ring is optionally substituted with 1 substituent selected from C₁₋₄alkyl, hydroxy, halo and C(O)Me;
   R⁸, R⁹, R¹⁰, R¹⁴, R²³, R²⁴, R²⁵, R³², R³³, R³⁴, R³⁵, R³⁶, R³⁷, R³⁸, R³⁹, R⁴⁰, R⁴¹, R⁴³, R⁴⁴, R⁴⁵, R⁴⁶, R⁴⁷, R⁴⁸, R⁵⁰, R⁵², R⁵³, R⁵⁴, R⁵⁵, R⁵⁶, R⁵⁷, R⁵⁸, R⁶¹ and R⁶² are independently selected from H and C₁₋₄alkyl;
   or a pharmaceutically acceptable salt thereof.

In one embodiment there is provided a compound of Formula (Ic), or a pharmaceutically acceptable salt thereof, wherein Ring A is phenyl.

In one embodiment there is provided a compound of Formula (Ic), or a pharmaceutically acceptable salt thereof, wherein Ring A is bicyclic heteroaryl selected from the group consisting of:

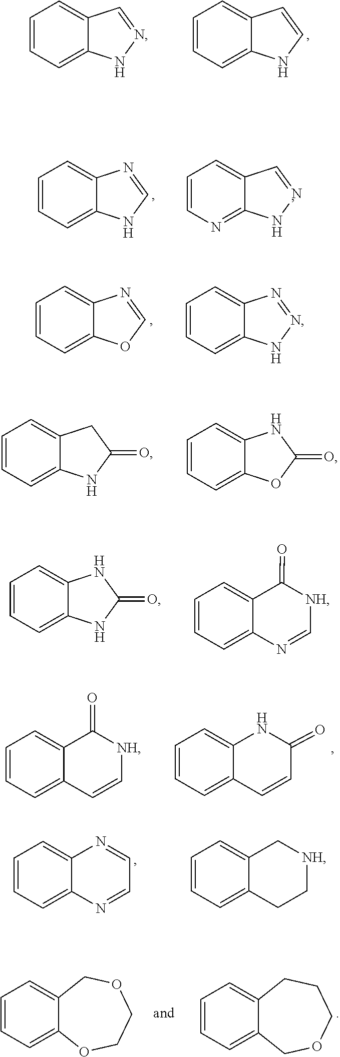

In one embodiment there is provided a compound of Formula (Ic), or a pharmaceutically acceptable salt thereof, wherein Ring A is bicyclic heteroaryl selected from the group consisting of:

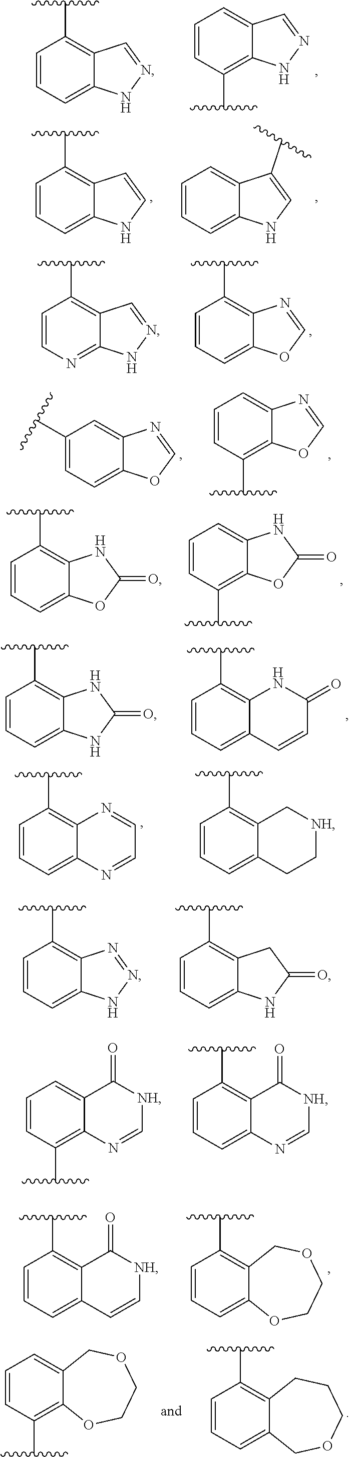

In one embodiment there is provided a compound of Formula (Ic), or a pharmaceutically acceptable salt thereof, wherein R¹⁷ is H and R¹⁸ is H.

In one embodiment there is provided a compound of Formula (Ic), or a pharmaceutically acceptable salt thereof, wherein R¹⁷, R¹⁸, R¹⁹ and R²⁰ are H.

In one embodiment there is provided a compound of Formula (Ic), or a pharmaceutically acceptable salt thereof, wherein R⁶ is H.

In a further aspect of the specification there is provided the compound of Formula (Id):

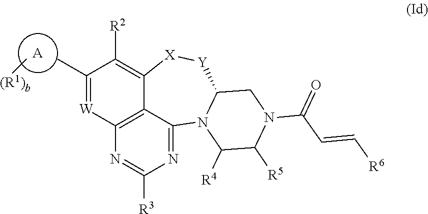

wherein:
Ring A is selected from aryl, monocyclic heteroaryl and bicyclic heteroaryl;
R¹ is independently selected from C₁₋₄alkyl, halo, hydroxy, C₁₋₄alkoxy, C₁₋₃fluoroalkyl, C₁₋₃fluoroalkoxy, cyano, acetylenyl, NR⁷R⁸, C(O)NR⁹R¹⁰, CH₂R¹¹, N═S(O)Me₂, S(O)Me and SO₂R¹²;
b is 0, 1, 2 or 3;

### W is N or CR¹³;

### X is O or NR¹⁴;

Y is CR¹⁵R¹⁶, CR¹⁷R¹⁸CR¹⁹R²⁰, C═O, or C(O)CR²¹R²²;
R² is H, cyano, halo, C₁₋₄alkyl, C₁₋₄alkoxy, C₁₋₃fluoroalkyl, NR²³R²⁴, acetylenyl or CH₂OR²⁵;
R³ is H, C₁₋₃fluoroalkyl, OR²⁶, NR²⁷R²⁸, CH₂R²⁹, SR³⁰ or C(O)R³¹;

### R⁴ is H or Me;

### R⁵ is H or Me;

R⁶ is H or CH₂NMe₂;
R⁷ is H, C₁₋₄alkyl, C(O)C₁₋₃alkyl or CO₂C₁₋₃alkyl;
R¹¹ is hydroxy, cyano, heterocyclyl, NR³²R³³, C(O)NR³⁴R³⁵ or SO₂C₁₋₃alkyl;
R¹² is C₁₋₃alkyl, C₁₋₃fluoroalkyl or NR³⁶R³⁷;
R¹³ is H, C₁₋₄alkyl, halo, C₁₋₃fluoroalkyl or C₁₋₄alkoxy;
R¹⁵, R¹⁶, R¹⁷ and R¹⁸ are independently selected from H and C₁₋₃alkyl;
R¹⁹, R²⁰, R²¹ and R²² are independently selected from H, C₁₋₃alkyl, and fluoro;
R²⁶ is selected from the group consisting of:
H;
C₁₋₄alkyl optionally substituted with 1 or 2 substituents independently selected from hydroxy, C₁₋₃alkoxy, halo, NR³⁸R³⁹, C(O)NR⁴⁰R⁴¹, SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl;
C₃₋₇cycloalkyl optionally substituted with 1 substituent selected from C₁₋₄alkyl, hydroxy and halo;
heterocyclyl optionally substituted with 1 or 2 substituents independently selected from C₁₋₄ alkyl, hydroxy, halo, C(O)Me, C₁₋₃ alkoxy, C₁₋₃fluoroalkyl, C₃₋₇cycloalkyl, heterocyclyl and heteroaryl; and
heteroaryl optionally substituted with 1 substituent selected from C₁₋₄alkyl, hydroxy, halo, cyano and C₁₋₄alkoxy;
   R²⁷ is selected from the group consisting of:
H;
C(O)R⁴²;
C₁₋₄alkyl optionally substituted with 1 or 2 substituents independently selected from hydroxy, C₁₋₃alkoxy, halo, NR⁴³R⁴⁴, C(O)NR⁴⁵R⁴⁶, SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl;
C₃₋₇cycloalkyl optionally substituted with 1 substituent selected from C₁₋₄alkyl, hydroxy and halo;
heterocyclyl optionally substituted with 1 or 2 substituents independently selected from C₁₋₄ alkyl, hydroxy, halo, C(O)Me, C₁₋₃ alkoxy, C₁₋₃fluoroalkyl, C₃₋₇cycloalkyl, CH₂cyclopropyl, heterocyclyl and heteroaryl; and
heteroaryl optionally substituted with 1 substituent selected from C₁₋₄alkyl, hydroxy, halo, cyano and C₁₋₄alkoxy;

### R²⁸ is H or Me; or

R²⁷ and R²⁸ taken together with the nitrogen atom to which they are attached form a 4-, 5-, 6- or 7-membered heterocyclic ring, wherein said ring is optionally substituted with 1 or 2 substituents independently selected from C₁₋₄alkyl, hydroxy, halo, C(O)Me, NR⁴⁷R⁴⁸, C₁₋₃alkoxy, C₁₋₃fluoroalkyl, C₃₋₇cycloalkyl, CH₂cyclopropyl, heterocyclyl and heteroaryl;
R²⁹ is selected from the group consisting of:
H;
NR⁴⁹R⁵⁰;
C₁₋₃alkyl optionally substituted with 1 or 2 substituents independently selected from hydroxy, C₁₋₃alkoxy, halo, NR⁵¹R⁵², C(O)NR⁵³R⁵⁴, SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl;
C₃₋₇cycloalkyl optionally substituted with 1 substituent selected from C₁₋₄alkyl, hydroxy and halo;
heterocyclyl optionally substituted with 1 or 2 substituents independently selected from C₁₋₄ alkyl, hydroxy, halo, C(O)Me, C₁₋₃ alkoxy, C₁₋₃fluoroalkyl, C₃₋₇cycloalkyl, CH₂cyclopropyl, heterocyclyl and heteroaryl; and
heteroaryl optionally substituted with 1 substituent selected from C₁₋₄alkyl, hydroxy, halo, cyano and C₁₋₄alkoxy;
   R³⁰ is selected from the group consisting of:
C₁₋₄alkyl optionally substituted with 1 or 2 substituents independently selected from hydroxy, C₁₋₃alkoxy, halo, NR⁵⁵R⁵⁶, C(O)NR⁵⁷R⁵⁸, SO₂Me, heteroaryl, C₃₋₇cycloalkyl and heterocyclyl, wherein said heteroaryl or C₃₋₇cycloalkyl is optionally further substituted with 1 or 2 substituents independently selected from C₁₋₄alkyl, hydroxy, halo, cyano, and C₁₋₄alkoxy and said heterocyclyl is optionally further substituted with 1 or 2 substituents independently selected from C₁₋₄alkyl, hydroxy, halo, C(O)Me, C₁₋₃alkoxy, C₁₋₃fluoroalkyl, C₃₋₇cycloalkyl, heterocyclyl and heteroaryl;
C₃₋₇cycloalkyl optionally substituted with 1 substituent selected from C₁₋₄alkyl, hydroxy and halo;
heterocyclyl optionally substituted with 1 or 2 substituents independently selected from C₁₋₄ alkyl, hydroxy, halo, C(O)Me, C₁₋₃ alkoxy, C₁₋₃fluoroalkyl, C₃₋₇cycloalkyl, CH₂cyclopropyl, heterocyclyl and heteroaryl; and
heteroaryl optionally substituted with 1 substituent selected from C₁₋₄alkyl, hydroxy, halo, cyano and C₁₋₄alkoxy;
   R³¹ is NR⁵⁹R⁶⁰;
   R⁴² is heteroaryl optionally substituted with 1 or 2 substituents independently selected from C₁₋₄alkyl, hydroxy, halo, cyano and C₁₋₄alkoxy, or is C₁₋₄alkyl optionally substituted with 1 or 2 substituents independently selected from hydroxy, C₁₋₃alkoxy, halo and NR⁶¹R⁶²;
   R⁴⁹ and R⁵¹ are independently selected from H, C₁₋₄alkyl, heterocyclyl and heteroaryl;
   R⁵⁹ and R⁶⁰ are independently selected from H and C₁₋₄alkyl; or
   R⁵⁹ and R⁶⁰ taken together with the nitrogen atom to which they are attached form a 4-, 5- or 6-membered heterocyclic ring, wherein said ring is optionally substituted with 1 substituent selected from C₁₋₄alkyl, hydroxy, halo and C(O)Me;
   R⁸, R⁹, R¹⁰, R¹⁴, R²³, R²⁴, R²⁵, R³², R³³, R³⁴, R³⁵, R³⁶, R³⁷, R³⁸, R³⁹, R⁴⁰, R⁴¹, R⁴³, R⁴⁴, R⁴⁵, R⁴⁶, R⁴⁷, R⁴⁸, R⁵⁰, R⁵², R⁵³, R⁵⁴, R⁵⁵, R⁵⁶, R⁵⁷, R⁵⁸, R⁶¹ and R⁶² are independently selected from H and C₁₋₄alkyl;
   or a pharmaceutically acceptable salt thereof.

In embodiments, the compound of Formula (Id) is a compound of Formula (Ie) in which the group R³ is H.

In embodiments, the compound of Formula (Id) or (Ie) is a compound of Formula (If) in which the group X is O.

In embodiments, the compound of Formula (Id), (Ie) or (If) is a compound of Formula (Ig) in which the group Y is CR¹⁵R¹⁶ or CR¹⁷R¹⁸CR¹⁹R²⁰, optionally wherein each of the groups R¹⁵ to R²⁰ are H.

In embodiments, the compound of Formula (Id), (Ie), (If) or (Ig) is a compound of Formula (Ih) in which the group W is CR¹³.

In embodiments, the compound of Formula (Ie), (If), (Ig) or (Ih) is a compound of Formula (Ii) in which R² is selected from H, Cl, Me or cyano.

In embodiments, the compound of Formula (Ie), (If), (Ig) or (Ih) is a compound of Formula (Ij) in which R² is Cl.

In embodiments, the compound of Formula (Ie), (If), (Ig), (Ih), (Ii) or (Ij) is a compound of Formula (Ik) in which R⁶ is H.

In embodiments, the compound of Formula (Ie), (If), (Ig), (Ih), (Ii) or (Ij) or (Ik) is a compound of Formula (Il) in which R¹³ is selected from F, Me or MeO.

In embodiments, the compound of Formula (Ie), (If), (Ig), (Ih), (Ii), (Ij), (Ik) or (Il) is a compound of Formula (Im) in which A is phenyl.

In embodiments, the compound of Formula (Im) is a compound of Formula (In) in which at least one R¹ group is hydroxy.

In embodiments, the compound of Formula (Im) or (In) is a compound of Formula (Io) in which at least one R¹ group is selected from F, Cl, MeO or CN.

In embodiments, the compound of Formula (Im), (In) or (Io) is a compound of Formula (Ip) in which any R¹ group present is located ortho- to the biaryl bond.

In embodiments, the compound of Formula (Im) is a compound of Formula (Iq) wherein the group A is

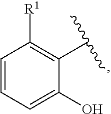

and R¹ is selected from F, Cl, MeO or CN.

In embodiments, the compound of Formula (Ie), (If), (Ig), (Ih), (Ii), (Ij), (Ik) or (Il) is a compound of Formula (Ir) in which A is bicyclic heteroaryl.

In embodiments, the compound of Formula (Ir) is a compound of Formula (Is) in which the bicyclic heteroaryl group A is selected from:

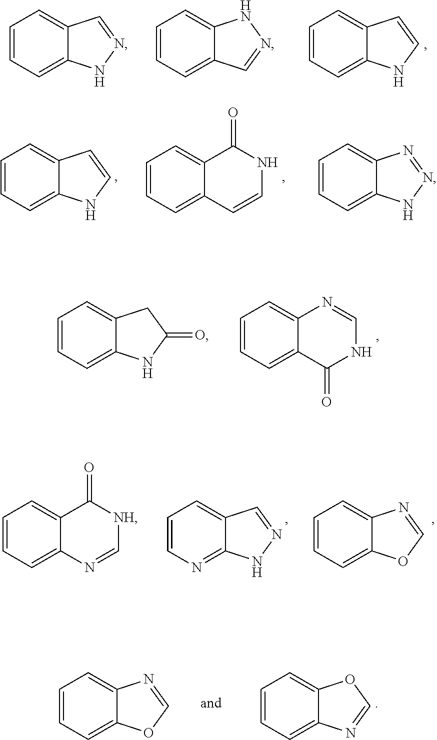

In embodiments, the compound of Formula (Iq) is a compound of Formula (It) in which the bicyclic heteroaryl group A is selected from:

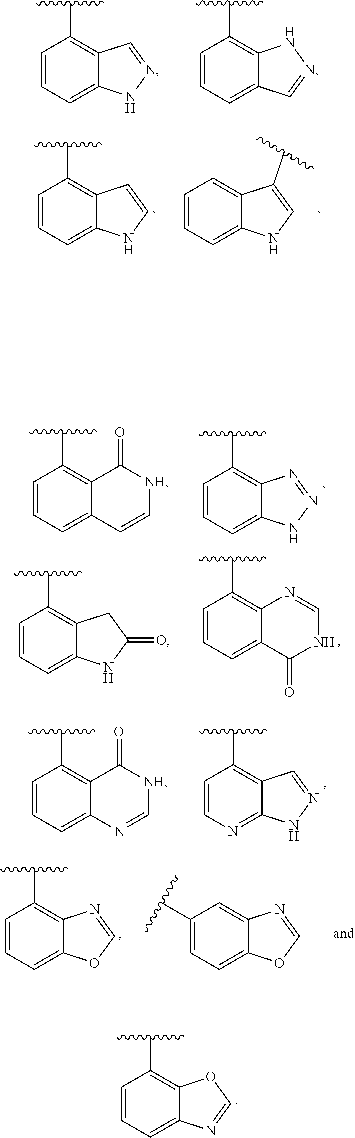

In embodiments, the compound of Formula (Iq) is a compound of Formula (Iu) in which the bicyclic heteroaryl group A is

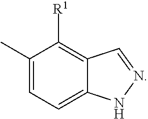

In one embodiment there is provided a compound of Formula (I), or a pharmaceutically acceptable salt thereof, wherein the compound is selected from the group consisting of:
1-[(8aS)-6-Chloro-5-(5-methyl-1H-indazol-4-yl)-8a,9,11,12-tetrahydropyrazino[2′,1′:3,4][1,4]oxazepino[5,6,7-de]quinazolin-10 (8H)-yl]-2-propen-1-one;
(E)-1-((8aS)-6-Chloro-5-(5-methyl-1H-indazol-4-yl)-8a,9,11,12-tetrahydropyrazino [2′,1′:3,4][1,4]oxazepino[5,6,7-de]quinazolin-10 (8H)-yl)-4-(dimethylamino)but-2-en-1-one;
1-[(8aS)-5-(5-Methyl-1H-indazol-4-yl)-8a,9,11,12-tetrahydropyrazino[2′,1′:3,4][1,4]oxazepino[5,6,7-de]quinazolin-10 (8H)-yl]prop-2-en-1-one;
1-[(8aR)-5-(5-Methyl-1H-indazol-4-yl)-8a,9,11,12-tetrahydropyrazino[2′,1′:3,4][1,4]oxazepino[5,6,7-de]quinazolin-10 (8H)-yl]prop-2-en-1-one;
1-[(8aR)-6-Chloro-5-(5-methyl-1H-indazol-4-yl)-8a,9,11,12-tetrahydropyrazino [2′,1′:3,4][1,4]oxazepino[5,6,7-de]quinazolin-10 (8H)-yl]prop-2-en-1-one;
1-[(8aS)-4-Chloro-5-(5-methyl-1H-indazol-4-yl)-8a,9,11,12-tetrahydropyrazino[2′,1′:3,4][1,4]oxazepino[5,6,7-de]quinazolin-10 (8H)-yl]prop-2-en-1-one;
1-[(8aS)-6-Chloro-5-(2-fluoro-6-hydroxyphenyl)-8a,9,11,12-tetrahydropyrazino[2′,1′:3,4][1,4]oxazepino[5,6,7-de]quinazolin-10 (8H)-yl]prop-2-en-1-one;
(E)-1-((8aS)-6-Chloro-5-(2-fluoro-6-hydroxyphenyl)-8a,9,11,12-tetrahydropyrazino [2′,1′:3,4][1,4]oxazepino[5,6,7-de]quinazolin-10 (8H)-yl)-4-(dimethylamino)but-2-en-1-one;
4-[(8aS)-10-Acryloyl-6-chloro-8,8a,9,10,11,12-hexahydropyrazino[2′,1′:3,4][1,4]oxazepino[5,6,7-de]quinazolin-5-yl]-1,3-dihydro-2H-benzimidazol-2-one;
1-[(8aS)-6-Chloro-5-(5-methyl-1H-pyrazolo[3,4-b]pyridin-4-yl)-8a,9,11,12-tetrahydropyrazino[2′,1′:3,4][1,4]oxazepino[5,6,7-de]quinazolin-10 (8H)-yl]prop-2-en-1-one;
1-[(8aS)-6-Chloro-5-(2-chloro-6-hydroxyphenyl)-8a,9,11,12-tetrahydropyrazino[2′,1′:3,4][1,4]oxazepino[5,6,7-de]quinazolin-10 (8H)-yl]prop-2-en-1-one;
1-[(8aS)-6-chloro-5-(2-hydroxy-6-methoxyphenyl)-8a,9,11,12-tetrahydropyrazino [2′,1′:3,4][1,4]oxazepino[5,6,7-de]quinazolin-10 (8H)-yl]prop-2-en-1-one;
2-[(8aS)-10-acryloyl-6-chloro-8,8a,9,10,11,12-hexahydropyrazino[2′,1′:3,4][1,4]oxazepino[5,6,7-de]quinazolin-5-yl]-3-hydroxybenzonitrile;
1-[(8aS)-5-(2-Amino-1,3-benzoxazol-5-yl)-6-chloro-8a,9,11,12-tetrahydropyrazino [2′,1′:3,4][1,4]oxazepino[5,6,7-de]quinazolin-10 (8H)-yl]prop-2-en-1-one;
7-[(8aS)-10-Acryloyl-6-chloro-8,8a,9,10,11,12-hexahydropyrazino[2′,1′:3,4][1,4]oxazepino[5,6,7-de]quinazolin-5-yl]-3-methyl-1,3-benzoxazol-2 (3H)-one;
N-{3-[(8aS)-10-Acryloyl-6-chloro-8,8a,9,10,11,12-hexahydropyrazino[2′,1′:3,4][1,4]oxazepino[5,6,7-de]quinazolin-5-yl]phenyl}acetamide;
1-[(8aS)-6-Chloro-5-(2,3-dihydro-5H-1,4-benzodioxepin-9-yl)-8a,9,11,12-tetrahydropyrazino[2′,1′:3,4][1,4]oxazepino[5,6,7-de]quinazolin-10 (8H)-yl]prop-2-en-1-one;
1-[(8aS)-6-Chloro-5-(2-fluoro-6-methoxyphenyl)-8a,9,11,12-tetrahydropyrazino [2′,1′:3,4][1,4]oxazepino[5,6,7-de]quinazolin-10 (8H)-yl]prop-2-en-1-one;
1-[(8aS)-6-Chloro-5-(3-fluoro-2-methoxyphenyl)-8a,9,11,12-tetrahydropyrazino [2′,1′:3,4][1,4]oxazepino[5,6,7-de]quinazolin-10 (8H)-yl]prop-2-en-1-one;
1-[(8aS)-6-Chloro-5-(2-hydroxy-3-methoxyphenyl)-8a,9,11,12-tetrahydropyrazino [2′,1′:3,4][1,4]oxazepino[5,6,7-de]quinazolin-10 (8H)-yl]prop-2-en-1-one;
1-[(8aS)-6-Chloro-5-(1,3,4,5-tetrahydro-2-benzoxepin-6-yl)-8a,9,11,12-tetrahydropyrazino [2′,1′:3,4][1,4]oxazepino[5,6,7-de]quinazolin-10 (8H)-yl]prop-2-en-1-one;
1-[(8aS)-6-Chloro-5-[2-(methylsulfonyl)phenyl]-8a,9,11,12-tetrahydropyrazino[2′,1′:3,4][1,4]oxazepino[5,6,7-de]quinazolin-10 (8H)-yl]prop-2-en-1-one;
2-[(8aS)-10-Acryloyl-6-chloro-8,8a,9,10,11,12-hexahydropyrazino[2′,1′:3,4][1,4]oxazepino[5,6,7-de]quinazolin-5-yl]-N-methylbenzamide;
3-[(8aS)-10-Acryloyl-6-chloro-8,8a,9,10,11,12-hexahydropyrazino[2′,1′:3,4][1,4]oxazepino[5,6,7-de]quinazolin-5-yl]benzenesulfonamide;
1-[(8aS)-6-Chloro-5-(quinoxalin-5-yl)-8a,9,11,12-tetrahydropyrazino[2′,1′:3,4][1,4]oxazepino[5,6,7-de]quinazolin-10 (8H)-yl]prop-2-en-1-one;
Methyl {3-[(8aS)-10-Acryloyl-6-chloro-8,8a,9,10,11,12-hexahydropyrazino[2′,1′:3,4][1,4]oxazepino[5,6,7-de]quinazolin-5-yl]phenyl}carbamate;
1-[(8aS)-6-Chloro-5-(2-methyl-1,2,3,4-tetrahydroisoquinolin-8-yl)-8a,9,11,12-tetrahydropyrazino[2′,1′:3,4][1,4]oxazepino[5,6,7-de]quinazolin-10 (8H)-yl]prop-2-en-1-one;
1-[(8aS)-6-Chloro-5-[2-(trifluoromethoxy)phenyl]-8a,9,11,12-tetrahydropyrazino[2′,1′:3,4][1,4]oxazepino[5,6,7-de]quinazolin-10 (8H)-yl]prop-2-en-1-one;
1-[(8aS)-6-Chloro-5-(3-{[dimethyl(oxido)-lambda˜6˜sulfanylidene]amino}phenyl)-8a,9,11,12-tetrahydropyrazino[2′,1′:3,4][1,4]oxazepino[5,6,7-de]quinazolin-10 (8H)-yl]prop-2-en-1-one;
1-[(8aS)-6-Chloro-5-(2,3-dimethyl-2H-indazol-7-yl)-8a,9,11,12-tetrahydropyrazino [2′,1′:3,4][1,4]oxazepino[5,6,7-de]quinazolin-10 (8H)-yl]prop-2-en-1-one;
1-[(8aS)-6-Chloro-5-[2-fluoro-5-(hydroxymethyl)phenyl]-8a,9,11,12-tetrahydropyrazino [2′,1′:3,4][1,4]oxazepino[5,6,7-de]quinazolin-10 (8H)-yl]prop-2-en-1-one;
1-[(8aS)-6-Chloro-5-(1-methyl-1H-indazol-7-yl)-8a,9,11,12-tetrahydropyrazino[2′,1′:3,4][1,4]oxazepino[5,6,7-de]quinazolin-10 (8H)-yl]prop-2-en-1-one;
1-[(8aS)-6-Chloro-5-(2,5-difluorophenyl)-8a,9,11,12-tetrahydropyrazino[2′,1′:3,4][1,4]oxazepino[5,6,7-de]quinazolin-10 (8H)-yl]prop-2-en-1-one;
1-[(8aS)-6-Chloro-5-(2-methylphenyl)-8a,9,11,12-tetrahydropyrazino[2′,1′:3,4][1,4]oxazepino[5,6,7-de]quinazolin-10 (8H)-yl]prop-2-en-1-one;
1-[(8aS)-6-Chloro-5-(2-chlorophenyl)-8a,9,11,12-tetrahydropyrazino[2′,1′:3,4][1,4]oxazepino[5,6,7-de]quinazolin-10 (8H)-yl]prop-2-en-1-one;
1-[(8aS)-6-Chloro-5-(2,3-dihydro-5H-1,4-benzodioxepin-6-yl)-8a,9,11,12-tetrahydropyrazino[2′,1′:3,4][1,4]oxazepino[5,6,7-de]quinazolin-10 (8H)-yl]prop-2-en-1-one;
8-[(8aS)-10-Acryloyl-6-chloro-8,8a,9,10,11,12-hexahydropyrazino[2′,1′:3,4][1,4]oxazepino[5,6,7-de]quinazolin-5-yl]-1-methylquinolin-2 (1H)-one;
1-[(8aS)-6-Chloro-5-(1-methyl-1H-benzimidazol-4-yl)-8a,9,11,12-tetrahydropyrazino [2′,1′:3,4][1,4]oxazepino[5,6,7-de]quinazolin-10 (8H)-yl]prop-2-en-1-one;
1-[(8aS)-6-Chloro-5-(1-methyl-1H-indol-3-yl)-8a,9,11,12-tetrahydropyrazino[2′,1′:3,4][1,4]oxazepino[5,6,7-de]quinazolin-10 (8H)-yl]prop-2-en-1-one;
4-[(8aS)-10-Acryloyl-6-chloro-8,8a,9,10,11,12-hexahydropyrazino[2′,1′:3,4][1,4]oxazepino[5,6,7-de]quinazolin-5-yl]-3-methyl-1,3-benzoxazol-2 (3H)-one;
1-[(8aS)-6-Chloro-5-(5-chloro-2-methoxyphenyl)-8a,9,11,12-tetrahydropyrazino[2′,1′:3,4][1,4]oxazepino[5,6,7-de]quinazolin-10 (8H)-yl]prop-2-en-1-one;
2-{2-[(8aS)-10-Acryloyl-6-chloro-8,8a,9,10,11,12-hexahydropyrazino[2′,1′:3,4][1,4]oxazepino[5,6,7-de]quinazolin-5-yl]phenyl}acetamide;
1-[(8aS)-6-Chloro-5-(2-chloro-4-fluorophenyl)-8a,9,11,12-tetrahydropyrazino[2′,1′:3,4][1,4]oxazepino[5,6,7-de]quinazolin-10 (8H)-yl]prop-2-en-1-one;
1-[(8aS)-6-Chloro-5-(5-fluoro-2-methoxyphenyl)-8a,9,11,12-tetrahydropyrazino[2′,1′:3,4][1,4]oxazepino[5,6,7-de]quinazolin-10 (8H)-yl]prop-2-en-1-one;
1-[(8aS)-6-Chloro-5-(4-hydroxyphenyl)-8a,9,11,12-tetrahydropyrazino[2′,1′:3,4][1,4]oxazepino[5,6,7-de]quinazolin-10 (8H)-yl]prop-2-en-1-one;
1-[(8aS)-6-Chloro-5-(1H-indol-4-yl)-8a,9,11,12-tetrahydropyrazino[2′,1′:3,4][1,4]oxazepino[5,6,7-de]quinazolin-10 (8H)-yl]prop-2-en-1-one;
1-[(8aS)-5-(4-Amino-2-methylphenyl)-6-chloro-8a,9,11,12-tetrahydropyrazino[2′,1′:3,4][1,4]oxazepino[5,6,7-de]quinazolin-10 (8H)-yl]prop-2-en-1-one;
1-[(8aS)-6-Chloro-5-(4-methylpyridin-3-yl)-8a,9,11,12-tetrahydropyrazino[2′,1′:3,4][1,4]oxazepino[5,6,7-de]quinazolin-10 (8H)-yl]prop-2-en-1-one;
5-[(8aS)-10-Acryloyl-6-chloro-8,8a,9,10,11,12-hexahydropyrazino[2′,1′:3,4][1,4]oxazepino[5,6,7-de]quinazolin-5-yl]-2-hydroxybenzonitrile;
1-[(8aS)-5-(1,3-Benzoxazol-4-yl)-6-chloro-8a,9,11,12-tetrahydropyrazino[2′,1′:3,4][1,4]oxazepino[5,6,7-de]quinazolin-10 (8H)-yl]prop-2-en-1-one;
1-[(8aS)-5-(1,3-Benzoxazol-7-yl)-6-chloro-8a,9,11,12-tetrahydropyrazino[2′,1′:3,4][1,4]oxazepino[5,6,7-de]quinazolin-10 (8H)-yl]prop-2-en-1-one;
{3-[(8aS)-10-Acryloyl-6-chloro-8,8a,9,10,11,12-hexahydropyrazino[2′,1′:3,4][1,4]oxazepino[5,6,7-de]quinazolin-5-yl]phenyl}acetonitrile;
1-[(8aS)-6-Chloro-5-(2-fluorophenyl)-8a,9,11,12-tetrahydropyrazino[2′,1′:3,4][1,4]oxazepino[5,6,7-de]quinazolin-10 (8H)-yl]prop-2-en-1-one;
1-[(8aS)-6-Chloro-5-(1H-indazol-4-yl)-8a,9,11,12-tetrahydropyrazino[2′,1′:3,4][1,4]oxazepino[5,6,7-de]quinazolin-10 (8H)-yl]prop-2-en-1-one;
1-[(8aS)-6-Chloro-5-(2,3-difluorophenyl)-8a,9,11,12-tetrahydropyrazino[2′,1′:3,4][1,4]oxazepino[5,6,7-de]quinazolin-10 (8H)-yl]prop-2-en-1-one;
1-[(8aS)-6-Chloro-5-(2-hydroxyphenyl)-8a,9,11,12-tetrahydropyrazino[2′,1′:3,4][1,4]oxazepino[5,6,7-de]quinazolin-10 (8H)-yl]prop-2-en-1-one;
1-[(8aS)-6-Chloro-5-(4-fluoro-2-methylphenyl)-8a,9,11,12-tetrahydropyrazino[2′,1′:3,4][1,4]oxazepino[5,6,7-de]quinazolin-10 (8H)-yl]prop-2-en-1-one;
1-[(8aS)-6-Chloro-5-[2-(hydroxymethyl)phenyl]-8a,9,11,12-tetrahydropyrazino[2′,1′:3,4][1,4]oxazepino[5,6,7-de]quinazolin-10 (8H)-yl]prop-2-en-1-one;
1-[(8aS)-6-Chloro-5-(2,4-difluorophenyl)-8a,9,11,12-tetrahydropyrazino[2′,1′:3,4][1,4]oxazepino[5,6,7-de]quinazolin-10 (8H)-yl]prop-2-en-1-one;
3-[(8aS)-10-Acryloyl-6-chloro-8,8a,9,10,11,12-hexahydropyrazino[2′,1′:3,4][1,4]oxazepino[5,6,7-de]quinazolin-5-yl]pyridine-4-carbonitrile;
2-[(8aS)-10-Acryloyl-6-chloro-8,8a,9,10,11,12-hexahydropyrazino[2′,1′:3,4][1,4]oxazepino[5,6,7-de]quinazolin-5-yl]benzonitrile;
1-((8aS)-6-chloro-4-fluoro-5-(2-fluoro-6-hydroxyphenyl)-8a,9,11,12-tetrahydropyrazino [2′,1′:3,4][1,4]oxazepino[5,6,7-de]quinazolin-10 (8H)-yl)prop-2-en-1-one;
1-[(8aS)-6-chloro-5-(5-methyl-1H-indazol-4-yl)-2-(morpholin-4-yl)-8a,9,11,12-tetrahydropyrazino[2′,1′:3,4][1,4]oxazepino[5,6,7-de]quinazolin-10 (8H)-yl]prop-2-en-1-one;
1-[(8aS)-6-chloro-2-[(1-cyclopropylpiperidin-4-yl)amino]-5-(5-methyl-1H-indazol-4-yl)-8a,9,11,12-tetrahydropyrazino[2′,1′:3,4][1,4]oxazepino[5,6,7-de]quinazolin-10 (8H)-yl]prop-2-en-1-one;
1-((8aS)-6-chloro-2-(3-(dimethylamino)azetidin-1-yl)-5-(5-methyl-1H-indazol-4-yl)-8a,9,11,12-tetrahydropyrazino[2′,1′:3,4][1,4]oxazepino[5,6,7-de]quinazolin-10 (8H)-yl)prop-2-en-1-one;
1-((8aS)-6-Chloro-2-((2-(dimethylamino)ethyl)amino)-5-(5-methyl-1H-indazol-4-yl)-8a,9,11,12-tetrahydropyrazino[2′,1′:3,4][1,4]oxazepino[5,6,7-de]quinazolin-10 (8H)-yl)prop-2-en-1-one; and
1-[(6aR)-3-chloro-2-(5-methyl-1H-indazol-4-yl)-5,6,6a,7,9,10-hexahydro-8H-pyrazino[1′,2′:5,6][1,5]oxazocino[4,3,2-de]quinazolin-8-yl]prop-2-en-1-one.

A further feature is any of the embodiments described above with the proviso that any of the specific Examples are individually disclaimed. For example, a further feature is any of the embodiments described above with the proviso that any of the compounds selected from the above list of examples of compounds of the invention are individually disclaimed.

In some embodiments, the compound is a compound of formula (I) excluding at least one compound recited in the Examples below. To illustrate, in some such embodiments, the compound is a compound of formula (I) excluding the compound disclosed in Example X, wherein X may be 1, 2, 3, etc. In other embodiments, the compound is a compound of formula (I) excluding the compounds disclosed in Examples Y, wherein Y may be any combination of 1, 2, 3, etc.

Unless otherwise stated, halo is selected from Cl, F, Br and I;

Cycloalkyl is a non-aromatic carbocyclic ring. The carbocyclic ring may be saturated or unsaturated, and may be bridged or unbridged. C₃₋₇ cycloalkyl is any such carbocyclic ring containing 3 to 7 carbon atoms. An example of C₃₋₇ cycloalkyl is an unsaturated non-aromatic carbocyclic ring containing 3 to 7 carbon atoms. Examples of suitable cycloalkyl groups are cyclopropyl, cyclobutyl, cyclopentyl, cyclohexyl, cycloheptyl and bicyclopentyl, such as cyclopropyl and cyclobutyl.

Heterocyclyl is a 3 to 9 membered non-aromatic, mono- or bi-cyclic ring comprising one or two heteroatoms independently selected from nitrogen, oxygen or sulphur; or an N-oxide thereof, or an S-oxide or S-dioxide thereof. The ring may be bridged or unbridged. An example of a heterocyclic ring is an unsaturated 4 to 7 membered non-aromatic, monocyclic ring comprising or two heteroatoms independently selected from nitrogen or oxygen; or an N-oxide thereof. Examples of suitable heterocyclyl groups include oxiranyl, aziridinyl, azetidinyl, oxetanyl, tetrahydrofuranyl, pyrrolidinyl, tetrahydropyranyl, piperidinyl, morpholinyl, thiomorpholinyl, and piperazinyl, such as azetidinyl, oxetanyl, pyrrolidinyl, tetrahydropyranyl, piperidinyl or morpholinyl, for example piperidinyl or morpholinyl. For the avoidance of doubt, substituents on the heterocyclyl ring may be linked via either a carbon atom or a heteroatom.

Aryl is an aromatic ring containing 6 or 10 carbon atoms. Examples of suitable aryl groups include phenyl and naphthyl, such as phenyl.

Heteroaryl is a 5, 6, 9 or 10 membered aromatic group comprising one ring or two fused rings and containing 1, 2, 3 or 4 N atoms, or one O atom, or one S atom, or 1 N atom and one S atom, or 1 N atom and one O atom, or 2 N atoms and one S atom, or 2 N atoms and one O atom. Examples of suitable heteroaryl groups include thienyl, furanyl, pyrrolyl, pyrazolyl, imidazolyl, oxazolyl, isoxazolyl, thiazolyl, isothiazolyl, triazolyl, oxadiazolyl, thiadiazolyl, tetrazolyl, pyridinyl, pyrimidinyl, pyridazinyl, pyrazinyl, indolyl, benzofuranyl, benzothienyl, benzoxazolyl, benzimidazolyl, benzotriazolyl, indazolyl, azaindolyl, azaindazolyl, quinolinyl, isoquinolinyl, quinoxalinyl, pyrrolo[1,2-b]pyridazinyl and pyrrolo[2,3-b]pyridinyl.

Monocyclic heteroaryl is an aromatic group comprising one ring and containing 1, 2, 3 or 4 N atoms, or one O atom, or one S atom, or 1 N atom and one S atom, or 1 N atom and one O atom, or 2 N atoms and one S atom, or 2 N atoms and one O atom. Examples of suitable monocyclic heteroaryl groups include thienyl, furanyl, pyrrolyl, pyrazolyl, imidazolyl, oxazolyl, isoxazolyl, thiazolyl, isothiazolyl, triazolyl, oxadiazolyl, thiadiazolyl, tetrazolyl, pyridinyl, pyrimidinyl, pyridazinyl and pyrazinyl.

Bicyclic heteroaryl is an aromatic group comprising two fused rings and containing 1, 2, 3 or 4 N atoms, or one O atom, or one S atom, or 1 N atom and one S atom, or 1 N atom and one O atom, or 2 N atoms and one S atom, or 2 N atoms and one O atom. Bicyclic heteroaryl groups include those groups where both fused rings are aromatic, or where one fused ring is aromatic and the other fused ring is partially or fully saturated. The said partially or fully saturated fused ring may also comprise a carbonyl group. Examples of suitable bicyclic heteroaryl groups include indolyl, benzofuranyl, benzothienyl, benzoxazolyl, benzimidazolyl, benzotriazolyl, indazolyl, azaindolyl, azaindazolyl, pyrrolo[1,2-b]pyridazinyl and pyrrolo[2,3-b]pyridinyl, quinolinyl, isoquinolinyl, quinazolinyl, cinnolinyl, phthalazinyl, quinoxalinyl and naphthyridinyl.

Further examples of bicyclic heteroaryl groups include the following:

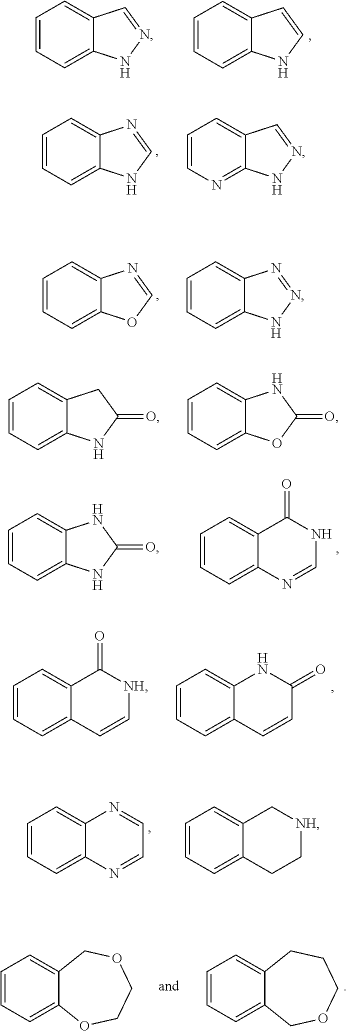

Unless otherwise stated alkyl, alkoxy, fluoroalkyl and fluoroalkoxy groups containing the requisite number of carbon atoms can be branched or unbranched. Examples of suitable C₁₋₄alkyl groups include methyl, ethyl, n-propyl, i-propyl, n-butyl, sec-butyl, i-butyl and t-butyl. Examples of suitable C₁₋₃alkyl groups include methyl, ethyl, n-propyl, and i-propyl. Examples of suitable C₁₋₄alkoxy groups include methoxy, ethoxy, n-propoxy, i-propoxy, n-butoxy, sec-butoxy and t-butoxy. Examples of suitable C₁₋₃alkoxy groups include methoxy, ethoxy, n-propoxy and i-propoxy. Examples of suitable C₁₋₃ fluoroalkyl groups include fluoromethyl, difluoromethyl, trifluoromethyl and 2,2,2-trifluoroethyl. Examples of suitable C₁₋₃ fluoroalkoxy groups include fluoromethoxy, difluoromethoxy, trifluoromethoxy and 2,2,2-trifluoroethoxy.

For the avoidance of doubt, where multiple substituents are independently selected from a given group, the selected substituents may comprise the same substituents or different substituents from within the given group. By way of example only, where ring A is aryl substituted with (R¹)_{b}, and where b is 2, the two R¹ substituents could be the same, for instance both fluoro, or could be different, for instance one fluoro and one hydroxy.

For the further avoidance of doubt, the use of “ [symbol] ” in formulas of this specification denotes the point of attachment between different groups.

Where any embodiment within this specification includes a group which is said to be “optionally substituted”, then a further embodiment will include that embodiment wherein the said group is unsubstituted.

The compounds of Formula (I) have one or more chiral centres and it will be recognised that the compound of Formula (I) may be prepared, isolated and/or supplied with or without the presence, in addition, of one or more of the other possible stereoisomeric forms of the compound of Formula (I) in any relative proportions. The preparation of stereoenriched or stereopure compounds may be carried out by standard techniques of organic chemistry that are well known in the art, for example by synthesis from stereoenriched or stereopure starting materials, use of an appropriate stereoenriched or stereopure catalyst during synthesis, and/or by resolution of a racemic or partially enriched mixture of stereoisomers, for example via chiral chromatography.

It has been found that the compounds of Formula (I) having the stereochemistry shown in the compounds of Formula (Id) and (Ie) exhibit higher activity as inhibitors of G12C Ras mutant protein than compounds having the opposite stereochemistry or compounds lacking the group X-Y, that in compounds of Formula (I) tethers C-5 of the quinazoline ring to the piperazine ring. In particular, compounds of Formula (Id) and (Ie) in which the group X-Y is OCH₂ or OCH₂CH₂ have proven to be particularly active inhibitors of G12C Ras mutant protein. It is believed that this improved potency derives from the tethering group X-Y holding the piperazine ring in a conformation close to, or in, its optimal conformation for binding to G12C Ras mutant protein thus lowering the energy required for binding of the inhibitor to the target protein.

In particular, the compounds of Formula (I) may possess axial chirality, by virtue of restricted rotation around a biaryl bond and as such may exist as mixtures of atropisomers with enantiomeric excess between about 0% and >98% e.e. When a compound is a pure atropisomer, the stereochemistry at each chiral center may be specified by either aR or aS. Such designations may also be used for mixtures that are enriched in one atropisomer. By way of example only, the following moiety may exhibit atropisomerism and be capable of resolution into the OR and aS atropisomers by chiral chromatography (NB. the identity of R will dictate which isomer is the aR/aS isomer):

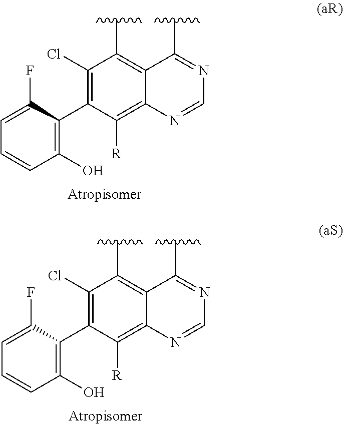

Further description of atropisomerism and axial chirality and rules for assignment of configuration can be found in Eliel, E. L. & Wilen, S. H. ‘Stereochemistry of Organic Compounds’ John Wiley and Sons, Inc. 1994.

For use in a pharmaceutical context it may be preferable to provide a compound of Formula (I) or a pharmaceutically acceptable salt thereof without large amounts of the other stereoisomeric forms being present.

Accordingly, in one embodiment there is provided a composition comprising a compound of Formula (I) or a pharmaceutically acceptable salt thereof, optionally together with one or more of the other stereoisomeric forms of the compound of Formula (I) or pharmaceutically acceptable salt thereof, wherein the compound of Formula (I) or pharmaceutically acceptable salt thereof is present within the composition with a diastereomeric excess (% de) of 90%.

In a further embodiment the % de in the above-mentioned composition is 95%.

In a further embodiment the % de in the above-mentioned composition is 98%.

In a further embodiment the % de in the above-mentioned composition is 99%.

In a further embodiment there is provided a composition comprising a compound of Formula (I) or a pharmaceutically acceptable salt thereof, optionally together with one or more of the other stereoisomeric forms of the compound of Formula (I) or pharmaceutically acceptable salt thereof, wherein the compound of Formula (I) or pharmaceutically acceptable salt thereof is present within the composition with an enantiomeric excess (% ee) of 90%.

In a further embodiment the % ee in the above-mentioned composition is 95%.

In a further embodiment the % ee in the above-mentioned composition is 98%.

In a further embodiment the % ee in the above-mentioned composition is 99%.

In a further embodiment there is provided a composition comprising a compound of Formula (I) or a pharmaceutically acceptable salt thereof, optionally together with one or more of the other stereoisomeric forms of the compound of Formula (I) or pharmaceutically acceptable salt thereof, wherein the compound of Formula (I) or pharmaceutically acceptable salt thereof is present within the composition with an enantiomeric excess (% ee) of 90% and a diastereomeric excess (% de) of ≥90%.

In further embodiments of the above-mentioned composition the % ee and % de may take any combination of values as listed below:
The % ee is ≤5% and the % de is ≥80%.
The % ee is ≤5% and the % de is ≥90%.
The % ee is ≤5% and the % de is ≥95%.
The % ee is ≤5% and the % de is ≥98%.
The % ee is ≥95% and the % de is ≥95%.
The % ee is ≥98% and the % de is ≥98%.
The % ee is ≥99% and the % de is ≥99%.

In a further embodiment there is provided a pharmaceutical composition which comprises a compound of the Formula (I) or a pharmaceutically acceptable salt thereof, in association with a pharmaceutically acceptable excipient.

In one embodiment there is provided a pharmaceutical composition which comprises a compound of the Formula (I) or a pharmaceutically acceptable salt thereof, in association with a pharmaceutically acceptable excipient, optionally further comprising one or more of the other stereoisomeric forms of the compound of Formula (I) or pharmaceutically acceptable salt thereof, wherein the compound of Formula (I) or pharmaceutically acceptable salt thereof is present within the composition with an enantiomeric excess (% ee) of 90%.

In a further embodiment the % ee in the above-mentioned composition is 95%.

In a further embodiment the % ee in the above-mentioned composition is 98%.

In a further embodiment the % ee in the above-mentioned composition is 99%.

In one embodiment there is provided a pharmaceutical composition which comprises a compound of the Formula (I) or a pharmaceutically acceptable salt thereof, in association with a pharmaceutically acceptable excipient, optionally further comprising one or more of the other stereoisomeric forms of the compound of Formula (I) or pharmaceutically acceptable salt thereof, wherein the compound of Formula (I) or pharmaceutically acceptable salt thereof is present within the composition with a diastereomeric excess (% de) of 90%.

In a further embodiment the % de in the above-mentioned composition is 95%.

In a further embodiment the % de in the above-mentioned composition is 98%.

In a further embodiment the % de in the above-mentioned composition is 99%.

In one embodiment there is provided a pharmaceutical composition which comprises a compound of the Formula (I) or a pharmaceutically acceptable salt thereof, in association with a pharmaceutically acceptable excipient, optionally further comprising one or more of the other stereoisomeric forms of the compound of Formula (I) or pharmaceutically acceptable salt thereof, wherein the compound of Formula (I) or pharmaceutically acceptable salt thereof is present within the composition with an enantiomeric excess (% ee) of 90% and a diastereomeric excess (% de) of ≥90%.

In further embodiments of the above-mentioned pharmaceutical composition the % ee and % de may take any combination of values as listed below:
The % ee is ≥95% and the % de is ≥95%.
The % ee is ≥98% and the % de is ≥98%.
The % ee is ≥99% and the % de is ≥99%.

The compounds of Formula (I) and pharmaceutically acceptable salts thereof may be prepared, used or supplied in amorphous form, crystalline form, or semi-crystalline form and any given compound of Formula (I) or pharmaceutically acceptable salt thereof may be capable of being formed into more than one crystalline/polymorphic form, including hydrated (e.g. hemi-hydrate, a mono-hydrate, a di-hydrate, a tri-hydrate or other stoichiometry of hydrate) and/or solvated forms. It is to be understood that the present specification encompasses any and all such solid forms of the compound of Formula (I) and pharmaceutically acceptable salts thereof.

In further embodiments there is provided a compound of Formula (I), which is obtainable by the methods described in the ‘Examples’ section hereinafter.

The present specification is intended to include all isotopes of atoms occurring in the present compounds. Isotopes will be understood to include those atoms having the same atomic number but different mass numbers. For example, isotopes of hydrogen include tritium and deuterium. Isotopes of carbon include ¹³C and ¹⁴C. Isotopically labelled compounds of Formula (I) can generally be prepared by conventional techniques known to those skilled in the art or by processes analogous to those described in the accompanying Examples using appropriate isotopically labelled reagents in place of the non-labelled reagents previously employed.

A suitable pharmaceutically acceptable salt of a compound of the Formula (I) is, for example, an acid addition salt. A suitable pharmaceutically acceptable salt of a compound of the Formula (I) may be, for example, an acid-addition salt of a compound of the Formula (I), for example an acid-addition salt with an inorganic or organic acid.

A further suitable pharmaceutically acceptable salt of a compound of the Formula (I) is, for example, a salt formed within the human or animal body after administration of a compound of the Formula (I) to said human or animal body.

The compound of Formula (I) or pharmaceutically acceptable salt thereof may be prepared as a co-crystal solid form. It is to be understood that a pharmaceutically acceptable co-crystal of a compound of the Formula (I) or pharmaceutically acceptable salts thereof, form an aspect of the present specification.

For the avoidance of doubt it is to be understood that where in this specification a group is qualified by ‘hereinbefore defined’ or ‘defined herein’ the said group encompasses the first occurring and broadest definition as well as each and all of the alternative definitions for that group.

Another aspect of the present specification provides a process for preparing a compound of the Formula (I), or a pharmaceutically acceptable salt thereof. A suitable process is illustrated by the following representative process variants in which, unless otherwise stated, ring A, W, X, Y and R¹ to R⁶ have any of the meanings defined hereinbefore. Necessary starting materials may be obtained by standard procedures of organic chemistry. The preparation of such starting materials is described in conjunction with the following representative process variants and within the accompanying Examples. Alternatively, necessary starting materials are obtainable by analogous procedures to those illustrated which are within the ordinary skill of an organic chemist.

Compounds of formula (I) may be made by, for example, reaction of a suitable compound of formula (II) with a compound of formula (III), where L is a leaving group, for example halo (such as chloro) in the presence of a suitable base (such as triethylamine); or where L is OH and the reaction is carried out under standard amide bond forming conditions (for example in the presence of an amide coupling reagent (such as HBTU) and a suitable base (DIPEA)).

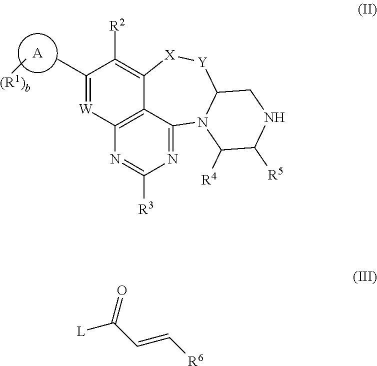

Compounds of formula (II) may be made by, for example, de-protection of a compound of formula (IV), where P is a suitable protecting group (for example Boc, which may be removed by treatment with acid).

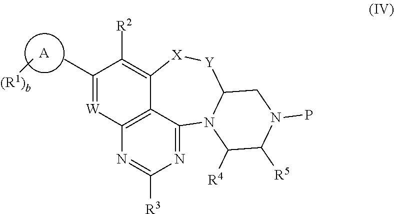

Compounds of formula (IV) may be made by, for example, a Suzuki-Miyaura coupling reaction between a compound of formula (V) and;
an aryl or heteroaryl boronic acid/ester (when Q is iodo, bromo or chloro); or
an aryl or heteroaryl bromide/iodide/chloride (when Q is a boronic acid/ester);
   in the presence of a suitable palladium catalyst (such as Pd-118), and a suitable base (such as caesium carbonate) in a suitable solvent (such as dioxane/water) at a suitable temperature (such as 80-110° C.).

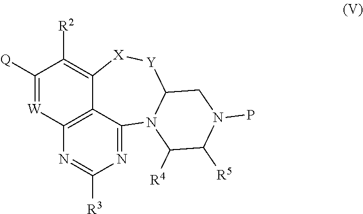

Compounds of formula (V) may be made by, for example, reaction of a compound of formula (VI) with a suitable coupling reagent (such as BOP reagent—1H-benzo[d]-[1,2,3]triazol-1-yl)oxy)tris(dimethylamino)phosphonium hexafluorophosphate) in the presence of a strong base (such as DBU) in a suitable solvent (such as acetonitrile).

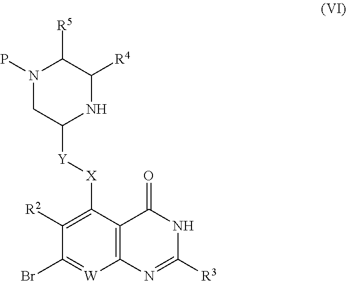

Compounds of formula (VI) may be made by, for example, reaction of a compound of formula (VII) with a compound of formula (VIII) in the presence of a suitable base (such as sodium hydride) in a suitable solvent (such as THF).

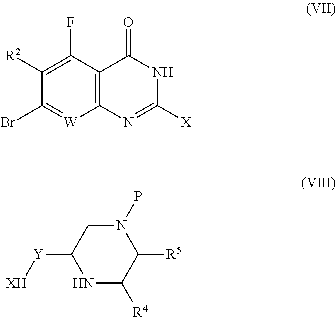

Compounds of formula (VI) where R³ is NR²⁷R²⁸ may be made, for example, from a compound of formula (IX), where X is halo (such as chloro) by reaction with an amine R²⁷R²⁸NH in the presence of a suitable base (such as DIPEA), in a suitable solvent (such as isopropanol) at a suitable temperature (such as 80-100° C.). Compounds of formula (IX) may be made by reaction of compounds of formula (X) with a compound of formula (VIII) as described above.

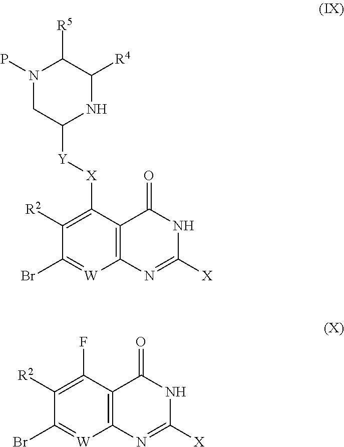

Compounds of formula (X) where X is Cl may be made, for example, by reaction of a compound of formula (XI) with thiophosgene in a suitable solvent (such as dioxan) at a suitable temperature (such as 80-110° C.). Compounds of formula (XI) may be prepared, for example, from aryl nitriles of formula (XII), by reaction with a suitable oxidising agent (such as hydrogen peroxide) in the presence of a suitable base (such as potassium carbonate) in a suitable solvent (such as DMSO).

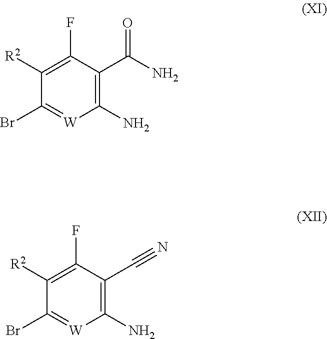

Compounds of formula (VII) may be made by, for example, reaction of a compound of formula (XII) with an acid of formula R³CO₂H (for example formic acid when R³ is hydrogen) in the presence of a suitable acid (such as sulfuric acid) at a suitable temperature (such as 80-100° C.). Alternatively, compounds of formula (VII) may be made by, for example, reaction of an aryl acid of formula (XIII) with a reagent of formula R³C(═NH)NH₂ (for example formamidine acetate when R³ is hydrogen) in a suitable solvent (such as ethanol) at a suitable temperature (such as 70-90° C.).

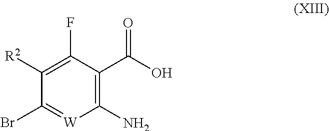

It is to be understood that other permutations of the process steps in the process variants described above are also possible.

When a pharmaceutically acceptable salt of a compound of Formula (I) is required it may be obtained by, for example, reaction of said compound with a suitable acid or suitable base. When a pharmaceutically acceptable pro-drug of a compound of Formula (I) is required, it may be obtained using a conventional procedure.

It will also be appreciated that, in some of the reactions mentioned hereinbefore, it may be necessary or desirable to protect any sensitive functionalities in the compounds. The instances where protection is necessary or desirable, and suitable methods for protection, are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy, it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an alkoxycarbonyl group such as a t-butoxycarbonyl group may be removed, for example, by treatment with a suitable acid as hydrochloric, sulphuric, formic, phosphoric or trifluoroacetic acid, and an arylmethoxycarbonyl group such as a benzyloxycarbonyl group may be removed, for example, by hydrogenation over a catalyst such as palladium-on-carbon, or by treatment with a Lewis acid, such as boron tris(trifluoroacetate). A suitable alternative protecting group for a primary amino group is, for example, a phthaloyl group, which may be removed by treatment with an alkylamine, for example dimethylaminopropylamine, or hydrazine.

A suitable protecting group for a hydroxy group is, for example, an acyl group, for example an alkanoyl group such as acetyl, an aroyl group, for example benzoyl, an arylmethyl group, for example benzyl, or a trialkyl or diarylalkyl silane, such as TBDMS or TBDPS. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

Certain of the intermediates defined herein are novel and these are provided as further features of the specification.

Biological Assays

The following assays were used to measure the effects of the compounds of the present specification.

### KRasG12C Functional Assay

The inactive GDP loaded biotinylated KRas^{G12C} protein was expressed, purified and GDP loaded in house. All enzyme and substrate solutions were prepared in assay buffer containing 20 mM HEPES (pH 7.5), 5 mM MgCl2, 150 mM NaCl, and 0.01% Tween 20. 10 nM GDP loaded biotinylated KRasG12c and 37.5 ng/ml Streptavidin Europium Cryptate (Cisbio) were prepared in assay buffer, 5 μl was dispensed into each well of a 384 polystyrene, Hibase, medium binding white assay plate (Greiner, #784075) containing test and reference samples prepared in DMSO and the samples incubated for 4 hrs. In a separate mix 20 nM GST-Raf Ras binding domain (GST-Raf RBD, purified in house) and 4 μg/ml anti-GST XL665 antibody (Cisbio) was prepared in assay buffer containing 50 mM Potassium Fluoride and 0.05 mg/ml BSA and equilibrated for 4 hours before adding 0.6 μM Guanosine 5′-[γ-thio]triphosphate (GTPγS, Sigma) and 0.08 μM SOS (purified in house). 5 μl of the GST-RAF RBD mix was then dispensed into each well of the assay plate. This addition initiates the nucleotide exchange reaction and transition of inactive GDP loaded KRas^{G12C} to active GTPγS KRas^{G12C}. This is detected simultaneously via the specific binding interaction between active GTPγS KRas^{G12C} with GST-Raf RBD which brings the europium and XL665 into close proximity enabling an increased FRET signal to be detected on a Pherastar (BMG) plate reader equipped with the HTRF filter module. Any compound which prevents the activation of KRas via inhibiting the nucleotide exchange process, or inhibits the active KRas:Raf RBD binding interaction, will result in a reduced FRET signal. IC₅₀ values were calculated from normalised dose-response response FRET data curve fitted in Genedata screener (Basel, Switzerland).

### KRasG12C Mass Spectrometry Adducting Assay

The inactive GDP loaded biotinylated KRas^{G12C} protein was expressed, purified and GDP loaded in house. Enzyme solutions were prepared in assay buffer containing 20 mM HEPES (pH 7.5), 5 mM MgCl2, and 150 mM NaCl. 4 μM GDP loaded biotinylated KRas^{G12C} was prepared in assay buffer and 50 μl added into each well of a 96 well polypropylene assay plate (Greiner, #651201) containing 500n1 of 1 mM test compounds (final concentration 10 μM), this was allowed to react for 4 hours before the addition of 50 μl 1% Formic acid to quench the reaction. The plate was sealed before reading on a Xevo G2 QTOF (Waters) and Acquity LC system (Waters). 101 of sample was injected onto a Xbridge BEH300; C4; 3.5 um; 2.1×50 mm column (Waters) running a 3 minute gradient. Blank samples were run in between each test sample.

Data was analysed in Mass Lynx software (Waters), the Total ion count (TIC) trace was used and the eluted protein peak data combined. Using the combined spectrum the data was deconvoluted using MaxEnt1 method. The peak area for apo-protein KRas^{G12C} (APO) and KRAS+relative cmpd mass (adduct) were measured, and a percentage adduct was calculated using the following calculation:

Percent adduct=100*(area of adduct peak/(sum of APO+adduct peaks)

The data shown in Table A were generated for the Examples (the data below may be a result from a single experiment or an average of two or more experiments).

TABLE A

| | KRasG12C functional assay | KRasG12C M.S. Binding |
|---|---|---|
| Example | IC₅₀ value (μM) | Mean adduct % |
| 1 | 0.102 | 95 |
| 2 | 38.857 | 2 |
| 3 | 0.082 | 96 |
| 4 | 0.518 | 74 |
| 5 | 0.798 | 91 |
| 6 | 2.368 | 68 |
| 7 | 0.214 | 95 |
| 8 | 0.324 | 81 |
| 9 | 0.124 | 100 |
| 10 | 0.244 | 30 |
| 11 | 0.097 | 92 |
| 12 | 0.381 | 76 |
| 13 | 2.004 | 64 |
| 14 | 2.535 | 53 |
| 15 | 22.661 | 12 |
| 16 | 0.032 | 100 |
| 17 | >100.000 | 0 |
| 18 | 0.868 | 100 |
| 19 | 41.83 | 0 |
| 20 | 0.191 | 100 |
| 21 | 7.180 | 20 |
| 22 | 1.774 | 34 |
| 23 | 8.838 | 20 |
| 24 | 4.409 | 27 |
| 25 | 0.616 | 89 |
| 26 | 2.055 | 71 |
| 27 | 3.033 | 73 |
| 28 | 9.927 | 26 |
| 29 | 5.016 | 45 |
| 30 | 9.414 | 22 |
| 31 | 6.969 | 14 |
| 32 | 8.319 | 33 |
| 33 | 6.910 | 24 |
| 34 | 3.466 | 8 |
| 35 | 1.482 | 87 |
| 36 | 6.377 | 21 |
| 37 | 2.730 | 49 |
| 38 | 1.276 | 82 |
| 39 | 0.803 | 79 |
| 40 | 0.955 | 67 |
| 41 | 0.479 | 100 |
| 42 | 0.247 | 100 |
| 43 | 1.495 | 56 |
| 44 | 3.620 | 51 |
| 45 | 1.576 | 72 |
| 46 | 1.508 | 65 |
| 47 | 9.825 | 9 |
| 48 | 6.306 | 68 |
| 49 | 7.529 | 28 |
| 50 | 0.956 | 100 |
| 51 | 5.095 | 57 |
| 52 | 5.363 | 17 |
| 53 | 1.694 | 74 |
| 54 | 2.570 | 60 |
| 55 | 4.029 | 26 |
| 56 | 4.386 | 0 |
| 57 | 2.416 | 68 |
| 58 | 7.091 | 42 |
| 59 | 5.848 | 42 |
| 60 | 0.437 | 100 |
| 61 | 2.219 | 64 |
| 62 | 0.355 | 100 |
| 63 | 0.896 | 87 |
| 64 | 1.570 | 75 |
| 65 | 3.457 | 49 |
| 66 | 1.447 | 84 |
| 67 | 6.530 | 17 |
| 68 | 1.472 | 62 |
| 69 | 0.029 | 100 |
| 70 | 0.445 | 67 |
| 71 | 0.226 | 83 |
| 72 | 39.391 | 5 |
| 73 | 1.898 | 8 |
| 74 | 0.012 | 94 |
| 75 | 0.019 | 57 |
| 76 | 22.385 | 0 |
| 77 | 0.021 | 100 |
| 78 | 0.037 | 81 |
| 79 | 1.721 | 0 |
| 80 | 0.018 | 90 |
| 81 | 0.012 | 100 |
| 82 | 0.166 | 26 |

According to a further aspect of the specification there is provided a pharmaceutical composition, which comprises a compound of the Formula (I), or a pharmaceutically acceptable salt thereof, as defined hereinbefore in association with a pharmaceutically acceptable excipient.

Suitable pharmaceutically acceptable excipients for a tablet formulation include, for example, inert diluents, granulating and disintegrating agents, binding agents, lubricating agents, preservative agents and antioxidants. A further suitable pharmaceutically acceptable excipient may be a chelating agent. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may alternatively be in the form of hard gelatin capsules in which the active ingredient is mixed with an inert solid diluent, or as soft gelatin capsules in which the active ingredient is mixed with water or an oil.

Aqueous suspensions generally contain the active ingredient in finely powdered form together with one or more suspending agents, dispersing or wetting agents. The aqueous suspensions may also contain one or more preservatives, anti-oxidants, colouring agents, flavouring agents, and/or sweetening agents.

Oily suspensions may be formulated by suspending the active ingredient in a vegetable oil or in a mineral oil. The oily suspensions may also contain a thickening agent. Sweetening agents such as those set out above, and flavouring agents may be added to provide a palatable oral preparation. These compositions may be preserved by the addition of an anti-oxidant.

Dispersible powders and granules suitable for preparation of an aqueous suspension by the addition of water generally contain the active ingredient together with a dispersing or wetting agent, suspending agent and one or more preservatives. Additional excipients such as sweetening, flavouring and colouring agents, may also be present.

The pharmaceutical compositions of the specification may also be in the form of oil-in-water emulsions. The oily phase may be a vegetable oil or a mineral oil or a mixture of any of these. The emulsions may also contain sweetening, flavouring and preservative agents.

Syrups and elixirs may be formulated with sweetening agents, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent system.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, oral administration to humans will generally require, for example, from 1 mg to 2 g of active agent to be administered compounded with an appropriate and convenient amount of excipients which may vary from about 3 to about 98 percent by weight of the total composition. It will be understood that, if a large dosage is required, multiple dosage forms may be required, for example two or more tablets or capsules, with the dose of active ingredient divided conveniently between them. Typically, unit dosage forms will contain about 10 mg to 0.5 g of a compound of this specification, although a unit dosage form may contain up to 1 g. Conveniently, a single solid dosage form may contain between 1 and 300 mg of active ingredient.

The size of the dose for therapeutic or prophylactic purposes of compounds of the present specification will naturally vary according to the nature and severity of the disease state, the age and sex of the animal or patient and the route of administration, according to well-known principles of medicine.

In using compounds of the present specification for therapeutic or prophylactic purposes it will generally be administered so that a daily dose in the range, for example, 1 mg/kg to 100 mg/kg body weight is received, given if required in divided doses. In general, lower doses will be administered when a parenteral route is employed. Thus, for example, for intravenous administration, a dose in the range, for example, 1 mg/kg to 25 mg/kg body weight will generally be used. Oral administration is however preferred, particularly in tablet form.

We have found that the compounds of the present specification possess potent anti-tumour activity which it is believed is obtained by way of inhibition of G12C mutant RAS proteins, which are involved in cell-signalling leading to the proliferation and survival of tumour cells.

Accordingly, the compounds of the present specification may be of value as anti-tumour agents, in particular as selective inhibitors of the proliferation, survival, motility, dissemination and invasiveness of mammalian cancer cells leading to inhibition of tumour growth and survival and to inhibition of metastatic tumour growth. Particularly, the compounds of the present specification may be of value as anti-proliferative and anti-invasive agents in the containment and/or treatment of solid tumour disease. Particularly, the compounds of the present specification may be useful in the prevention or treatment of those tumours which are sensitive to inhibition of G12C mutant Ras and that are involved in the cell-signalling leading to the proliferation and survival of tumour cells.

According to a further aspect of the specification there is provided a compound of the Formula (I), or a pharmaceutically acceptable salt thereof, as defined hereinbefore for use as a medicament in a warm-blooded animal such as man.

According to a further aspect of the specification, there is provided a compound of the Formula (I), or a pharmaceutically acceptable salt thereof, as defined hereinbefore for use in the production of an anti-proliferative effect in a warm-blooded animal such as man.

According to a further aspect of the specification there is provided a compound of the Formula (I), or a pharmaceutically acceptable salt thereof, as defined hereinbefore for use in a warm-blooded animal such as man as an anti-invasive agent in the containment and/or treatment of solid tumour disease.

According to a further aspect of the specification, there is provided the use of a compound of the Formula (I), or a pharmaceutically acceptable salt thereof, as defined hereinbefore, for the production of an anti-proliferative effect in a warm-blooded animal such as man.

According to a further aspect of the specification there is provided the use of a compound of the Formula (I), or a pharmaceutically acceptable salt thereof, as defined hereinbefore, in the manufacture of a medicament for use in the production of an anti-proliferative effect in a warm-blooded animal such as man.

According to a further aspect of the specification there is provided the use of a compound of the Formula (I), or a pharmaceutically acceptable salt thereof, as defined hereinbefore, in the manufacture of a medicament for use in a warm-blooded animal such as man as an anti-invasive agent in the containment and/or treatment of solid tumour disease.

According to a further aspect of the specification there is provided a method for producing an anti-proliferative effect in a warm-blooded animal, such as man, in need of such treatment which comprises administering to said animal an effective amount of a compound of the Formula (I), or a pharmaceutically acceptable salt thereof, as defined hereinbefore.

According to a further aspect of the specification there is provided a method for producing an anti-invasive effect by the containment and/or treatment of solid tumour disease in a warm-blooded animal, such as man, in need of such treatment which comprises administering to said animal an effective amount of a compound of the Formula (I), or a pharmaceutically acceptable salt thereof, as defined hereinbefore.

According to a further aspect of the specification, there is provided a compound of the Formula (I), or a pharmaceutically acceptable salt thereof, as defined hereinbefore, for use in the prevention or treatment of cancer in a warm-blooded animal such as man.

According to a further aspect of the specification there is provided the use of a compound of the Formula (I), or a pharmaceutically acceptable salt thereof, as defined hereinbefore in the manufacture of a medicament for use in the prevention or treatment of cancer in a warm-blooded animal such as man.

According to a further aspect of the specification there is provided a method for the prevention or treatment of cancer in a warm-blooded animal, such as man, in need of such treatment which comprises administering to said animal an effective amount of a compound of the Formula (I), or a pharmaceutically acceptable salt thereof, as defined hereinbefore.

According to a further aspect of the specification, there is provided a compound of the Formula (I), or a pharmaceutically acceptable salt thereof, as defined hereinbefore for use in the prevention or treatment of solid tumour disease in a warm-blooded animal such as man.

According to a further aspect of the specification there is provided the use of a compound of the Formula (I), or a pharmaceutically acceptable salt thereof, as defined hereinbefore, in the manufacture of a medicament for use in the prevention or treatment of solid tumour disease in a warm-blooded animal such as man.

According to a further aspect of the specification there is provided a method for the prevention or treatment of solid tumour disease in a warm-blooded animal, such as man, in need of such treatment which comprises administering to said animal an effective amount of a compound of the Formula (I), or a pharmaceutically acceptable salt thereof, as defined hereinbefore.

According to a further aspect of the specification there is provided a compound of the Formula (I), or a pharmaceutically acceptable salt thereof, as defined hereinbefore, for use in the prevention or treatment of tumours which are sensitive to inhibition of G12C mutant Ras.

According to a further aspect of the specification there is provided the use of a compound of the Formula (I), or a pharmaceutically acceptable salt thereof, as defined hereinbefore, in the manufacture of a medicament for use in the prevention or treatment of those tumours which are sensitive to inhibition of G12C mutant Ras.

According to a further aspect of the specification there is provided a method for the prevention or treatment of those tumours which are sensitive to inhibition of G12C mutant RAS, which comprises administering to said animal an effective amount of a compound of the Formula (I), or a pharmaceutically acceptable salt thereof, as defined hereinbefore.

According to a further aspect of the specification there is provided a compound of the Formula (I), or a pharmaceutically acceptable salt thereof, as defined hereinbefore for use in providing an inhibitory effect on G12C mutant Ras.

According to a further aspect of the specification there is provided the use of a compound of the Formula (I), or a pharmaceutically acceptable salt thereof, as defined hereinbefore in the manufacture of a medicament for use in providing an inhibitory effect on G12C mutant Ras.

According to a further aspect of the specification there is also provided a method for providing an inhibitory effect on G12C mutant RAS which comprises administering an effective amount of a compound of the Formula (I), or a pharmaceutically acceptable salt thereof, as defined hereinbefore.

According to a further aspect of the specification there is provided a compound of the Formula (I), or a pharmaceutically acceptable salt thereof, as defined hereinbefore, for use in providing a selective inhibitory effect on G12C mutant Ras

According to a further aspect of the specification there is provided the use of a compound of the Formula (I), or a pharmaceutically acceptable salt thereof, as defined hereinbefore, in the manufacture of a medicament for use in providing a selective inhibitory effect on G12C mutant Ras.

According to a further aspect of the specification there is also provided a method for providing a selective inhibitory effect on G12C mutant Ras which comprises administering an effective amount of a compound of the Formula (I), or a pharmaceutically acceptable salt thereof, as defined hereinbefore.

Described herein are compounds that can bind to G12C mutant Ras. In biochemical and cell based assays the compounds of the present specification are shown to be potent G12C mutant Ras protein binders and may therefore be useful in the treatment of disorders mediated by KRas, NRas or HRas G12C mutations, in particular in the treatment of cancers expressing G12C mutated KRas, NRas or HRas proteins, such as pancreatic, colorectal, uterine, bile duct, stomach, bladder, cervical, testicular germ cell and non-small cell lung cancer and multiple myeloma, diffuse large B cell lymphoma, rhabdomyosarcoma and cutaneous squamous cell carcinoma.

According to a further aspect of the specification there is provided a compound of the Formula (I), or a pharmaceutically acceptable salt thereof, as defined hereinbefore, for use in the treatment of disorders mediated by KRas, NRas or HRas G12C mutations.

According to a further aspect of the specification there is provided a method for treating disorders mediated by KRas, NRas or HRas G12C mutations, which comprises administering an effective amount of a compound of the Formula (I), or a pharmaceutically acceptable salt thereof, as defined hereinbefore.

According to a further aspect of the specification there is provided the use of a compound of the Formula (I), or a pharmaceutically acceptable salt thereof, as defined hereinbefore, in the manufacture of a medicament for use in the treatment of disorders mediated by KRas, NRas or HRas G12C mutations.

According to a further aspect of the specification there is provided a compound of the Formula (I), or a pharmaceutically acceptable salt thereof, as defined hereinbefore, for use in the treatment of non-small cell lung cancer or colorectal cancer.

According to a further aspect of the specification there is provided a compound of the Formula (I), or a pharmaceutically acceptable salt thereof, as defined hereinbefore, for use in the treatment of non-small cell lung cancer.

According to a further aspect of the specification there is provided a method for treating non-small cell lung cancer or colorectal cancer, which comprises administering an effective amount of a compound of the Formula (I), or a pharmaceutically acceptable salt thereof, as defined hereinbefore.

According to a further aspect of the specification there is provided a method for treating non-small cell lung cancer, which comprises administering an effective amount of a compound of the Formula (I), or a pharmaceutically acceptable salt thereof, as defined hereinbefore.

According to a further aspect of the specification there is provided the use of a compound of the Formula (I), or a pharmaceutically acceptable salt thereof, as defined hereinbefore, in the manufacture of a medicament for use in the treatment of breast or gynaecological cancers.

According to a further aspect of the specification there is provided the use of a compound of the Formula (I), or a pharmaceutically acceptable salt thereof, as defined hereinbefore, in the manufacture of a medicament for use in the treatment of non-small cell lung cancer or colorectal cancer.

According to a further aspect of the specification there is provided the use of a compound of the Formula (I), or a pharmaceutically acceptable salt thereof, as defined hereinbefore, in the manufacture of a medicament for use in the treatment of non-small cell lung cancer.

The anti-cancer treatment defined herein may be applied as a sole therapy or may involve, in addition to the compounds of the specification, conventional surgery or radiotherapy or chemotherapy.

Accordingly, in one embodiment there is provided a compound of Formula (I), or a pharmaceutically acceptable salt thereof, and an additional anti-tumour substance for the conjoint treatment of cancer.

According to this aspect of the specification there is provided a combination suitable for use in the treatment of cancer comprising a compound of the Formula (I) or a pharmaceutically acceptable salt thereof and another anti-tumour agent.

Therefore in a further aspect of the specification there is provided a compound of the Formula (I), or a pharmaceutically acceptable salt thereof, in combination with another anti-tumour agent.

Although the compounds of the Formula (I) are primarily of value as therapeutic agents for use in warm-blooded animals (including man), they are also useful whenever it is required to inhibit G12C mutant Ras. Thus, they are useful as pharmacological standards for use in the development of new biological tests and in the search for new pharmacological agents.

Another aspect of the present specification is based on identifying a link between the G12C KRas, HRas or NRas mutation status of a patient and potential susceptibility to treatment with a compound of Formula (I). A Ras inhibitor, such as a compound of Formula (I), may then advantageously be used to treat patients with G12C KRas, HRas or NRas mutations who may be resistant to other therapies. This therefore provides opportunities, methods and tools for selecting patients for treatment with a compound of Formula (I), particularly cancer patients. The selection is based on whether the tumour cells to be treated possess wild-type or G12C mutant KRAS, HRAS or NRAS gene. The G12C KRAS, HRAS or NRAS gene status could therefore be used as a biomarker to indicate that selecting treatment with a compound of Formula (I) may be advantageous.

According to one aspect of the specification there is provided a method for selecting a patient for treatment with a compound of Formula (I), the method comprising providing a tumour cell-containing sample from a patient; determining whether the RAS gene in the patient's tumour cell-containing sample encodes for wild-type (glycine at position 12) or mutant (cysteine at position 12) KRas, HRas or NRas protein; and selecting a patient for treatment with a compound of Formula (I) based thereon.

The method may include or exclude the actual patient sample isolation step. Thus, according to one aspect of the specification there is provided a method for selecting a patient for treatment with a compound of Formula (I), the method comprising determining whether the RAS gene in a tumour cell-containing sample previously isolated from the patient encodes for wild-type (glycine at position 12) or mutant (cysteine at position 12) KRas, HRas or NRas protein; and selecting a patient for treatment with a compound of Formula (I) based thereon.

In one embodiment, the patient is selected for treatment with a compound of Formula (I) if the tumour cell DNA has a G12C mutant KRAS gene.

In one embodiment, the patient is selected for treatment with a compound of Formula (I) if the tumour cell DNA has a G12C mutant HRAS gene.

In one embodiment, the patient is selected for treatment with a compound of Formula (I) if the tumour cell DNA has a G12C mutant NRAS gene.

According to another aspect of the specification there is provided a compound of Formula (I), or a pharmaceutically acceptable salt thereof, for use in treating cancers with tumour cells identified as harbouring a G12C mutant KRAS gene.

According to another aspect of the specification there is provided a compound of Formula (I), or a pharmaceutically acceptable salt thereof, for use in treating cancers with tumour cells identified as harbouring a G12C mutant HRAS gene.

According to another aspect of the specification there is provided a compound of Formula (I), or a pharmaceutically acceptable salt thereof, for use in treating cancers with tumour cells identified as harbouring a G12C mutant NRAS gene.

According to another aspect of the specification there is provided a method of treating cancers with tumour cells identified as harbouring a G12C mutant KRAS, HRAS or NRAS gene comprising administering an effective amount of a compound of Formula (I) or a pharmaceutically acceptable salt thereof.

In still further embodiments, the specification relates to a pharmaceutical composition comprising a compound of Formula (I) for use in the prevention and treatment of cancer with tumour cells identified as harbouring a G12C mutant KRAS, HRAS or NRAS gene.

### EXAMPLES

The specification will now be illustrated in the following Examples in which, unless stated otherwise:

(i) all syntheses were carried out at ambient temperature, i.e. in the range 17 to 25 [symbol] C and under an atmosphere of an inert gas such as nitrogen unless otherwise stated;

(ii) evaporations were carried out by rotary evaporation or utilising Genevac equipment or Biotage v10 evaporator in vacuo and work up procedures were carried out after removal of residual solids by filtration;

(iii) flash column chromatography was performed on Merck Kieselgel silica (Art. 9385) or on reversed phase silica (Fluka silica gel 90 C18) or on Silicycle cartridges (40-63 μm silica, 4 to 330 g weight) or on Grace Resolv™ cartridges (4-120 g) or on RediSep Rf 1.5 Flash columns or on RediSep Rf high performance Gold Flash columns (150-415 g weight) or on RediSep Rf Gold C18 Reversed-phase columns (20-40 μm silica) or on Interchim puriFlash cartridges (50 μm silica, 4-800 g) either manually or automated using an Isco CombiFlash Companion system or similar system;

(iv) preparative reverse phase HPLC was performed on a Waters instrument (600/2700 or 2525) fitted with a ZMD or ZQ ESCi mass spectrometers and a Waters X-Terra or a Waters X-Bridge or a Waters SunFire reverse-phase column (C-18, 5 microns silica, 19 mm or 50 mm diameter, 100 mm length, flow rate of 40 mL/minute) using decreasingly polar mixtures of water (containing 1% ammonia) and acetonitrile or decreasingly polar mixtures of water (containing 0.1% formic acid) and acetonitrile as eluents;

(vi) yields, where present, are not necessarily the maximum attainable;

(vii) in general, the structures of end products of the Formula I were confirmed by nuclear magnetic resonance (NMR) spectroscopy; NMR chemical shift values were measured on the delta scale [proton magnetic resonance spectra were determined using a Bruker Avance 500 (500 MHz), Bruker Avance 400 (400 MHz), Bruker Avance 300 (300 MHz) or Bruker DRX (300 MHz) instrument]; measurements were taken at ambient temperature unless otherwise specified; the following abbreviations have been used: s, singlet; d, doublet; t, triplet; q, quartet; m, multiplet; dd, doublet of doublets; ddd, doublet of doublet of doublet; dt, doublet of triplets; bs, broad signal;

(viii) in general, end products of the Formula I were also characterized by mass spectroscopy following liquid chromatography (LCMS or UPLC); in general, reverse-phase C18 silica was used with a flow rate of 1 mL/minute and detection was by Electrospray Mass Spectrometry and by UV absorbance recording a wavelength range of 220-320 nm. Analytical UPLC was performed on CSH C18 reverse-phase silica, using a Waters XSelect CSH C18 column with dimensions 2.1×50 mm and particle size 1.7 micron). Gradient analysis was employed using decreasingly polar mixtures as eluent, for example decreasingly polar mixtures of water (containing 0.1% formic acid or 0.1% ammonia) as solvent A and acetonitrile as solvent B. A typical 2 minute analytical UPLC method would employ a solvent gradient over 1.3 minutes, at approximately 1 mL per minute, from a 97:3 mixture of solvents A and B respectively to a 3:97 mixture of solvents A and B. The reported molecular ion corresponds to the [M+H]+ unless otherwise specified; for molecules with multiple isotopic patterns (Br, Cl, etc.) the reported value is the one obtained for the lowest isotope mass unless otherwise specified;

(ix) ion exchange purification was generally performed using an SCX-2 (Biotage) cartridge;

(x) where reactions refer to the use of a microwave, one of the following microwave reactors were used: Biotage Initiator, Personal Chemistry Emrys Optimizer, Personal Chemistry Smithcreator or CEM Explorer;

(xi) intermediate purity was assessed by thin layer chromatographic, mass spectroscopy, LCMS, UPLC/MS, HPLC and/or NMR analysis;

(xii) the following abbreviations have been used:
DCM dichloromethane
DEA diethylamine
DIPEA diisopropylethylamine
DMA N,N-dimethylacetamide
DMF N,N-dimethylformamide
DMSO dimethylsulfoxide
d.e. diastereomeric excess
EtOAc ethyl acetate
EtOH ethanol
HATU (1-[bis(dimethylamino)methylene]-1H-1,2,3-triazolo[4,5-b]pyridinium 3-oxid hexafluorophosphate)
HCl hydrochloric acid
HPLC high performance liquid chromatography
MeCN acetonitrile
MecOH methanol
NMR nuclear magnetic resonance
i-PrOH isopropanol
SFC supercritical fluid chromatography
TBME tert-butyl methyl ether
TEA triethylamine
TFA trifluoroacetic acid
THF tetrahydrofuran
tR retention time
   Compounds are otherwise referred to by their IUPAC names or were named with 2015 ACD/Chem Sketch from ACD Labs.

### 2-Amino-4-bromo-6-fluorobenzonitrile

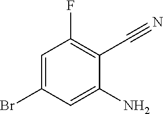 [SMILES: N#Cc1c(N)cc(Br)cc1F]

28% Ammonium hydroxide (8 ml, 64 mmol) was added to a microwave vial containing 4-bromo-2,6-difluorobenzonitrile (2 g, 9.17 mmol) in i-PrOH (5 ml). The resulting solution was capped and stirred at 80° C. for 4 hours. This reaction was repeated 4 more times, then the reaction vials carefully vented. The reaction mixtures were combined and poured into water (300 ml) and stirred for 15 minutes. A white solid was filtered which was washed with water and dried by suction to give 2-amino-4-bromo-6-fluorobenzonitrile (9.82 g, 100%). 1H NMR (500 MHz, DMSO, 27° C.) 6.73 (2H, s), 6.76 (1H, dd), 6.81 (1H, dd). m/z: ES− [M−H]− 213

### 6-Amino-4-bromo-3-chloro-2-fluorobenzonitrile

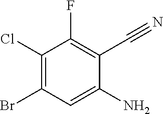 [SMILES: N#Cc1c(N)cc(Br)c(Cl)c1F]

1-Chloropyrrolidine-2,5-dione (7.26 g, 54.36 mmol) was added in one portion to a solution of 2-amino-4-bromo-6-fluorobenzonitrile (9.74 g, 45.3 mmol) in i-PrOH (45 ml) preheated to 60° C. The reaction mixture was brought gradually to 80° C. and stirred for twenty minutes then allowed to cool. The reaction mixture was evaporated and the residue dissolved in dichloromethane (300 ml), washed with water (150 ml) then dried (MgSO₄) and evaporated. The residue was purified by flash silica chromatography, elution gradient 0 to 25% ethyl acetate/heptane. Fractions containing desired compound were combined and evaporated to afford 6-amino-4-bromo-3-chloro-2-fluorobenzonitrile (3.97 g, 35%). NMR (500 MHz, DMSO) 6.83 (s, 2H), 7.02 (d, J=1.7 Hz, 1H). m/z: ES− [M−H]− 247

### 7-Bromo-6-chloro-5-fluoroquinazolin-4 (3H)-one

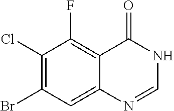 [SMILES: O=c1[nH]cnc2cc(Br)c(Cl)c(F)c12]

Sulfuric acid (1.27 ml, 23.87 mmol) was added to 6-amino-4-bromo-3-chloro-2-fluorobenzonitrile (3.97 g, 15.91 mmol) in formic acid (40 ml). The resulting solution was stirred at 100° C. for 30 minutes. The reaction mixture was cooled in an ice bath then diluted with water (80 ml). The resulting suspension was stirred for 10 minutes then filtered. The solid was washed sequentially with water:i-PrOH (16 ml, 1:1), i-PrOH:TBME (16 ml, 1:1) then TBME (16 ml). The solid was air dried for 10 minutes then dried in a vacuum oven to afford 7-bromo-6-chloro-5-fluoroquinazolin-4 (3H)-one (3.87 g, 88%) as a white solid. 1H NMR (500 MHz, DMSO, 27° C.) 7.92 (1H, d), 8.14 (1H, s), 12.53 (1H, s). m/z: ES− [M−H]− 275

### Tert-butyl (S)-3-(((7-bromo-6-chloro-4-hydroxyquinazolin-5-yl)oxy)methyl)piperazine-1-carboxylate

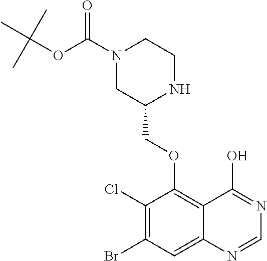 [SMILES: CC(C)(C)OC(=O)N1CCN[C@H](COc2c(Cl)c(Br)cc3ncnc(O)c23)C1]

60% Sodium hydride (0.7 g, 17.51 mmol) was added portionwise to tert-butyl (S)-3-(hydroxymethyl)piperazine-1-carboxylate (1.89 g, 8.76 mmol) and 7-bromo-6-chloro-5-fluoroquinazolin-4-ol (2.03 g, 7.3 mmol) in THF (50 ml) cooled to 0° C. The resulting mixture was stirred at 0° C. for 5 minutes, allowed to warm to room temperature then heated to 65° C. and stirred for 2 hours. A further 60% sodium hydride (0.07 g, 1.75 mmol) was added to tert-butyl (S)-3-(hydroxymethyl)piperazine-1-carboxylate (0.19 g, 0.88 mmol) in THF (2 ml) at room temperature. This was stirred for 10 minutes then this solution was added to the reaction mixture then stirred for a further 1 hour at 65° C. and allowed to cool to room temperature with stirring overnight. The reaction mixture was diluted with EtOAc (200 ml), and water (20 ml). The aqueous phase was taken to pH5 with acetic acid, then taken to pH 8 with NaHCO₃ and the two phases separated. The aqueous phase was extracted with EtOAc (100 ml). The organic phases were combined, dried and reduced. The residue was purified by flash silica chromatography, elution gradient 0 to 20% MeOH in DCM. Pure fractions were evaporated to dryness to afford tert-butyl (S)-3-(((7-bromo-6-chloro-4-hydroxyquinazolin-5-yl)oxy)methyl)piperazine-1-carboxylate (2.64 g, 76%) as a white foam. 1H NMR (500 MHz, DMSO, 27° C.) 1.39 (9H, s), 2.52-2.84 (3H, m), 2.88 (1H, dt), 2.96 (1H, dd), 3.74 (1H, d), 3.93 (2H, d), 4.05 (2H, d), 7.84 (1H, s), 8.09 (1H, s). m/z: ES+ [M+H]+ 473

### 2-Methyl-2-propanyl (8aS)-5-bromo-6-chloro-8a,9,11,12-tetrahydropyrazino[2′,1′:3,4][1,4]oxazepino[5,6,7-de]quinazoline-10 (8H)-carboxylate

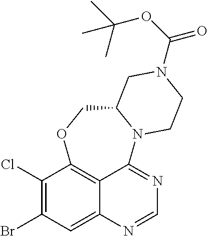 [SMILES: CC(C)(C)OC(=O)N1CCN2c3ncnc4cc(Br)c(Cl)c(c34)OC[C@@H]2C1]

2,3,4,6,7,8,9,10-Octahydropyrimido[1,2-a]azepine (2.08 ml, 13.93 mmol) was added portionwise to tert-butyl (S)-3-(((7-bromo-6-chloro-4-hydroxyquinazolin-5-yl)oxy)methyl)piperazine-1-carboxylate (2.64 g, 5.57 mmol) and ((1H-benzo[d][1,2,3]triazol-1-yl)oxy)tris(dimethylamino)phosphonium hexafluorophosphate(V) (3.2 g, 7.24 mmol) in acetonitrile (50 ml) cooled at 0° C. over a period of 5 minutes. The resulting mixture was stirred at 0° C. for 10 minutes then at room temperature for 16 hours. Further ((1H-benzo[d][1,2,3]triazol-1-yl)oxy)tris(dimethylamino)phosphonium hexafluorophosphate(V) (3.2 g, 7.24 mmol) and 2,3,4,6,7,8,9,10-octahydropyrimido[1,2-a]azepine (2.08 ml, 13.93 mmol) added. The reaction was stirred for a further 54 hours at room temperature then absorbed onto silica and purified by flash silica chromatography, elution gradient 0 to 50% EtOAc in heptane. Pure fractions were evaporated to dryness to afford 2-methyl-2-propanyl (8aS)-5-bromo-6-chloro-8a,9,11,12-tetrahydropyrazino[2′,1′:3,4][1,4]oxazepino[5,6,7-de]quinazoline-10 (8H)-carboxylate (1.92 g, 76%) as a white foam. 1H NMR (500 MHz, DMSO, 27° C.) 1.43 (9H, s), 3.05 (2H, s), 3.21 (1H, ddd), 3.90 (1H, d), 3.96-4.1 (2H, m), 4.61 (2H, qd), 4.78 (1H, d), 7.78 (1H, d), 8.51 (1H, d). m/z: ES+ [M+H]+ 455

### 2-Methyl-2-propanyl (8aS)-6-chloro-5-(5-methyl-1H-indazol-4-yl)-8a,9,11,12-tetrahydropyrazino[2′,1′:3,4][1,4]oxazepino[5,6,7-de]quinazoline-10 (8H)-carboxylate

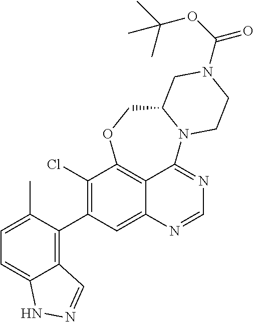 [SMILES: Cc1ccc2[nH]ncc2c1-c1cc2ncnc3c2c(c1Cl)OC[C@@H]1CN(C(=O)OC(C)(C)C)CCN31]

Pd(PPh₃)₄ (0.3 g, 0.26 mmol) was added to 2-methyl-2-propanyl (8aS)-5-bromo-6-chloro-8a,9,11,12-tetrahydropyrazino[2′,1′:3,4][1,4]oxazepino[5,6,7-de]quinazoline-10 (8H)-carboxylate (1.18 g, 2.58 mmol) and (5-methyl-1H-indazol-4-yl)boronic acid (0.68 g, 3.87 mmol) in a degassed mixture of 2M Na₂CO₃ (3 ml) and dioxane (12 ml). The resulting suspension was stirred at 100° C. for 15 hours in a microwave reactor. The mixture was diluted with DCM (150 ml), and washed with water (20 ml), then brine (20 ml). The organic phase was dried with MgSO₄, filtered and evaporated to afford crude product. The crude product was purified by flash silica chromatography, elution gradient 0 to 10% MeOH in DCM. Pure fractions were evaporated to dryness to afford 2-methyl-2-propanyl (8aS)-6-chloro-5-(5-methyl-1H-indazol-4-yl)-8a,9,11,12-tetrahydropyrazino[2′,1′:3,4][1,4]oxazepino[5,6,7-de]quinazoline-10 (8H)-carboxylate (1.04 g, 80%) as a white solid. 1H NMR (500 MHz, DMSO, 27° C.) 1.44 (9H, s), 2.13 (3H, d), 3.16 (2H, s), 3.24 (1H, td), 3.93 (1H, d), 3.99-4.18 (2H, m), 4.58-4.76 (2H, m), 4.82 (1H, d), 7.31-7.34 (2H, m), 7.47 (1H, dt), 7.52 (1H, d), 8.56 (1H, d), 13.10 (1H, s). m/z: ES+ [M+H]+ 507.

### (8aS)-6-chloro-5-(5-methyl-1H-indazol-4-yl)-8,8a,9,10,11,12-hexahydropyrazino[2′,1′:3,4][1,4]oxazepino[5,6,7-de]quinazoline

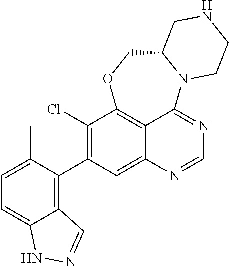 [SMILES: Cc1ccc2[nH]ncc2c1-c1cc2ncnc3c2c(c1Cl)OC[C@@H]1CNCCN31]

2,2,2-Trifluoroacetic acid (3 ml, 39.2 mmol) was added to 2-methyl-2-propanyl (8aS)-6-chloro-5-(5-methyl-1H-indazol-4-yl)-8a,9,11,12-tetrahydropyrazino[2′,1′:3,4][1,4]oxazepino[5,6,7-de]quinazoline-10 (8H)-carboxylate (1.04 g, 2.05 mmol) in DCM (15 ml) cooled at 0° C. The resulting solution was stirred at room temperature for 16 hours then evaporated to dryness. The residue was purified by ion exchange chromatography, using an SCX2 column. The desired product was eluted from the column using 1M NH₃ in MecOH. Pure fractions were evaporated to dryness to afford (8aS)-6-chloro-5-(5-methyl-1H-indazol-4-yl)-8,8a,9,10,11,12-hexahydropyrazino[2′,1′:3,4][1,4]oxazepino[5,6,7-de]quinazoline (0.76 g, 91%) as a yellow film. 1H NMR (500 MHz, DMSO, 27° C.) 2.13 (3H, s), 2.65-2.77 (2H, m), 2.97 (1H, d), 3.03 (2H, t), 3.82-3.97 (1H, m), 4.45-4.56 (1H, m), 4.60 (1H, dd), 4.89 (1H, d), 7.29 (1H, s), 7.32 (1H, d), 7.48 (1H, d), 7.51 (1H, d), 8.51 (1H, s), 13.10 (1H, s). m/z: ES+ [M+H]+ 407

### Example 1, 1-[(8aS)-6-Chloro-5-(5-methyl-1H-indazol-4-yl)-8a,9,11,12-tetrahydropyrazino-[2′,1′:3,4][1,4]oxazepino[5,6,7-de]quinazolin-10 (8H)-yl]-2-propen-1-one

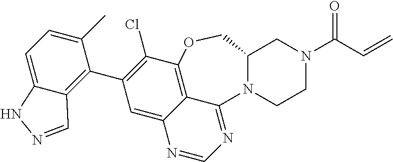 [SMILES: C=CC(=O)N1CCN2c3ncnc4cc(-c5c(C)ccc6[nH]ncc56)c(Cl)c(c34)OC[C@@H]2C1]

A solution of acryloyl chloride (99 mg, 1.09 mmol) in DMA (0.5 ml) was added to a stirred suspension of (8aS)-6-chloro-5-(5-methyl-1H-indazol-4-yl)-8,8a,9,10,11,12-hexahydropyrazino[2′,1′:3,4][1,4]oxazepino[5,6,7-de]quinazoline (405 mg, 1 mmol), and triethylamine (0.42 ml, 3 mmol) in DMA (1 ml) cooled at 0° C. The resulting mixture was stirred at 0° C. for 30 minutes. The reaction mixture was diluted with a few drops of MeOH and DMSO (1 ml) then filtered. The filtrate was purified by preparative HPLC (Waters CSH C18 OBD column, 5μ silica, 30 mm diameter, 100 mm length), using decreasingly polar mixtures of water (containing 1% NH₃) and MeCN as eluents. Fractions containing the desired compound were evaporated to give 1-[(8aS)-6-chloro-5-(5-methyl-1H-indazol-4-yl)-8a,9,11,12-tetrahydropyrazino[2′,1:3,4]-[1,4]oxazepino[5,6,7-de]quinazolin-10 (8H)-yl]-2-propen-1-one (125 mg, 27%) as a solid. 1H NMR (500 MHz, DMSO, 27° C.) 2.14 (3H, d), 2.99-3.14 (1H, m), 3.21-3.39 (1H+H2O, m), 3.39-3.51 (1H, m), 4.05-4.12 (1H, m), 4.24 (1H, dd), 4.44 (1H, dd), 4.59-4.91 (3H, m), 5.75 (1H, dd), 6.18 (1H, d), 6.8-6.96 (1H, m), 7.27-7.39 (2H, m), 7.47 (1H, d), 7.52 (1H, d), 8.57 (1H, s), 13.11 (1H, s). m/z: ES+ [M+H]+ 461

### Example 2, 1-[(8aS)-6-Chloro-5-(5-methyl-1H-indazol-4-yl)-8a,9,11,12-tetrahydropyrazino[2′,1′: 3,4][1,4]oxazepino[5,6,7-de]quinazolin-10 (8H)-yl]-2-propen-1-one, Atropisomer 1; and

### Example 3, 1-[(8aS)-6-chloro-5-(5-methyl-1H-indazol-4-yl)-8a,9,11,12-tetrahydropyrazino[2′,1′: 3,4][1,4]oxazepino[5,6,7-de]quinazolin-10 (8H)-yl]-2-propen-1-one, Atropisomer 2

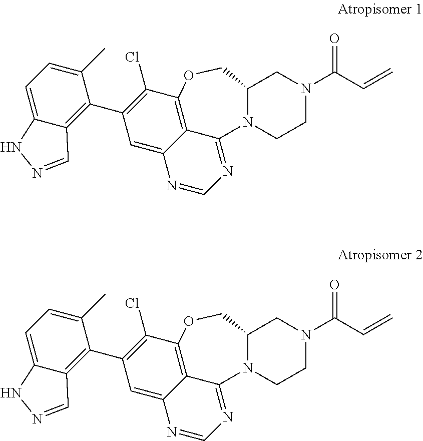 [SMILES: C=CC(=O)N1CCN2c3ncnc4cc(-c5c(C)ccc6[nH]ncc56)c(Cl)c(c34)OC[C@@H]2C1 ; C=CC(=O)N1CCN2c3ncnc4cc(-c5c(C)ccc6[nH]ncc56)c(Cl)c(c34)OC[C@@H]2C1]

1-[(8aS)-6-Chloro-5-(5-methyl-1H-indazol-4-yl)-8a,9,11,12-tetrahydropyrazino-[2′,1′:3,4][1,4]oxazepino[5,6,7-de]quinazolin-10 (8H)-yl]-2-propen-1-one, Example 1, (40 mg of a mixture of atropisomers) was chirally separated using SFC conditions on Chiralcel OD-H, 20×250 mm, 5 micron column, using mobile phase: 50% MecOH+0.1% NH₃/50% scCO₂ Flow rate 60 ml/min. Detection at UV @ 220 nm. Two peaks were observed, and collected. The first eluted peak (Atropisomer 1) was isolated (6.1 mg, d.e. 100%). 1H NMR (500 MHz, CDCl₃, 27° C.) 2.24 (3H, s), 3.03-3.22 (1H, m), 3.2-3.38 (1H, m), 3.51-3.73 (1H, m), 3.89-4.21 (2H, m), 4.45-4.85 (3H, m), 4.95-5.12 (1H, m), 5.82 (1H, dd), 6.40 (1H, d), 6.57-6.69 (1H, m), 7.36 (1H, d), 7.49 (1H, dd), 7.56 (1H, s), 7.58 (1H, s), 8.67 (1H, s), 10.14 (1H, s). m/z: ES+ [M+H]+ 461. Chiral analysis method Phenomonex Lux C1, 100×3.0 mm id, 3 micron, Mobile phase A=scCO₂, B-MeOH+0.1% NH₃, Flow rate 2.0 ml/min, Isocratic 50% B. Retention time 1.82 minutes. This was followed by the second eluted peak (Atropisomer 2; 8.5 mg, d.e. 97.2%). 1H NMR (500 MHz, CDCl₃, 27° C.) 2.22 (3H, s), 3.05-3.25 (1H, m), 3.23-3.35 (1H, m), 3.51-3.77 (1H, m), 3.88-4.04 (1H, m), 4.04-4.17 (1H, m), 4.54-4.81 (3H, m), 4.97-5.08 (1H, m), 5.82 (1H, dd), 6.40 (1H, d), 6.56-6.69 (1H, m), 7.36 (1H, d), 7.49 (1H, d), 7.57 (1H, s), 7.60 (1H, s), 8.67 (1H, s), 10.10 (1H, s). m/z: ES+ [M+H]+ 461. Chiral analysis method Phenomonex Lux C1, 100×3.0 mm id, 3 micron, Mobile phase A=scCO₂, B-MeOH+0.1% NH₃, Flow rate 2.0 ml/min, Isocratic 50% B. Retention time 3.13 minutes.

### Example 4, (E)-1-((8aS)-6-Chloro-5-(5-methyl-1H-indazol-4-yl)-8a,9,11,12-tetrahydropyrazino[2′,1′:3,4][1,4]oxazepino[5,6,7-de]quinazolin-10 (8H)-yl)-4-(dimethylamino)but-2-en-1-one

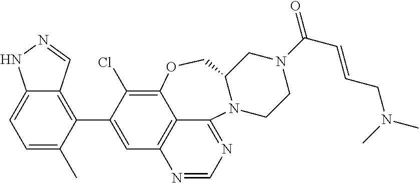 [SMILES: Cc1ccc2[nH]ncc2c1-c1cc2ncnc3c2c(c1Cl)OC[C@@H]1CN(C(=O)/C=C/CN(C)C)CCN31]

DIPEA (232 μl, 1.33 mmol) was added to (8aS)-6-chloro-5-(5-methyl-1H-indazol-4-yl)-8,8a,9,10,11,12-hexahydropyrazino[2′,1′:3,4][1,4]oxazepino[5,6,7-de]quinazoline (181 mg, 0.44 mmol), 0-(7-Azabenzotriazol-1-yl)-N,N,N′,N′-tetramethyluronium hexafluorophosphate (203 mg, 0.53 mmol) and (E)-4-(dimethylamino)but-2-enoic acid. HCl salt (81 mg, 0.49 mmol) in DMA (2 ml). The resulting solution was stirred at room temperature for 2 hours. The reaction mixture was poured into water, extracted with EtOAc, washed with brine, dried over MgSO₄, filtered and evaporated to afford crude product. The crude product was purified by preparative HPLC (Waters XSelect CSH C18 column, 5p silica, 50 mm diameter, 100 mm length), using decreasingly polar mixtures of water (containing 0.1% NH₃) and MeCN as eluents. Fractions containing the desired compound were evaporated to dryness to afford (E)-1-((8aS)-6-chloro-5-(5-methyl-1H-indazol-4-yl)-8a,9,11,12-tetrahydropyrazino[2′,1′:3,4][1,4]oxazepino[5,6,7-de]quinazolin-10 (8H)-yl)-4-(dimethylamino)but-2-en-1-one (73 mg, 32%) as a solid. 1H NMR (500 MHz, DMSO, 27° C.) 2.14 (3H, d), 2.16 (6H, s), 2.97-3.13 (3H, m), 3.2-3.52 (3H+H₂O, m), 4.22 (1H, dd), 4.42 (1H, dd), 4.6-4.88 (3H, m), 6.57-6.79 (2H, m), 7.32 (1H, d), 7.34 (1H, s), 7.48 (1H, d), 7.52 (1H, d), 8.57 (1H, d), 13.11 (1H, s). m/z: ES+ [M+H]+ 518

### 7-Bromo-5-fluoroquinazolin-4-ol

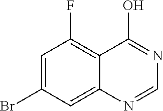 [SMILES: Oc1ncnc2cc(Br)cc(F)c12]

2-Amino-4-bromo-6-fluorobenzonitrile (550 mg, 2.56 mmol) was added portionwise to a stirred mixture of formic acid (9.94 ml, 263.46 mmol), and sulfuric acid (0.65 ml, 11.51 mmol) warmed at 100° C. over a period of 5 minutes. The resulting solution was stirred at 100° C. for 2 hours. The mixture was reduced in volume under vacuum. The residue was cooled to 0° C. and ice-water added. This was made basic with saturated NaHCO₃ (aq) and then extracted with ethyl acetate (2×75 mL). The combined organic phases were dried over MgSO₄, filtered and the solvent removed to give 7-bromo-5-fluoroquinazolin-4-ol (590 mg, 95%) as a white solid that was used without further purification. 1H NMR (500 MHz, DMSO, 27° C.) 7.59 (1H, dd), 7.69 (1H, d), 8.11 (1H, s), 12.41 (1H, s). m/z: ES− [M−H]− 241

### Tert-butyl (S)-3-(((7-bromo-4-hydroxyquinazolin-5-yl)oxy)methyl)piperazine-1-carboxylate

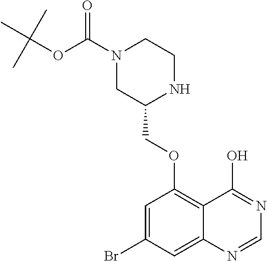 [SMILES: CC(C)(C)OC(=O)N1CCN[C@H](COc2cc(Br)cc3ncnc(O)c23)C1]

60% Sodium hydride (104 mg, 2.61 mmol) was added portionwise to tert-butyl (S)-3-(hydroxymethyl)piperazine-1-carboxylate (414 mg, 1.91 mmol) in THF (5 ml) cooled to 0° C. over a period of 5 minutes, under nitrogen. The resulting mixture was stirred at 0° C. for 10 minutes then allowed to warm to room temperature and stirred for 30 minutes. 7-Bromo-5-fluoroquinazolin-4-ol (423 mg, 1.74 mmol) was added and the mixture heated at 65° C. and stirred for 4 hours. The reaction mixture was cooled to room temperature then 60% sodium hydride (104 mg, 2.61 mmol) added, then heated to 65° C. and stirred for a further 16 hours. The reaction mixture was diluted with EtOAc (100 ml), washed with water (10 ml) and the aqueous washing was extracted with EtOAc (50 ml). The organic phases were combined, dried with MgSO₄, filtered and evaporated to afford crude product. This was purified by flash silica chromatography, elution gradient 0 to 80% EtOAc in heptane, then 0-20% MeOH in DCM. Pure fractions were evaporated to dryness to afford tert-butyl (S)-3-(((7-bromo-4-hydroxyquinazolin-5-yl)oxy)methyl)piperazine-1-carboxylate (491 mg, 64%) as a white solid. 1H NMR (500 MHz, DMSO, 27° C.) 1.38 (9H, s), 2.53-2.67 (2H, m), 2.78 (2H, s), 2.90 (2H, dd), 3.72 (1H, d), 3.86-3.98 (2H, m), 4.13 (1H, s), 7.19 (1H, d), 7.36 (1H, d), 8.00 (1H, s), 11.96 (1H, s). m/z: ES+ [M+H]+ 439

### Tert-butyl (8aS)-5-bromo-8a,9,11,12-tetrahydropyrazino[2′,1′:3,4][1,4]-oxazepino[5,6,7-de]quinazoline-10 (8H)-carboxylate

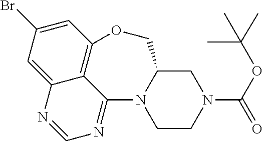 [SMILES: CC(C)(C)OC(=O)N1CCN2c3ncnc4cc(Br)cc(c34)OC[C@@H]2C1]

2,3,4,6,7,8,9,10-Octahydropyrimido[1,2-a]azepine (0.42 ml, 2.79 mmol) was added portionwise to tert-butyl (S)-3-(((7-bromo-4-hydroxyquinazolin-5-yl)oxy)methyl)-piperazine-1-carboxylate (491 mg, 1.12 mmol) and ((1H-benzo[d][1,2,3]triazol-1-yl)oxy)tris(dimethylamino)phosphonium hexafluorophosphate(V) (643 mg, 1.45 mmol) in acetonitrile (10 ml), cooled to 0° C. over a period of 5 minutes. The resulting suspension was stirred at 0° C. for 10 minutes then at room temperature for 16 hours. The reaction mixture was absorbed onto silica and purified by flash silica chromatography, elution gradient 0 to 40% EtOAc in heptane. Pure fractions were evaporated to dryness to afford tert-butyl (8aS)-5-bromo-8a,9,11,12-tetrahydropyrazino[2′,1′:3,4][1,4]oxazepino[5,6,7-de]quinazoline-10 (8H)-carboxylate (373 mg, 79%) as a white foam. 1H NMR (500 MHz, DMSO, 27° C.) 1.42 (9H, s), 3.03 (2H, s), 3.19 (1H, ddd), 3.88 (1H, d), 3.92-3.98 (1H, m), 4.02 (1H, q), 4.45 (1H, dd), 4.50 (1H, dd), 4.82 (1H, d), 7.19 (1H, d), 7.55 (1H, d), 8.48 (1H, s). m/z: ES− [M−H]− 419

### Tert-butyl (8aS)-5-(5-methyl-1H-indazol-4-yl)-8a,9,11,12-tetrahydropyrazino-[2′,1′:3,4][1,4]oxazepino[5,6,7-de]quinazoline-10 (8H)-carboxylate

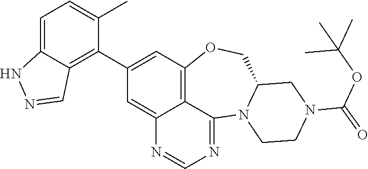 [SMILES: Cc1ccc2[nH]ncc2c1-c1cc2c3c(ncnc3c1)N1CCN(C(=O)OC(C)(C)C)C[C@H]1CO2]

Pd(PPh₃)₄ (102 mg, 0.09 mmol) was added to tert-butyl (8aS)-5-bromo-8a,9,11,12-tetrahydropyrazino[2′,1′:3,4][1,4]oxazepino[5,6,7-de]quinazoline-10 (8H)-carboxylate (373 mg, 0.89 mmol), (5-methyl-1H-indazol-4-yl)boronic acid (203 mg, 1.15 mmol) in a degassed mixture of 2M Na₂CO₃ (3 ml) and dioxane (12 ml). The resulting suspension was stirred at 100° C. for 16 hours in microwave. The mixture was diluted with DCM (150 ml), and washed with water (20 ml), then brine (20 ml). The organic phases was dried with MgSO₄, filtered and evaporated to afford crude product. The crude product was purified by flash silica chromatography, elution gradient 0 to 10% MeOH in DCM. Pure fractions were evaporated to dryness to afford tert-butyl (8aS)-5-(5-methyl-1H-indazol-4-yl)-8a,9,11,12-tetrahydropyrazino[2′,1′:3,4][1,4]oxazepino[5,6,7-de]quinazoline-10 (8H)-carboxylate (395 mg, 94%) as a yellow foam. 1H NMR (500 MHz, CDCl₃, 27° C.) 1.52 (9H, s), 2.37 (3H, s), 2.98-3.29 (3H, m), 3.82-3.98 (1H, m), 4-4.31 (2H, m), 4.41 (1H, dd), 4.51 (1H, dd), 4.95-5.15 (1H, m), 7.11 (1H, d), 7.3-7.35 (1H, m), 7.43 (1H, dd), 7.60 (1H, d), 7.80 (1H, d), 8.65 (1H, s), 10.44 (1H, s). m/z: ES+ [M+H]+ 473

### (8aS)-5-(5-Methyl-1H-indazol-4-yl)-8,8a,9,10,11,12-hexahydropyrazino[2′,1′:3,4][1,4]-oxazepino[5,6,7-de]quinazoline

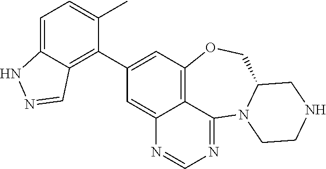 [SMILES: Cc1ccc2[nH]ncc2c1-c1cc2c3c(ncnc3c1)N1CCNC[C@H]1CO2]

TFA (2 ml, 0.84 mmol) was added to tert-butyl (8aS)-5-(5-methyl-1H-indazol-4-yl)-8a,9,11,12-tetrahydropyrazino[2′,1′:3,4][1,4]oxazepino[5,6,7-de]quinazoline-10 (8H)-carboxylate (395 mg, 0.84 mmol) in DCM (5 ml), cooled at 0° C. The resulting solution was stirred at room temperature for 2 hours. The reaction mixture was evaporated to dryness and the residue was purified by ion exchange chromatography, using an SCX2 column. The desired product was eluted from the column using 1M NH₃ in MeOH and pure fractions were evaporated to dryness to afford crude product. This was purified by flash silica chromatography, elution gradient 0 to 20% 1N NH₃ in MeOH in DCM. Pure fractions were evaporated to dryness to afford (8aS)-5-(5-methyl-1H-indazol-4-yl)-8,8a,9,10,11,12-hexahydropyrazino[2′,1′:3,4][1,4]oxazepino[5,6,7-de]quinazoline (242 mg, 78%) as a white film. 1H NMR (500 MHz, DMSO, 27° C.) 2.30 (3H, s), 2.61-2.75 (2H, m), 2.91-3.05 (3H, m), 3.86 (1H, dq), 4.38 (1H, dd), 4.47 (1H, dd), 4.97 (1H, d), 7.01 (1H, d), 7.31 (1H, d), 7.35 (1H, d), 7.49 (1H, d), 7.65 (1H, s), 8.48 (1H, s), 13.11 (1H, s). m/z: ES+ [M+H]+ 373

### Example 5, 1-[(8aS)-5-(5-Methyl-1H-indazol-4-yl)-8a,9,11,12-tetrahydropyrazino[2′,1′:3,4][1,4]-oxazepino[5,6,7-de]quinazolin-10 (8H)-yl]prop-2-en-1-one

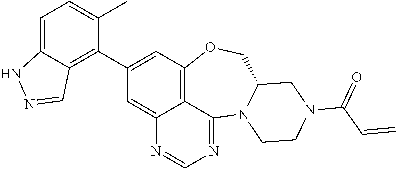 [SMILES: C=CC(=O)N1CCN2c3ncnc4cc(-c5c(C)ccc6[nH]ncc56)cc(c34)OC[C@@H]2C1]

A solution of acryloyl chloride (58 mg, 0.64 mmol) in DMA (0.5 ml) was added to a stirred suspension of (8aS)-5-(5-methyl-1H-indazol-4-yl)-8,8a,9,10,11,12-hexahydropyrazino[2′,1′:3,4][1,4]oxazepino[5,6,7-de]quinazoline (238 mg, 0.64 mmol), and triethylamine (0.18 ml, 1.28 mmol) in DMA (1 ml), cooled at 0° C. The resulting mixture was stirred at 0° C. for 30 minutes. The reaction mixture was diluted with DMA (1.5 ml) and filtered. The solution was purified by preparative HPLC (Waters CSH C18 OBD column, 5μ silica, 30 mm diameter, 100 mm length), using decreasingly polar mixtures of water (containing 1% NH₃) and MeCN as eluents. Fractions containing the desired compound were evaporated to dryness to afford 1-[(8aS)-5-(5-methyl-1H-indazol-4-yl)-8a,9,11,12-tetrahydropyrazino[2′,1′:3,4][1,4]oxazepino[5,6,7-de]quinazolin-10 (8H)-yl]prop-2-en-1-one (68 mg, 25%) as a white solid. 1H NMR (500 MHz, DMSO, 27° C.) 2.31 (3H, s), 2.98-3.13 (1H+H2O, m), 3.19-3.36 (1H, m), 3.42 (1H, d), 4.03 (1H, s), 4.09-4.34 (1H, m), 4.34-4.67 (3H, m), 4.81-5 (1H, m), 5.74 (1H, dd), 6.18 (1H, d), 6.76-6.96 (1H, m), 7.07 (1H, d), 7.32 (1H, d), 7.40 (1H, d), 7.49 (1H, d), 7.65 (1H, s), 8.54 (1H, s), 13.11 (1H, s). m/z: ES+ [M+H]+ 427.

### Tert-butyl (R)-3-(((7-bromo-4-hydroxyquinazolin-5-yl)oxy)methyl)piperazine-1-carboxylate

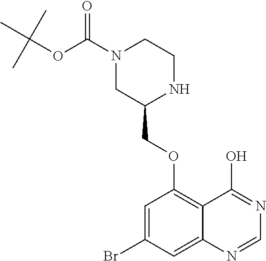 [SMILES: CC(C)(C)OC(=O)N1CCN[C@@H](COc2cc(Br)cc3ncnc(O)c23)C1]

60% Sodium hydride (123 mg, 3.09 mmol) was added portionwise to tert-butyl (R)-3-(hydroxymethyl)piperazine-1-carboxylate (489 mg, 2.26 mmol) in THF (5 ml) cooled at 0° C. over a period of 5 minutes under nitrogen. The resulting mixture was stirred at 0° C. for 10 minutes then allowed to warm to room temperature and stirred for 30 minutes. 7-Bromo-5-fluoroquinazolin-4-ol (500 mg, 2.06 mmol) was added and the mixture heated at 65° C. and stirred for 4 hours. The mixture was cooled to room temperature, then 60% sodium hydride (123 mg, 3.09 mmol) added and heated to 65° C. and stirred for a further 16 hours. The reaction mixture was cooled and filtered to give a solid. This solid was partitioned between water (10 ml) and DCM (75 ml). The aqueous phase was extracted with EtOAc (75 ml). The organic phases were combined, dried over MgSO₄ and concentrated. The residue was purified by flash silica chromatography, elution gradient 0 to 20% MeOH in DCM. Pure fractions were evaporated to dryness to afford tert-butyl (R)-3-(((7-bromo-4-hydroxyquinazolin-5-yl)oxy)methyl)piperazine-1-carboxylate (406 mg, 45%) as a white foam. 1H NMR (500 MHz, DMSO, 27° C.) 1.38 (9H, s), 2.58 (1H, td), 2.71-2.84 (1H, m), 2.90 (2H, dd), 3.72 (1H, d), 3.83-4 (2H, m), 4.13 (2H, s), 7.18 (1H, d), 7.35 (1H, d), 8.00 (1H, s), 11.94 (1H, s). m/z: ES− [M−H]− 437

### Tert-butyl (8aR)-5-bromo-8a,9,11,12-tetrahydropyrazino[2′,1′:3,4][1,4]oxazepino-[5,6,7-de]quinazoline-10 (8H)-carboxylate

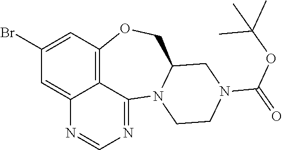 [SMILES: CC(C)(C)OC(=O)N1CCN2c3ncnc4cc(Br)cc(c34)OC[C@H]2C1]

2,3,4,6,7,8,9,10-Octahydropyrimido[1,2-a]azepine (0.35 ml, 2.31 mmol) was added portionwise to tert-butyl (R)-3-(((7-bromo-4-hydroxyquinazolin-5-yl)oxy)methyl)-piperazine-1-carboxylate (406 mg, 0.92 mmol) and ((1H-benzo[d][1,2,3]triazol-1-yl)oxy)tris(dimethylamino)phosphonium hexafluorophosphate(V) (531 mg, 1.2 mmol) in acetonitrile (10 ml) cooled to 0° C. over a period of 5 minutes. The resulting suspension was stirred at 0° C. for 10 minutes then at room temperature for 16 hours. The reaction mixture was absorbed onto silica and purified by flash silica chromatography, elution gradient 0 to 40% EtOAc in heptane. Pure fractions were evaporated to dryness to afford tert-butyl (8aR)-5-bromo-8a,9,11,12-tetrahydropyrazino[2′,1′:3,4][1,4]oxazepino[5,6,7-de]-quinazoline-10 (8H)-carboxylate (256 mg, 66%) as a white foam. 1H NMR (500 MHz, CDCl₃, 27° C.) 1.50 (9H, s), 2.95-3.21 (3H, m), 3.77-3.89 (1H, m), 4.12 (2H, bs), 4.33 (1H, dd), 4.41 (1H, dd), 5.00 (1H, bd), 7.16 (1H, d), 7.69 (1H, d), 8.57 (1H, s). m/z: ES+ [M+H]+ 423.

### Tert-butyl (8aR)-5-(5-methyl-1H-indazol-4-yl)-8a,9,11,12-tetrahydropyrazino-[2′,1′:3,4][1,4]oxazepino[5,6,7-de]quinazoline-10 (8H)-carboxylate

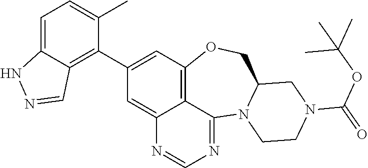 [SMILES: Cc1ccc2[nH]ncc2c1-c1cc2c3c(ncnc3c1)N1CCN(C(=O)OC(C)(C)C)C[C@@H]1CO2]

Pd(PPh₃)₄ (70.2 mg, 0.06 mmol) was added to tert-butyl (8aR)-5-bromo-8a,9,11,12-tetrahydropyrazino[2′,1′:3,4][1,4]oxazepino[5,6,7-de]quinazoline-10 (8H)-carboxylate (256 mg, 0.61 mmol) and (5-methyl-1H-indazol-4-yl)boronic acid (160 mg, 0.91 mmol) in a degassed mixture of 2M Na₂CO₃ (3 ml) and dioxane (12 ml). The resulting suspension was stirred at 100° C. for 16 hours in a microwave. The reaction mixture was diluted with DCM (150 ml), and washed with water (20 ml), then brine (20 ml). The organic phase was dried with MgSO₄, filtered and evaporated to afford crude product. The crude product was purified by flash silica chromatography, elution gradient 0 to 10% MeOH in DCM. Pure fractions were evaporated to dryness to afford tert-butyl (8aR)-5-(5-methyl-1H-indazol-4-yl)-8a,9,11,12-tetrahydropyrazino[2′,1′:3,4][1,4]oxazepino[5,6,7-de]quinazoline-10 (8H)-carboxylate (307 mg, >100%) as a yellow foam. 1H NMR (500 MHz, CDCl₃, 27° C.) 1.52 (9H, s), 2.37 (3H, s), 2.99-3.31 (3H, m), 3.81-3.95 (1H, m), 4.12 (2H, bs), 4.41 (1H, dd), 4.51 (1H, dd), 5.09 (1H, bd), 7.11 (1H, d), 7.31-7.35 (1H, m), 7.43 (1H, dd), 7.61 (1H, d), 7.80 (1H, d), 8.65 (1H, s), 10.50 (1H, s). m/z: ES+ [M+H]+ 473

### (8aR)-5-(5-Methyl-1H-indazol-4-yl)-8,8a,9,10,11,12-hexahydropyrazino[2′,1′:3,4][1,4]-oxazepino[5,67-de]quinazoline

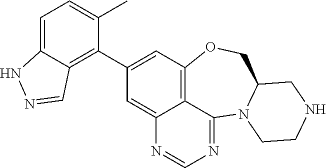 [SMILES: Cc1ccc2[nH]ncc2c1-c1cc2c3c(ncnc3c1)N1CCNC[C@@H]1CO2]

TFA (1.5 ml, 0.65 mmol) was added to tert-butyl (8aR)-5-(5-methyl-1H-indazol-4-yl)-8a,9,11,12-tetrahydropyrazino[2′,1′:3,4][1,4]oxazepino[5,6,7-de]quinazoline-10 (8H)-carboxylate (307 mg, 0.65 mmol) in DCM (5 ml) cooled to 0° C. The resulting solution was stirred at room temperature for 2 hours. The reaction mixture was evaporated to dryness. The residue was purified by ion exchange chromatography, using an SCX2 column. The desired product was eluted from the column using 1M NH₃ in MeOH and pure fractions were evaporated to dryness to afford crude product. This was purified by flash silica chromatography, elution gradient 0 to 20% 1M NH₃/MeOH in DCM. Pure fractions were evaporated to dryness to afford (8aR)-5-(5-methyl-1H-indazol-4-yl)-8,8a,9,10,11,12-hexahydropyrazino[2′,1′:3,4][1,4]oxazepino[5,6,7-de]quinazoline (155 mg, 64%) as yellow film. 1H NMR (500 MHz, DMSO, 27° C.) 2.30 (3H, s), 2.61-2.77 (2H, m), 2.89-3.07 (3H, m), 3.86 (1H, dq), 4.38 (1H, dd), 4.47 (1H, dd), 4.97 (1H, d), 7.01 (1H, d), 7.31 (1H, d), 7.35 (1H, d), 7.44-7.52 (1H, m), 7.65 (1H, s), 8.48 (1H, s), 13.11 (1H, s). m/z: ES+ [M+H]+ 373.

### Example 6, 1-[(8aR)-5-(5-Methyl-1H-indazol-4-yl)-8a,9,11,12-tetrahydropyrazino[2′,1′:3,4]-[1,4]oxazepino[5,6,7-de]quinazolin-10 (8H)-yl]prop-2-en-1-one

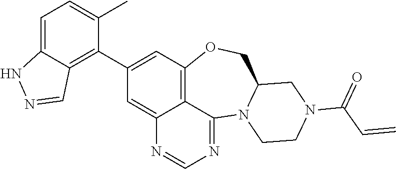 [SMILES: C=CC(=O)N1CCN2c3ncnc4cc(-c5c(C)ccc6[nH]ncc56)cc(c34)OC[C@H]2C1]

A solution of acryloyl chloride (38 mg, 0.42 mmol) in DMA (0.5 ml) was added to a stirred suspension of (8aR)-5-(5-methyl-1H-indazol-4-yl)-8,8a,9,10,11,12-hexahydropyrazino[2′,1′:3,4][1,4]oxazepino [5,6,7-de]quinazoline (155 mg, 0.42 mmol), and triethylamine (0.174 ml, 1.25 mmol) in DMA (1 ml) cooled to −78° C. The resulting mixture was stirred at −78° C. for 30 minutes. The reaction mixture was diluted with DMA (1.5 ml) and filtered. The filtrate was purified by preparative HPLC (Waters CSH C18 OBD column, 5μ silica, 30 mm diameter, 100 mm length), using decreasingly polar mixtures of water (containing 1% NH₃) and MeCN as eluents. Fractions containing the desired compound were evaporated to dryness to afford 1-[(8aR)-5-(5-methyl-1H-indazol-4-yl)-8a,9,11,12-tetrahydropyrazino[2′,1′:3,4][1,4]-oxazepino[5,6,7-de]quinazolin-10 (8H)-yl]prop-2-en-1-one (78 mg, 44%) as a solid. 1H NMR (500 MHz, DMSO, 27° C.) 2.31 (3H, s), 2.98-3.13 (1H, m), 3.2-3.33 (1H+H2O, m), 3.36-3.49 (1H, m), 4.02 (1H, s), 4.09-4.33 (1H, m), 4.33-4.65 (3H, m), 4.81-4.97 (1H, m), 5.7-5.78 (1H, m), 6.18 (1H, d), 6.8-6.94 (1H, m), 7.07 (1H, d), 7.31 (1H, d), 7.40 (1H, d), 7.49 (1H, d), 7.65 (1H, s), 8.54 (1H, s), 13.11 (1H, s). m/z: ES+ [M+H]+ 427.

### Tert-butyl (R)-3-(((7-bromo-6-chloro-4-hydroxyquinazolin-5-yl)oxy)methyl)-piperazine-1-carboxylate

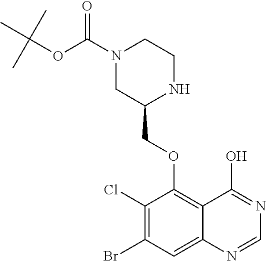 [SMILES: CC(C)(C)OC(=O)N1CCN[C@@H](COc2c(Cl)c(Br)cc3ncnc(O)c23)C1]

60% Sodium hydride (136 mg, 3.41 mmol) was added portionwise to tert-butyl (R)-3-(hydroxymethyl)piperazine-1-carboxylate (540 mg, 2.5 mmol) in THF (10 ml) cooled to 0° C. over a period of 5 minutes under nitrogen. The resulting mixture was stirred at 0° C. for 10 minutes then allowed to warm to room temperature and stirred for 20 minutes. 7-Bromo-6-chloro-5-fluoroquinazolin-4-ol (630 mg, 2.27 mmol) was added and the mixture heated at 65° C. and stirred for 2 hours. The reaction mixture was cooled to room temperature, then NaH (50 mg) added and heated at 65° C. and stirred for a further 3 hours. The reaction mixture was diluted with EtOAc (75 ml), and washed with water (25 ml). The washings were extracted with EtOAc (75 ml). The combined organic layers were dried with MgSO₄, filtered and evaporated to afford crude product. This was purified by flash silica chromatography, elution gradient 0 to 20% MeOH in DCM. Pure fractions were evaporated to dryness to afford tert-butyl (R)-3-(((7-bromo-6-chloro-4-hydroxyquinazolin-5-yl)oxy)methyl)piperazine-1-carboxylate (340 mg, 32%) as a white foam. 1H NMR (500 MHz, DMSO, 27° C.) 1.39 (9H, s), 2.52-2.85 (4H, m), 2.85-2.92 (1H, m), 2.92-3 (1H, m), 3.74 (1H, d), 3.93 (2H, d), 4.05 (1H, d), 7.84 (1H, s), 8.09 (1H, s). m/z: ES+ [M+H]+ 473.

### Tert-butyl (8aR)-5-bromo-6-chloro-8a,9,11,12-tetrahydropyrazino[2′,1′:3,4][1,4]-oxazepino[5,6,7-de]quinazoline-10 (8H)-carboxylate

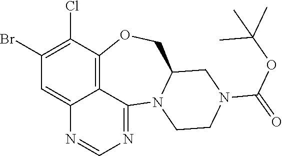 [SMILES: CC(C)(C)OC(=O)N1CCN2c3ncnc4cc(Br)c(Cl)c(c34)OC[C@H]2C1]

2,3,4,6,7,8,9,10-Octahydropyrimido[1,2-a]azepine (0.27 ml, 1.79 mmol) was added portionwise to tert-butyl (R)-3-(((7-bromo-6-chloro-4-hydroxyquinazolin-5-yl)oxy)methyl)piperazine-1-carboxylate (340 mg, 0.72 mmol) and ((1H-benzo[d][1,2,3]-triazol-1-yl)oxy)tris(dimethylamino)phosphonium hexafluorophosphate(V) (413 mg, 0.93 mmol) in acetonitrile (10 ml) cooled to 0° C. over a period of 5 minutes. The resulting suspension was stirred at 0° C. for 10 minutes then at room temperature for 16 hours. The reaction mixture was absorbed onto silica and this was purified by flash silica chromatography, elution gradient 0 to 40% EtOAc in heptane. Pure fractions were evaporated to dryness to afford tert-butyl (8aR)-5-bromo-6-chloro-8a,9,11,12-tetrahydropyrazino[2′,1′:3,4][1,4]oxazepino[5,6,7-de]quinazoline-10 (8H)-carboxylate (260 mg, 79%) as a white foam. 1H NMR (500 MHz, DMSO, 27° C.) 1.43 (9H, s), 3.05 (2H, s), 3.15-3.26 (1H, m), 3.89 (1H, d), 3.96-4.1 (2H, m), 4.54-4.68 (2H, m), 4.78 (1H, d), 7.79 (1H, d), 8.52 (1H, d). m/z: ES+ [M+H]+ 455.

### Tert-butyl (8aR)-6-chloro-5-(5-methyl-1H-indazol-4-yl)-8a,9,11,12-tetrahydropyrazino[2′,1′:3,4][1,4]oxazepino[5,6,7-de]quinazoline-10 (8H)-carboxylate

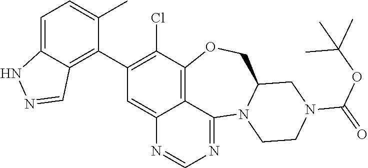 [SMILES: Cc1ccc2[nH]ncc2c1-c1cc2ncnc3c2c(c1Cl)OC[C@H]1CN(C(=O)OC(C)(C)C)CCN31]

Pd(PPh₃)₄ (66 mg, 0.06 mmol) was added to tert-butyl (8aR)-5-bromo-6-chloro-8a,9,11,12-tetrahydropyrazino[2′,1′:3,4][1,4]oxazepino[5,6,7-de]quinazoline-10 (8H)-carboxylate (260 mg, 0.57 mmol) and (5-methyl-1H-indazol-4-yl)boronic acid (151 mg, 0.86 mmol) in a degassed mixture of 2M Na₂CO₃ (3 ml) and dioxane (12 ml). The resulting suspension was stirred at 100° C. for 16 hours in a microwave. The mixture was diluted with DCM (150 ml), and washed with water (20 ml), then brine (20 ml). The organic phase was dried with MgSO₄, filtered and evaporated to afford crude product. The crude product was purified by flash silica chromatography, elution gradient 0 to 10% MecOH in DCM. Pure fractions were evaporated to dryness to afford tert-butyl (8aR)-6-chloro-5-(5-methyl-1H-indazol-4-yl)-8a,9,11,12-tetrahydropyrazino[2′,1′:3,4][1,4]oxazepino[5,6,7-de]quinazoline-10 (8H)-carboxylate (186 mg, 64%) as a yellow foam. m/z: ES+ [M+H]+ 507.

### (8aR)-6-Chloro-5-(5-methyl-1H-indazol-4-yl)-8,8a,9,10,11,12-hexahydropyrazino-[2′,1′:3,4][1,4]oxazepino[5,6,7-de]quinazoline

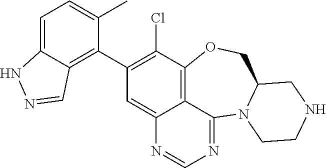 [SMILES: Cc1ccc2[nH]ncc2c1-c1cc2ncnc3c2c(c1Cl)OC[C@H]1CNCCN31]

TFA (1 ml, 0.37 mmol) was added to tert-butyl (8aR)-6-chloro-5-(5-methyl-1H-indazol-4-yl)-8a,9,11,12-tetrahydropyrazino[2′,1′:3,4][1,4]oxazepino[5,6,7-de]quinazoline-10 (8H)-carboxylate (186 mg, 0.37 mmol) in DCM (4 ml) cooled at 0° C. The resulting solution was stirred at room temperature for 2 hours. The reaction mixture was evaporated to dryness. The residue was purified by ion exchange chromatography, using an SCX2 column. The desired product was eluted from the column using 1M NH₃/MeOH and pure fractions were evaporated to dryness to afford crude product, which was purified by flash silica chromatography, elution gradient 0 to 20% 1M NH₃/MeOH in DCM. Pure fractions were evaporated to dryness to afford (8aR)-6-chloro-5-(5-methyl-1H-indazol-4-yl)-8,8a,9,10,11,12-hexahydropyrazino[2′,1′:3,4][1,4]oxazepino[5,6,7-de]quinazoline (128 mg, 86%) as a yellow film. 1H NMR (500 MHz, DMSO, 27° C.) 2.13 (3H, s), 2.63-2.77 (3H, m), 2.92-3 (1H, m), 3-3.08 (2H, m), 3.85-3.97 (1H, m), 4.51 (1H, dt), 4.60 (1H, dd), 4.89 (1H, d), 7.29 (1H, s), 7.32 (1H, d), 7.48 (1H, d), 7.51 (1H, d), 8.51 (1H, s), 13.10 (1H, s). m/z: ES+ [M+H]+ 407.

### Example 7, 1-[(8aR)-6-Chloro-5-(5-methyl-1H-indazol-4-yl)-8a,9,11,12-tetrahydropyrazino-[2′,1′:3,4][1,4]oxazepino[5,6,7-de]quinazolin-10 (8H)-yl]prop-2-en-1-one

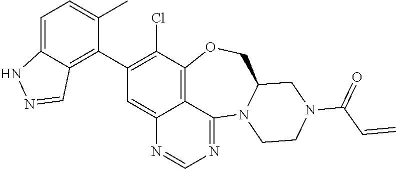 [SMILES: C=CC(=O)N1CCN2c3ncnc4cc(-c5c(C)ccc6[nH]ncc56)c(Cl)c(c34)OC[C@H]2C1]

A solution of acryloyl chloride (28.5 mg, 0.31 mmol) in DMA (0.5 ml) was added to a stirred suspension of (8aR)-6-chloro-5-(5-methyl-1H-indazol-4-yl)-8,8a,9,10,11,12-hexahydropyrazino[2′,1′:3,4][1,4]oxazepino[5,6,7-de]quinazoline (128 mg, 0.31 mmol), and triethylamine (0.13 ml, 0.94 mmol) in DMA (1 ml) cooled at 0° C. The resulting mixture was stirred at 0° C. for 30 minutes. The reaction mixture was diluted with a few drops of MeOH and DMSO (1 ml) then filtered. The filtrate was purified by preparative HPLC (Waters CSH C18 OBD column, 5μ silica, 30 mm diameter, 100 mm length), using decreasingly polar mixtures of water (containing 1% NH₃) and MeCN as eluents. Fractions containing the desired compound were evaporated to dryness to afford 1-[(8aR)-6-chloro-5-(5-methyl-1H-indazol-4-yl)-8a,9,11,12-tetrahydropyrazino[2′,1′:3,4][1,4]oxazepino-[5,6,7-de]quinazolin-10 (8H)-yl]prop-2-en-1-one (17 mg, 12%) as a white film. 1H NMR (500 MHz, CDCl₃, 27° C.) 2.23 (3H, d), 3.03-3.38 (2H, m), 3.43-3.78 (1H+MeOH, m), 3.98 (1H, s), 4.04-4.19 (1H, m), 4.47-4.87 (3H, m), 5.02 (1H, d), 5.82 (1H, dd), 6.40 (1H, d), 6.64 (1H, dd), 7.34-7.38 (1H, m), 7.49 (1H, d), 7.54-7.62 (2H, m), 8.67 (1H, s), 10.16 (1H, s). m/z: ES+ [M+H]+ 461.

### 2-Amino-4-bromo-3-chloro-6-fluorobenzonitrile

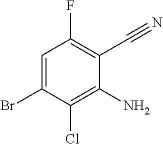 [SMILES: N#Cc1c(F)cc(Br)c(Cl)c1N]

1-Chloropyrrolidine-2,5-dione (1.38 g, 10.33 mmol) was added in one portion to 2-amino-4-bromo-6-fluorobenzonitrile (2.02 g, 9.39 mmol), in i-PrOH (15 ml) warmed at 60° C. The resulting suspension was stirred at 82° C. for 2 hours then allowed to cool to room temperature. The reaction mixture was evaporated to dryness and re-dissolved in DCM (150 ml), and washed with water (25 ml). The organic layer was dried with MgSO₄, filtered and evaporated to afford crude product. The crude product was purified by flash silica chromatography, elution gradient 0 to 40% EtOAc in heptane. Pure fractions were evaporated to dryness to afford 2-amino-4-bromo-3-chloro-6-fluorobenzonitrile (0.92 g, 39%) as a white solid. 1H NMR (500 MHz, DMSO, 27° C.) 6.94 (2H, s), 7.09 (1H, d). m/z: ES− [M−H]− 247.

### 7-Bromo-8-chloro-5-fluoroquinazolin-4-ol

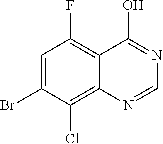 [SMILES: Oc1ncnc2c(Cl)c(Br)cc(F)c12]

Sulfuric acid (0.31 ml, 5.53 mmol) was added to a suspension of 2-amino-4-bromo-3-chloro-6-fluorobenzonitrile (920 mg, 3.69 mmol), in formic acid (9 ml, 238.57 mmol) at room temperature. The resulting solution was stirred at 100° C. for 2 hours. The mixture was reduced under vacuum to give a solid. Water (50 ml) and Me-THF (100 ml) added then cooled to 0° C. and the aqueous was made basic by cautious addition of saturated aqueous NaHCO₃. The mixture was extracted with warm ethyl acetate (47° C., 2×200 ml). The organic extracts were combined, dried over MgSO₄, filtered and the solvent removed to afford 7-bromo-8-chloro-5-fluoroquinazolin-4-ol (970 mg, 95%) as a pale yellow solid. 1H NMR (500 MHz, DMSO, 27° C.) 7.80 (1H, d), 8.23 (1H, s), 12.66 (1H, s). m/z: ES− [M−H]− 275.

### Tert-butyl (S)-3-(((7-bromo-8-chloro-4-hydroxyquinazolin-5-yl)oxy)methyl)-piperazine-1-carboxylate

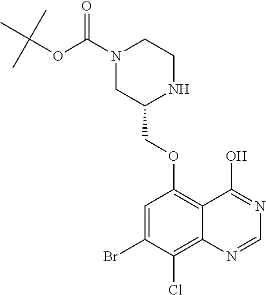 [SMILES: CC(C)(C)OC(=O)N1CCN[C@H](COc2cc(Br)c(Cl)c3ncnc(O)c23)C1]

60% Sodium hydride (154 mg, 3.85 mmol) was added portionwise to tert-butyl (S)-3-(hydroxymethyl)piperazine-1-carboxylate (832 mg, 3.85 mmol) in THF (15 ml) cooled to 0° C. over a period of 5 minutes under nitrogen. The resulting mixture was stirred at 0° C. for 10 minutes then allowed to warm to room temperature and stirred for 20 minutes. 7-Bromo-8-chloro-5-fluoroquinazolin-4-ol (970 mg, 3.5 mmol) was added and the mixture heated at 65° C. and stirred for 2 hours then cooled to room temperature. Further 60% sodium hydride (154 mg, 3.85 mmol) was added and then heated at 65° C. and stirred for a further 2 hours before cooling to room temperature. The reaction mixture was diluted with EtOAc (100 ml), and water (25 ml). The aqueous phase was taken to pH 5 with acetic acid and separated. The aqueous phase was extracted with EtOAc (100 ml) and the organic phases combined, dried and evaporated. The residue was purified by flash silica chromatography, elution gradient 0 to 20% MeOH in DCM. Pure fractions were evaporated to dryness to afford tert-butyl (S)-3-(((7-bromo-8-chloro-4-hydroxyquinazolin-5-yl)oxy)methyl)piperazine-1-carboxylate (1.35 mg, 82%) as pale yellow foam. 1H NMR (500 MHz, DMSO, 27° C.) 1.38 (9H, s), 2.53-2.68 (2H, m), 2.68-2.85 (2H, m), 2.85-2.97 (2H, m), 3.72 (1H, d), 3.87-3.99 (2H, m), 4.1-4.19 (1H, m), 7.38 (1H, s), 8.14 (1H, s). m/z: ES+ [M+H]+ 473.

### Tert-butyl (8aS)-5-bromo-4-chloro-8a,9,11,12-tetrahydropyrazino[2′,1′:3,4][1,4]-oxazepino[5,6,7-de]quinazoline-10 (8H)-carboxylate

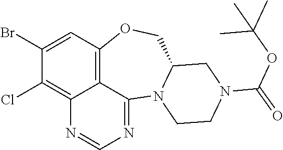 [SMILES: CC(C)(C)OC(=O)N1CCN2c3ncnc4c(Cl)c(Br)cc(c34)OC[C@@H]2C1]

2,3,4,6,7,8,9,10-Octahydropyrimido[1,2-a]azepine (1.06 ml, 7.12 mmol) was added dropwise to a mixture of tert-butyl (S)-3-(((7-bromo-8-chloro-4-hydroxyquinazolin-5-yl)oxy)methyl)piperazine-1-carboxylate (1.35 g, 2.85 mmol) and ((1H-benzo[d][1,2,3]-triazol-1-yl)oxy)tris(dimethylamino)phosphonium hexafluorophosphate(V) (1.638 g, 3.70 mmol) in acetonitrile (35 ml) cooled to 0° C. The reaction was stirred at 0° C. for 15 minutes then allowed to warm to room temperature and stirred for 16 hours. MeOH (50 ml) was added and a solid was filtered off and dried to give tert-butyl (8aS)-5-bromo-4-chloro-8a,9,11,12-tetrahydropyrazino[2′,1′:3,4][1,4]oxazepino[5,6,7-de]quinazoline-10 (8H)-carboxylate (0.16 g, 12%). The filtrate was absorbed onto silica and this was purified by flash silica chromatography, elution gradient 0 to 40% EtOAc in heptane. Pure fractions were evaporated to dryness to afford tert-butyl (8aS)-5-bromo-4-chloro-8a,9,11,12-tetrahydropyrazino[2′,1′:3,4][1,4]oxazepino[5,6,7-de]quinazoline-10 (8H)-carboxylate (0.5 g, 39%) as a white solid. 1H NMR (500 MHz, DMSO, 27° C.) 1.42 (9H, s), 3.07 (2H, bs), 3.2-3.28 (1H, m), 3.89 (1H, d), 3.94-4.09 (2H, m), 4.38-4.58 (2H, m), 4.81 (1H, d), 7.40 (1H, s), 8.60 (1H, s). m/z: ES+ [M+H]+ 455.

### Tert-butyl (8aS)-4-chloro-5-(5-methyl-1H-indazol-4-yl)-8a,9,11,12-tetrahydropyrazino[2′,1′:3,4][1,4]oxazepino[5,6,7-de]quinazoline-10 (8H)-carboxylate

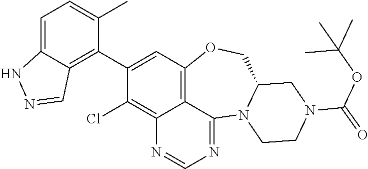 [SMILES: Cc1ccc2[nH]ncc2c1-c1cc2c3c(ncnc3c1Cl)N1CCN(C(=O)OC(C)(C)C)C[C@H]1CO2]

Pd(PPh₃)₄ (167 mg, 0.14 mmol) was added to tert-butyl (8aS)-5-bromo-4-chloro-8a,9,11,12-tetrahydropyrazino[2′,1′:3,4][1,4]oxazepino[5,6,7-de]quinazoline-10 (8H)-carboxylate (659 mg, 1.45 mmol) and (5-methyl-1H-indazol-4-yl)boronic acid (382 mg, 2.17 mmol) in a degassed mixture of 2M Na₂CO₃ (3 ml) and dioxane (12 ml). The resulting suspension was stirred at 100° C. for 18 hours in a microwave. The mixture was diluted with DCM (150 ml), and washed with water (20 ml), then brine (20 ml). The organic phase was dried with MgSO₄, filtered and evaporated to afford crude product which was purified by flash silica chromatography, elution gradient 0 to 10% MeOH in DCM. Fractions were evaporated to dryness to afford crude product. This crude product was purified by flash silica chromatography, elution gradient 0 to 5% 2N NH₃/MeOH in DCM. Pure fractions were evaporated to dryness to afford tert-butyl (8aS)-4-chloro-5-(5-methyl-1H-indazol-4-yl)-8a,9,11,12-tetrahydropyrazino[2′,1′:3,4][1,4]oxazepino[5,6,7-de]quinazoline-10 (8H)-carboxylate (540 mg, 74%) as film. 1H NMR (500 MHz, DMSO, 27° C.) 1.39-1.45 (9H, m), 2.13 (3H, d), 3.22-3.3 (1H, m), 3.31 (2H, s), 3.93 (1H, d), 3.97-4.13 (2H, m), 4.43-4.64 (2H, m), 4.86 (1H, d), 6.97 (1H, s), 7.31 (1H, d), 7.47 (1H, d), 7.51 (1H, d), 8.65 (1H, s), 13.10 (1H, s). m/z: ES+ [M+H]+ 507.

### (8aS)-4-Chloro-5-(5-methyl-1H-indazol-4-yl)-8,8a,9,10,11,12-hexahydropyrazino-[2′,1′:3,4][1,4]oxazepino[5,6,7-de]quinazoline

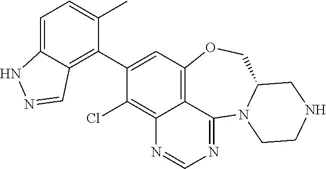 [SMILES: Cc1ccc2[nH]ncc2c1-c1cc2c3c(ncnc3c1Cl)N1CCNC[C@H]1CO2]

TFA (2 ml, 1.07 mmol) was added to tert-butyl (8aS)-4-chloro-5-(5-methyl-1H-indazol-4-yl)-8a,9,11,12-tetrahydropyrazino[2′,1′:3,4][1,4]oxazepino[5,6,7-de]quinazoline-10 (8H)-carboxylate (540 mg, 1.07 mmol) in DCM (8 ml) cooled at 0° C. The resulting solution was stirred at room temperature for 2 hours then evaporated to dryness. The residue was purified by ion exchange chromatography, using an SCX2 column. The desired product was eluted from the column using 1M NH₃/MeOH. Pure fractions were evaporated to dryness to afford (8aS)-4-chloro-5-(5-methyl-1H-indazol-4-yl)-8,8a,9,10,11,12-hexahydropyrazino[2′,1′:3,4][1,4] oxazepino[5,6,7-de]quinazoline (374 mg, 86%) as a yellow film. 1H NMR (500 MHz, DMSO, 27° C.) 2.13 (3H, s), 2.64-2.76 (2H, m), 2.93-3.1 (3H, m), 3.8-3.94 (1H, m), 4.08 (1H, s), 4.39 (1H, ddd), 4.48 (1H, ddd), 4.95 (1H, dd), 6.92 (1H, s), 7.31 (1H, d), 7.48 (1H, s), 7.51 (1H, d), 8.61 (1H, s), 13.10 (1H, s). m/z: ES+ [M+H]+ 407.

### Example 8, 1-[(8aS)-4-Chloro-5-(5-methyl-1H-indazol-4-yl)-8a,9,11,12-tetrahydropyrazino-[2′,1′:3,4][1,4]oxazepino[5,6,7-de]quinazolin-10 (8H)-yl]prop-2-en-1-one

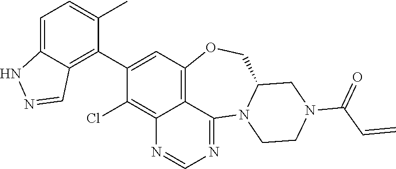 [SMILES: C=CC(=O)N1CCN2c3ncnc4c(Cl)c(-c5c(C)ccc6[nH]ncc56)cc(c34)OC[C@@H]2C1]

A solution of acryloyl chloride (45.4 mg, 0.5 mmol) in DMA (0.5 ml) was added to a stirred suspension of (8aS)-4-chloro-5-(5-methyl-1H-indazol-4-yl)-8,8a,9,10,11,12-hexahydropyrazino[2′,1′:3,4][1,4]oxazepino[5,6,7-de]quinazoline (204 mg, 0.50 mmol), and triethylamine (0.21 ml, 1.5 mmol) in DMA (1 ml) cooled at 0° C. The resulting mixture was stirred at 0° C. for 30 minutes. The reaction mixture was diluted with a few drops of MecOH and DMSO (1 ml) then filtered. The filtrate was purified by preparative HPLC (Waters CSH C18 OBD column, 5μ silica, 30 mm diameter, 100 mm length), using decreasingly polar mixtures of water (containing 1% NH₃) and MeCN as eluents. Fractions containing the desired compound were evaporated to afford 1-[(8aS)-4-chloro-5-(5-methyl-1H-indazol-4-yl)-8a,9,11,12-tetrahydropyrazino[2′,1′:3,4][1,4]oxazepino[5,6,7-de]quinazolin-10 (8H)-yl]prop-2-en-1-one (52 mg, 23%) as a solid. 1H NMR (500 MHz, DMSO, 27° C.) 2.14 (3H, d), 2.99-3.18 (1H, m), 3.35-3.49 (2H, m), 4.06 (1H, s), 4.1-4.33 (1H, m), 4.33-4.5 (1H, m), 4.5-4.65 (2H, m), 4.76-4.95 (1H, m), 5.68-5.78 (1H, m), 6.11-6.22 (1H, m), 6.79-6.92 (1H, m), 6.98 (1H, s), 7.33 (1H, d), 7.47 (1H, d), 7.51 (1H, d), 8.66 (1H, s), 13.10 (1H, s). m/z: ES+ [M+H]+ 461.

### Tert-butyl (8aS)-6-chloro-5-(2-fluoro-6-hydroxyphenyl)-8a,9,11,12-tetrahydropyrazino[2′,1′:3,4][1,4]oxazepino[5,6,7-de]quinazoline-10 (8H)-carboxylate

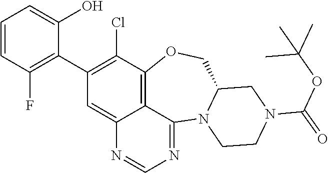 [SMILES: CC(C)(C)OC(=O)N1CCN2c3ncnc4cc(-c5c(O)cccc5F)c(Cl)c(c34)OC[C@@H]2C1]

Pd(PPh₃)₄ (31.7 mg, 0.03 mmol) was added to tert-butyl (8aR)-5-bromo-8a,9,11,12-tetrahydropyrazino[2′,1′:3,4][1,4]oxazepino[5,6,7-de]quinazoline-10 (8H)-carboxylate (125 mg, 0.27 mmol) and (2-fluoro-6-hydroxyphenyl)boronic acid (64.1 mg, 0.41 mmol) in a degassed mixture of 2M Na₂CO₃ (0.8 ml) and dioxane (3 ml). The resulting suspension was stirred at 100° C. for 15 hours in a microwave reactor. The mixture was diluted with DCM (25 ml), and washed with water (5 ml), then brine (5 ml). The organic phase was dried over MgSO₄, filtered and evaporated to afford crude product. The crude product was purified by flash silica chromatography, elution gradient 0 to 10% MecOH in DCM. Pure fractions were evaporated to dryness to afford tert-butyl (8aS)-6-chloro-5-(2-fluoro-6-hydroxyphenyl)-8a,9,11,12-tetrahydropyrazino[2′,1′:3,4][1,4]oxazepino[5,6,7-de]quinazoline-10 (8H)-carboxylate (107 mg, 80%) as a white solid. 1H NMR (500 MHz, DMSO, 27° C.) 1.44 (9H, s), 2.91-3.26 (3H, m), 3.92 (1H, d), 3.95-4.02 (1H, m), 4.07 (1H, d), 4.57-4.71 (2H, m), 4.81 (1H, d), 6.7-6.78 (1H, m), 6.80 (1H, dd), 7.27 (1H, td), 7.33 (1H, d), 8.53 (1H, s), 10.01 (1H, d). m/z: ES+ [M+H]+ 487.

### 2-[(8aS)-6-chloro-8,8a,9,10,11,12-hexahydropyrazino[2′,1′:3,4][1,4]oxazepino[5,6,7-de]quinazolin-5-yl]-3-fluorophenol

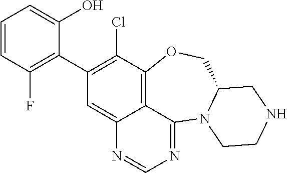 [SMILES: Oc1cccc(F)c1-c1cc2ncnc3c2c(c1Cl)OC[C@@H]1CNCCN31]

TFA (2 ml) was added to tert-butyl (8aS)-6-chloro-5-(2-fluoro-6-hydroxyphenyl)-8a,9,11,12-tetrahydropyrazino[2′,1′:3,4][1,4]oxazepino[5,6,7-de]quinazoline-10 (8H)-carboxylate (499 mg, 1.02 mmol) in DCM (5 ml). The resulting solution was stirred at room temperature for 1 hour. The reaction mixture was evaporated to dryness then dissolved in MeOH (5 ml). This was purified by ion exchange chromatography, using an SCX2 (10 g) column. The desired product was eluted from the column using 1N NH₃/MeOH and pure fractions were evaporated to dryness to afford 2-[(8aS)-6-chloro-8,8a,9,10,11,12-hexahydropyrazino[2′,1′:3,4][1,4] oxazepino[5,6,7-de]quinazolin-5-yl]-3-fluorophenol (300 mg, 76%) as a yellow film. 1H NMR (500 MHz, DMSO, 27° C.) 2.6-2.78 (3H, m), 2.88-3.1 (3H, m), 3.78-3.96 (1H, m), 4.48 (1H, dd), 4.56 (1H, ddd), 4.87 (1H, dd), 6.68-6.77 (1H, m), 6.80 (1H, dd), 7.23-7.32 (2H, m), 8.49 (1H, s), 9.82 (1H, s). m/z: ES+ [M+H]+ 387.

### Example 9, 1-[(8aS)-6-Chloro-5-(2-fluoro-6-hydroxyphenyl)-8a,9,11,12-tetrahydropyrazino-[2′,1′:3,4][1,4]oxazepino[5,6,7-de]quinazolin-10 (8H)-yl]prop-2-en-1-one

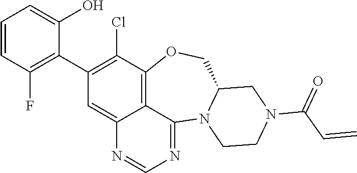 [SMILES: C=CC(=O)N1CCN2c3ncnc4cc(-c5c(O)cccc5F)c(Cl)c(c34)OC[C@@H]2C1]

A solution of acryloyl chloride (58 mg, 0.64 mmol) in DMA (0.5 ml) was added to a stirred suspension of 2-[(8aS)-6-chloro-8,8a,9,10,11,12-hexahydropyrazino-[2′,1′:3,4][1,4]oxazepino [5,6,7-de]quinazolin-5-yl]-3-fluorophenol (226 mg, 0.58 mmol), and triethylamine (0.244 ml, 1.75 mmol) in DMA (0.5 ml) cooled to 0° C. The resulting mixture was stirred at room temperature for 30 minutes. The reaction mixture was diluted with DMSO (1 ml), then filtered. The filtrate was purified by preparative HPLC (Waters CSH C18 OBD column, 5μ silica, 30 mm diameter, 100 mm length), using decreasingly polar mixtures of water (containing 1% NH₃) and MeCN as eluents. Fractions containing the desired compound were evaporated to dryness to afford 1-[(8aS)-6-chloro-5-(2-fluoro-6-hydroxyphenyl)-8a,9,11,12-tetrahydropyrazino [2′,1′:3,4][1,4]oxazepino[5,6,7-de]quinazolin-10 (8H)-yl]prop-2-en-1-one (31 mg, 12%) as a solid. 1H NMR (500 MHz, DMSO, 27° C.) 2.96-3.14 (1H, m), 3.18-3.49 (2H+H2O, m), 3.99-4.07 (1H, m), 4.11-4.34 (1H, m), 4.35-4.53 (1H, m), 4.66 (2H, s), 4.81 (1H, d), 5.71-5.78 (1H, m), 6.18 (1H, d), 6.7-6.77 (1H, m), 6.81 (1H, dd), 6.84-6.93 (1H, m), 7.27 (1H, td), 7.33 (1H, d), 8.54 (1H, s), 10.06 (1H, s). m/z: ES+ [M+H]+ 441.

### Example 10, 1-[(8aS)-6-Chloro-5-(2-fluoro-6-hydroxyphenyl)-8a,9,11,12-tetrahydropyrazino[2′,1′: 3,4][1,4]oxazepino[5,6,7-de]quinazolin-10 (8H)-yl]prop-2-en-1-one, Atropisomer 1; and

### Example 11, 1-[(8aS)-6-chloro-5-(2-fluoro-6-hydroxyphenyl)-8a,9,11,12-tetrahydropyrazino[2′,1′: 3,4][1,4]oxazepino[5,6,7-de]quinazolin-10 (8H)-yl]prop-2-en-1-one, Atropisomer 2

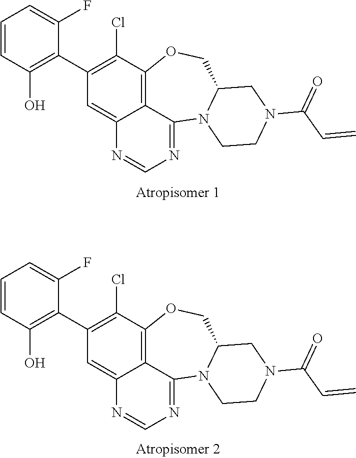 [SMILES: C=CC(=O)N1CCN2c3ncnc4cc(-c5c(O)cccc5F)c(Cl)c(c34)OC[C@@H]2C1 ; C=CC(=O)N1CCN2c3ncnc4cc(-c5c(O)cccc5F)c(Cl)c(c34)OC[C@@H]2C1]

A mixture of the atropisomers (Example 9, 31 mg) was dissolved in MeOH separated using the SFC conditions: Column: Chiralcel OJ-H, 20×250 mm, 5 micron Mobile phase: 35% MeOH+0.1% NH₃/65% scCO₂ Flow rate: 60 ml/min BPR: 120 bar Column temperature: 40° C. This afforded the first atropisomer of 1-[(8aS)-6-chloro-5-(2-fluoro-6-hydroxyphenyl)-8a,9,11,12-tetrahydropyrazino[2′,1′:3,4][1,4]oxazepino[5,6,7-de]quinazolin-10 (8H)-yl]prop-2-en-1-one (Atropisomer 1, 10.4 mg, 96% d.e.) as a white solid. 1H NMR (500 MHz, CDCl₃, 27° C.) 2.93-5.02 (9H, m), 5.75 (1H, dd), 6.33 (1H, d), 6.52 (1H, dd), 6.63 (1H, t), 6.84 (1H, d), 7.13-7.26 (1H+CHCl3, m), 7.51 (1H, s), 8.32 (1H, s), 10.04 (1H, s). m/z: ES+ [M+H]+ 441. Chiral analysis Phenomonex Lux C3, 150×3.0 mm id, 3 micron, Mobile phase 70%=scCO₂, 30%=MeOH+0.1% NH₃, Flow rate 2.0 ml/min, retention time 1.19 minutes. This was followed by the second eluted peak 1-[(8aS)-6-chloro-5-(2-fluoro-6-hydroxyphenyl)-8a,9,11,12-tetrahydropyrazino [2′,1′:3,4][1,4]oxazepino[5,6,7-de]quinazolin-10 (8H)-yl]prop-2-en-1-one (Atropisomer 2, 10.3 mg, 94% d.e.) as a white solid. 1H NMR (500 MHz, CDCl₃, 27° C.) 2.84-3.15 (2H, m), 3.19-3.57 (1H, m), 3.78 (1H, d), 3.97 (1H, d), 4.23-4.77 (3H, m), 4.97 (1H, d), 5.74 (1H, d), 6.31 (1H, d), 6.43-6.58 (1H, m), 6.62 (1H, t), 6.84 (1H, d), 7.11-7.3 (1H+CHCl3, m), 7.51 (1H, s), 8.30 (1H, s), 10.34 (1H, s). m/z: ES+ [M+H]+ 441. Chiral analysis Phenomonex Lux C3, 150×3.0 mm id, 3 micron, Mobile phase 70%=scCO₂, 30%=MeOH+0.1% NH₃, Flow rate 2.0 ml/min, retention time 2.25 minutes.

### Example 12, (E)-1-((8aS)-6-Chloro-5-(2-fluoro-6-hydroxyphenyl)-8a,9,11,12-tetrahydropyrazino[2′,1′:3,4][1,4]oxazepino[5,6,7-de]quinazolin-10 (8H)-yl)-4-(dimethylamino)but-2-en-1-one

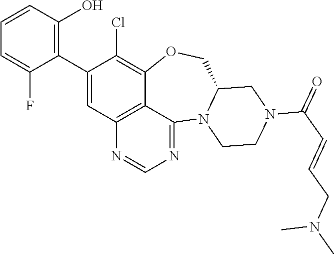 [SMILES: CN(C)C/C=C/C(=O)N1CCN2c3ncnc4cc(-c5c(O)cccc5F)c(Cl)c(c34)OC[C@@H]2C1]

DIPEA (91 μl, 0.52 mmol) was added in one portion to 2-[(8aS)-6-chloro-8,8a,9,10,11,12-hexahydropyrazino[2′,1′:3,4][1,4]oxazepino[5,6,7-de]quinazolin-5-yl]-3-fluorophenol (67 mg, 0.17 mmol), O-(7-azabenzotriazol-1-yl)-N,N,N′,N′-tetramethyluronium hexafluorophosphate (79 mg, 0.21 mmol) and (E)-4-(dimethylamino)but-2-enoic acid. HCl salt (31.6 mg, 0.19 mmol) in DMA (776 μl). The resulting solution was stirred at room temperature for 1 hour. The reaction mixture was poured into water, extracted into EtOAc and washed with brine. The organic layer dried over MgSO₄, filtered and evaporated to afford crude product. The crude product was purified by preparative HPLC (Waters XSelect CSH C18 column, 5μ silica, 50 mm diameter, 100 mm length), using decreasingly polar mixtures of water (containing 0.1% NH₃) and MeCN as eluents. Fractions containing the desired compound were evaporated to dryness to afford (E)-1-((8aS)-6-chloro-5-(2-fluoro-6-hydroxyphenyl)-8a,9,11,12-tetrahydropyrazino [2′,1′:3,4][1,4]oxazepino[5,6,7-de]quinazolin-10 (8H)-yl)-4-(dimethylamino)but-2-en-1-one (48 mg, 55%) as a white solid. 1H NMR (500 MHz, DMSO, 27° C.) 2.16 (6H, s), 2.95-3.12 (3H, in), 3.96-4.9 (8H, in), 6.68 (2H, s), 6.74 (1H, td), 6.81 (1H, dd), 7.27 (1H, td), 7.33 (1H, d), 8.54 (1H, s), 10.05 (1H, s). m/z: ES+ [M+H]+ 498.

### [(8aS)-10-(Tert-butoxycarbonyl)-6-chloro-8,8a,9,10,11,12-hexahydropyrazino-[2′,1′:3,4][1,4]oxazepino[5,6,7-de]quinazolin-5-yl]boronic acid

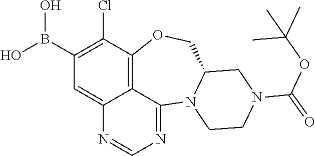 [SMILES: CC(C)(C)OC(=O)N1CCN2c3ncnc4cc(B(O)O)c(Cl)c(c34)OC[C@@H]2C1]

PdCl₂(dppf) DCM (0.23 g, 0.28 mmol) was added to tert-butyl (8aR)-5-bromo-8a,9,11,12-tetrahydropyrazino[2′,1′:3,4][1,4]oxazepino[5,6,7-de]quinazoline-10 (8H)-carboxylate (1.15 g, 2.52 mmol), 4,4,4,4,5,5,5,5-octamethyl-2,2′-bi(1,3,2-dioxaborolane) (1.47 g, 5.80 mmol) and potassium acetate (1.24 g, 12.62 mmol) in degassed dioxane (15 ml) under nitrogen. The resulting suspension was degassed further before being stirred at reflux for 16 hours. then allowed to cool to room temperature. The reaction was diluted with EtOAc (100 ml) and filtered through celite, then washed with water (40 ml), brine (40 ml), dried over MgSO₄ and reduce under vacuum to give crude [(8aS)-10-(tert-butoxycarbonyl)-6-chloro-8,8a,9,10,11,12-hexahydropyrazino[2′,1′:3,4][1,4]oxazepino[5,6,7-de]quinazolin-5-yl]boronic acid (2.68 g, >100%) which was used without further purification. m/z: ES+ [M+H]+ 421.

### Tert-butyl (8aS)-6-chloro-5-(2-oxo-2,3-dihydro-1H-benzimidazol-4-yl)-8a,9,11,12-tetrahydropyrazino[2′,1′:3,4][1,4]oxazepino[5,6,7-de]quinazoline-10 (8H)-carboxylate and tert-butyl (8aS)-5-(2-oxo-2,3-dihydro-1H-benzimidazol-4-yl)-8a,9,11,12-tetrahydropyrazino[2′,1′:3,4][1,4]oxazepino[5,6,7-de]quinazoline-10 (8H)-carboxylate

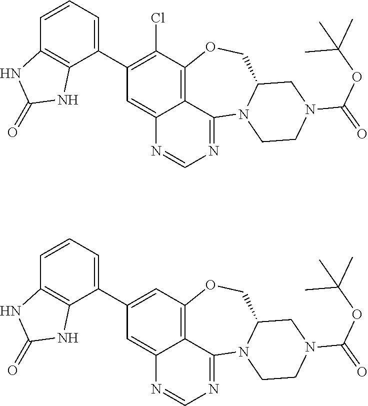 [SMILES: CC(C)(C)OC(=O)N1CCN2c3ncnc4cc(-c5cccc6[nH]c(=O)[nH]c56)c(Cl)c(c34)OC[C@@H]2C1 ; CC(C)(C)OC(=O)N1CCN2c3ncnc4cc(-c5cccc6[nH]c(=O)[nH]c56)cc(c34)OC[C@@H]2C1]

1,1 Bis(di-tert-butylphosphino)ferrocene palladium dichloride (38 mg, 0.06 mmol) was added to 4-bromo-1,3-dihydro-2H-benzo[d]imidazol-2-one (137 mg, 0.64 mmol), crude [(8aS)-10-(tert-butoxycarbonyl)-6-chloro-8,8a,9,10,11,12-hexahydropyrazino-[2′,1′:3,4][1,4]oxazepino [5,6,7-de]quinazolin-5-yl]boronic acid (665 mg, 0.58 mmol) and potassium carbonate (162 mg, 1.17 mmol) in degassed dioxane (2 ml)/water (2 ml) and sealed into a microwave tube. The reaction was heated at 100° C. for 12 hours in a microwave reactor then cooled to room temperature. The reaction mixture was concentrated and diluted with EtOAc (50 ml), and washed with water (25 ml). The organic layer was dried with MgSO₄, filtered and evaporated to afford crude product. The crude product was purified by flash silica chromatography, elution gradient 0 to 10% MeOH in DCM. Pure fractions were evaporated to dryness to afford a mixture of tert-butyl (8aS)-6-chloro-5-(2-oxo-2,3-dihydro-1H-benzimidazol-4-yl)-8a,9,11,12-tetrahydropyrazino-[2′,1′:3,4][1,4]oxazepino[5,6,7-de]quinazoline-10 (8H)-carboxylate and tert-butyl (8aS)-5-(2-oxo-2,3-dihydro-1H-benzimidazol-4-yl)-8a,9,11,12-tetrahydropyrazino [2′,1′:3,4]-[1,4]oxazepino[5,6,7-de]quinazoline-10 (8H)-carboxylate (122 mg). m/z: ES+ [M+H]+ 509 (30%); ES+ [M+H]+ 475 (70%).

### 4-[(8aS)-6-Chloro-8,8a,9,10,11,12-hexahydropyrazino[2′,1′:3,4][1,4]oxazepino[5,6,7-de]quinazolin-5-yl]-1,3-dihydro-2H-benzimidazol-2-one and 4-[(8aS)-8,8a,9,10,11,12-hexahydropyrazino[2′,1′:3,4][1,4]oxazepino[5,6,7-de]-quinazolin-5-yl]-1,3-dihydro-2H-benzimidazol-2-one

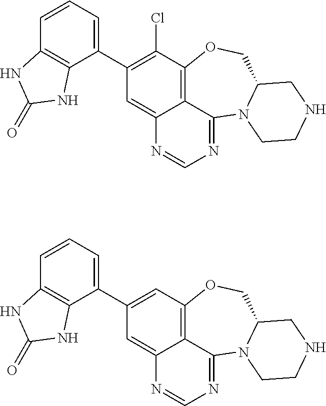 [SMILES: O=c1[nH]c2cccc(-c3cc4ncnc5c4c(c3Cl)OC[C@@H]3CNCCN53)c2[nH]1 ; O=c1[nH]c2cccc(-c3cc4c5c(ncnc5c3)N3CCNC[C@H]3CO4)c2[nH]1]

TFA (0.25 ml, 3.27 mmol) was added to a mixture of tert-butyl (8aS)-6-chloro-5-(2-oxo-2,3-dihydro-1H-benzimidazol-4-yl)-8a,9,11,12-tetrahydropyrazino[2′,1′:3,4][1,4]-oxazepino[5,6,7-de]quinazoline-10 (8H)-carboxylate and tert-butyl (8aS)-5-(2-oxo-2,3-dihydro-1H-benzimidazol-4-yl)-8a,9,11,12-tetrahydropyrazino[2′,1′:3,4][1,4]-oxazepino[5,6,7-de]quinazoline-10 (8H)-carboxylate (122 mg) in DCM (1 ml). The resulting solution was stirred at room temperature for 1 hour. The reaction mixture was evaporated to dryness and re-dissolved in MeOH (2 ml).This was purified by ion exchange chromatography, using an SCX2 (5 g) column. The desired product was eluted from the column using 1M NH₃/MeOH and pure fractions were evaporated to dryness to afford a mixture of 4-[(8aS)-6-chloro-8,8a,9,10,11,12-hexahydropyrazino[2′,1′:3,4][1,4]-oxazepino[5,6,7-de]quinazolin-5-yl]-1,3-dihydro-2H-benzimidazol-2-one and 4-[(8aS)-8,8a,9,10,11,12-hexahydropyrazino[2′,1′:3,4][1,4]oxazepino[5,6,7-de]quinazolin-5-yl]-1,3-dihydro-2H-benzimidazol-2-one (84 mg) which was used directly in the next synthetic step. m/z: ES+ [M+H]+ 375 (69%) and ES+ [M+H]+ 409 (31%).

### Example 13, 4-[(8aS)-10-Acryloyl-6-chloro-8,8a,9,10,11,12-hexahydropyrazino[2′,1′:3,4][1,4]-oxazepino[5,6,7-de]quinazolin-5-yl]-1,3-dihydro-2H-benzimidazol-2-one

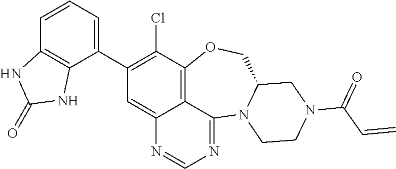 [SMILES: C=CC(=O)N1CCN2c3ncnc4cc(-c5cccc6[nH]c(=O)[nH]c56)c(Cl)c(c34)OC[C@@H]2C1]

A solution of acryloyl chloride (21 mg, 0.23 mmol) in DMA (0.25 ml) was added to a stirred suspension of a mixture of 4-[(8aS)-6-chloro-8,8a,9,10,11,12-hexahydropyrazino-[2′,1′:3,4][1,4]oxazepino[5,6,7-de]quinazolin-5-yl]-1,3-dihydro-2H-benzimidazol-2-one, 4-[(8aS)-8,8a,9,10,11,12-hexahydropyrazino[2′,1′:3,4][1,4]oxazepino[5,6,7-de]quinazolin-5-yl]-1,3-dihydro-2H-benzimidazol-2-one (84 mg) and triethylamine (0.084 ml, 0.6 mmol) in DMA (0.75 ml) cooled at 0° C. The resulting mixture was stirred at room temperature for 30 minutes. The reaction mixture was diluted with DMSO (1 ml), then filtered. The filtrate were purified by preparative HPLC (Waters CSH C18 OBD column, 5μ silica, 30 mm diameter, 100 mm length), using decreasingly polar mixtures of water (containing 1% NH₃) and MeCN as eluents. Fractions containing the desired compound were evaporated to dryness to afford 4-[(8aS)-10-acryloyl-6-chloro-8,8a,9,10,11,12-hexahydropyrazino-[2′,1′:3,4][1,4]oxazepino[5,6,7-de]quinazolin-5-yl]-1,3-dihydro-2H-benzimidazol-2-one (11 mg, 15%) as a solid. 1H NMR (500 MHz, MeOD, 27° C.) 3.13-3.29 (1H, m), 3.29-3.47 (1H+MeOH, m), 3.48-3.66 (1H, m), 4.10 (1H, s), 4.17-4.36 (1H, m), 4.47-4.7 (3H, m), 5.01 (1H, d), 5.82 (1H, dd), 6.29 (1H, dd), 6.73-6.92 (1H, m), 6.98 (1H, dd), 7.08-7.24 (2H, m), 7.44 (1H, s), 8.51 (1H, s). m/z: ES+ [M+H]+ 463.

### Tert-butyl (8aS)-6-chloro-5-(5-methyl-1H-pyrazolo[3,4-b]pyridin-4-yl)-8a,9,11,12-tetrahydropyrazino[2′,1′:3,4][1,4]oxazepino[5,6,7-de]quinazoline-10 (8H)-carboxylate

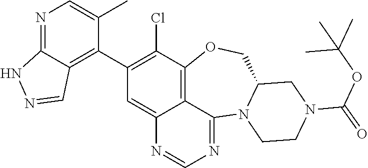 [SMILES: Cc1cnc2[nH]ncc2c1-c1cc2ncnc3c2c(c1Cl)OC[C@@H]1CN(C(=O)OC(C)(C)C)CCN31]

1,1 Bis(di-tert-butylphosphino)ferrocene palladium dichloride (23.5 mg, 0.04 mmol) was added to, 4-iodo-5-methyl-1H-pyrazolo[3,4-b]pyridine (93 mg, 0.36 mmol), crude [(8aS)-10-(tert-butoxycarbonyl)-6-chloro-8,8a,9,10,11,12-hexahydropyrazino[2′,1′:3,4]-[1,4]oxazepino[5,6,7-de]quinazolin-5-yl]boronic acid (410 mg, 0.36 mmol) and potassium carbonate (100 mg, 0.72 mmol) in degassed dioxane (2 ml)/water (2 ml) and sealed into a microwave tube. The reaction was heated at 100° C. for 12 hours in a microwave reactor then cooled to room temperature. The reaction mixture was concentrated and diluted with EtOAc (50 ml), and washed with water (25 ml). The organic layer was dried with MgSO₄, filtered and evaporated to afford crude product. The crude product was purified by flash silica chromatography, elution gradient 0 to 10% MeOH in DCM. Pure fractions were evaporated to dryness to afford tert-butyl (8aS)-6-chloro-5-(5-methyl-1H-pyrazolo[3,4-b]pyridin-4-yl)-8a,9,11,12-tetrahydropyrazino[2′,1′:3,4][1,4]oxazepino[5,6,7-de]-quinazoline-10 (8H)-carboxylate (45 mg, 25%) as a brown solid, which was used without further purification. m/z: ES+ [M+H]+ 508.

### (8aS)-6-Chloro-5-(5-methyl-1H-pyrazolo[3,4-b]pyridin-4-yl)-8,8a,9,10,11,12-hexahydropyrazino[2′,1′:3,4][1,4]oxazepino[5,6,7-de]quinazoline

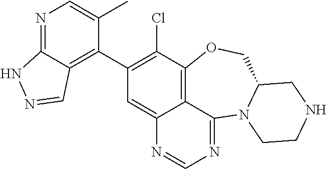 [SMILES: Cc1cnc2[nH]ncc2c1-c1cc2ncnc3c2c(c1Cl)OC[C@@H]1CNCCN31]

TFA (0.2 ml, 2.61 mmol) was added to tert-butyl (8aS)-6-chloro-5-(5-methyl-1H-pyrazolo[3,4-b]pyridin-4-yl)-8a,9,11,12-tetrahydropyrazino[2′,1′:3,4][1,4]oxazepino[5,6,7-de]quinazoline-10 (8H)-carboxylate (45 mg, 0.09 mmol) in DCM (1 ml). The resulting solution was stirred at room temperature for 1 hour. The reaction mixture was evaporated to dryness and the residue was purified by ion exchange chromatography, using an SCX2 (5 g) column. The desired product was eluted from the column using 1M NH₃/MeOH and pure fractions were evaporated to dryness to afford (8aS)-6-chloro-5-(5-methyl-1H-pyrazolo[3,4-b]pyridin-4-yl)-8,8a,9,10,11,12-hexahydropyrazino[2′,1′:3,4][1,4]oxazepino-[5,6,7-de]quinazoline (21 mg, 58%) as a brown film, which was used without further purification. m/z: ES+ [M+H]+ 408.

### Example 14, 1-[(8aS)-6-Chloro-5-(5-methyl-1H-pyrazolo[3,4-b]pyridin-4-yl)-8a,9,11,12-tetrahydro-pyrazino[2′,1′:3,4][1,4]oxazepino[5,6,7-de]quinazolin-10 (8H)-yl]prop-2-en-1-one

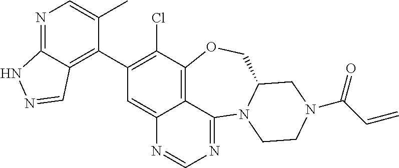 [SMILES: C=CC(=O)N1CCN2c3ncnc4cc(-c5c(C)cnc6[nH]ncc56)c(Cl)c(c34)OC[C@@H]2C1]

A solution of acryloyl chloride (5.1 mg, 0.06 mmol) in DMA (0.25 ml) was added to a stirred suspension of (8aS)-6-chloro-5-(5-methyl-1H-pyrazolo[3,4-b]pyridin-4-yl)-8,8a,9,10,11,12-hexahydropyrazino[2′,1′:3,4][1,4]oxazepino[5,6,7-de]quinazoline (21 mg, 0.05 mmol), and triethylamine (0.022 ml, 0.15 mmol) in DMA (0.25 ml) cooled at 0° C. The resulting mixture was stirred at room temperature for 30 minutes. The reaction mixture was diluted with DMSO (1 ml), then filtered. The filtrate was purified by preparative HPLC (Waters CSH C18 OBD column, 5μ silica, 30 mm diameter, 100 mm length), using decreasingly polar mixtures of water (containing 1% NH₃) and MeCN as eluents. Fractions containing the desired compound were evaporated to dryness to afford 1-[(8aS)-6-chloro-5-(5-methyl-1H-pyrazolo[3,4-b]pyridin-4-yl)-8a,9,11,12-tetrahydropyrazino[2′,1′:3,4][1,4]-oxazepino[5,6,7-de]quinazolin-10 (8H)-yl]prop-2-en-1-one (4 mg, 17%) as a solid. 1H NMR (500 MHz, CDCl₃, 27° C.) 2.20 (3H, d), 2.93-3.33 (2H, m), 3.36-3.7 (1H+MeOH, m), 3.76-4.18 (2H, m), 4.4-4.79 (3H, m), 4.96 (1H, d), 5.63-5.83 (1H, m), 6.34 (1H, d), 6.57 (1H, dd), 7.48 (1H, s), 7.58-7.66 (1H, m), 8.51 (1H, s), 8.62 (1H, s), 11.78 (1H, s). m/z: ES+ [M+H]+ 462.

### Tert-butyl (8aS)-6-chloro-5-(2-chloro-6-methoxyphenyl)-8a,9,11,12-tetrahydropyrazino[2′,1′:3,4][1,4]oxazepino[5,6,7-de]quinazoline-10 (8H)-carboxylate

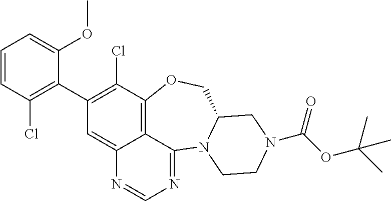 [SMILES: COc1cccc(Cl)c1-c1cc2ncnc3c2c(c1Cl)OC[C@@H]1CN(C(=O)OC(C)(C)C)CCN31]

K₂CO₃ (218 mg, 1.58 mmol) was added to tert-butyl (8aS)-5-bromo-6-chloro-8a,9,11,12-tetrahydropyrazino[2′,1′:3,4][1,4]oxazepino[5,6,7-de]quinazoline-10 (8H)-carboxylate (360 mg, 0.79 mmol), (2-chloro-6-methoxyphenyl)boronic acid (177 mg, 0.95 mmol) and K₂CO₃ (218 mg, 1.58 mmol) in dioxane/H₂O (5 ml) at 25° C. under nitrogen. The resulting mixture was stirred at 100° C. for 2 hours. The solvent was removed under reduced pressure. The crude product was purified by flash silica chromatography, elution gradient 0 to 30% EtOAc in petroleum ether. Pure fractions were evaporated to dryness to afford tert-butyl (8aS)-6-chloro-5-(2-chloro-6-methoxyphenyl)-8a,9,11,12-tetrahydropyrazino-[2′,1′:3,4][1,4]oxazepino[5,6,7-de]quinazoline-10 (8H)-carboxylate (460 mg, >100%) as a white foam. 1H NMR (DMSO-d6, 300 MHz) δ 1.45 (9H, s), 3.10 (2H, brs), 3.14-3.27 (1H, m), 3.72 (3H, s), 3.84-3.97 (2H, m), 4.06-4.13 (1H, m), 4.57-4.73 (2H, m), 4.81 (1H, d), 7.13-7.23 (2H, m), 7.27 (1H, s), 7.47 (1H, t), 8.55 (1H, s). m/z (ES+), [M+H]+=517.

### (8aS)-6-Chloro-5-(2-chloro-6-hydroxyphenyl)-8,8a,9,10,11,12-hexahydropyrazino-[2′,1′:3,4][1,4]oxazepino[5,6,7-de]quinazoline hydrogen bromide

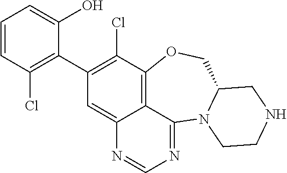 [SMILES: Oc1cccc(Cl)c1-c1cc2ncnc3c2c(c1Cl)OC[C@@H]1CNCCN31]

BBr₃ (0.49 ml, 5.22 mmol) was added to tert-butyl (8aS)-6-chloro-5-(2-chloro-6-methoxyphenyl)-8a,9,11,12-tetrahydropyrazino[2′,1′:3,4][1,4]oxazepino[5,6,7-de]quinazoline-10 (8H)-carboxylate (450 mg, 0.87 mmol) in DCM (5 ml) at 0° C. under nitrogen. The resulting suspension was stirred at room temperature for 1 hour. The reaction mixture was quenched with MeOH (2 ml). The solvent was removed under reduced pressure to afford (8aS)-6-chloro-5-(2-chloro-6-hydroxyphenyl)-8,8a,9,10,11,12-hexahydropyrazino[2′,1′:3,4][1,4]oxazepino[5,6,7-de]quinazoline hydrogen bromide (430 mg, >100%) as a brown gum. The product was used in the next step directly without further purification. m/z (ES+), [M+H]+=403.

### Example 15, 1-[(8aS)-6-Chloro-5-(2-chloro-6-hydroxyphenyl)-8a,9,11,12-tetrahydropyrazino[2′,1′: 3,4][1,4]oxazepino[5,6,7-de]quinazolin-10 (8H)-yl]prop-2-en-1-one, Atropisomer 1; and

### Example 16, 1-[(8aS)-6-chloro-5-(2-chloro-6-hydroxyphenyl)-8a,9,11,12-tetrahydropyrazino[2′,1′: 3,4][1,4]oxazepino[5,6,7-de]quinazolin-10 (8H)-yl]prop-2-en-1-one, Atropisomer 2

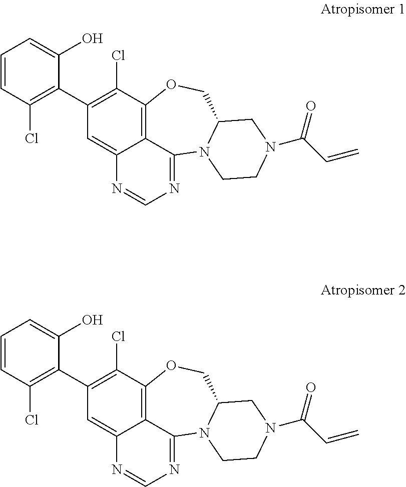 [SMILES: C=CC(=O)N1CCN2c3ncnc4cc(-c5c(O)cccc5Cl)c(Cl)c(c34)OC[C@@H]2C1 ; C=CC(=O)N1CCN2c3ncnc4cc(-c5c(O)cccc5Cl)c(Cl)c(c34)OC[C@@H]2C1]

Acryloyl chloride (52.3 mg, 0.58 mmol) was added to (8aS)-6-chloro-5-(2-chloro-6-hydroxyphenyl)-8,8a,9,10,11,12-hexahydropyrazino[2′,1′:3,4][1,4]oxazepino[5,6,7-de]quinazoline hydrogen bromide (400 mg, 0.58 mmol) and DIPEA (0.202 ml, 1.16 mmol) in DMF (4 ml) at −10° C. under nitrogen. The temperature was increased to room temperature and the resulting mixture was stirred at room temperature for 1 hour. The crude product was purified by preparative HPLC (Column: XBridge Prep OBD C18 Column 30×150 mm 5 um; Mobile Phase A: Water (10 mmol/1 NH₄HCO₃+0.1% NH₃.H₂O), Mobile Phase B: ACN; Flow rate: 60 ml/min; Gradient: 30% B to 49% B in 8 min; 254/220 nm; Rt: 7.40 min). Fractions containing the desired compound were evaporated to dryness to afford 1-[(8aS)-6-chloro-5-(2-chloro-6-hydroxyphenyl)-8a,9,11,12-tetrahydro-pyrazino[2′,1′:3,4][1,4]oxazepino[5,6,7-de]quinazolin-10 (8H)-yl]prop-2-en-1-one (117 mg) as a white solid. The product was purified by preparative chiral-HPLC on a Column: Chiralpak ID-2, 2*25 cm, 5 um; Mobile Phase A: Hex (1% TFA)-HPLC, Mobile Phase B: EtOH-HPLC; Flow rate: 20 ml/min; isocratic 50% B over 22 min; 220/254 nm. The fractions containing the desired compound were evaporated to dryness to afford the first atropisomer (retention time 14.97 min) 1-[(8aS)-6-chloro-5-(2-chloro-6-hydroxy-phenyl)-8a,9,11,12-tetrahydropyrazino[2′,1′:3,4][1,4]oxazepino[5,6,7-de]quinazolin-10 (8H)-yl]prop-2-en-1-one (Atropisomer 1, 35 mg, 13%, d.e. 100%) as a white solid. 1H NMR (DMSO-d6, 300 MHz) δ 2.94-3.17 (1H, m), 3.18-3.31 (1H, m), 3.35-3.52 (1H, m), 4.06 (1H, dd), 4.12-4.56 (2H, m), 4.58-4.89 (3H, m), 5.76 (1H, dd), 6.20 (1H, dd), 6.80-6.98 (2H, m), 7.03 (1H, dd), 7.22-7.34 (2H, m), 8.56 (1H, s), 10.00 (1H, s). m/z (ES+), [M+H]+=457; base, HPLC tR=1.029 min. Analytical chiral HPLC method CHIRALPAK ID-3 (50×4.6 mm 3 um) a flow rate of 1 ml/minute and detection was by UV absorbance at wavelength of 254 nm. Oven temperature of 25 [symbol] C. Mobile phase: Hex (0.1% DEA):IPA=50:50, retention time 1.759 minutes. This was followed by the second atropisomer (retention time 18.69 min) 1-[(8aS)-6-chloro-5-(2-chloro-6-hydroxyphenyl)-8a,9,11,12-tetrahydropyrazino[2′,1′:3,4][1,4]oxazepino[5,6,7-de]quinazolin-10 (8H)-yl]prop-2-en-1-one (Atropisomer 2, 35 mg, 13%, d.e. 99.1%) as a white solid. 1H NMR (DMSO-d6, 300 MHz) δ 2.96-3.32 (2H, m), 3.36-3.53 (1H, m), 3.98-4.11 (1H, m), 4.11-4.57 (2H, m), 4.67 (2H, d), 4.82 (1H, t), 5.76 (1H, dd), 6.20 (1H, dd), 6.80-6.99 (2H, m), 7.02 (1H, dd), 7.22-7.34 (2H, m), 8.57 (1H, s), 10.00 (1H, s). m/z (ES+), [M+H]+=457; base, HPLC tR=1.02 min. Analytical chiral HPLC method CHIRALPAK ID-3 (50×4.6 mm 3 um) a flow rate of 1 ml/minute and detection was by UV absorbance at wavelength of 254 nm. Oven temperature of 25 [symbol] C. Mobile phase: Hex (0.1% DEA):IPA=50:50, retention time 3.00 minutes.

### 2-Bromo-1-methoxy-3-((4-methoxybenzyl)oxy)benzene

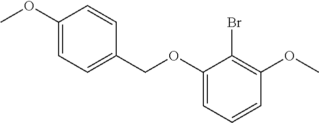

1-(Chloromethyl)-4-methoxybenzene (3.7 g, 23.64 mmol) in DMF was added to 2-bromo-3-methoxyphenol (4 g, 19.7 mmol), K₂CO₃ (5.45 g, 39.4 mmol) and KI (1.64 g, 9.85 mmol) in DMF (30 ml) at room temperature under nitrogen. The resulting mixture was stirred at 80° C. for 2 hours. The reaction mixture was diluted with EtOAc (200 ml), and washed sequentially with saturated NH₄Cl (100 ml), saturated brine (150 ml×3). The organic layer was dried over Na₂SO₄, filtered and evaporated to afford crude product. The crude product was purified by flash silica chromatography, elution gradient 0 to 100%, 39% EtOAc in petroleum ether. Pure fractions were evaporated to dryness to afford 2-bromo-1-methoxy-3-((4-methoxybenzyl)oxy)benzene (5.6 g, 88%) as a yellow gum. 1H NMR (DMSO-d6, 300 MHz) δ 3.76 (3H, s), 3.83 (3H, s), 5.11 (2H, s), 6.77 (2H, dd), 6.89-7.02 (2H, m), 7.20-7.49 (3H, m).

### 2-(2-Methoxy-6-((4-methoxybenzyl)oxy)phenyl)-4,4,5,5-tetramethyl-1,3,2-dioxaborolane

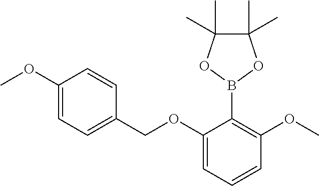

n-Butyllithium, 2.5M solution in hexanes (7.18 ml, 17.94 mmol) was added to 2-bromo-1-((4-methoxybenzyl)oxy)-3-(trifluoromethyl)benzene (5.4 g, 14.95 mmol) in THF (50 ml) at −78° C. under nitrogen. After 30 minutes, 2-isopropoxy-4,4,5,5-tetramethyl-1,3,2-dioxaborolane (3.06 g, 16.45 mmol) was added to the mixture. The resulting suspension was stirred at room temperature for 16 hours. The reaction mixture was quenched with water (100 ml), extracted with EtOAc (3×200 ml), the organic layer was dried over Na₂SO₄, filtered and evaporated to afford a white gum. The crude product was purified by flash silica chromatography, elution gradient 8 to 20%, 11% EtOAc in petroleum ether. Pure fractions were evaporated to dryness to afford 2-(2-methoxy-6-((4-methoxybenzyl)-oxy)phenyl)-4,4,5,5-tetramethyl-1,3,2-dioxaborolane (1.16 g, 21%) as a white solid. 1H NMR (DMSO-d6, 300 MHz) δ 1.21 (12H, s), 3.69 (3H, s), 3.75 (3H, s), 4.95 (2H, s), 6.60 (2H, dd), 6.92 (2H, d), 7.20-7.42 (3H, m). m/z (ES+), [M+H]+=371.

### Tert-butyl (8aS)-6-chloro-5-{2-methoxy-6-[(4-methoxyphenyl)methoxy]phenyl}-8a,9,11,12-tetrahydropyrazino[2′,1′:3,4][1,4]oxazepino[5,6,7-de]quinazoline-10 (8H)-carboxylate

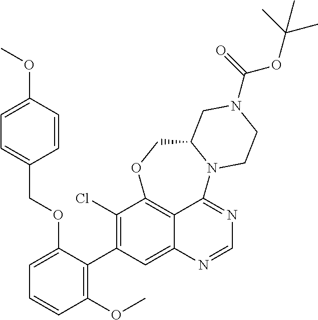 [SMILES: COc1ccc(COc2cccc(OC)c2-c2cc3ncnc4c3c(c2Cl)OC[C@@H]2CN(C(=O)OC(C)(C)C)CCN42)cc1]

Pd(Ph₃P)₄ (101 mg, 0.09 mmol) was added to 2-(2-methoxy-6-((4-methoxybenzyl)oxy)-phenyl)-4,4,5,5-tetramethyl-1,3,2-dioxaborolane (650 mg, 1.76 mmol), tert-butyl (8aS)-5-bromo-6-chloro-8a,9,11,12-tetrahydropyrazino[2′,1′:3,4][1,4]oxazepino[5,6,7-de]-quinazoline-10 (8H)-carboxylate (400 mg, 0.88 mmol) and K₂CO₃ (243 mg, 1.76 mmol) in 1,4-dioxane/H₂O (15 ml) at room temperature under nitrogen. The resulting suspension was stirred at 100° C. for 16 hours. The solvent was removed under reduced pressure. The crude product was purified by flash silica chromatography, elution gradient 0 to 100%, 98% EtOAc in petroleum ether. Pure fractions were evaporated to dryness to afford tert-butyl (8aS)-6-chloro-5-{2-methoxy-6-[(4-methoxyphenyl)methoxy]phenyl}-8a,9,11,12-tetrahydropyrazino[2′,1′:3,4][1,4]oxazepino [5,6,7-de]quinazoline-10 (8H)-carboxylate (540 mg, 99%) as a pale yellow solid. 1H NMR (DMSO-d6, 300 MHz) δ 1.45 (9H, s), 3.05-3.12 (2H, m), 3.11-3.27 (1H, m), 3.68 (3H, d), 3.70 (3H, d), 3.87-3.99 (2H, m), 4.09 (1H, d), 4.51-4.73 (2H, m), 4.82 (1H, d), 4.90-5.09 (2H, m), 6.73-6.88 (4H, m), 7.10-7.20 (2H, m), 7.23-7.40 (2H, m), 8.52 (1H, s). m/z (ES+), [M+H]+=619.

### 2-[(8aS)-6-Chloro-8,8a,9,10,11,12-hexahydropyrazino[2′,1′:3,4][1,4]oxazepino[5,6,7-de]quinazolin-5-yl]-3-methoxyphenol hydrogen chloride

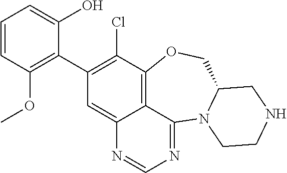 [SMILES: COc1cccc(O)c1-c1cc2ncnc3c2c(c1Cl)OC[C@@H]1CNCCN31]

HCl in dioxane (3 ml, 12 mmol) was added to tert-butyl (8aS)-6-chloro-5-{2-methoxy-6-[(4-methoxyphenyl)methoxy]phenyl}-8a,9,11,12-tetrahydropyrazino[2′,1′:3,4][1,4]-oxazepino[5,6,7-de]quinazoline-10 (8H)-carboxylate (500 mg, 0.81 mmol) in MeOH (3 ml) at room temperature. The resulting mixture was stirred at 60° C. for 2 hours. The solvent was removed under reduced pressure to afford 2-[(8aS)-6-chloro-8,8a,9,10,11,12-hexahydropyrazino[2′,1′:3,4][1,4]oxazepino[5,6,7-de]quinazolin-5-yl]-3-methoxyphenol hydrogen chloride (550 mg, >100%) as a pale yellow solid. The product was used in the next step directly without further purification. 1H NMR (DMSO-d6, 300 MHz) δ 3.46-3.53 (3H, m), 3.76 (3H, s), 4.64-4.69 (1H, m), 4.72-4.81 (3H, m), 4.83-4.97 (1H, m), 5.37-5.48 (1H, m), 6.93 (2H, dd), 7.24 (1H, d), 7.36 (1H, d), 8.95 (1H, s), 9.83 (1H, s). m/z (ES+), [M+H]+=399.

### Example 17, 1-[(8aS)-6-Chloro-5-(2-hydroxy-6-methoxyphenyl)-8a,9,11,12-tetrahydropyrazino[2′,1′:3,4][1,4]oxazepino[5,6,7-de]quinazolin-10 (8H)-yl]prop-2-en-1-one, Atropisomer 1; and

### Example 18, 1-[(8aS)-6-chloro-5-(2-hydroxy-6-methoxyphenyl)-8a,9,11,12-tetrahydropyrazino[2′,1′:3,4][1,4]oxazepino[5,6,7-de]quinazolin-10 (8H)-yl]prop-2-en-1-one, Atropisomer 2

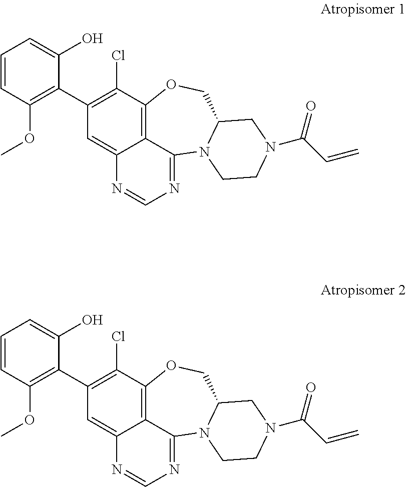 [SMILES: C=CC(=O)N1CCN2c3ncnc4cc(-c5c(O)cccc5OC)c(Cl)c(c34)OC[C@@H]2C1 ; C=CC(=O)N1CCN2c3ncnc4cc(-c5c(O)cccc5OC)c(Cl)c(c34)OC[C@@H]2C1]

Acryloyl chloride (83 mg, 0.92 mmol) was added to 2-[(8aS)-6-chloro-8,8a,9,10,11,12-hexahydropyrazino[2′,1′:3,4][1,4]oxazepino[5,6,7-de]quinazolin-5-yl]-3-methoxyphenol hydrogen chloride (500 mg, 0.92 mmol) and DIPEA (0.32 ml, 1.84 mmol) in DMF (3 ml) at −10° C. under nitrogen. The temperature was increased to room temperature. The resulting mixture was stirred at room temperature for 1 hour. The crude product was purified by flash C18-flash chromatography, elution gradient 0 to 100%, 20 minutes, 56% MeCN in water (0.05% NH₄HCO₃). Pure fractions were evaporated to dryness to afford 1-[(8aS)-6-chloro-5-(2-hydroxy-6-methoxyphenyl)-8a,9,11,12-tetrahydropyrazino[2′,1′:3,4][1,4]-oxazepino[5,6,7-de]quinazolin-10 (8H)-yl]prop-2-en-1-one (230 mg) as a white solid. The product was purified by preparative chiral-HPLC on a Column: CHIRAL A room temperature Cellulose-SB, 2*25 cm, 5 um; Mobile Phase A: hexane:DCM=3:1-HPLC, Mobile Phase B: EtOH-HPLC; Flow rate: 20 ml/min; isocratic 50% B over 12 min; 220/254 nm. The fractions containing the first eluted product were evaporated to dryness to afford Example 17, 1-[(8aS)-6-chloro-5-(2-hydroxy-6-methoxyphenyl)-8a,9,11,12-tetrahydropyrazino-[2′,1′:3,4][1,4]oxazepino[5,6,7-de]quinazolin-10 (8H)-yl]prop-2-en-1-one (retention time 5.71 min) (Atropisomer 1, 73 mg, 18%, d.e. 99.7%), as an off-white solid 1H NMR (DMSO-d6, 300 MHz) δ 2.96-3.15 (1H, m), 3.15-3.31 (1H, m), 3.35-3.51 (1H, m), 3.65 (3H, s), 4.03 (1H, dd), 4.09-4.90 (5H, m), 5.76 (1H, dd), 6.20 (1H, dd), 6.58 (2H, d), 6.80-6.99 (1H, m), 7.14-7.26 (2H, m), 8.53 (1H, s), 9.45 (1H, s). m/z (ES+), [M+H]+=453; base, HPLC tR=0.995 min. Analytical chiral HPLC method CHIRALCEL Cellulose-SB (150 mm×4.6 mm 3 um) a flow rate of 1 ml/minute and detection was by UV absorbance at wavelength of 254 nm. Oven temperature of 25° C. Mobile phase: Hex:DCM=3:1 (0.1% DEA):EtOH=50:50, retention time 4.285 minutes. This was followed by the second eluted product, Example 18, 1-[(8aS)-6-chloro-5-(2-hydroxy-6-methoxyphenyl)-8a,9,11,12-tetrahydropyrazino[2′,1′:3,4]-[1,4]oxazepino[5,6,7-de]quinazolin-10 (8H)-yl]prop-2-en-1-one (retention time 7.832 min) (Atropisomer 2, 80 mg, 19%, d.e. 99.7%) as an off-white solid. 1H NMR (DMSO-d6, 300 MHz) δ 2.97-3.30 (2H, m), 3.34-3.53 (1H, m), 3.64 (3H, s), 3.96-4.09 (1H, m), 4.09-4.56 (2H, m), 4.65 (2H, d), 4.74-4.91 (1H, m), 5.76 (1H, dd), 6.19 (1H, dd), 6.59 (2H, dd), 6.80-6.99 (1H, m), 7.14-7.28 (2H, m), 8.54 (1H, s), 9.45 (1H, s). m/z (ES+), [M+H]+=453; base, HPLC tR=1.00 min. Analytical chiral HPLC method CHIRALCEL Cellulose-SB (150 mm×4.6 mm 3 um) a flow rate of 1 ml/minute and detection was by UV absorbance at wavelength of 254 nm. Oven temperature of 25° C. Mobile phase: Hex:DCM=3:1 (0.1% DEA):EtOH=50:50, retention time 6.242 minutes.

### 2-Bromo-3-((4-methoxybenzyl)oxy)benzonitrile

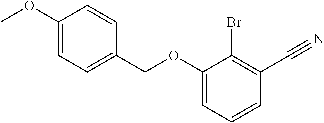 [SMILES: COc1ccc(COc2cccc(C#N)c2Br)cc1]

1-(Chloromethyl)-4-methoxybenzene (3.8 g, 24.24 mmol) was added to 2-bromo-3-hydroxybenzonitrile (4 g, 20.2 mmol), K₂CO₃ (5.58 g, 40.40 mmol) and KI (1.68 g, 10.1 mmol) in DMF (12 ml) at room temperature under nitrogen. The resulting suspended was stirred at 80° C. for 2 hours. The reaction mixture was diluted with DCM (100 ml), and washed sequentially with saturated NH₄Cl (100 ml), saturated brine (100 ml×3). The organic layer was dried over Na₂SO₄, filtered and evaporated to afford crude product. The crude product was purified by flash silica chromatography, elution gradient 0 to 100%, 63% EtOAc in petroleum ether. Pure fractions were evaporated to dryness to afford 2-bromo-3-((4-methoxybenzyl)oxy)benzonitrile (6.2 g, 96%) as a pale yellow solid. 1H NMR (DMSO-d6, 300 MHz) δ 3.76 (3H, s), 5.20 (2H, s), 6.92-7.03 (2H, m), 7.36-7.45 (2H, m), 7.45-7.61 (3H, m).

### 3-((4-Methoxybenzyl)oxy)-2-(4,4,5,5-tetramethyl-1,3,2-dioxaborolan-2-yl)benzonitrile

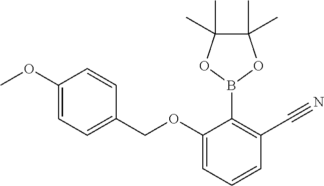 [SMILES: COc1ccc(COc2cccc(C#N)c2B2OC(C)(C)C(C)(C)O2)cc1]

Bis(dibenzylideneacetone)palladium (0.54 g, 0.94 mmol) was added to 2-bromo-3-((4-methoxybenzyl)oxy)benzonitrile (3 g, 9.43 mmol), 4,4,4′,4′,5,5,5′,5′-octamethyl-2,2′-bi(1,3,2-dioxaborolane) (4.79 g, 18.86 mmol), Potassium acetate (1.85 g, 18.86 mmol) and tricyclohexylphosphine (0.26 g, 0.94 mmol) in 1,4-dioxane (60 ml) at room temperature under nitrogen. The resulting mixture was stirred at 100° C. for 16 hours. The solvent was removed under reduced pressure. The crude product was purified by flash silica chromatography, elution gradient 5 to 20%, 15% EtOAc in petroleum ether. Pure fractions were evaporated to dryness to afford 3-((4-methoxybenzyl)oxy)-2-(4,4,5,5-tetramethyl-1,3,2-dioxaborolan-2-yl)benzonitrile (1.17 g, 34%) as a pale orange solid. 1H NMR (DMSO-d6, 300 MHz) δ 1.26 (12H, s), 3.76 (3H, s), 5.07 (2H, s), 6.89-7.00 (2H, m), 7.33-7.46 (4H, m), 7.56 (1H, dd).

Tert-butyl (8aS)-6-chloro-5-{2-cyano-6-[(4-methoxyphenyl)methoxy]phenyl}-8a,9,11,12-tetrahydropyrazino[2′,1′:3,4][1,4]oxazepino[5,6,7-de]quinazoline-10 (8H)-carboxylate

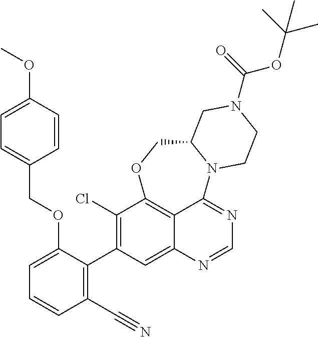 [SMILES: COc1ccc(COc2cccc(C#N)c2-c2cc3ncnc4c3c(c2Cl)OC[C@@H]2CN(C(=O)OC(C)(C)C)CCN42)cc1]

1,1′-Bis(di-tert-butylphosphino)ferrocene palladium dichloride (57.2 mg, 0.09 mmol) was added to tert-butyl (8aS)-5-bromo-6-chloro-8a,9,11,12-tetrahydropyrazino[2′,1′:3,4]-[1,4]oxazepino[5,6,7-de]quinazoline-10 (8H)-carboxylate (400 mg, 0.88 mmol), 3-((4-methoxybenzyl)oxy)-2-(4,4,5,5-tetramethyl-1,3,2-dioxaborolan-2-yl)benzonitrile (641 mg, 1.76 mmol) and K₂CO₃ (243 mg, 1.76 mmol) in 1,4-dioxane/H₂O (20 ml) at room temperature under nitrogen. The resulting mixture was stirred at 100° C. for 16 hours. The solvent was removed under reduced pressure. The crude product was purified by flash silica chromatography, elution gradient 0 to 100%, 98% EtOAc in petroleum ether. Pure fractions were evaporated to dryness to afford tert-butyl (8aS)-6-chloro-5-{2-cyano-6-[(4-methoxyphenyl)methoxy]phenyl}-8a,9,11,12-tetrahydropyrazino[2′,1′:3,4][1,4]oxazepino-[5,6,7-de]quinazoline-10 (8H)-carboxylate (540 mg, 100%) as a brown solid. 1H NMR (DMSO-d6, 300 MHz) δ 1.45 (9H, s), 3.11 (2H, s), 3.15-3.31 (1H, m), 3.71 (3H, d), 3.94 (2H, s), 4.11 (1H, s), 4.61-4.76 (2H, m), 4.82 (1H, d), 5.02-5.20 (2H, m), 6.78-6.90 (2H, m), 7.10-7.24 (2H, m), 7.44 (1H, s), 7.51-7.70 (3H, m), 8.58 (1H, s). m/z (ES+), [M+H]+=614.

### 2-[(8aS)-6-Chloro-8,8a,9,10,11,12-hexahydropyrazino[2′,1′:3,4][1,4]oxazepino[5,6,7-de]quinazolin-5-yl]-3-hydroxybenzonitrile TFA Salt

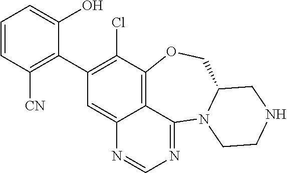 [SMILES: N#Cc1cccc(O)c1-c1cc2ncnc3c2c(c1Cl)OC[C@@H]1CNCCN31]

TFA (2 ml, 25.96 mmol) was added to tert-butyl (8aS)-6-chloro-5-{2-cyano-6-[(4-methoxyphenyl)methoxy]phenyl}-8a,9,11,12-tetrahydropyrazino[2′,1′:3,4][1,4]oxazepino-[5,6,7-de]quinazoline-10 (8H)-carboxylate (570 mg, 0.93 mmol) in DCM (6 ml) at room temperature. The resulting mixture was stirred at room temperature for 1 hour. The solvent was removed under reduced pressure to afford 2-[(8aS)-6-chloro-8,8a,9,10,11,12-hexahydropyrazino[2′,1′:3,4][1,4]oxazepino[5,6,7-de]quinazolin-5-yl]-3-hydroxy-benzonitrile TFA salt (730 mg, >100%) as a brown solid. The product was used in the next step directly without further purification. 1H NMR (DMSO-d6, 300 MHz) δ 3.24 (1H, s), 3.45-3.52 (3H, m), 3.69-3.83 (2H, m), 4.67-4.90 (2H, m), 5.27 (1H, d), 6.67-6.88 (2H, m), 7.34 (1H, dd), 7.52 (1H, s), 8.84 (1H, s), 10.67 (1H, s). m/z (ES+), [M+H]+=394.

### Example 19, 2-[(8aS)-10-Acryloyl-6-chloro-8,8a,9,10,11,12-hexahydropyrazino[2′,1′:3,4]-[1,4]oxazepino[5,6,7-de]quinazolin-5-yl]-3-hydroxybenzonitrile, Atropisomer 1; and

Example 20, 2-[(8aS)-10-acryloyl-6-chloro-8,8a,9,10,11,12-hexahydropyrazino[2′,1′:3,4]-[1,4]oxazepino[5,6,7-de]quinazolin-5-yl]-3-hydroxybenzonitrile, Atropisomer 2

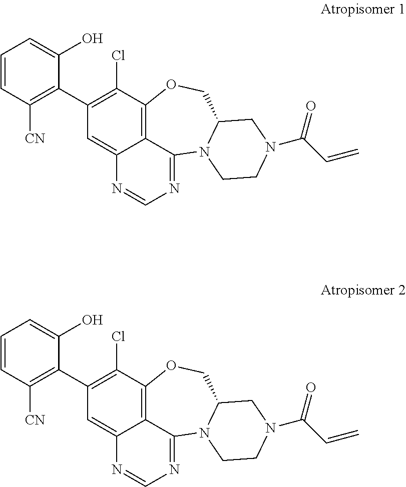 [SMILES: C=CC(=O)N1CCN2c3ncnc4cc(-c5c(O)cccc5C#N)c(Cl)c(c34)OC[C@@H]2C1 ; C=CC(=O)N1CCN2c3ncnc4cc(-c5c(O)cccc5C#N)c(Cl)c(c34)OC[C@@H]2C1]

Acryloyl chloride (53.6 mg, 0.59 mmol) was added to 2-[(8aS)-6-chloro-8,8a,9,10,11,12-hexahydropyrazino[2′,1′:3,4][1,4]oxazepino[5,6,7-de]quinazolin-5-yl]-3-hydroxy-benzonitrile TFA salt (600 mg, 0.59 mmol) and DIEA (0.21 ml, 1.18 mmol) in DMF (5 ml) at −10° C. under nitrogen. The temperature was increased to room temperature. The resulting mixture was stirred at room temperature for 1 hour. The crude product was purified by flash C18-flash chromatography, elution gradient 0 to 100%, 56% MeCN in water (0.05% NH₄HCO₃). Pure fractions were evaporated to dryness to afford 2-[(8aS)-10-acryloyl-6-chloro-8,8a,9,10,11,12-hexahydropyrazino[2′,1′:3,4][1,4]oxazepino [5,6,7-de]quinazolin-5-yl]-3-hydroxybenzonitrile (170 mg) as a white solid. The crude product was purified by preparative chiral-HPLC on a Column: CHIRALPAK AD-H, 2.0 cm I.D.*25 cm L; Mobile Phase A:Hex-HPLC, Mobile Phase B: IPA-HPLC; Flow rate: 20 ml/min; isocratic 35% B over 24 min; 220/254 nm. The fractions containing the first eluted product were evaporated to dryness to afford Example 19, 2-[(8aS)-10-acryloyl-6-chloro-8,8a,9,10,11,12-hexahydropyrazino[2′,1′:3,4][1,4]oxazepino[5,6,7-de]quinazolin-5-yl]-3-hydroxybenzonitrile (retention time 11.71 min) (Atropisomer 1, 50 mg, 19%, 100% d.e.), as a white solid. 1H NMR (DMSO-d6, 300 MHz) δ 3.02-3.17 (1H, m), 3.19-3.30 (1H, m), 3.36-3.53 (1H, m), 4.02-4.57 (3H, m), 4.59-4.91 (3H, m), 5.77 (1H, dd), 6.20 (1H, dd), 6.80-6.99 (1H, m), 7.28 (1H, dd), 7.35-7.53 (3H, m), 8.59 (1H, s), 10.43 (1H, s). m/z (ES+), [M+H]+=448; base, HPLC tR=0.75 min. Analytical chiral HPLC method Repaired ADH (100 mm×4.6 mm 5 m) a flow rate of 1 ml/minute and detection was by UV absorbance at wavelength of 254 nm. Oven temperature of 25° C. Mobile phase: Hex (0.1% DEA):IPA=70:30, retention time 4.081 minutes. Example 20, 2-[(8aS)-10-acryloyl-6-chloro-8,8a,9,10,11,12-hexahydropyrazino-[2′,1′:3,4][1,4]oxazepino [5,6,7-de]quinazolin-5-yl]-3-hydroxybenzonitrile (retention time 17.81 min) (Atropisomer 2, 46 mg, 17%, 99% d.e.), was also isolated as a white solid. 1H NMR (DMSO-d6, 300 MHz) δ 3.02-3.28 (2H, m), 3.34-3.56 (1H, m), 3.97-4.60 (3H, m), 4.70 (2H, d), 4.75-4.92 (1H, m), 5.76 (1H, dd), 6.20 (1H, dd), 6.80-6.99 (1H, m), 7.30 (1H, dd), 7.35-7.54 (3H, m), 8.59 (1H, s), 10.41 (1H, s). m/z (ES+), [M+H]+=448; base, HPLC tR=0.764 min. Analytical chiral HPLC method Repaired ADH (100 mm×4.6 mm 5 um) a flow rate of 1 ml/minute and detection was by UV absorbance at wavelength of 254 nm. Oven temperature of 25° C. Mobile phase: Hex (0.1% DEA):IPA=70:30, retention time 5.692 minutes.

### (8aS)-5-Bromo-6-chloro-8,8a,9,10,11,12-hexahydropyrazino[2′,1′:3,4][1,4]-oxazepino[5,6,7-de]quinazoline

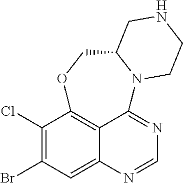 [SMILES: Clc1c(Br)cc2ncnc3c2c1OC[C@@H]1CNCCN31]

Tert-butyl (S)-10-bromo-11-chloro-3,4,13,13a-tetrahydropyrazino[2′,1′:3,4][1,4]oxazepino-[5,6,7-de]quinazoline-2 (1H)-carboxylate (21 g, 46.08 mmol), DCM (200 ml) and formic acid (20 ml) were stirred for 2 hours at room temperature under nitrogen. The resulting mixture was concentrated under vacuum to afford a solid (16 g) that was used without further purification. m/z: ES+ [M+H]+ 355.

### 1-[(8aS)-5-Bromo-6-chloro-8a,9,11,12-tetrahydropyrazino[2′,1′:3,4][1,4]oxazepino-[5,6,7-de]quinazolin-10 (8H)-yl]-3-(methylsulfonyl)propan-1-one

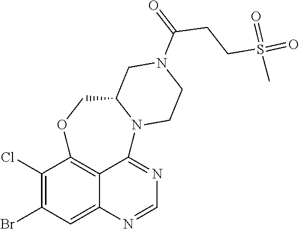 [SMILES: CS(=O)(=O)CCC(=O)N1CCN2c3ncnc4cc(Br)c(Cl)c(c34)OC[C@@H]2C1]

(8aS)-5-Bromo-6-chloro-8,8a,9,10,11,12-hexahydropyrazino[2′,1′:3,4][1,4]oxazepino-[5,6,7-de]quinazoline (16 g, 44.99 mmol), N,N-dimethylformamide (160 ml), 3-methanesulfonylpropanoic acid (7.6 g, 49.94 mmol), HATU (342 g, 899.46 mmol) and DIPEA (18.06 g, 139.74 mmol) were stirred for 2 hours at room temperature. The resulting solution was diluted with water and extracted with 3×100 ml of EtOAc and the organic layers combined. The resulting mixture was washed with 3×50 ml of water. The mixture was dried over anhydrous sodium sulfate. The residue was purified by silica gel chromatography with 10% MeOH in DCM to afford 1-[(8aS)-5-bromo-6-chloro-8a,9,11,12-tetrahydropyrazino[2′,1′:3,4][1,4]oxazepino[5,6,7-de]quinazolin-10 (8H)-yl]-3-(methylsulfonyl)propan-1-one (20 g, 91%) as a yellow solid. 1H NMR (300 MHz, DMSO, 299K) δ 2.74 (s, 1H), 2.90 (s, 1H), 3.05 (s, 1H), 3.18 (s, 3H), 3.38-3.43 (m, 3H), 3.49 (d, 1H), 4.23 (d, 2H), 4.45 (d, 1H), 4.74 (s, 2H), 4.80 (t, 1H), 7.82 (s, 1H), 8.73 (s, 1H). m/z: ES+ [M+H]+ 489.

### Parallel Synthesis Examples

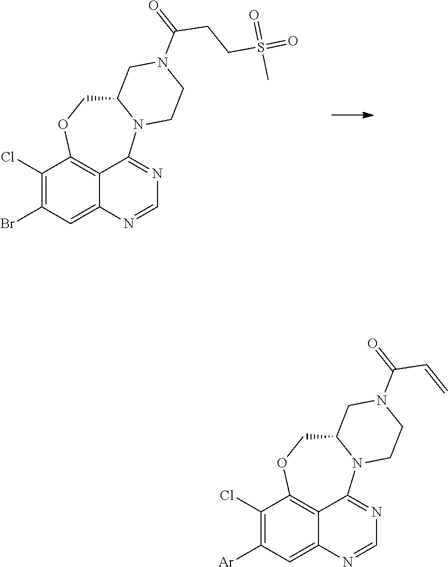

1-[(8aS)-5-bromo-6-chloro-8a,9,11,12-tetrahydropyrazino[2′,1′:3,4][1,4]oxazepino[5,6,7-de]quinazolin-10 (8H)-yl]-3-(methylsulfonyl)propan-1-one (0.06 mmol), a boronic acid (0.09 mmol, 1.5 equiv), Cs₂CO₃ (0.18 mmol, 3 equiv), Pd-118 (catalytic), dioxane (2 ml) and water (0.2 ml) were placed into a 40 ml vial and the mixture stirred at 100° C. for 16 hours. The crude product was purified by Prep-HPLC and lyophilized. This method was used to synthesize the examples shown in Table B.

TABLE B

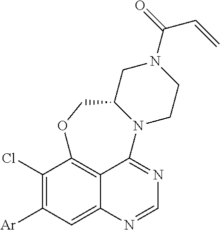

| | | | m/z: ES+ |
|---|---|---|---|
| Example | Name | Ar | [M + H]+ |
| 21 | 1-[(8aS)-5-(2-Amino-1,3-benzoxazol-5-yl)-6-chloro- 8a,9,11,12-tetrahydropyrazino[2′,1′:3,4][1,4]- oxazepino[5,6,7-de]quinazolin-10(8H)-yl]prop-2-en- 1-one | 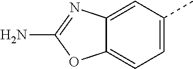 | 463 |
| 22 | 7-[(8aS)-10-Acryloyl-6-chloro-8,8a,9,10,11,12- hexahydropyrazino[2′,1′:3,4][1,4]oxazepino[5,6,7- de]quinazolin-5-yl]-3-methyl-1,3-benzoxazol-2(3H)- one | 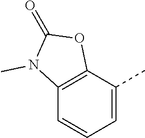 | 478 |
| 23 | N-{3-[(8aS)-10-Acryloyl-6-chloro-8,8a,9,10,11,12- hexahydropyrazino[2′,1′:3,4][1,4]oxazepino[5,6,7- de]quinazolin-5-yl]phenyl}acetamide | 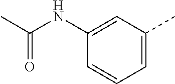 | 464 |
| 24 | 1-[(8aS)-6-Chloro-5-(2,3-dihydro-5H-1,4- benzodioxepin-9-yl)-8a,9,11,12- tetrahydropyrazino[2′,1′:3,4][1,4]oxazepino[5,6,7- de]quinazolin-10(8H)-yl]prop-2-en-1-one | 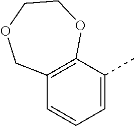 | 479 |
| 25 | 1-[(8aS)-6-Chloro-5-(2-fluoro-6-methoxyphenyl)- 8a,9,11,12- tetrahydropyrazino[2′,1′:3,4][1,4]oxazepino[5,6,7- de]quinazolin-10(8H)-yl]prop-2-en-1-one | 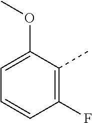 | 455 |
| 26 | 1-[(8aS)-6-Chloro-5-(3-fluoro-2-methoxyphenyl)- 8a,9,11,12- tetrahydropyrazino[2′,1′:3,4][1,4]oxazepino[5,6,7- de]quinazolin-10(8H)-yl]prop-2-en-1-one | 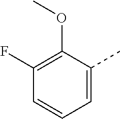 | 455 |
| 27 | 1-[(8aS)-6-Chloro-5-(2-hydroxy-3-methoxyphenyl)- 8a,9,11,12- tetrahydropyrazino[2′,1′:3,4][1,4]oxazepino[5,6,7- de]quinazolin-10(8H)-yl]prop-2-en-1-one | 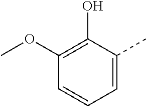 | 453 |
| 28 | 1-[(8aS)-6-Chloro-5-(1,3,4,5-tetrahydro-2- benzoxepin-6-yl)-8a,9,11,12- tetrahydropyrazino[2′,1′:3,4][1,4]oxazepino[5,6,7- de]quinazolin-10(8H)-yl]prop-2-en-1-one | 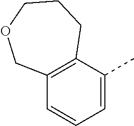 | 477 |
| 29 | 1-[(8aS)-6-Chloro-5-[2-(methylsulfonyl)phenyl]- 8a,9,11,12- tetrahydropyrazino[2′,1′:3,4][1,4]oxazepino[5,6,7- de]quinazolin-10(8H)-yl]prop-2-en-1-one | 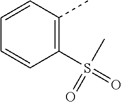 | 485 |
| 30 | 2-[(8aS)-10-Acryloyl-6-chloro-8,8a,9,10,11,12- hexahydropyrazino[2′,1′:3,4][1,4]oxazepino[5,6,7- de]quinazolin-5-yl]-N-methylbenzamide | 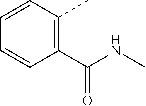 | 464 |
| 31 | 3-[(8aS)-10-Acryloyl-6-chloro-8,8a,9,10,11,12- hexahydropyrazino[2′,1′:3,4][1,4]oxazepino[5,6,7- de]quinazolin-5-yl]benzenesulfonamide | 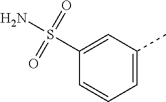 | 486 |
| 32 | 1-[(8aS)-6-Chloro-5-(quinoxalin-5-yl)-8a,9,11,12- tetrahydropyrazino[2′,1′:3,4][1,4]oxazepino[5,6,7- de]quinazolin-10(8H)-yl]prop-2-en-1-one | 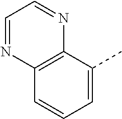 | 459 |
| 33 | Methyl {3-[(8aS)-10-Acryloyl-6-chloro- 8,8a,9,10,11,12- hexahydropyrazino[2′,1′:3,4][1,4]oxazepino[5,6,7- de]quinazolin-5-yl]phenyl}carbamate | 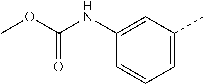 | 480 |
| 34 | 1-[(8aS)-6-Chloro-5-(2-methyl-1,2,3,4- tetrahydroisoquinolin-8-yl)-8a,9,11,12- tetrahydropyrazino[2′,1′:3,4][1,4]oxazepino[5,6,7- de]quinazolin-10(8H)-yl]prop-2-en-1-one | 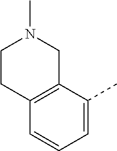 | 476 |
| 35 | 1-[(8aS)-6-Chloro-5-[2-(trifluoromethoxy)phenyl]- 8a,9,11,12- tetrahydropyrazino[2′,1′:3,4][1,4]oxazepino[5,6,7- de]quinazolin-10(8H)-yl]prop-2-en-1-one | 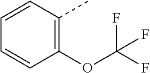 | 491 |
| 36 | 1-[(8aS)-6-Chloro-5-(3-{[dimethyl(oxido)-lambda~6~- sulfanylidene]amino}phenyl)-8a,9,11,12- tetrahydropyrazino[2′,1′:3,4][1,4]oxazepino[5,6,7- de]quinazolin-10(8H)-yl]prop-2-en-1-one | 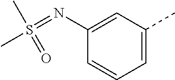 | 498 |
| 37 | 1-[(8aS)-6-Chloro-5-(2,3-dimethyl-2H-indazol-7-yl)- 8a,9,11,12- tetrahydropyrazino[2′,1′:3,4][1,4]oxazepino[5,6,7- de]quinazolin-10(8H)-yl]prop-2-en-1-one | 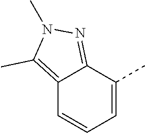 | 475 |
| 38 | 1-[(8aS)-6-Chloro-5-[2-fluoro-5- (hydroxymethyl)phenyl]-8a,9,11,12- tetrahydropyrazino[2′,1′:3,4][1,4]oxazepino[5,6,7- de]quinazolin-10(8H)-yl]prop-2-en-1-one | 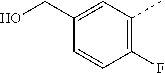 | 455 |
| 39 | 1-[(8aS)-6-Chloro-5-(1-methyl-1H-indazol-7-yl)- 8a,9,11,12- tetrahydropyrazino[2′,1′:3,4][1,4]oxazepino[5,6,7- de]quinazolin-10(8H)-yl]prop-2-en-1-one | 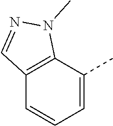 | 461 |
| 40 | 1-[(8aS)-6-Chloro-5-(2,5-difluorophenyl)-8a,9,11,12- tetrahydropyrazino[2′,1′:3,4][1,4]oxazepino[5,6,7- de]quinazolin-10(8H)-yl]prop-2-en-1-one | 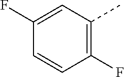 | 443 |
| 41 | 1-[(8aS)-6-Chloro-5-(2-methylphenyl)-8a,9,11,12- tetrahydropyrazino[2′,1′:3,4][1,4]oxazepino[5,6,7- de]quinazolin-10(8H)-yl]prop-2-en-1-one | 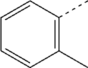 | 421 |
| 42 | 1-[(8aS)-6-Chloro-5-(2-chlorophenyl)-8a,9,11,12- tetrahydropyrazino[2′,1′:3,4][1,4]oxazepino[5,6,7- de]quinazolin-10(8H)-yl]prop-2-en-1-one | 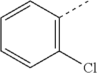 | 441 |
| 43 | 1-[(8aS)-6-Chloro-5-(2,3-dihydro-5H-1,4- benzodioxepin-6-yl)-8a,9,11,12- tetrahydropyrazino[2′,1′:3,4][1,4]oxazepino[5,6,7- de]quinazolin-10(8H)-yl]prop-2-en-1-one | 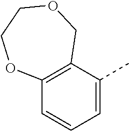 | 479 |
| 44 | 8-[(8aS)-10-Acryloyl-6-chloro-8,8a,9,10,11,12- hexahydropyrazino[2′,1′:3,4][1,4]oxazepino[5,6,7- de]quinazolin-5-yl]-1-methylquinolin-2(1H)-one | 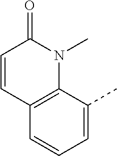 | 488 |
| 45 | 1-[(8aS)-6-Chloro-5-(1-methyl-1H-benzimidazol-4-yl)- 8a,9,11,12- tetrahydropyrazino[2′,1′:3,4][1,4]oxazepino[5,6,7- de]quinazolin-10(8H)-yl]prop-2-en-1-one | 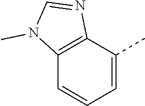 | 461 |
| 46 | 1-[(8aS)-6-Chloro-5-(1-methyl-1H-indol-3-yl)- 8a,9,11,12- tetrahydropyrazino[2′,1′:3,4][1,4]oxazepino[5,6,7- de]quinazolin-10(8H)-yl]prop-2-en-1-one | 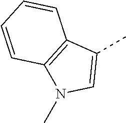 | 460 |
| 47 | 4-[(8aS)-10-Acryloyl-6-chloro-8,8a,9,10,11,12- hexahydropyrazino[2′,1′:3,4][1,4]oxazepino[5,6,7- de]quinazolin-5-yl]-3-methyl-1,3-benzoxazol-2(3H)- one | 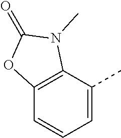 | 478 |
| 48 | 1-[(8aS)-6-Chloro-5-(5-chloro-2-methoxyphenyl)- 8a,9,11,12- tetrahydropyrazino[2′,1′:3,4][1,4]oxazepino[5,6,7- de]quinazolin-10(8H)-yl]prop-2-en-1-one | 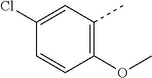 | 471 |
| 49 | 2-{2-[(8aS)-10-Acryloyl-6-chloro-8,8a,9,10,11,12- hexahydropyrazino[2′,1′:3,4][1,4]oxazepino[5,6,7- de]quinazolin-5-yl]phenyl}acetamide | 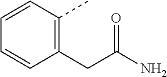 | 464 |
| 50 | 1-[(8aS)-6-Chloro-5-(2-chloro-4-fluorophenyl)- 8a,9,11,12- tetrahydropyrazino[2′,1′:3,4][1,4]oxazepino[5,6,7- de]quinazolin-10(8H)-yl]prop-2-en-1-one | 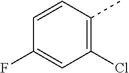 | 459 |
| 51 | 1-[(8aS)-6-Chloro-5-(5-fluoro-2-methoxyphenyl)- 8a,9,11,12- tetrahydropyrazino[2′,1′:3,4][1,4]oxazepino[5,6,7- de]quinazolin-10(8H)-yl]prop-2-en-1-one | 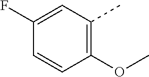 | 455 |
| 52 | 1-[(8aS)-6-Chloro-5-(4-hydroxyphenyl)-8a,9,11,12- tetrahydropyrazino[2′,1′:3,4][1,4]oxazepino[5,6,7- de]quinazolin-10(8H)-yl]prop-2-en-1-one | 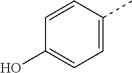 | 423 |
| 53 | 1-[(8aS)-6-Chloro-5-(1H-indol-4-yl)-8a,9,11,12- tetrahydropyrazino[2′,1′:3,4][1,4]oxazepino[5,6,7- de]quinazolin-10(8H)-yl]prop-2-en-1-one | 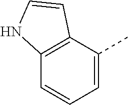 | 446 |
| 54 | 1-[(8aS)-5-(4-Amino-2-methylphenyl)-6-chloro- 8a,9,11,12- tetrahydropyrazino[2′,1′:3,4][1,4]oxazepino[5,6,7- de]quinazolin-10(8H)-yl]prop-2-en-1-one | 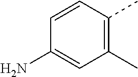 | 436 |
| 55 | 1-[(8aS)-6-Chloro-5-(4-methylpyridin-3-yl)- 8a,9,11,12- tetrahydropyrazino[2′,1′:3,4][1,4]oxazepino[5,6,7- de]quinazolin-10(8H)-yl]prop-2-en-1-one | 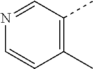 | 422 |
| 56 | 5-[(8aS)-10-Acryloyl-6-chloro-8,8a,9,10,11,12- hexahydropyrazino[2′,1′:3,4][1,4]oxazepino[5,6,7- de]quinazolin-5-yl]-2-hydroxybenzonitrile | 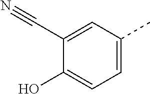 | 448 |
| 57 | 1-[(8aS)-5-(1,3-Benzoxazol-4-yl)-6-chloro-8a,9,11,12- tetrahydropyrazino[2′,1′:3,4][1,4]oxazepino[5,6,7- de]quinazolin-10(8H)-yl]prop-2-en-1-one | 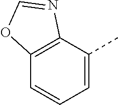 | 448 |
| 58 | 1-[(8aS)-5-(1,3-Benzoxazol-7-yl)-6-chloro-8a,9,11,12- tetrahydropyrazino[2′,1′:3,4][1,4]oxazepino[5,6,7- de]quinazolin-10(8H)-yl]prop-2-en-1-one | 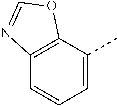 | 448 |
| 59 | {3-[(8aS)-10-Acryloyl-6-chloro-8,8a,9,10,11,12- hexahydropyrazino[2′,1′:3,4][1,4]oxazepino[5,6,7- de]quinazolin-5-yl]phenyl}acetonitrile | 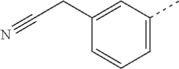 | 446 |
| 60 | 1-[(8aS)-6-Chloro-5-(2-fluorophenyl)-8a,9,11,12- tetrahydropyrazino[2′,1′:3,4][1,4]oxazepino[5,6,7- de]quinazolin-10(8H)-yl]prop-2-en-1-one | 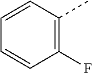 | 425 |
| 61 | 1-[(8aS)-6-Chloro-5-(1H-indazol-4-yl)-8a,9,11,12- tetrahydropyrazino[2′,1′:3,4][1,4]oxazepino[5,6,7- de]quinazolin-10(8H)-yl]prop-2-en-1-one | 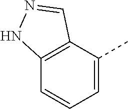 | 447 |
| 62 | 1-[(8aS)-6-Chloro-5-(2,3-difluorophenyl)-8a,9,11,12- tetrahydropyrazino[2′,1′:3,4][1,4]oxazepino[5,6,7- de]quinazolin-10(8H)-yl]prop-2-en-1-one | 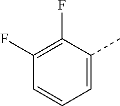 | 443 |
| 63 | 1-[(8aS)-6-Chloro-5-(2-hydroxyphenyl)-8a,9,11,12- tetrahydropyrazino[2′,1′:3,4][1,4]oxazepino[5,6,7- de]quinazolin-10(8H)-yl]prop-2-en-1-one | 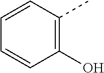 | 423 |
| 64 | 1-[(8aS)-6-Chloro-5-(4-fluoro-2-methylphenyl)- 8a,9,11,12- tetrahydropyrazino[2′,1′:3,4][1,4]oxazepino[5,6,7- de]quinazolin-10(8H)-yl]prop-2-en-1-one | 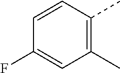 | 439 |
| 65 | 1-[(8aS)-6-Chloro-5-[2-(hydroxymethyl)phenyl]- 8a,9,11,12- tetrahydropyrazino[2′,1′:3,4][1,4]oxazepino[5,6,7- de]quinazolin-10(8H)-yl]prop-2-en-1-one | 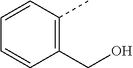 | 437 |
| 66 | 1-[(8aS)-6-Chloro-5-(2,4-difluorophenyl)-8a,9,11,12- tetrahydropyrazino[2′,1′:3,4][1,4]oxazepino[5,6,7- de]quinazolin-10(8H)-yl]prop-2-en-1-one | 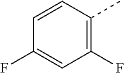 | 443 |
| 67 | 3-[(8aS)-10-Acryloyl-6-chloro-8,8a,9,10,11,12- hexahydropyrazino[2′,1′:3,4][1,4]oxazepino[5,6,7- de]quinazolin-5-yl]pyridine-4-carbonitrile | 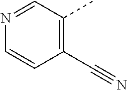 | 433 |
| 68 | 2-[(8aS)-10-Acryloyl-6-chloro-8,8a,9,10,11,12- hexahydropyrazino[2′,1′:3,4][1,4]oxazepino[5,6,7- de]quinazolin-5-yl]benzonitrile | 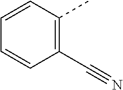 | 432 |

### Tert-Butyl (S)-3-(((tert-butyldimethylsilyl)oxy)methyl)piperazine-1-carboxylate

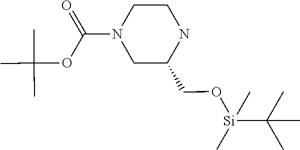

A solution of tert-butyldimethylsilyl chloride (1.53 g, 10.17 mmol) in DCM (10 ml) was added dropwise to (S)-4-N-Boc-2-hydroxymethyl-piperazine (2 g, 9.25 mmol) and triethylamine (2.58 ml, 18.49 mmol) in DCM (50 ml) at 20° C. over a period of 5 minutes under air. The resulting solution was stirred at 20° C. for 16 hours then evaporated to dryness. The residue was purified by flash silica chromatography, elution gradient 0 to 5% EtOH in EtOAc. Pure fractions were evaporated to dryness to afford tert-butyl (S)-3-(((tert-butyldimethylsilyl)oxy)methyl)piperazine-1-carboxylate (2.84 g, 93%) as a colourless oil. 1H NMR (500 MHz, CDCl₃) 0.00 (s, 6H), 0.84 (s, 9H), 1.40 (s, 9H), 2.48 (s, 1H), 2.6-2.87 (m, 3H), 2.92 (d, J=11.5 Hz, 1H), 3.41 (dd, J=7.2, 9.8 Hz, 1H), 3.52 (s, 1H), 3.85 (s, 2H).

### (E)-N-(3-bromo-2,5-difluorophenyl)-2-(hydroxyimino)acetamide

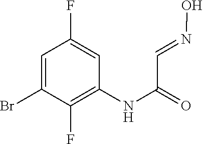 [SMILES: O=C(/C=N/O)Nc1cc(F)cc(Br)c1F]

Sodium sulfate (23.24 g, 163.62 mmol), hydroxylamine hydrochloride (4.97 g, 71.59 mmol) and 2,2,2-trichloroethane-1,1-diol (5.07 g, 30.68 mmol) were dissolved in water (103 ml). A solution of 3-bromo-2,5-difluoroaniline hydrochloride (5 g, 20.45 mmol) in water (8.21 ml), EtOH (14.36 ml) and conc. HCl (3.49 ml) was added and the reaction was stirred overnight at 60° C., forming a precipitate. The precipitate was collected by filtration and washed with water, then dried under vacuum to afford (E)-N-(3-bromo-2,5-difluoro-phenyl)-2-(hydroxyimino)acetamide (5.3 g, 93%) as a beige solid. This was used without further purification. 1H NMR (500 MHz, DMSO) 7.51 (ddd, J=3.1, 5.1, 8.1 Hz, 1H), 7.78 (s, 1H), 7.85 (ddd, J=3.1, 5.7, 10.1 Hz, 1H), 10.08 (s, 1H), 12.43 (s, 1H). m/z: ES− [M−H]− 277.

### 6-Bromo-4,7-difluoroindoline-2,3-dione

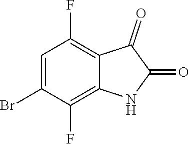 [SMILES: O=C1Nc2c(F)c(Br)cc(F)c2C1=O]

(E)-N-(3-Bromo-2,5-difluorophenyl)-2-(hydroxyimino)acetamide (7.62 g, 27.31 mmol) was added portionwise to sulfuric acid (68.3 ml) heated at 60° C. The reaction was stirred at 90° C. for 1 hour. The reaction mixture was cooled to room temperature and slowly added to ice water. The resulting precipitate was collected by filtration, washing with water and dried under vacuum to afford 6-bromo-4,7-difluoroindoline-2,3-dione (5.1 g, 71%) as a dark red solid. This was used without further purification. 1H NMR (500 MHz, DMSO) 7.38 (dd, J=4.4, 8.0 Hz, 1H), 11.91 (s, 1H). m/z: ES− [M−H]-260/262.

### 2-Amino-4-bromo-3,6-difluorobenzoic acid

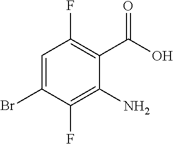 [SMILES: Nc1c(F)c(Br)cc(F)c1C(=O)O]

Hydrogen peroxide (30% in H₂O) (9.70 ml, 95 mmol) was added dropwise to 6-bromo-4,7-difluoroindoline-2,3-dione (4.98 g, 19 mmol) in sodium hydroxide (2M in H₂O) (86 ml, 171 mmol). The reaction was stirred at room temperature for 16 hours. Excess hydrogen peroxide was quenched with excess sodium sulfite, and the mixture was neutralised to pH7. The resulting brown precipitate filtered off and the remaining solution was acidified to pH2 with conc. HCl. The resulting cream precipitate was collected by filtration, washed with water and dried under vacuum to afford 2-amino-4-bromo-3,6-difluorobenzoic acid (3.10 g, 65%) as a brown solid. This was used without further purification. 1H NMR (500 MHz, DMSO) 6.71 (dd, J=5.2, 10.6 Hz, 1H), 6.85 (s, 1H), 13.40 (s, 1H). m/z: ES− [M−H]− 250/252.

### 7-Bromo-5,8-difluoroquinazolin-4 (3H)-one

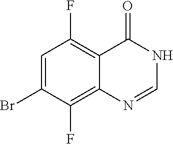 [SMILES: O=c1[nH]cnc2c(F)c(Br)cc(F)c12]

Formimidamide acetate (15.35 g, 147.47 mmol) and 2-amino-4-bromo-3,6-difluorobenzoic acid (3.1 g, 12.29 mmol) in ethanol (49 ml) were stirred at reflux for 16 hours. The reaction mixture was evaporated to dryness and redissolved in EtOAc (100 ml), and washed sequentially with saturated brine (2×150 ml). The organic layer was dried with MgSO₄, filtered and evaporated to afford 7-bromo-5,8-difluoroquinazolin-4 (3H)-one (2.9 g, 90%) as a yellow solid. This was used without further purification. 1H NMR (500 MHz, DMSO) 7.73 (dd, J=5.1, 10.3 Hz, 1H), 8.17 (s, 1H), 12.62 (s, 1H). m/z: ES− [M−H]-258/260.

### Tert-butyl (S)-4-(7-bromo-5,8-difluoroquinazolin-4-yl)-3-(((tert-butyldimethylsilyl)-oxy)methyl)piperazine-1-carboxylate

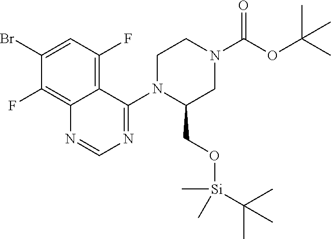 [SMILES: CC(C)(C)OC(=O)N1CCN(c2ncnc3c(F)c(Br)cc(F)c23)[C@H](CO[Si](C)(C)C(C)(C)C)C1]

((1H-Benzo[d][1,2,3]triazol-1-yl)oxy)tri(pyrrolidin-1-yl)phosphonium hexafluorophosphate(V) (2.59 g, 4.98 mmol) was added to 7-bromo-5,8-difluoroquinazolin-4 (3H)-one (1 g, 3.83 mmol) and DIPEA (1.61 ml, 9.19 mmol) in DMA (13.72 ml). The resulting solution was stirred at room temperature overnight and the reaction mixture poured into water, extracted with EtOAc (100 ml), washed with saturated brine (100 ml), dried over MgSO₄, filtered and evaporated to afford crude product. The crude product was purified by flash silica chromatography, elution gradient 0 to 100% EtOAc in heptane. Pure fractions were evaporated to dryness to afford tert-butyl (S)-4-(7-bromo-5,8-difluoroquinazolin-4-yl)-3-(((tert-butyldimethylsilyl)oxy)methyl)piperazine-1-carboxylate (0.66 g, 30%) as a pale yellow oil. 1H NMR (500 MHz, CDCl₃) −0.10 (s, 6H), 0.72 (s, 9H), 1.49 (s, 9H), 3.02 (s, 1H), 3.27 (d, J=10.9 Hz, 1H), 3.35-3.47 (m, 1H), 3.66 (s, 1H), 3.77-3.85 (m, 1H), 3.91 (d, J=13.6 Hz, 1H), 4.17 (d, J=13.4 Hz, 2H), 4.32 (s, 1H), 7.22-7.31 (m, 1H), 8.65 (s, 1H). m/z: ES+ [M+H]+ 573/575.

### Tert-butyl (S)-10-bromo-9-fluoro-3,4,13,13a-tetrahydropyrazino[2′,1′:3,4][1,4]-oxazepino[5,6,7-de]quinazoline-2 (1H)-carboxylate

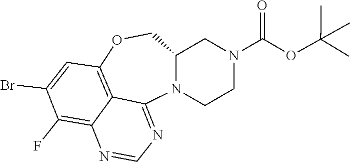 [SMILES: CC(C)(C)OC(=O)N1CCN2c3ncnc4c(F)c(Br)cc(c34)OC[C@@H]2C1]

Tetra-butylammonium fluoride (1M in THF) (1.37 ml, 1.37 mmol) was added to tert-butyl (S)-4-(7-bromo-5,8-difluoroquiazolin-4-yl)-3-(((tert-butyldimethylsilyl)oxy)methyl)-piperazine-1-carboxylate (0.66 g. 1.14 mmol) in THF (3.2 ml). The resulting solution was stirred at room temperature for 1 hour. The reaction was heated at 65° C. for 1 hour then cooled to room temperature, diluted with EtOAc (100 ml), washed with water (100 ml), saturated brine (100 ml), the organic layer dried over MgSO₄, filtered and evaporated to afford tert-butyl (S)-10-bromo-9-fluoro-3,4,13,13a-tetrahydropyrazino[2′,1′:3,4][1,4]-oxazepino[5,6,7-de]quinazoline-2 (1H)-carboxylate (0.54 g, >100%) as a beige foam. This was used without further purification. 1H NMR (500 MHz, CDCl₃) 1.49 (s, 9H), 3.07 (s, 2H), 3.1-3.2 (m, 1H), 3.84 (ddt, J=2.9, 5.6, 10.8 Hz, 1H), 3.98-4.24 (m, 2H), 4.30 (dd, J=5.1, 13.3 Hz, 1H), 4.38 (dd, J=3.1, 13.3 Hz, 1H), 5.06 (d, J=12.1 Hz, 1H), 7.14 (d, J=5.9 Hz, 1H), 8.65 (s, 1H). m/z: ES+ [M+H]+ 439/441.

### Tert-butyl (S)-10-bromo-11-chloro-9-fluoro-3,4,13,13a-tetrahydropyrazino[2′,1′:3,4]-[1,4]oxazepino[5,6,7-de]quinazoline-2 (1H)-carboxylate

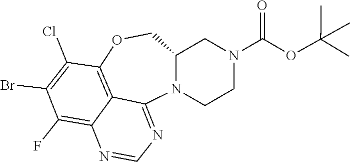 [SMILES: CC(C)(C)OC(=O)N1CCN2c3ncnc4c(F)c(Br)c(Cl)c(c34)OC[C@@H]2C1]

1-Chloropyrrolidine-2,5-dione (157 mg, 1.18 mmol) was added to tert-butyl (S)-10-bromo-9-fluoro-3,4,13,13a-tetrahydropyrazino[2′,1′:3,4][1,4]oxazepino[5,6,7-de]quinazoline-2 (1H)-carboxylate (470 mg, 1.07 mmol) in DMF (4.3 ml). The resulting solution was stirred at 70° C. for 1 hours. The reaction mixture was cooled to room temperature, poured into water (50 ml) and the resulting yellow precipitate was collected by filtration, washed with water and dried under vacuum to afford crude product which was purified by flash silica chromatography, elution gradient 0 to 50% EtOAc in heptane. Pure fractions were evaporated to dryness to afford tert-butyl (S)-10-bromo-11-chloro-9-fluoro-3,4,13,13a-tetrahydropyrazino[2′,1′:3,4][1,4] oxazepino [5,6,7-de]quinazoline-2 (1H)-carboxylate (327 mg, 65%) as a yellow solid. 1H NMR (500 MHz, DMSO) 1.43 (s, 9H), 3.06 (s, 2H), 3.23 (ddd, J=3.2, 11.1, 13.5 Hz, 1H), 3.91 (d, J=12.7 Hz, 1H), 3.97-4.09 (m, 2H), 4.56 (dd, J=5.3, 13.1 Hz, 1H), 4.61 (dd, J=3.2, 13.1 Hz, 1H), 4.82 (d, J=13.6 Hz, 1H), 8.58 (s, 1H). m/z: ES+ [M+H]+ 473/475/477.

### Tert-butyl (8aS)-6-chloro-4-fluoro-5-(2-fluoro-6-hydroxyphenyl)-8a,9,11,12-tetrahydropyrazino[2′,1′:3,4][1,4]oxazepino[5,6,7-de]quinazoline-10 (8H)-carboxylate

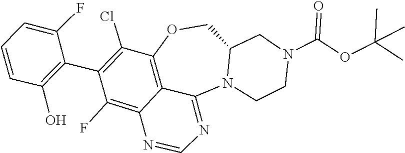 [SMILES: CC(C)(C)OC(=O)N1CCN2c3ncnc4c(F)c(-c5c(O)cccc5F)c(Cl)c(c34)OC[C@@H]2C1]

(2-Fluoro-6-hydroxyphenyl)boronic acid (0.165 g, 1.06 mmol), tert-butyl (S)-10-bromo-11-chloro-9-fluoro-3,4,13,13a-tetrahydropyrazino[2′,1′:3,4][1,4]oxazepino[5,6,7-de]quinazoline-2 (1H)-carboxylate (0.25 g, 0.53 mmol) and Pd(PPh₃)₄ (0.061 g, 0.05 mmol) were suspended in 1,4-dioxane (degassed) (9.76 ml) and Na₂CO₃ (2M in water) (0.79 ml, 1.58 mmol) and sealed into a microwave tube. The reaction was heated to 100° C. for 16 hours in the microwave reactor and cooled to room temperature. The reaction mixture was diluted with EtOAc (50 ml), washed with water (20 ml) and brine (50 ml), dried over MgSO₄, filtered and evaporated to afford crude product. The crude product was purified by flash silica chromatography, elution gradient 0 to 50% EtOAc in heptane. Pure fractions were evaporated to dryness to afford tert-butyl (8aS)-6-chloro-4-fluoro-5-(2-fluoro-6-hydroxyphenyl)-8a,9,11,12-tetrahydropyrazino [2′,1′:3,4][1,4]oxazepino[5,6,7-de]quinazoline-10 (8H)-carboxylate (0.134 g, 50%) as a colourless oil which solidified on standing. 1H NMR (500 MHz, DMSO, 27° C.) 1.44 (9H, s), 2.99-3.15 (1H, m), 3.17 (1H, d), 3.19-3.3 (1H, m), 3.85-4.15 (3H, m), 4.60 (1H, dd), 4.65 (1H, dd), 4.85 (1H, d), 6.76-6.82 (1H, m), 6.84 (1H, d), 7.34 (1H, td), 8.60 (1H, s), 10.19 (1H, s). m/z: ES+ [M+H]+ 505.

### 2-((8aS)-6-Chloro-4-fluoro-8,8a,9,10,11,12-hexahydropyrazino[2′,1′:3,4][1,4]-oxazepino[5,6,7-de]quinazolin-5-yl)-3-fluorophenol

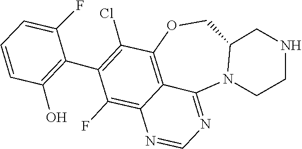 [SMILES: Oc1cccc(F)c1-c1c(Cl)c2c3c(ncnc3c1F)N1CCNC[C@H]1CO2]

Tert-butyl (8aS)-6-chloro-4-fluoro-5-(2-fluoro-6-hydroxyphenyl)-8a,9,11,12-tetrahydropyrazino [2′,1′:3,4][1,4]oxazepino[5,6,7-de]quinazoline-10 (8H)-carboxylate (134 mg, 0.27 mmol) was dissolved in DCM (663 μl) and treated with TFA (663 μl). The mixture was stirred at room temperature for 30 minutes. The solution was diluted with MeOH (10 ml) and loaded onto an SCX column. The column was washed with MeOH (2 column volumes) and the desired product was eluted from the column using 7M NH₃ in MeOH and evaporated to afford 2-((8aS)-6-chloro-4-fluoro-8,8a,9,10,11,12-hexahydropyrazino[2′,1′:3,4][1,4]oxazepino[5,6,7-de]quinazolin-5-yl)-3-fluorophenol (83 mg, 77%) as a colourless gum. This was used without further purification. 1H NMR (500 MHz, DMSO) 2.65-2.9 (m, 2H), 3.00 (d, J=12.3 Hz, 1H), 3.03-3.13 (m, 2H), 3.88-3.99 (m, 1H), 4.49 (dd, J=4.6, 13.1 Hz, 1H), 4.56 (ddd, J=3.2, 6.9, 13.1 Hz, 1H), 4.91-4.99 (m, 1H), 6.71-6.81 (m, 1H), 6.84 (dd, J=2.3, 8.3 Hz, 1H), 7.34 (td, J=7.0, 8.3 Hz, 1H), 7.52-7.72 (m, 1H), 8.56 (s, 1H), 10.20 (s, 1H). m/z: ES+ [M+H]+ 405.

### Example 69, 1-((8aS)-6-chloro-4-fluoro-5-(2-fluoro-6-hydroxyphenyl)-8a,9,11,12-tetrahydro-pyrazino[2′,1′:3,4][1,4]oxazepino[5,6,7-de]quinazolin-10 (8H)-yl)prop-2-en-1-one

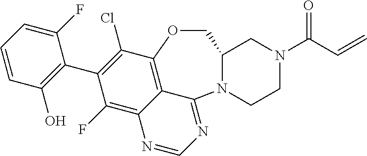 [SMILES: C=CC(=O)N1CCN2c3ncnc4c(F)c(-c5c(O)cccc5F)c(Cl)c(c34)OC[C@@H]2C1]

Acryloyl chloride (17.40 μl, 0.22 mmol) was added to 2-((8aS)-6-chloro-4-fluoro-8,8a,9,10,11,12-hexahydropyrazino[2′,1′:3,4][1,4]oxazepino[5,6,7-de]quinazolin-5-yl)-3-fluorophenol (83 mg, 0.21 mmol) and N-ethyl-N-isopropylpropan-2-amine (39.4 μl, 0.23 mmol) in DMA (763 μl) cooled at 0° C. The resulting solution was stirred at room temperature for 1 hour. The reaction mixture was poured into water (5 ml) and the resulting white precipitate was collected by filtration, washed with water and dried under vacuum. 49 mg was reserved for chiral separation of the atropisomers. The remainder was purified by preparative HPLC (Waters XBridge Prep C18 OBD column, 5μ silica, 50 mm diameter, 100 mm length), using decreasingly polar mixtures of water (containing 1% NH₃) and MeCN as eluents. Fractions containing the desired compound were evaporated to dryness to afford 1-((8aS)-6-chloro-4-fluoro-5-(2-fluoro-6-hydroxyphenyl)-8a,9,11,12-tetrahydropyrazino[2′,1′:3,4][1,4]oxazepino [5,6,7-de]quinazolin-10 (8H)-yl)prop-2-en-1-one (5 mg, 5%) as a white dry film. 1H NMR (500 MHz, DMSO) 3.15-3.33 (m, 1H), 3.45 (d, J=11.4 Hz, 1H), 3.52-3.77 (m, 1H), 4.14 (s, 1H), 4.19-4.38 (m, 1H), 4.5-4.73 (m, 3H), 5.11 (s, 1H), 5.85 (dd, J=1.9, 10.6 Hz, 1H), 6.32 (dd, J=1.9, 16.8 Hz, 1H), 6.69-6.78 (m, 1H), 6.81 (dd, J=2.7, 8.3 Hz, 1H), 6.87 (dd, J=10.7, 16.8 Hz, 1H), 7.35 (td, J=6.8, 8.3 Hz, 1H), 7.72-7.99 (m, 1H), 8.59 (s, 1H). m/z: ES+ [M+H]+ 459.

### 6-Amino-4-bromo-3-chloro-2-fluorobenzamide

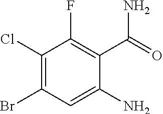 [SMILES: NC(=O)c1c(N)cc(Br)c(Cl)c1F]

To a solution of 6-amino-4-bromo-3-chloro-2-fluorobenzonitrile (1.23 g, 4.93 mmol) in DMSO (20 ml) was added potassium carbonate (1.36 g, 9.86 mmol) and hydrogen peroxide (1.8 ml, 17.62 mmol) (exotherm) and the reaction mixture stirred at room temperature for one hour. The reaction mixture was quenched with 10% aqueous sodium thiosulphate (10 ml) (exotherm), poured into water (200 ml), stirred for one hour and the solid filtered off, washing thoroughly with water and dried to afford 6-amino-4-bromo-3-chloro-2-fluorobenzamide (1.13 g, 85%) as a pale brown solid which was used without further purification. 1H NMR (400 MHz, DMSO) 6.24 (s, 2H), 6.95 (d, J=1.7 Hz, 1H), 7.76 (d, J=22.9 Hz, 2H). m/z: ES+ [M+H]+ 267/269.

### 7-Bromo-6-chloro-5-fluoro-2-morpholinoquinazolin-4 (3H)-one

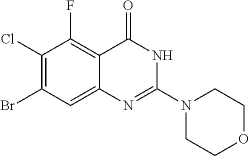 [SMILES: O=c1[nH]c(N2CCOCC2)nc2cc(Br)c(Cl)c(F)c12]

To a solution of 6-amino-4-bromo-3-chloro-2-fluorobenzamide (170 mg, 0.64 mmol) in 1,4-dioxane (5 ml) was added thiophosgene (0.102 ml, 1.33 mmol) (precipitate formed) and the reaction mixture stirred at room temperature for 1 hour then at 105° C. for 1 hour. The mixture was allowed to cool then the solvent evaporated and the residue suspended in dichloromethane (10 ml) and treated with morpholine (0.164 ml, 1.91 mmol) stirring at 40° C. for 4 hours. Added acetonitrile (2 ml), increased the temperature to 50° C. and continued stirring for 23 hours. The solvent was evaporated and the residue mixed with water, filtered off, washed thoroughly with water and dried to afford 7-bromo-6-chloro-5-fluoro-2-morpholinoquinazolin-4 (3H)-one (212 mg, 92%) as a white solid. 1H NMR (400 MHz, DMSO) 3.65 (s, 8H), 7.48 (d, J=1.6 Hz, 1H), 11.48 (s, 1H). m/z: ES+ [M+H]+ 362/364.

### Tert-butyl (S)-3-(((7-bromo-6-chloro-2-morpholino-4-oxo-3,4-dihydroquinazolin-5-yl)oxy)methyl)piperazine-1-carboxylate

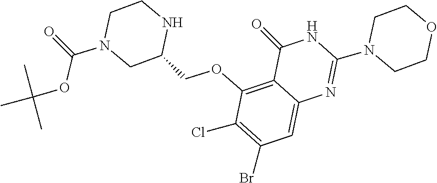 [SMILES: CC(C)(C)OC(=O)N1CCN[C@H](COc2c(Cl)c(Br)cc3nc(N4CCOCC4)[nH]c(=O)c23)C1]

60% Sodium hydride (60 mg, 1.49 mmol) was added to a suspension of tert-butyl (S)-3-(hydroxymethyl)piperazine-1-carboxylate (161 mg, 0.75 mmol) and 7-bromo-6-chloro-5-fluoro-2-morpholinoquinazolin-4 (3H)-one (208 mg, 0.57 mmol) in THF (10 ml) at 0° C. under nitrogen and stirred for 5 minutes. The reaction mixture was allowed to warm to room temperature then stirred at 65° C. for 1 hour, allowed to cool, then quenched at 0° C. with acetic acid (0.1 ml). The reaction mixture was diluted with ethyl acetate (50 ml), washed with aqueous 2M potassium carbonate solution (10 ml) then dried (MgSO₄) and the solvent evaporated. The residue was purified by flash silica chromatography, elution gradient 0 to 10% 2N methanolic ammonia in DCM. Pure fractions were evaporated to dryness to afford tert-butyl (S)-3-(((7-bromo-6-chloro-2-morpholino-4-oxo-3,4-dihydroquinazolin-5-yl)oxy)methyl)piperazine-1-carboxylate (215 mg, 67%) as a white foam. m/z: ES+ [M+H]+ 558/560.

### Tert-butyl (S)-5-bromo-6-chloro-2-morpholino-8a,9,11,12-tetrahydropyrazino-[2′,1′:3,4][1,4]oxazepino[5,6,7-de]quinazoline-10 (8H)-carboxylate

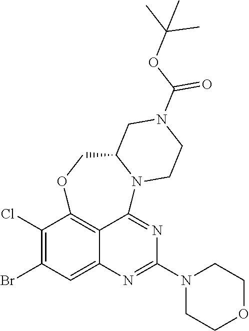 [SMILES: CC(C)(C)OC(=O)N1CCN2c3nc(N4CCOCC4)nc4cc(Br)c(Cl)c(c34)OC[C@@H]2C1]

To a stirred solution of tert-butyl (S)-3-(((7-bromo-6-chloro-2-morpholino-4-oxo-3,4-dihydroquinazolin-5-yl)oxy)methyl)piperazine-1-carboxylate (209 mg, 0.37 mmol) and ((1H-benzo[d][1,2,3]triazol-1-yl)oxy)tris(dimethylamino)phosphonium hexafluorophosphate(V) (248 mg, 0.56 mmol) in acetonitrile (5 ml) at 0° C. under nitrogen was added 2,3,4,6,7,8,9,10-octahydropyrimido[1,2-a]azepine (0.17 ml, 1.12 mmol) dropwise. The solution was stirred at 0° C. for 10 minutes then at room temperature for 4.5 hours. The solvent was evaporated and the residue was purified by flash silica chromatography, elution gradient 0 to 30% ethyl acetate/heptane. Pure fractions were evaporated to dryness to afford tert-butyl (S)-5-bromo-6-chloro-2-morpholino-8a,9,11,12-tetrahydropyrazino[2′,1′:3,4][1,4]oxazepino[5,6,7-de]quinazoline-10 (8H)-carboxylate (160 mg, 79%) as a white solid. 1H NMR (400 MHz, DMSO) 1.44 (s, 9H), 3.05 (s, 2H), 3.1-3.22 (m, 1H), 3.61-3.69 (m, 4H), 3.69-3.79 (m, 4H), 3.91 (t, J=10.8 Hz, 2H), 4.01 (d, J=12.9 Hz, 1H), 4.41-4.69 (m, 3H), 7.37 (s, 1H). m/z: ES+ [M+H]+ 540/542.

### Tert-butyl (8aS)-6-chloro-5-(5-methyl-1H-indazol-4-yl)-2-morpholino-8a,9,11,12-tetrahydropyrazino[2′,1′:3,4][1,4]oxazepino[5,6,7-de]quinazoline-10 (8H)-carboxylate

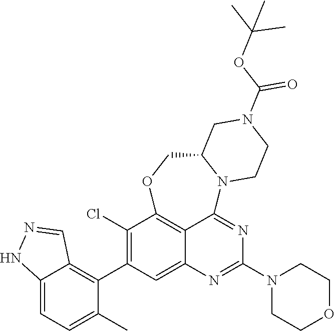 [SMILES: Cc1ccc2[nH]ncc2c1-c1cc2nc(N3CCOCC3)nc3c2c(c1Cl)OC[C@@H]1CN(C(=O)OC(C)(C)C)CCN31]

Pd-118 (20 mg, 0.03 mmol) was added to a degassed mixture of tert-butyl (S)-5-bromo-6-chloro-2-morpholino-8a,9,11,12-tetrahydropyrazino[2′,1′:3,4][1,4]oxazepino[5,6,7-de]quinazoline-10 (8H)-carboxylate (153 mg, 0.28 mmol), (5-methyl-1H-indazol-4-yl)boronic acid (75 mg, 0.43 mmol) and 2N sodium carbonate (0.5 ml, 1 mmol) in 1,4-dioxane (4 ml). The reaction mixture was heated to 100° C. for 1 h then at 90° C. for 16 h then allowed to cool. The reaction mixture was diluted with ethyl acetate (50 ml) and the organic layer was washed with aqueous saturated sodium bicarbonate solution (25 ml), water (25 ml) and brine (25 ml) then dried over MgSO₄, filtered and concentrated. The residue was purified by flash silica chromatography, elution gradient 0 to 3% 2N methanolic ammonia in DCM. Pure fractions were evaporated to dryness to afford tert-butyl (8aS)-6-chloro-5-(5-methyl-1H-indazol-4-yl)-2-morpholino-8a,9,11,12-tetrahydropyrazino[2′,1′:3,4][1,4]oxazepino[5,6,7-de]quinazoline-10 (8H)-carboxylate (140 mg, 84%) as a pale yellow solid. 1H NMR (400 MHz, DMSO) 1.46 (s, 9H), 2.15 (d, J=1.5 Hz, 3H), 2.98-3.25 (m, 3H), 3.58-3.7 (m, 4H), 3.7-3.82 (m, 4H), 3.87-4.17 (m, 3H), 4.46-4.77 (m, 3H), 6.93 (s, 1H), 7.31 (d, J=8.5 Hz, 1H), 7.43-7.6 (m, 2H), 13.06 (s, 1H). m/z: ES+ [M+H]+ 592/594.

### (8aS)-6-Chloro-5-(5-methyl-1H-indazol-4-yl)-2-morpholino-8,8a,9,10,11,12-hexahydropyrazino[2′,1′:3,4][1,4]oxazepino[5,6,7-de]quinazoline

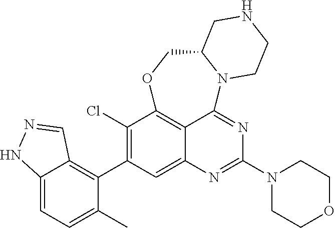 [SMILES: Cc1ccc2[nH]ncc2c1-c1cc2nc(N3CCOCC3)nc3c2c(c1Cl)OC[C@@H]1CNCCN31]

To a solution of tert-butyl (8aS)-6-chloro-5-(5-methyl-1H-indazol-4-yl)-2-morpholino-8a,9,11,12-tetrahydropyrazino[2′,1′:3,4][1,4]oxazepino[5,6,7-de]quinazoline-10 (8H)-carboxylate (137 mg, 0.23 mmol) in dichloromethane (4 ml) at 0° C. under nitrogen was added TFA (1 ml, 13.06 mmol) and the reaction mixture stirred for 90 minutes then the solvents evaporated. The residue was dissolved in methanol and applied to a 10 g SCX column washing thoroughly with methanol then the product was eluted using 1M ammonia in methanol. The solvent was evaporated to afford (8aS)-6-chloro-5-(5-methyl-1H-indazol-4-yl)-2-morpholino-8,8a,9,10,11,12-hexahydropyrazino[2′,1′:3,4][1,4]oxazepino[5,6,7-de]quinazoline (114 mg, 100%) as a pale yellow solid. 1H NMR (400 MHz, DMSO) 2.15 (s, 3H), 2.62-2.84 (m, 2H), 2.87-3.12 (m, 3H), 3.66 (t, J=4.2 Hz, 4H), 3.72 (d, J=5.0 Hz, 4H), 3.83 (d, J=5.5 Hz, 1H), 4.43 (ddd, J=2.1, 5.1, 13.2 Hz, 1H), 4.53 (dd, J=2.8, 13.2 Hz, 1H), 4.69 (d, J=11.5 Hz, 1H), 6.90 (s, 1H), 7.31 (d, J=8.7 Hz, 1H), 7.36-7.63 (m, 2H), 13.06 (s, 1H). m/z: ES+ [M+H]+ 492/494.

### Example 70, 1-[(8aS)-6-chloro-5-(5-methyl-1H-indazol-4-yl)-2-(morpholin-4-yl)-8a,9,11,12-tetrahydropyrazino[2′,1′:3,4][1,4]oxazepino[5,6,7-de]quinazolin-10 (8H)-yl]prop-2-en-1-one

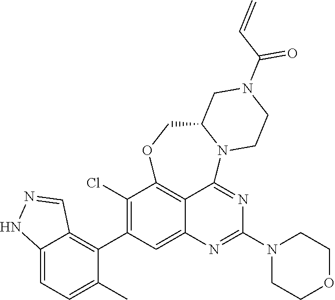 [SMILES: C=CC(=O)N1CCN2c3nc(N4CCOCC4)nc4cc(-c5c(C)ccc6[nH]ncc56)c(Cl)c(c34)OC[C@@H]2C1]

To a solution of (8aS)-6-chloro-5-(5-methyl-1H-indazol-4-yl)-2-morpholino-8,8a,9,10,11,12-hexahydropyrazino[2′,1′:3,4][1,4]oxazepino[5,6,7-de]quinazoline (109 mg, 0.22 mmol) in dichloromethane (10 ml) at 0° C. was added DIPEA (0.077 ml, 0.44 mmol), followed by a solution of acryloyl chloride (21 mg, 0.23 mmol) in dichloromethane (2 ml) and the reaction mixture stirred at 0° C. for 15 minutes. The reaction mixture was diluted with dichloromethane (50 ml) and washed with water (2×25 ml), the organic layer was dried MgSO4 and the solvent evaporated. The crude product was purified by preparative HPLC (Waters XSelect CSH C18 column, 5μ silica, 30 mm diameter, 100 mm length), using decreasingly polar mixtures of water (containing 0.3% NH₃) and MeCN as eluents. Fractions containing the desired compound were evaporated to dryness to afford 1-[(8aS)-6-chloro-5-(5-methyl-1H-indazol-4-yl)-2-(morpholin-4-yl)-8a,9,11,12-tetrahydropyrazino[2′,1′:3,4][1,4]oxazepino [5,6,7-de]quinazolin-10 (8H)-yl]prop-2-en-1-one (82 mg, 68%) as a pale orange solid. m/z: ES+ [M+H]+ 546/548.

### Example 71, 1-[(8aS)-6-chloro-5-(5-methyl-1H-indazol-4-yl)-2-(morpholin-4-yl)-8a,9,11,12-tetrahydropyrazino[2′,1′:3,4][1,4]oxazepino[5,6,7-de]quinazolin-10 (8H)-yl]prop-2-en-1-one, Atropisomer 1; and

### Example 72, 1-[(8aS)-6-chloro-5-(5-methyl-1H-indazol-4-yl)-2-(morpholin-4-yl)-8a,9,11,12-tetrahydropyrazino[2′,1′:3,4][1,4]oxazepino[5,6,7-de]quinazolin-10 (8H)-yl]prop-2-en-1-one, Atropisomer 2

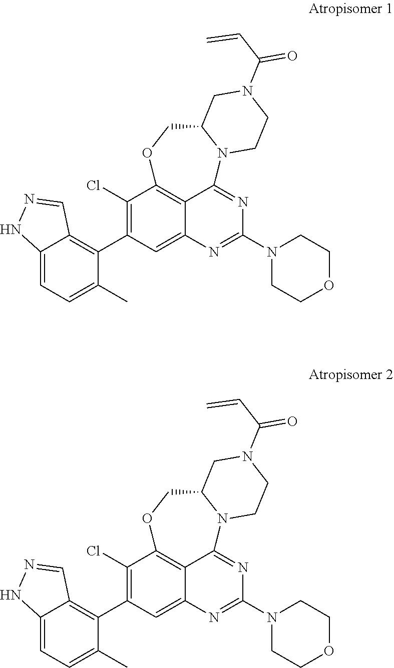 [SMILES: C=CC(=O)N1CCN2c3nc(N4CCOCC4)nc4cc(-c5c(C)ccc6[nH]ncc56)c(Cl)c(c34)OC[C@@H]2C1 ; C=CC(=O)N1CCN2c3nc(N4CCOCC4)nc4cc(-c5c(C)ccc6[nH]ncc56)c(Cl)c(c34)OC[C@@H]2C1]

The sample (Example 70) was dissolved in MeOH and separated using the SFC conditions detailed below: Column: Phenomonex Lux C1, 30×250 mm, 5 micron Mobile phase: 40% 2-Propanol+0.1% DEA/60% scCO2 Flow rate: 100 ml/min BPR: 120 bar, Column temperature: 40° C. Separated atropisomers were further purified using SFC conditions detailed below: Column: Princeton DEAP, 30×250 mm, 5 micron, Mobile phase: 10-30% MeOH+0.1% DEA/scCO2, Flow rate: 100 ml/min BPR: 120 bar, Temperature: 40° C. This gave Example 71, 1-[(8aS)-6-chloro-5-(5-methyl-1H-indazol-4-yl)-2-(morpholin-4-yl)-8a,9,11,12-tetrahydropyrazino[2′,1′:3,4][1,4]oxazepino[5,6,7-de]quinazolin-10 (8H)-yl]prop-2-en-1-one (Atropisomer 1, 11 mg, >99% d.e.), m/z: ES+ [M+H]+ 546/548. This was followed by Example 72, 1-[(8aS)-6-chloro-5-(5-methyl-1H-indazol-4-yl)-2-(morpholin-4-yl)-8a,9,11,12-tetrahydropyrazino[2′,1′:3,4][1,4]oxazepino-[5,6,7-de]quinazolin-10 (8H)-yl]prop-2-en-1-one (Atropisomer 2, 10 mg, >99% d.e.), m/z: ES+ [M+H]+ 546/548.

### Tert-butyl (S)-3-(((7-bromo-2,6-dichloro-4-oxo-3,4-dihydroquinazolin-5-yl)oxy)methyl)piperazine-1-carboxylate

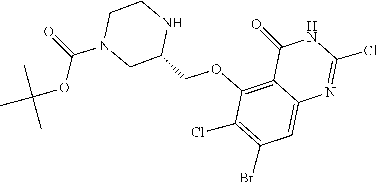 [SMILES: CC(C)(C)OC(=O)N1CCN[C@H](COc2c(Cl)c(Br)cc3nc(Cl)[nH]c(=O)c23)C1]

60% Sodium hydride (160 mg, 4 mmol) was added to a solution of tert-butyl (S)-3-(hydroxymethyl)piperazine-1-carboxylate (381 mg, 1.76 mmol) and 7-bromo-2,6-dichloro-5-fluoroquinazolin-4 (3H)-one (549 mg, 1.76 mmol) in THF (23 ml) under nitrogen and stirred for 30 minutes at room temperature then at 65° C. for 30 minutes and allowed to cool. The reaction mixture was diluted with ethyl acetate (200 ml), washed with water (3×100 ml) then dried (MgSO₄) and the solvent evaporated. The residue was stirred in diethyl ether (30 ml) for 1 hour then the solid filtered, washed with diethyl ether and dried to afford tert-butyl (S)-3-(((7-bromo-2,6-dichloro-4-oxo-3,4-dihydroquinazolin-5-yl)oxy)methyl)piperazine-1-carboxylate (588 mg, 66%) as an off-white solid. 1H NMR (400 MHz, DMSO) 1.42 (s, 9H), 3.02 (t, J=10.1 Hz, 1H), 3.17 (d, J=11.3 Hz, 2H), 3.34 (s, 1H), 3.52 (s, 1H), 3.91 (d, J=13.8 Hz, 1H), 3.98-4.13 (m, 1H), 4.22 (d, J=6.4 Hz, 1H), 4.35 (dd, J=3.2, 10.4 Hz, 1H), 7.63 (s, 1H), 9.42 (s, 2H). m/z: ES+ [M+H]+ 507/509.

### Tert-butyl (S)-3-(((7-bromo-6-chloro-2-((1-cyclopropylpiperidin-4-yl)amino)-4-oxo-3,4-dihydroquinazolin-5-yl)oxy)methyl)piperazine-1-carboxylate

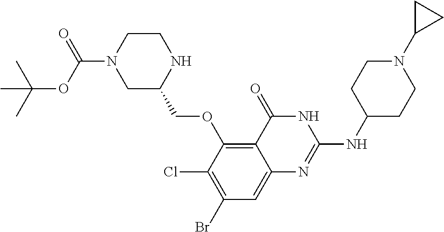 [SMILES: CC(C)(C)OC(=O)N1CCN[C@H](COc2c(Cl)c(Br)cc3nc(NC4CCN(C5CC5)CC4)[nH]c(=O)c23)C1]

To a suspension of tert-butyl (S)-3-(((7-bromo-2,6-dichloro-4-oxo-3,4-dihydroquinazolin-5-yl)oxy)methyl)piperazine-1-carboxylate (300 mg, 0.59 mmol) in i-PrOH (12 ml) was added DIPEA (0.31 ml, 1.77 mmol) and 1-cyclopropylpiperidin-4-amine (212 mg, 1.51 mmol) and the reaction mixture stirred at 100° C. for 42 hours and allowed to cool then evaporated to dryness. The residue was purified by flash silica chromatography, elution gradient 0 to 10% 2N methanolic ammonia in DCM. Pure fractions were evaporated to dryness to afford tert-butyl (S)-3-(((7-bromo-6-chloro-2-((1-cyclopropylpiperidin-4-yl)amino)-4-oxo-3,4-dihydroquinazolin-5-yl)oxy)methyl)piperazine-1-carboxylate (338 mg, 94%) as an orange oil. ms detection: m/z: ES+ [M+H]+ 611/613.

### Tert-butyl (S)-5-bromo-6-chloro-2-((1-cyclopropylpiperidin-4-yl)amino)-8a,9,11,12-tetrahydropyrazino[2′,1′:3,4][1,4]oxazepino[5,6,7-de]quinazoline-10 (8H)-carboxylate

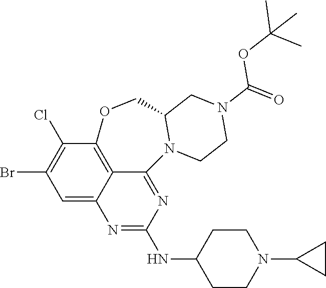 [SMILES: CC(C)(C)OC(=O)N1CCN2c3nc(NC4CCN(C5CC5)CC4)nc4cc(Br)c(Cl)c(c34)OC[C@@H]2C1]

To a stirred suspension of tert-butyl (S)-3-(((7-bromo-6-chloro-2-((1-cyclopropylpiperidin-4-yl)amino)-4-oxo-3,4-dihydroquinazolin-5-yl)oxy)methyl)piperazine-1-carboxylate (338 mg, 0.55 mmol) and ((1H-benzo[d][1,2,3]triazol-1-yl)oxy)tris(dimethylamino)-phosphonium hexafluorophosphate(V) (366 mg, 0.83 mmol) in acetonitrile (8 ml) at 0° C. under nitrogen was added 2,3,4,6,7,8,9,10-octahydropyrimido[1,2-a]azepine (0.25 ml, 1.66 mmol) dropwise. The solution was stirred at 0° C. for ten minutes then at room temperature for 2.5 hours. The solvent was evaporated and the residue was purified by flash silica chromatography, elution gradient 0 to 5% 2N methanolic ammonia in DCM. Pure fractions were evaporated to dryness to afford tert-butyl (S)-5-bromo-6-chloro-2-((1-cyclopropylpiperidin-4-yl)amino)-8a,9,11,12-tetrahydropyrazino[2′,1′:3,4][1,4]oxazepino-[5,6,7-de]quinazoline-10 (8H)-carboxylate (223 mg, 68.0%) as an off-white solid. m/z: ES+ [M+H]+ 593/595.

### Tert-butyl (8aS)-6-chloro-2-((1-cyclopropylpiperidin-4-yl)amino)-5-(5-methyl-1H-indazol-4-yl)-8a,9,11,12-tetrahydropyrazino[2′,1′:3,4][1,4]oxazepino[5,6,7-de]quinazoline-10 (8H)-carboxylate

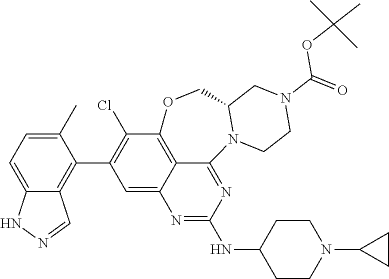 [SMILES: Cc1ccc2[nH]ncc2c1-c1cc2nc(NC3CCN(C4CC4)CC3)nc3c2c(c1Cl)OC[C@@H]1CN(C(=O)OC(C)(C)C)CCN31]

Pd-118 (25.6 mg, 0.04 mmol) was added to a degassed mixture of tert-butyl (S)-10-bromo-11-chloro-7-((1-cyclopropylpiperidin-4-yl)amino)-3,4,13,13a-tetrahydropyrazino-[2′,1′:3,4][1,4]oxazepino[5,6,7-de]quinazoline-2 (1H)-carboxylate (216 mg, 0.36 mmol), (5-methyl-1H-indazol-4-yl)boronic acid (128 mg, 0.73 mmol) and 2N sodium carbonate (1 ml, 2 mmol) in 1,4-dioxane (6 ml). The reaction mixture was heated at 100° C. for 18 hours then allowed to cool. The reaction mixture was diluted with ethyl acetate (50 ml) and the organic layer was washed with aqueous 2M sodium carbonate solution (2×25 ml), water (25 ml) and brine (25 ml) then dried over MgSO₄, filtered and concentrated. The residue was purified by flash silica chromatography, elution gradient 0 to 4% 2N methanolic ammonia in DCM. Pure fractions were evaporated to dryness to afford tert-butyl (8aS)-6-chloro-2-((1-cyclopropylpiperidin-4-yl)amino)-5-(5-methyl-1H-indazol-4-yl)-8a,9,11,12-tetrahydropyrazino[2′,1′:3,4][1,4]oxazepino[5,6,7-de]quinazoline-10 (8H)-carboxylate (80 mg, 34.1%) as a pale brown solid. 1H NMR (400 MHz, DMSO) 0.19-0.38 (m, 2H), 0.41 (d, J=4.9 Hz, 2H), 1.47 (s, 10H), 1.59 (s, 1H), 1.84 (d, J=10.4 Hz, 2H), 2.16 (d, J=2.8 Hz, 5H), 2.92 (d, J=10.7 Hz, 2H), 3.14 (d, J=31.6 Hz, 3H), 3.68-4.18 (m, 4H), 4.43-4.84 (m, 3H), 6.79 (d, J=53.4 Hz, 2H), 7.32 (d, J=8.6 Hz, 1H), 7.50 (d, J=7.6 Hz, 2H), 13.06 (s, 1H). m/z: ES+ [M+H]+ 645/646.

### (8aS)-6-chloro-N-(1-cyclopropylpiperidin-4-yl)-5-(5-methyl-1H-indazol-4-yl)-8,8a,9,10,11,12-hexahydropyrazino[2′,1′:3,4][1,4]oxazepino[5,6,7-de]quinazolin-2-amine

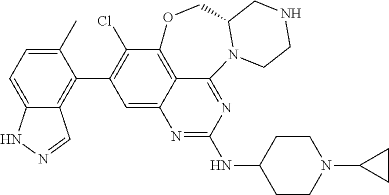 [SMILES: Cc1ccc2[nH]ncc2c1-c1cc2nc(NC3CCN(C4CC4)CC3)nc3c2c(c1Cl)OC[C@@H]1CNCCN31]

To a solution of tert-butyl (8aS)-6-chloro-2-((1-cyclopropylpiperidin-4-yl)amino)-5-(5-methyl-1H-indazol-4-yl)-8a,9,11,12-tetrahydropyrazino[2′,1′:3,4][1,4]oxazepino[5,6,7-de]quinazoline-10 (8H)-carboxylate (76 mg, 0.12 mmol) in DCM (2 ml) at 0° C. under nitrogen was added TFA (0.5 ml, 6.53 mmol) and the reaction mixture stirred for 2.5 hours then the solvents evaporated. The residue was dissolved in methanol and applied to a 5 g SCX column washing thoroughly with methanol then the product was eluted using 1M ammonia in methanol. The solvent was evaporated to afford (8aS)-6-chloro-N-(1-cyclopropylpiperidin-4-yl)-5-(5-methyl-1H-indazol-4-yl)-8,8a,9,10,11,12-hexahydropyrazino[2′,1′:3,4][1,4]oxazepino[5,6,7-de]quinazolin-2-amine (64 mg, 100%) as a pale brown solid. 1H NMR (400 MHz, DMSO) 0.29 (s, 2H), 0.41 (d, J=5.5 Hz, 2H), 1.44 (d, J=11.5 Hz, 2H), 1.60 (s, 1H), 1.75-1.91 (m, 2H), 2.16 (s, 3H), 2.18-2.32 (m, 2H), 2.65-3.02 (m, 4H), 2.98-3.26 (m, 4H), 3.83 (d, J=37.9 Hz, 2H), 4.45 (d, J=13.2 Hz, 1H), 4.55 (d, J=11.0 Hz, 1H), 4.75 (d, J=12.9 Hz, 1H), 6.68 (s, 1H), 6.84 (s, 1H), 7.32 (d, J=8.7 Hz, 1H), 7.39-7.59 (m, 2H), 13.06 (s, 1H). m/z: ES+ [M+H]+ 545/547.

### Example 73, 1-[(8aS)-6-chloro-2-[(1-cyclopropylpiperidin-4-yl)amino]-5-(5-methyl-1H-indazol-4-yl)-8a,9,11,12-tetrahydropyrazino[2′,1′:3,4][1,4]oxazepino[5,6,7-de]quinazolin-10 (8H)-yl]prop-2-en-1-one, Atropisomer 1; and

### Example 74, 1-[(8aS)-6-chloro-2-[(1-cyclopropylpiperidin-4-yl)amino]-5-(5-methyl-1H-indazol-4-yl)-8a,9,11,12-tetrahydropyrazino[2′,1′:3,4][1,4]oxazepino[5,6,7-de]quinazolin-10 (8H)-yl]prop-2-en-1-one, Atropisomer 2

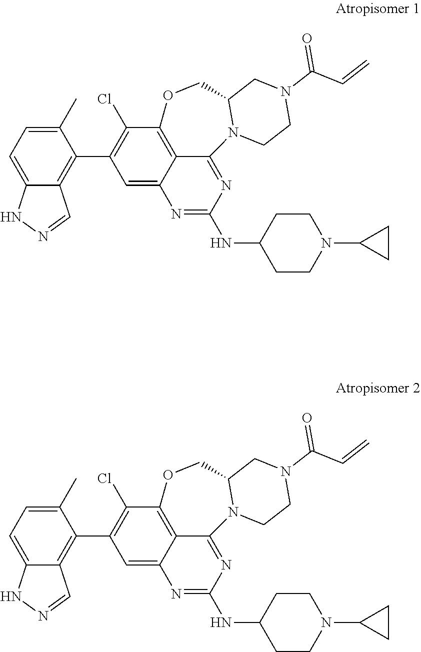 [SMILES: C=CC(=O)N1CCN2c3nc(NC4CCN(C5CC5)CC4)nc4cc(-c5c(C)ccc6[nH]ncc56)c(Cl)c(c34)OC[C@@H]2C1 ; C=CC(=O)N1CCN2c3nc(NC4CCN(C5CC5)CC4)nc4cc(-c5c(C)ccc6[nH]ncc56)c(Cl)c(c34)OC[C@@H]2C1]

To a solution of (8aS)-6-chloro-N-(1-cyclopropylpiperidin-4-yl)-5-(5-methyl-1H-indazol-4-yl)-8,8a,9,10,11,12-hexahydropyrazino[2′,1′:3,4][1,4]oxazepino[5,6,7-de]quinazolin-2-amine (61 mg, 0.11 mmol) in DMF (5 ml) at −15° C. was added N-ethyl-N-isopropylpropan-2-amine (0.039 ml, 0.22 mmol) followed by a solution of acryloyl chloride (10.6 mg, 0.12 mmol) in DMF (2 ml) and the reaction mixture stirred at −15° C. for fifteen minutes. The reaction mixture was diluted with ethyl acetate (50 ml) and washed with aqueous sodium bicarbonate solution (25 ml) and water (2×25 ml), the organic layer was dried over MgSO₄ and the solvent evaporated. The sample was dissolved in MeOH and separated using the SFC conditions detailed below: Column: Phenomonex C1, 30×250 mm, 5 micron Mobile phase: 40% MeOH (0.1% NH3)/60% scCO2 Flow rate: 80 ml/min BPR: 120 bar Column temp: 40° C. This afforded 1-[(8aS)-6-chloro-2-[(1-cyclopropylpiperidin-4-yl)amino]-5-(5-methyl-1H-indazol-4-yl)-8a,9,11,12-tetrahydropyrazino[2′,1′:3,4][1,4]-oxazepino[5,6,7-de]quinazolin-10 (8H)-yl]prop-2-en-1-one (Atropisomer 1, 14.7 mg, >99% d.e. Purity: 99%). 1H NMR (400 MHz, DMSO) 0.28 (s, 2H), 0.40 (d, J=4.6 Hz, 2H), 1.42 (dt, J=11.5, 22.7 Hz, 2H), 1.58 (s, 1H), 1.74-1.91 (m, 2H), 2.16 (s, 3H), 2.22 (s, 2H), 2.81-3.14 (m, 3H), 3.35-3.51 (m, 1H), 3.69-4.05 (m, 2H), 4.08-4.86 (m, 5H), 5.68-5.88 (m, 1H), 6.19 (dd, J=2.3, 16.7 Hz, 1H), 6.90 (dd, J=10.5, 16.7 Hz, 3H), 7.31 (d, J=8.7 Hz, 1H), 7.49 (d, J=7.8 Hz, 2H), 13.06 (s, 1H). m/z: ES+ [M+H]+ 599/601. This was followed by 1-[(8aS)-6-chloro-2-[(1-cyclopropylpiperidin-4-yl)amino]-5-(5-methyl-1H-indazol-4-yl)-8a,9,11,12-tetrahydropyrazino[2′,1′:3,4][1,4]oxazepino[5,6,7-de]quinazolin-10 (8H)-yl]prop-2-en-1-one (Atropisomer 2, 14.1 mg, >99% d.e. Purity: 98%). 1H NMR (400 MHz, DMSO) 0.28 (s, 2H), 0.40 (d, J=4.7 Hz, 2H), 1.44 (q, J=11.6 Hz, 2H), 1.58 (s, 1H), 1.76-1.94 (m, 2H), 2.15 (s, 3H), 2.23 (s, 2H), 2.77-3.13 (m, 3H), 3.38 (d, J=20.1 Hz, 1H), 3.71-4.01 (m, 2H), 4.08-4.75 (m, 5H), 5.67-5.85 (m, 1H), 6.19 (dd, J=2.3, 16.7 Hz, 1H), 6.55-7.01 (m, 3H), 7.31 (d, J=8.7 Hz, 1H), 7.4-7.58 (m, 2H), 13.05 (s, 1H). m/z: ES+ [M+H]+ 599/601.

### 7-Bromo-2,6-dichloro-5-fluoroquinazolin-4 (3H)-one

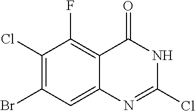 [SMILES: O=c1[nH]c(Cl)nc2cc(Br)c(Cl)c(F)c12]

To a solution of 6-amino-4-bromo-3-chloro-2-fluorobenzamide (940 mg, 3.51 mmol) in 1,4-dioxane (30 ml) was added thiophosgene (0.57 ml, 7.44 mmol) (precipitate formed) and the reaction mixture stirred at room temperature for 1 hour then at 105° C. for 1 hour. The mixture was allowed to cool, then the solvent evaporated to afford 7-bromo-2,6-dichloro-5-fluoroquinazolin-4 (3H)-one as a pale yellow solid. m/z: ES+ [M+H]+ 311/313. This was used without further purification in subsequent steps.

### Tert-butyl (S)-3-(((7-bromo-6-chloro-2-(3-(dimethylamino)azetidin-1-yl)-4-oxo-3,4-dihydroquinazolin-5-yl)oxy)methyl)piperazine-1-carboxylate

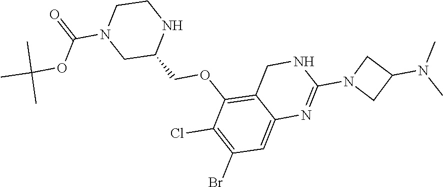 [SMILES: CN(C)C1CN(C2=Nc3cc(Br)c(Cl)c(OC[C@@H]4CN(C(=O)OC(C)(C)C)CCN4)c3CN2)C1]

To a suspension of tert-butyl (S)-3-(((7-bromo-2,6-dichloro-4-oxo-3,4-dihydroquinazolin-5-yl)oxy)methyl)piperazine-1-carboxylate (500 mg, 0.98 mmol) in i-PrOH (20 ml) was added N-ethyl-N-isopropylpropan-2-amine (1.37 ml, 7.87 mmol) and N,N-dimethyl-azetidin-3-amine dihydrochloride (511 mg, 2.95 mmol) and the reaction mixture stirred at 95° C. for 1 hour and allowed to cool. The reaction mixture was evaporated to dryness. The residue was purified by flash silica chromatography, elution 20% 2N methanolic ammonia in DCM. Pure fractions were evaporated to dryness then triturated with diethyl ether to afford tert-butyl (S)-3-(((7-bromo-6-chloro-2-(3-(dimethylamino)azetidin-1-yl)-4-oxo-3,4-dihydroquinazolin-5-yl)oxy)methyl)piperazine-1-carboxylate (377 mg, 67%) as a pale yellow solid. 1H NMR (400 MHz, DMSO) 1.43 (s, 9H), 2.20 (s, 6H), 2.85-3.05 (m, 1H), 3.06-3.27 (m, 4H), 3.42-3.58 (m, 1H), 3.82-4.05 (m, 3H), 4.05-4.32 (m, 5H), 7.47 (s, 1H). m/z: ES+ [M+H]+ 571/573.

### Tert-butyl (S)-5-bromo-6-chloro-2-(3-(dimethylamino)azetidin-1-yl)-8a,9,11,12-tetrahydropyrazino[2′,1′:3,4][1,4]oxazepino[5,6,7-de]quinazoline-10 (8H)-carboxylate

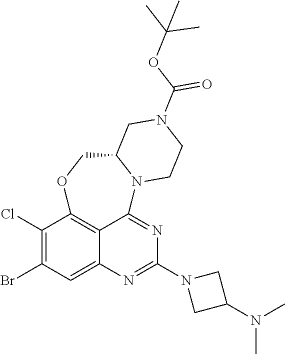 [SMILES: CN(C)C1CN(c2nc3c4c(c(Cl)c(Br)cc4n2)OC[C@@H]2CN(C(=O)OC(C)(C)C)CCN32)C1]

To a stirred solution of tert-butyl (S)-3-(((7-bromo-6-chloro-2-(3-(dimethylamino)azetidin-1-yl)-4-oxo-3,4-dihydroquinazolin-5-yl)oxy)methyl)piperazine-1-carboxylate (377 mg, 0.66 mmol) and ((1H-benzo[d][1,2,3]triazol-1-yl)oxy)tri(pyrrolidin-1-yl)phosphonium hexafluorophosphate(V) (515 mg, 0.99 mmol) in acetonitrile (14 ml) at 0° C. under nitrogen was added 2,3,4,6,7,8,9,10-octahydropyrimido[1,2-a]azepine (0.3 ml, 1.98 mmol) dropwise. The solution was stirred at 0° C. for 10 minutes then at room temperature for 1.5 hours. The resulting precipitate was filtered off and dried to afford tert-butyl (S)-5-bromo-6-chloro-2-(3-(dimethylamino)azetidin-1-yl)-8a,9,11,12-tetrahydropyrazino[2′,1′:3,4]-[1,4]oxazepino[5,6,7-de]quinazoline-10 (8H)-carboxylate (173 mg) as a white solid. The filtrate was evaporated and the residue was dissolved in ethyl acetate (100 ml) and washed with aqueous saturated sodium bicarbonate solution (50 ml), water (50 ml) and brine (50 ml) then dried (MgSO₄) and the solvent evaporated. The residue was purified by flash silica chromatography, elution gradient 0 to 4% 2N methanolic ammonia in DCM. Pure fractions were evaporated to dryness then triturated with acetonitrile to afford tert-butyl (S)-5-bromo-6-chloro-2-(3-(dimethylamino)azetidin-1-yl)-8a,9,11,12-tetrahydropyrazino-[2′,1′:3,4][1,4]oxazepino[5,6,7-de]quinazoline-10 (8)-carboxylate (65 mg, 65%) as a white solid. 1H NMR (400 MHz, DMSO) 1.44 (s, 9H), 2.12 (s, 6H), 2.89-3.21 (m, 4H), 3.75-3.95 (m, 4H), 3.96-4.13 (m, 3H), 4.41-4.6 (m, 2H), 4.67 (d, J=13.2 Hz, 1H), 7.36 (s, 1H). m/z: ES+ [M+H]+ 553/555.

### Tert-butyl (8aS)-6-chloro-2-(3-(dimethylamino)azetidin-1-yl)-5-(5-methyl-1H-indazol-4-yl)-8a,9,11,12-tetrahydropyrazino[2′,1′:3,4][1,4]oxazepino[5,6,7-de]quinazoline-10 (8H)-carboxylate

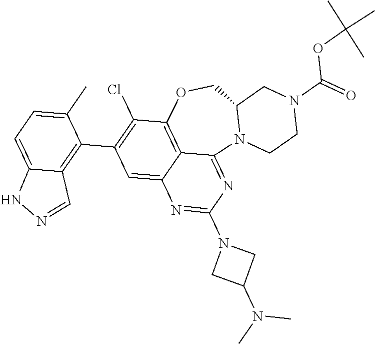 [SMILES: Cc1ccc2[nH]ncc2c1-c1cc2nc(N3CC(N(C)C)C3)nc3c2c(c1Cl)OC[C@@H]1CN(C(=O)OC(C)(C)C)CCN31]

Pd-118 (30 mg, 0.05 mmol) was added to a degassed mixture of tert-butyl (S)-10-bromo-11-chloro-7-(3-(dimethylamino)azetidin-1-yl)-3,4,13,13a-tetrahydropyrazino[2′,1′:3,4]-[1,4]oxazepino[5,6,7-de]quinazoline-2 (1H)-carboxylate (230 mg, 0.42 mmol), (5-methyl-1H-indazol-4-yl)boronic acid (150 mg, 0.85 mmol) and 2N sodium carbonate (1.14 ml, 2.28 mmol) in 1,4-dioxane (8 ml). The reaction mixture was heated at 100° C. for 17 hours then allowed to cool. The reaction mixture was diluted with ethyl acetate (100 ml) and the organic layer was washed with aqueous 2M sodium carbonate solution (2×50 ml) and brine (50 ml) then dried over MgSO₄, filtered and concentrated. The residue was purified by flash silica chromatography, elution gradient 0 to 5% 2N methanolic ammonia in DCM. Pure fractions were evaporated to dryness to afford tert-butyl (8aS)-6-chloro-2-(3-(dimethylamino)azetidin-1-yl)-5-(5-methyl-1H-indazol-4-yl)-8a,9,11,12-tetrahydropyrazino[2′,1′:3,4][1,4]oxazepino [5,6,7-de]quinazoline-10 (8H)-carboxylate (208 mg, 83%) as a pale brown solid. 1H NMR (400 MHz, DMSO) 1.45 (s, 9H), 2.1-2.17 (m, 9H), 3.04-3.2 (m, 4H), 3.8-3.89 (m, 2H), 3.93 (d, J=11.2 Hz, 2H), 3.99-4.14 (m, 3H), 4.52-4.64 (m, 2H), 4.71 (d, J=11.8 Hz, 1H), 6.91 (s, 1H), 7.31 (d, J=8.5 Hz, 1H), 7.44-7.53 (m, 2H), 13.05 (s, 1H). m/z: ES+ [M+H]+ 605/607.

### 1-((8aS)-6-Chloro-5-(5-methyl-1H-indazol-4-yl)-8,8a,9,10,11,12-hexahydropyrazino-[2′,1′:3,4][1,4]oxazepino[5,6,7-de]quinazolin-2-yl)-N,N-dimethylazetidin-3-amine

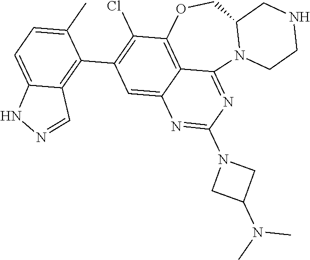 [SMILES: Cc1ccc2[nH]ncc2c1-c1cc2nc(N3CC(N(C)C)C3)nc3c2c(c1Cl)OC[C@@H]1CNCCN31]

To a solution of tert-butyl (8aS)-6-chloro-2-(3-(dimethylamino)azetidin-1-yl)-5-(5-methyl-1H-indazol-4-yl)-8a,9,11,12-tetrahydropyrazino[2′,1′:3,4][1,4]oxazepino[5,6,7-de]quinazoline-10 (8H)-carboxylate (202 mg, 0.33 mmol) in DCM (6 ml) at 0° C. under nitrogen was added TFA (1.5 ml, 19.59 mmol) and the reaction mixture stirred for 1.5 hours then the solvents were evaporated. The residue was dissolved in methanol and applied to a 20 g SCX column washing thoroughly with methanol, then the product was eluted using 1M ammonia in methanol. The solvent was evaporated to afford 1-((8aS)-6-chloro-5-(5-methyl-1H-indazol-4-yl)-8,8a,9,10,11,12-hexahydropyrazino[2′,1′:3,4]-[1,4]oxazepino[5,6,7-de]quinazolin-2-yl)-N,N-dimethylazetidin-3-amine (162 mg, 96%) as a pale brown solid. 1H NMR (400 MHz, DMSO) 2.09 (s, 6H), 2.13 (s, 3H), 2.59-2.78 (m, 2H), 2.96 (dd, J=10.9, 22.8 Hz, 3H), 3.09 (ddd, J=5.3, 6.8, 12.2 Hz, 1H), 3.80 (dd, J=5.2, 8.5 Hz, 3H), 4.03 (t, J=7.9 Hz, 2H), 4.33-4.45 (m, 1H), 4.45-4.58 (m, 1H), 4.74 (d, J=12.9 Hz, 1H), 6.86 (s, 1H), 7.28 (d, J=8.7 Hz, 1H), 7.36-7.68 (m, 2H), 13.04 (s, 1H). m/z: ES+ [M+H]+ 505/507.

### Example 75. 1-((8aS)-6-chloro-2-(3-(dimethylamino)azetidin-1-yl)-5-(5-methyl-1H-indazol-4-yl)-8a,9,11,12-tetrahydropyrazino[2′,1′:3,4][1,4]oxazepino[5,6,7-de]quinazolin-10 (8H)-yl)prop-2-en-1-one

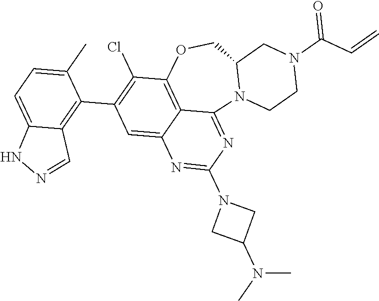 [SMILES: C=CC(=O)N1CCN2c3nc(N4CC(N(C)C)C4)nc4cc(-c5c(C)ccc6[nH]ncc56)c(Cl)c(c34)OC[C@@H]2C1]

To a solution of 1-((8aS)-6-chloro-5-(5-methyl-1H-indazol-4-yl)-8,8a,9,10,11,12-hexahydropyrazino[2′,1′:3,4][1,4]oxazepino[5,6,7-de]quinazolin-2-yl)-N,N-dimethylazetidin-3-amine (158 mg, 0.31 mmol) in DMF (10 ml) at −15° C. was added DIPEA (0.11 ml, 0.63 mmol) followed by a solution of acryloyl chloride (31 mg, 0.34 mmol) in DMF (4 ml)(added slowly dropwise) and the reaction mixture stirred at −15° C. for 15 minutes. The reaction mixture was diluted with ethyl acetate (100 ml) and washed with aqueous sodium bicarbonate solution (50 ml) and water (2×50 ml) and brine, the organic layer was dried over MgSO₄ and the solvent evaporated. The crude product was purified by preparative HPLC (Waters XSelect CSH C18 column, 5μ silica, 30 mm diameter, 100 mm length), using decreasingly polar mixtures of water (containing 1% NH₃) and MeCN as eluents. Fractions containing the desired compound were evaporated to dryness to afford 1-((8aS)-6-chloro-2-(3-(dimethylamino)azetidin-1-yl)-5-(5-methyl-1H-indazol-4-yl)-8a,9,11,12-tetrahydropyrazino[2′,1′:3,4][1,4]oxazepino[5,6,7-de]quinazolin-10 (8H)-yl)prop-2-en-1-one (49 mg, 28%) as a very pale yellow solid. 1H NMR (400 MHz, DMSO) 2.12 (s, 6H), 2.15 (d, J=1.5 Hz, 3H), 2.97-3.24 (m, 3H), 3.41 (s, 1H), 3.85 (dt, J=4.5, 8.8 Hz, 2H), 3.92-4.03 (m, 1H), 4.02-4.11 (m, 2H), 4.11-4.81 (m, 5H), 5.75 (dd, J=2.3, 10.5 Hz, 1H), 6.19 (dd, J=2.2, 16.7 Hz, 1H), 6.72-7.04 (m, 2H), 7.31 (d, J=8.5 Hz, 1H), 7.41-7.61 (m, 2H), 13.06 (s, 1H). m/z: ES+ [M+H]+ 559/561.

### Example 76, 1-((8aS)-6-Chloro-2-(3-(dimethylamino)azetidin-1-yl)-5-(5-methyl-1H-indazol-4-yl)-8a,9,11,12-tetrahydropyrazino[2′,1′:3,4][1,4]oxazepino[5,6,7-de]quinazolin-10 (8H)-yl)prop-2-en-1-one, Atropisomer 1; and

Example 77, 1-((8aS)-6-chloro-2-(3-(dimethylamino)azetidin-1-yl)-5-(5-methyl-1H-indazol-4-yl)-8a,9,11,12-tetrahydropyrazino[2′,1′:3,4][1,4]oxazepino[5,6,7-de]quinazolin-10 (8H)-yl)prop-2-en-1-one, Atropisomer 2

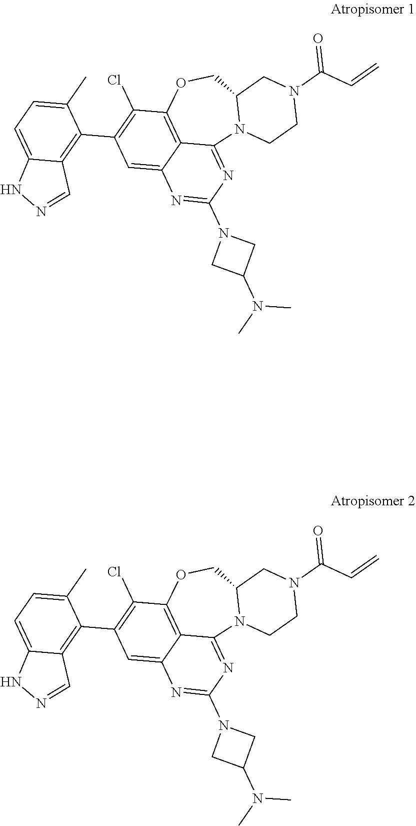 [SMILES: C=CC(=O)N1CCN2c3nc(N4CC(N(C)C)C4)nc4cc(-c5c(C)ccc6[nH]ncc56)c(Cl)c(c34)OC[C@@H]2C1 ; C=CC(=O)N1CCN2c3nc(N4CC(N(C)C)C4)nc4cc(-c5c(C)ccc6[nH]ncc56)c(Cl)c(c34)OC[C@@H]2C1]

The sample (Example 75) was dissolved in MecOH and separated using the SFC conditions detailed below: Column: Phenomonex C1, 30×250 mm, 5 micron Mobile phase: 40% MecOH (0.1% NH3)/60% scCO2 Flow rate: 80 ml/min BPR: 120 bar Column temp: 40° C. This afforded 1-((8aS)-6-chloro-2-(3-(dimethylamino)azetidin-1-yl)-5-(5-methyl-1H-indazol-4-yl)-8a,9,11,12-tetrahydropyrazino[2′,1′:3,4][1,4]oxazepino[5,6,7-de]quinazolin-10 (8H)-yl)prop-2-en-1-one (Atropisomer 1, 16 mg, >99% d.e.). 1H NMR (400 MHz, DMSO) 2.14 (d, J=11.0 Hz, 9H), 2.91-3.17 (m, 3H), 3.42 (s, 1H), 3.85 (dd, J=4.7, 8.8 Hz, 2H), 3.92-4.03 (m, 1H), 4.03-4.12 (m, 2H), 4.11-4.82 (m, 5H), 5.55-5.96 (m, 1H), 6.19 (dd, J=2.2, 16.7 Hz, 1H), 6.76-7.04 (m, 2H), 7.31 (d, J=8.6 Hz, 1H), 7.41-7.61 (m, 2H), 13.06 (s, 1H). m/z: ES+ [M+H]+ 559/561. This was followed by 1-((8aS)-6-chloro-2-(3-(dimethylamino)azetidin-1-yl)-5-(5-methyl-1H-indazol-4-yl)-8a,9,11,12-tetrahydropyrazino[2′,1′:3,4][1,4]oxazepino[5,6,7-de]quinazolin-10 (8H)-yl)prop-2-en-1-one (Atropisomer 2, 14.9 mg, >99% d.e.). 1H NMR (400 MHz, DMSO) 2.14 (d, J=6.1 Hz, 9H), 2.84-3.27 (m, 3H), 3.39 (d, J=32.9 Hz, 1H), 3.86 (d, J=4.2 Hz, 2H), 3.92-4.04 (m, 1H), 3.98-4.12 (m, 2H), 4.08-5.06 (m, 5H), 5.61-5.89 (m, 1H), 6.19 (dd, J=2.2, 16.7 Hz, 1H), 6.77-7.03 (m, 2H), 7.31 (d, J=8.5 Hz, 1H), 7.36-7.75 (m, 2H), 13.06 (s, 1H). m/z: ES+ [M+H]+ 559/561.

### Tert-butyl (S)-3-(((7-bromo-6-chloro-2-((2-(dimethylamino)ethyl)amino)-4-oxo-3,4-dihydroquinazolin-5-yl)oxy)methyl)piperazine-1-carboxylate

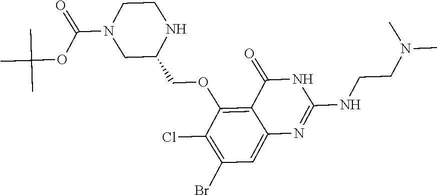 [SMILES: CN(C)CCNc1nc2cc(Br)c(Cl)c(OC[C@@H]3CN(C(=O)OC(C)(C)C)CCN3)c2c(=O)[nH]1]

To a suspension of tert-butyl (S)-3-(((7-bromo-2,6-dichloro-4-oxo-3,4-dihydroquinazolin-5-yl)oxy)methyl)piperazine-1-carboxylate (500 mg, 0.98 mmol) in i-PrOH (20 ml) was added DIPEA (0.86 ml, 4.92 mmol) and N1,N1-dimethylethane-1,2-diamine (260 mg, 2.95 mmol) and the reaction mixture stirred at 95° C. for 15.5 hours and allowed to cool. The reaction mixture was evaporated to dryness. The residue was purified by flash silica chromatography, elution 20% 2N methanolic ammonia in DCM. Pure fractions were evaporated to dryness to afford tert-butyl (S)-3-(((7-bromo-6-chloro-2-((2-(dimethylamino)ethyl)amino)-4-oxo-3,4-dihydroquinazolin-5-yl)oxy)methyl)-piperazine-1-carboxylate (415 mg, 75%) as a pale yellow solid. m/z: ES+ [M+H]+ 559/561.

### Tert-butyl (S)-10-bromo-11-chloro-7-((2-(dimethylamino)ethyl)amino)-3,4,13,13a-tetrahydropyrazino[2′,1′:3,4][1,4]oxazepino[5,6,7-de]quinazoline-2 (1H)-carboxylate

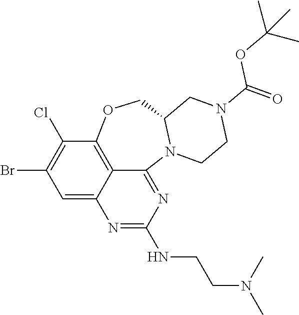 [SMILES: CN(C)CCNc1nc2c3c(c(Cl)c(Br)cc3n1)OC[C@@H]1CN(C(=O)OC(C)(C)C)CCN21]

To a stirred solution of tert-butyl (S)-3-(((7-bromo-6-chloro-2-((2-(dimethylamino)ethyl)-amino)-4-oxo-3,4-dihydroquinazolin-5-yl)oxy)methyl)piperazine-1-carboxylate (411 mg, 0.73 mmol) and ((1H-benzo[d][1,2,3]triazol-1-yl)oxy)tri(pyrrolidin-1-yl)phosphonium hexafluorophosphate(V) (574 mg, 1.1 mmol) in acetonitrile (16 ml) at 0° C. under nitrogen was added 2,3,4,6,7,8,9,10-octahydropyrimido[1,2-a]azepine (0.33 ml, 2.21 mmol) dropwise. The solution was stirred at 0° C. for 10 minutes then at room temperature for 2.5 hours. The reaction mixture was evaporated and the residue was dissolved in ethyl acetate (100 ml) and washed with aqueous saturated sodium bicarbonate solution (50 ml), water (50 ml) and brine (50 ml) then dried (MgSO₄) and the solvent evaporated. The residue was purified by flash silica chromatography, elution gradient 0 to 10% 2N methanolic ammonia in DCM. Pure fractions were evaporated to dryness to afford tert-butyl (S)-10-bromo-11-chloro-7-((2-(dimethylamino)ethyl)amino)-3,4,13,13a-tetrahydropyrazino[2′,1′:3,4][1,4]-oxazepino[5,6,7-de]quinazoline-2 (1H)-carboxylate (350 mg, 88%%) as a pale yellow solid. 1H NMR (400 MHz, DMSO) 1.44 (s, 9H), 2.35 (s, 6H), 2.55-2.74 (m, 2H), 2.91-3.2 (m, 3H), 3.44 (q, J=6.4 Hz, 2H), 3.89 (d, J=12.1 Hz, 2H), 3.96-4.12 (m, 1H), 4.51 (qd, J=4.2, 13.2 Hz, 2H), 4.62 (s, 1H), 6.84 (s, 1H), 7.33 (s, 1H). m/z: ES+ [M+H]+ 541/543.

### Tert-butyl (8aS)-6-chloro-2-((2-(dimethylamino)ethyl)amino)-5-(5-methyl-1H-indazol-4-yl)-8a,9,11,12-tetrahydropyrazino[2′,1′:3,4][1,4]oxazepino[5,6,7-de]quinazoline-10 (8H)-carboxylate

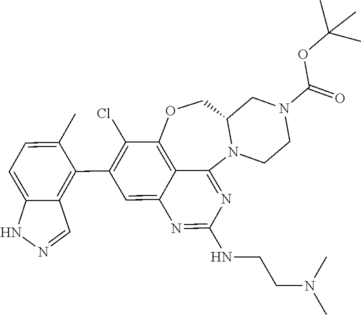 [SMILES: Cc1ccc2[nH]ncc2c1-c1cc2nc(NCCN(C)C)nc3c2c(c1Cl)OC[C@@H]1CN(C(=O)OC(C)(C)C)CCN31]

Pd-118 (45.5 mg, 0.07 mmol) was added to a degassed mixture of tert-butyl (S)-5-bromo-6-chloro-2-((2-(dimethylamino)ethyl)amino)-8a,9,11,12-tetrahydropyrazino[2′,1′:3,4]-[1,4]oxazepino[5,6,7-de]quinazoline-10 (8H)-carboxylate (350 mg, 0.65 mmol), (5-methyl-1H-indazol-4-yl)boronic acid (227 mg, 1.29 mmol) and 2N sodium carbonate (1.78 ml, 3.55 mmol) in 1,4-dioxane (11 ml). The reaction mixture was heated at 100° C. for 18 hours then further added Pd-118 (20 mg) and boronic acid (80 mg) were added and stirred at 100° C. for a further 7.5 hours, then allowed to cool. The reaction mixture was diluted with ethyl acetate (100 ml) and the organic layer was washed with aqueous 2M sodium carbonate solution (2×50 ml), water (50 ml) and brine (50 ml) then dried over MgSO₄, filtered and concentrated. The residue was purified by flash silica chromatography, elution gradient 0 to 10% 2N methanolic ammonia in DCM. Pure fractions were evaporated to dryness to afford tert-butyl (8aS)-6-chloro-2-((2-(dimethylamino)ethyl)amino)-5-(5-methyl-1H-indazol-4-yl)-8a,9,11,12-tetrahydropyrazino [2′,1′:3,4][1,4]oxazepino[5,6,7-de]quinazoline-10 (8H)-carboxylate (209 mg, 55%) as a pale brown solid. 1H NMR (400 MHz, DMSO) 1.46 (s, 9H), 2.15 (d, J=3.0 Hz, 3H), 2.19 (s, 6H), 2.44 (t, J=6.8 Hz, 2H), 3.11 (s, 3H), 3.40 (q, J=6.3 Hz, 2H), 3.93 (d, J=11.5 Hz, 2H), 4.04 (d, J=12.2 Hz, 1H), 4.46-4.63 (m, 2H), 4.66 (s, 1H), 6.61 (s, 1H), 6.86 (s, 1H), 7.31 (d, J=8.6 Hz, 1H), 7.49 (d, J=7.8 Hz, 2H), 13.06 (s, 1H). m/z: ES+ [M+H]+ 593/595.

### N1-((8aS)-6-Chloro-5-(5-methyl-1H-indazol-4-yl)-8,8a,9,10,11,12-hexahydropyrazino-[2′,1′:3,4][1,4]oxazepino[5,6,7-de]quinazolin-2-yl)-N2,N2-dimethylethane-1,2-diamine

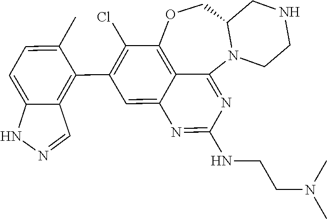 [SMILES: Cc1ccc2[nH]ncc2c1-c1cc2nc(NCCN(C)C)nc3c2c(c1Cl)OC[C@@H]1CNCCN31]

To a solution of tert-butyl (8aS)-6-chloro-2-((2-(dimethylamino)ethyl)amino)-5-(5-methyl-1H-indazol-4-yl)-8a,9,11,12-tetrahydropyrazino[2′,1′:3,4][1,4]oxazepino[5,6,7-de]-quinazoline-10 (8H)-carboxylate (205 mg, 0.35 mmol) in DCM (6 ml) at 0° C. under nitrogen was added TFA (1.5 ml, 19.59 mmol) and the reaction mixture stirred for 1.5 hours then the solvents evaporated. The residue was dissolved in methanol and applied to a 20 g SCX column washing thoroughly with methanol then the product was eluted using 1M ammonia in methanol. The solvent was evaporated to afford N1-((8aS)-6-chloro-5-(5-methyl-1H-indazol-4-yl)-8,8a,9,10,11,12-hexahydropyrazino[2′,1′:3,4][1,4]oxazepino-[5,6,7-de]quinazolin-2-yl)-N2,N2-dimethylethane-1,2-diamine (161 mg, 94%) as a very pale brown solid. 1H NMR (400 MHz, DMSO) 2.16 (d, J=7.9 Hz, 9H), 2.41 (t, J=6.8 Hz, 2H), 2.62-2.85 (m, 2H), 2.97 (dd, J=11.6, 21.4 Hz, 3H), 3.39 (q, J=7.0 Hz, 2H), 3.80 (s, 1H), 4.40 (ddd, J=2.2, 5.1, 13.1 Hz, 1H), 4.45-4.61 (m, 1H), 4.72 (s, 1H), 6.53 (s, 1H), 6.83 (s, 1H), 7.30 (d, J=8.6 Hz, 1H), 7.38-7.62 (m, 2H), 13.06 (s, 1H). m/z: ES+ [M+H]+ 493/495.

### Example 78, 1-((8aS)-6-Chloro-2-((2-(dimethylamino)ethyl)amino)-5-(5-methyl-1H-indazol-4-yl)-8a,9,11,12-tetrahydropyrazino[2′,1′:3,4][1,4]oxazepino[5,6,7-de]quinazolin-10 (8H)-yl)prop-2-en-1-one

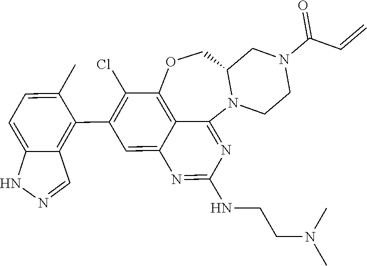 [SMILES: C=CC(=O)N1CCN2c3nc(NCCN(C)C)nc4cc(-c5c(C)ccc6[nH]ncc56)c(Cl)c(c34)OC[C@@H]2C1]

To a solution of N1-((8aS)-6-chloro-5-(5-methyl-1H-indazol-4-yl)-8,8a,9,10,11,12-hexahydropyrazino[2′,1′:3,4][1,4]oxazepino[5,6,7-de]quinazolin-2-yl)-N2,N2-dimethylethane-1,2-diamine (157 mg, 0.32 mmol) in DMF (14 ml) at −15° C. was added DIPEA (0.11 ml, 0.64 mmol) followed by a solution of acryloyl chloride (29 mg, 0.32 mmol) in DMF (4 ml)(added slowly dropwise) and the reaction mixture stirred at −15° C. for 15 minutes. The reaction mixture was diluted with ethyl acetate (100 ml) and washed with aqueous sodium bicarbonate solution (50 ml) and water (2×50 ml) and brine, the organic layer was dried over MgSO₄ and the solvent evaporated. The crude product (150 mg) was purified by preparative HPLC (Waters XSelect CSH C18 column, 5μ silica, 30 mm diameter, 100 mm length), using decreasingly polar mixtures of water (containing 1% NH₃) and MeCN as eluents. Fractions containing the desired compound were evaporated to dryness to afford 1-((8aS)-6-chloro-2-((2-(dimethylamino)ethyl)amino)-5-(5-methyl-1H-indazol-4-yl)-8a,9,11,12-tetrahydropyrazino [2′,1′:3,4][1,4]oxazepino[5,6,7-de]quinazolin-10 (8H)-yl)prop-2-en-1-one (25 mg, 14%) as an off-white solid. 1H NMR (400 MHz, DMSO) 2.08-2.28 (m, 9H), 2.43 (q, J=6.8, 7.8 Hz, 2H), 2.91-3.27 (m, 2H), 3.40 (q, J=6.3 Hz, 3H), 3.94 (s, 1H), 4.06-4.86 (m, 5H), 5.76 (dd, J=2.3, 10.4 Hz, 1H), 6.19 (dd, J=2.3, 16.7 Hz, 1H), 6.62 (s, 1H), 6.8-7.03 (m, 2H), 7.31 (d, J=8.7 Hz, 1H), 7.41-7.63 (m, 2H), 13.06 (s, 1H). m/z: ES+ [M+H]+ 547/549.

### Example 79, 1-((8aS)-6-Chloro-2-((2-(dimethylamino)ethyl)amino)-5-(5-methyl-1H-indazol-4-yl)-8a,9,11,12-tetrahydropyrazino[2′,1′:3,4][1,4]oxazepino[5,6,7-de]quinazolin-10 (8H)-yl)prop-2-en-1-one, Atropisomer 1; and

### Example 80, 1-((8aS)-6-chloro-2-((2-(dimethylamino)ethyl)amino)-5-(5-methyl-1H-indazol-4-yl)-8a,9,11,12-tetrahydropyrazino[2′,1′:3,4][1,4]oxazepino[5,6,7-de]quinazolin-10 (8H)-yl)prop-2-en-1-one, Atropisomer 2

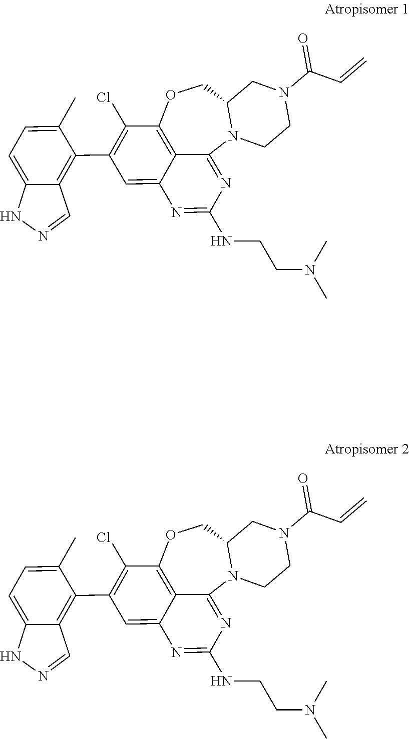 [SMILES: C=CC(=O)N1CCN2c3nc(NCCN(C)C)nc4cc(-c5c(C)ccc6[nH]ncc56)c(Cl)c(c34)OC[C@@H]2C1 ; C=CC(=O)N1CCN2c3nc(NCCN(C)C)nc4cc(-c5c(C)ccc6[nH]ncc56)c(Cl)c(c34)OC[C@@H]2C1]

The sample (Example 78) was dissolved in MeOH and separated using the SFC conditions detailed below: Column: Phenomonex C1 30×250 mm, 5 micron, Mobile phase: 40% MeOH+0.1% NH₃/60% scCO₂, Flow rate: 100 ml/min, BPR: 120 bar, Column temp: 40° C. This afforded 1-((8aS)-6-chloro-2-((2-(dimethylamino)ethyl)amino)-5-(5-methyl-1H-indazol-4-yl)-8a,9,11,12-tetrahydropyrazino[2′,1′:3,4][1,4]oxazepino[5,6,7-de]-quinazolin-10 (8H)-yl)prop-2-en-1-one (Atropisomer 1, 8 mg, >99% d.e.). m/z: ES+ [M+H]+ 547/549. This was followed by 1-((8aS)-6-chloro-2-((2-(dimethylamino)ethyl)-amino)-5-(5-methyl-1H-indazol-4-yl)-8a,9,11,12-tetrahydropyrazino[2′,1′:3,4][1,4]-oxazepino[5,6,7-de]quinazolin-10 (8H)-yl)prop-2-en-1-one (Atropisomer 2, 5 mg, >99% d.e.). m/z: ES+ [M+H]+ 547/549.

### Tert-butyl (R)-3-(2-((7-bromo-6-chloro-4-oxo-3,4-dihydroquinazolin-5-yl)oxy)ethyl)-piperazine-1-carboxylate

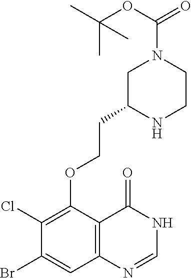 [SMILES: CC(C)(C)OC(=O)N1CCN[C@H](CCOc2c(Cl)c(Br)cc3nc[nH]c(=O)c23)C1]

Lithium tert-butoxide (263 mg, 3.29 mmol) was added to a stirred mixture of 7-bromo-6-chloro-5-fluoroquinazolin-4 (3H)-one (204 mg, 0.74 mmol) and tert-butyl (R)-3-(2-hydroxyethyl)piperazine-1-carboxylate (233 mg, 1.01 mmol) in DMA (4 ml). The mixture was stirred at 80° C. for 3 hours. After cooling to room temperature, the mixture was quenched with methanol and purified by preparative HPLC (Waters XSelect CSH C18 column, 5μ silica, 30 mm diameter, 100 mm length), using decreasingly polar mixtures of water (containing 0.3% NH₃) and MeCN as eluents. Fractions containing the desired compound were evaporated to dryness to afford tert-butyl (R)-3-(2-((7-bromo-6-chloro-4-oxo-3,4-dihydroquinazolin-5-yl)oxy)ethyl)piperazine-1-carboxylate (247 mg, 69%). 1H NMR (400 MHz, DMSO) 1.40 (s, 9H), 1.72-1.91 (m, 2H), 2.56 (dd, J=3.1, 11.4 Hz, 2H), 2.7-2.82 (m, 2H), 2.86 (d, J=11.9 Hz, 1H), 3.73 (d, J=12.5 Hz, 1H), 3.86 (d, J=11.6 Hz, 1H), 4-4.17 (m, 2H), 7.83 (s, 1H), 8.09 (s, 1H). m/z: ES+ [M+H]+ 487/489.

### Tert-butyl (6aR)-2-bromo-3-chloro-5,6,6a,7,9,10-hexahydro-8H-pyrazino[1′,2′:5,6]-[1,5]oxazocino[4,3,2-de]quinazoline-8-carboxylate

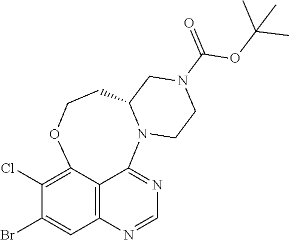 [SMILES: CC(C)(C)OC(=O)N1CCN2c3ncnc4cc(Br)c(Cl)c(c34)OCC[C@@H]2C1]

2,3,4,6,7,8,9,10-Octahydropyrimido[1,2-a]azepine (0.3 ml, 2.01 mmol) was added dropwise to tert-butyl (R)-3-(2-((7-bromo-6-chloro-4-oxo-3,4-dihydroquinazolin-5-yl)oxy)ethyl)piperazine-1-carboxylate (165 mg, 0.34 mmol) and ((1H-benzo[d][1,2,3]-triazol-1-yl)oxy)tri(pyrrolidin-1-yl)phosphonium hexafluorophosphate(V) (352 mg, 0.68 mmol) in THF (5 ml). The resulting solution was stirred at room temperature for 3 hours and diluted with ethyl acetate (50 ml). The mixture was washed with 1M citric acid (15 ml), twice with water and concentrated in vacuo. The crude product was purified by flash silica chromatography, elution gradient 0 to 100% EtOAc in heptane. Pure fractions were evaporated to dryness to afford tert-butyl (6aR)-2-bromo-3-chloro-5,6,6a,7,9,10-hexahydro-8H-pyrazino[1′,2′:5,6][1,5]oxazocino[4,3,2-de]quinazoline-8-carboxylate (158 mg, 100%) as a white solid. m/z: ES+ [M+H]+ 469/471.

### (6aR)-3-chloro-2-(5-methyl-1H-indazol-4-yl)-6,6a,7,8,9,10-hexahydro-5H-pyrazino-[1′,2′:5,6][1,5]oxazocino[4,3,2-de]quinazoline

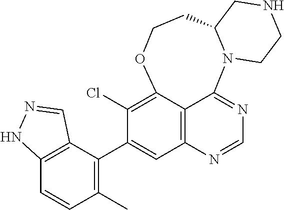 [SMILES: Cc1ccc2[nH]ncc2c1-c1cc2ncnc3c2c(c1Cl)OCC[C@@H]1CNCCN31]

Pd-118 (30 mg, 0.05 mmol was added to a degassed mixture of tert-butyl (6aR)-2-bromo-3-chloro-5,6,6a,7,9,10-hexahydro-8H-pyrazino[1,2′:5,6][1,5]oxazocino[4,3,2-de]quinazoline-8-carboxylate (225 mg, 0.48 mmol), (5-methyl-1H-indazol-4-yl)boronic acid (136 mg, 0.77 mmol), acetonitrile (4 ml) and 2M aq. K₂CO₃. The reaction mixture was heated at 100° C. for 1 hour in a microwave reactor and cooled to room temperature. The mixture was extracted with EtOAc and the organic phase was concentrated in vacuo. The residue was re-dissolved in MeOH (2 ml) and methanesulfonic acid (0.1 ml, 1.54 mmol) was added. The mixture was stirred at reflux for 60 min. The crude product was purified by ion exchange chromatography, using an SCX column. The desired product was eluted from the column using 1M NH₃/MeOH and pure fractions were evaporated to dryness to afford the crude amine as a mixture of 2 diasteroisomers. The crude product was purified by preparative HPLC (Waters XSelect CSH C18 column, 30×100 mm id, 5 micron particle size), using decreasingly polar mixtures of water (containing 0.3% NH₃) and MeCN as eluents. Fractions containing the desired compound were evaporated to dryness to afford (6aR)-3-chloro-2-(5-methyl-1H-indazol-4-yl)-6,6a,7,8,9,10-hexahydro-5H-pyrazino[1′,2′:5,6][1,5]oxazocino-[4,3,2-de]quinazoline (80 mg, 40%). m/z: ES+ [M+H]+ 421.

### Example 81, 1-[(6aR)-3-chloro-2-(5-methyl-1H-indazol-4-yl)-5,6,6a,7,9,10-hexahydro-8H-pyrazino[1′,2′:5,6][1,5]oxazocino[4,3,2-de]quinazolin-8-yl]prop-2-en-1-one, Atropisomer 1; and

### Example 82, 1-[(6aR)-3-chloro-2-(5-methyl-1H-indazol-4-yl)-5,6,6a,7,9,10-hexahydro-8H-pyrazino[1′,2′:5,6][1,5]oxazocino[4,3,2-de]quinazolin-8-yl]prop-2-en-1-one, Atropisomer 2

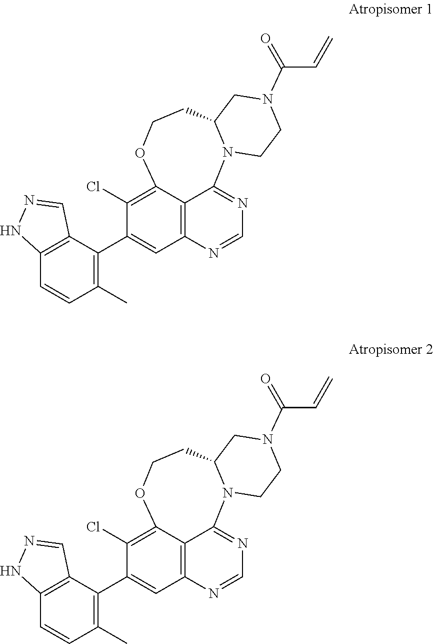 [SMILES: C=CC(=O)N1CCN2c3ncnc4cc(-c5c(C)ccc6[nH]ncc56)c(Cl)c(c34)OCC[C@@H]2C1 ; C=CC(=O)N1CCN2c3ncnc4cc(-c5c(C)ccc6[nH]ncc56)c(Cl)c(c34)OCC[C@@H]2C1]

A solution (0.3 ml) of acrylolyl chloride (0.05 ml) in DCM (1 ml) was added dropwise to a stirred solution of (6aR)-3-chloro-2-(5-methyl-1H-indazol-4-yl)-6,6a,7,8,9,10-hexahydro-5H-pyrazino[1′,2′:5,6][1,5]oxazocino-[4,3,2-de]quinazoline (80 mg, 0.19 mmol) and DIPEA (0.1 ml, 0.57 mmol) in i-PrOH (0.5 ml) and DCM (1.5 ml). The mixture was stirred for 10 min. The crude product was purified by preparative HPLC (Waters XSelect CSH C18 column, 5μ silica, 30 mm diameter, 100 mm length), using decreasingly polar mixtures of water (containing 0.3% NH₃) and MeCN as eluents. Fractions containing the desired compound were evaporated to dryness to 1-[(6aR)-3-chloro-2-(5-methyl-1H-indazol-4-yl)-5,6,6a,7,9,10-hexahydro-8H-pyrazino[1′,2′:5,6][1,5]oxazocino[4,3,2-de]quinazolin-8-yl]prop-2-en-1-one (Atropisomer 1, 29 mg, 29%) as a white solid. 1H NMR (400 MHz, DMSO) 1.96-2.06 (m, 1H), 2.16 (t, J=4.7 Hz, 1H), 2.19 (s, 3H), 2.2-2.31 (m, 1H), 3.69-3.87 (m, 5H), 3.91-4.01 (m, 2H), 4.36-4.71 (m, 4H), 5.70 (dd, J=2.3, 10.5 Hz, 1H), 6.13 (dd, J=2.3, 16.8 Hz, 1H), 6.69-6.79 (m, 1H), 7.32 (d, J=8.5 Hz, 1H), 7.40 (s, 1H), 7.50 (d, J=1.0 Hz, 1H), 7.53 (d, J=8.5 Hz, 1H), 8.49 (s, 1H). m/z: ES+ [M+H]+ 475. This was followed by 1-[(6aR)-3-chloro-2-(5-methyl-1H-indazol-4-yl)-5,6,6a,7,9,10-hexahydro-8H-pyrazino-[1′,2′:5,6][1,5]oxazocino[4,3,2-de]quinazolin-8-yl]prop-2-en-1-one (Atropisomer 2, 17 mg, 17%) as a white solid. 1H NMR (400 MHz, DMSO) 1.86-2.09 (m, 2H), 2.20 (s, 3H), 2.22-2.31 (m, 1H), 3.44-3.55 (m, 1H), 3.58-3.83 (m, 3H), 3.91 (dd, J=4.4, 13.8 Hz, 1H), 3.95-4.1 (m, 2H), 4.36-4.78 (m, 4H), 5.68-5.79 (m, 1H), 6.18 (dd, J=1.8, 16.7 Hz, 1H), 6.81 (dd, J=10.8, 16.0 Hz, 1H), 7.3-7.39 (m, 2H), 7.47-7.61 (m, 2H), 8.46 (s, 1H). m/z: ES+ [M+H]+ 475.

1. A compound of Formula (I): 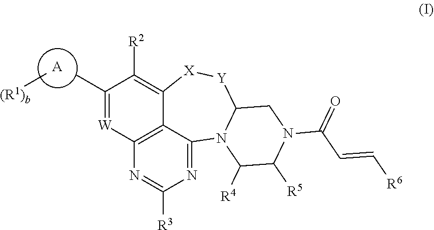
wherein:
Ring A is selected from aryl, monocyclic heteroaryl and bicyclic heteroaryl;
R¹ is independently selected from C₁₋₄alkyl, halo, hydroxy, C₁₋₄alkoxy, C₁₋₃fluoroalkyl, C₁₋₃fluoroalkoxy, cyano, acetylenyl, NR⁷R⁸, C(O)NR⁹R¹⁰, CH₂R¹¹, N═S(O)Me₂, S(O)Me and SO₂R¹²;
b is 0, 1, 2 or 3;
W is N or CR¹³;
X is O or NR¹⁴;
Y is CR¹⁵R¹⁶, CR¹⁷R¹⁸CR¹⁹R²⁰, C═O, or C(O)CR²¹R²²;
R² is H, cyano, halo, C₁₋₄alkyl, C₁₋₄alkoxy, C₁₋₃fluoroalkyl, NR²³R²⁴, acetylenyl or CH₂OR²⁵;
R³ is H, C₁₋₃fluoroalkyl, OR²⁶, NR²⁷R²⁸, CH₂R²⁹, SR³⁰ or C(O)R³¹;
R⁴ is H or Me;
R⁵ is H or Me;
R⁶ is H or CH₂NMe₂;
R⁷ is H, C₁₋₄alkyl, C(O)C₁₋₃alkyl or CO₂C₁₋₃alkyl;
R¹¹ is hydroxy, cyano, heterocyclyl, NR³²R³³, C(O)NR³⁴R³⁵ or SO₂C₁₋₃alkyl;
R¹² is C₁₋₃alkyl, C₁₋₃fluoroalkyl or NR³⁶R³⁷;
R¹³ is H, C₁₋₄alkyl, halo, C₁₋₃fluoroalkyl or C₁₋₄alkoxy;
R¹⁵, R¹⁶, R¹⁷ and R¹⁸ are independently selected from H and C₁₋₃alkyl;
R¹⁹, R²⁰, R²¹ and R²² are independently selected from H, C₁₋₃alkyl, and fluoro;
R²⁶ is selected from the group consisting of:
H;
C₁₋₄alkyl optionally substituted with 1 or 2 substituents selected from hydroxy, C₁₋₃ alkoxy, halo, NR³⁸R³⁹, C(O)NR⁴⁰R⁴¹, SO₂Me, heteroaryl, C₃₋₇cycloalkyl or heterocyclyl, wherein said heteroaryl or C₃₋₇cycloalkyl is optionally further substituted with C₁₋₄alkyl, hydroxy, halo, cyano, or C₁₋₄alkoxy and said heterocyclyl is optionally further substituted with C₁₋₄alkyl, hydroxy, halo, C(O)Me, C₁₋₃alkoxy, C₁₋₃fluoroalkyl, C₃₋₇cycloalkyl, heterocyclyl or heteroaryl;
C₃₋₇cycloalkyl optionally substituted with C₁₋₄alkyl, hydroxy or halo;
heterocyclyl optionally substituted with C₁₋₄alkyl, hydroxy, halo, C(O)Me, C₁₋₃ alkoxy, C₁₋₃fluoroalkyl, C₃₋₇cycloalkyl, heterocyclyl or heteroaryl; and
heteroaryl optionally substituted with C₁₋₄alkyl, hydroxy, halo, cyano or C₁₋₄alkoxy;
R²⁷ is selected from the group consisting of:
H;
C(O)R⁴²;
C₁₋₄alkyl optionally substituted with 1 or 2 substituents selected from hydroxy, C₁₋₃ alkoxy, halo, NR⁴³R⁴⁴, C(O)NR⁴⁵R⁴⁶, SO₂Me, heteroaryl, C₃₋₇cycloalkyl or heterocyclyl, wherein said heteroaryl or C₃₋₇cycloalkyl is optionally further substituted with C₁₋₄alkyl, hydroxy, halo, cyano, or C₁₋₄alkoxy and said heterocyclyl is optionally further substituted with C₁₋₄alkyl, hydroxy, halo, C(O)Me, C₁₋₃alkoxy, C₁₋₃fluoroalkyl, C₃₋₇cycloalkyl, heterocyclyl or heteroaryl;
C₃₋₇cycloalkyl optionally substituted with C₁₋₄alkyl, hydroxy or halo;
heterocyclyl optionally substituted with C₁₋₄alkyl, hydroxy, halo, C(O)Me, C₁₋₃ alkoxy, C₁₋₃fluoroalkyl, C₃₋₇cycloalkyl, CH₂cyclopropyl, heterocyclyl or heteroaryl; and
heteroaryl optionally substituted with C₁₋₄alkyl, hydroxy, halo, cyano or C₁₋₄alkoxy;
R²⁸ is H or Me; or
R²⁷ and R²⁸ taken together with the nitrogen atom to which they are attached form a 4-, 5-, 6- or 7-membered heterocyclic ring, wherein said ring is optionally substituted with C₁₋₄alkyl, hydroxy, halo, C(O)Me, NR⁴⁷R⁴⁸, C₁₋₃ alkoxy, C₁₋₃fluoroalkyl, C₃₋₇cycloalkyl, CH₂cyclopropyl, heterocyclyl or heteroaryl;
R²⁹ is selected from the group consisting of:
H;
NR⁴⁹R⁵⁰;
C₁₋₃alkyl optionally substituted with 1 or 2 substituents selected from hydroxy, C₁₋₃ alkoxy, halo, NR⁵¹R⁵², C(O)NR⁵³R⁵⁴, SO₂Me, heteroaryl, C₃₋₇cycloalkyl or heterocyclyl, wherein said heteroaryl or C₃₋₇cycloalkyl is optionally further substituted with C₁₋₄alkyl, hydroxy, halo, cyano, or C₁₋₄alkoxy and said heterocyclyl is optionally further substituted with C₁₋₄alkyl, hydroxy, halo, C(O)Me, C₁₋₃alkoxy, C₁₋₃fluoroalkyl, C₃₋₇cycloalkyl, heterocyclyl or heteroaryl;
C₃₋₇cycloalkyl optionally substituted with C₁₋₄alkyl, hydroxy or halo;
heterocyclyl optionally substituted with C₁₋₄alkyl, hydroxy, halo, C(O)Me, C₁₋₃ alkoxy, C₁₋₃fluoroalkyl, C₃₋₇cycloalkyl, CH₂cyclopropyl, heterocyclyl or heteroaryl; and
heteroaryl optionally substituted with C₁₋₄alkyl, hydroxy, halo, cyano or C₁₋₄alkoxy;
R³⁰ is selected from the group consisting of:
C₁₋₄alkyl optionally substituted with 1 or 2 substituents selected from hydroxy, C₁₋₃ alkoxy, halo, NR⁵⁵R⁵⁶, C(O)NR⁵⁷R⁵⁸, SO₂Me, heteroaryl, C₃₋₇cycloalkyl or heterocyclyl, wherein said heteroaryl or C₃₋₇cycloalkyl is optionally further substituted with C₁₋₄alkyl, hydroxy, halo, cyano, or C₁₋₄alkoxy and said heterocyclyl is optionally further substituted with C₁₋₄alkyl, hydroxy, halo, C(O)Me, C₁₋₃alkoxy, C₁₋₃fluoroalkyl, C₃₋₇cycloalkyl, heterocyclyl or heteroaryl;
C₃₋₇cycloalkyl optionally substituted with C₁₋₄alkyl, hydroxy or halo;
heterocyclyl optionally substituted with C₁₋₄alkyl, hydroxy, halo, C(O)Me, C₁₋₃ alkoxy, C₁₋₃fluoroalkyl, C₃₋₇cycloalkyl, heterocyclyl or heteroaryl; and
heteroaryl optionally substituted with C₁₋₄alkyl, hydroxy, halo, cyano or C₁₋₄alkoxy;
R³¹ is NR⁵⁹R⁶⁰;
R⁴² is optionally substituted heteroaryl or optionally substituted C₁₋₄alkyl;
R⁴⁹ and R⁵¹ are independently selected from H, C₁₋₄alkyl, heterocyclyl and heteroaryl;
R⁵⁹ and R⁶⁰ are independently selected from H and C₁₋₄alkyl; or
R⁵⁹ and R⁶⁰ taken together with the nitrogen atom to which they are attached form a 4-, 5- or 6-membered heterocyclic ring, wherein said ring is optionally substituted with C₁₋₄alkyl, hydroxy, halo or C(O)Me;
R⁸, R⁹, R¹⁰, R¹⁴, R²³, R²⁴, R²⁵, R³², R³³, R³⁴, R³⁵, R³⁶, R³⁷, R³⁸, R³⁹, R⁴⁰, R⁴¹, R⁴³, R⁴⁴, R⁴⁵, R⁴⁶, R⁴⁷, R⁴⁸, R⁵⁰, R⁵², R⁵³, R⁵⁴, R⁵⁵, R⁵⁶, R⁵⁷ and R⁵⁸ are independently selected from H and C₁₋₄alkyl;
or a pharmaceutically acceptable salt thereof.

2. The compound or pharmaceutically acceptable salt of claim 1, wherein ring A is bicyclic heteroaryl selected from the group consisting of:

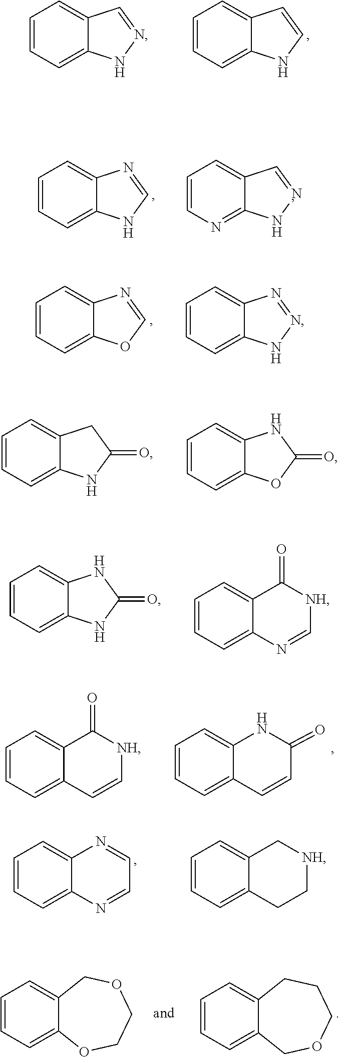

3. The compound or pharmaceutically acceptable salt of claim 1, wherein ring A is phenyl.

4. The compound or pharmaceutically acceptable salt of claim 1, wherein R⁶ is H.

5. The compound or pharmaceutically acceptable salt of claim 1, wherein X is O and Y is CH₂.

6. The compound or pharmaceutically acceptable salt of claim 1, wherein X is O and Y is CH₂CH₂.

7. The compound or pharmaceutically acceptable salt of claim 1, wherein R³ is H.

8. (canceled)

9. (canceled)

10. A method of treating a disorder mediated by KRAS, NRAS, or HRAS G12C mutations comprising administering a compound or pharmaceutically acceptable salt of claim 1 to a patient in need thereof.

11. The method of claim 10, wherein the disorder mediated by KRAS, NRAS, or HRAS G12C mutations is cancer.

12. The method of claim 11, wherein the cancer is non-small cell lung cancer or colorectal cancer.

13. A pharmaceutical composition comprising a compound or pharmaceutically acceptable salt of claim 1, and a pharmaceutically acceptable excipient.

14. (canceled)

15. (canceled)

16. The method of claim 11, further comprising administering another anti-tumour agent.
